SEQUENCE LISTING (175 sequences)

SEQ ID NO: 1 - protein, 220 aa, Homo sapiens
  human CD28
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 41 aa, Artificial Sequence
  synthetic sequence human CD28 intracellular costimulatory domain
RSKRSRLLHSDYMNMTPRRPGPTRKHYQPYAPPRDFAAYRS

SEQ ID NO: 3 - protein, 41 aa, Artificial Sequence
  synthetic sequence human CD28 intracellular costimulatory domain (L186G, L187G)]
RSKRSRGGHSDYMNMTPRRPGPTRKHYQPYAPPRDFAAYRS

SEQ ID NO: 4 - protein, 41 aa, Artificial Sequence
  synthetic sequence human CD28 intracellular costimulatory domain (Y218F)
RSKRSRGGHSDYMNMTPRRPGPTRKHYQPYAPPRDFAAFRS

SEQ ID NO: 5 - protein, 41 aa, Artificial Sequence
  synthetic sequence CD28 intracellular costimulatory domain (Y191F,Y206F, Y209F, Y218F)
RSKRSRGGHSDFMNMTPRRPGPTRKHFQPFAPPRDFAAFRS

SEQ ID NO: 6 - protein, 41 aa, Artificial Sequence
  synthetic sequence CD28 intracellular costimulatory domain (Y206F, Y209F, Y218F)
RSKRSRGGHSDYMNMTPRRPGPTRKHFQPFAPPRDFAAFRS

SEQ ID NO: 7 - protein, 22 aa, Artificial Sequence
  synthetic sequence human GM-CSF receptor signal peptide
MLLLVTSLLLCELPHPAFLLIP

SEQ ID NO: 8 - protein, 245 aa, Artificial Sequence
  Synthetic sequence FMC63 (anti-CD19) scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 9 - protein, 248 aa, Artificial Sequence
  Synthetic sequence R12 (anti-ROR1) scFv
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 10 - protein, 9 aa, Artificial Sequence
  Synthetic sequence Strep tag II peptide, 9-mer
NWSHPQFEK

SEQ ID NO: 11 - protein, 10 aa, Artificial Sequence
  Synthetic sequence (G4S)2 linker
GGGGSGGGGS

SEQ ID NO: 12 - protein, 12 aa, Artificial Sequence
  Synthetic sequence human IgG4 hinge
ESKYGPPCPPCP

SEQ ID NO: 13 - protein, 28 aa, Artificial Sequence
  Synthetic sequence human CD28 transmembrane domain
MFWVLVVVGGVLACYSLLVTVAFIIFWV

SEQ ID NO: 14 - protein, 42 aa, Artificial Sequence
  Synthetic sequence human 4-1BB costimulatory signaling domain
KRGRKKLLYIFKQPFMRPVQTTQEEDGCSCRFPEEEEGGCEL

SEQ ID NO: 15 - protein, 112 aa, Artificial Sequence
  Synthetic sequence human CD3zeta signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 24 aa, Artificial Sequence
  Synthetic sequence T2A viral peptide
LEGGGEGRGSLLTCGDVEENPGPR

SEQ ID NO: 17 - protein, 335 aa, Artificial Sequence
  Synthetic sequence truncated human EGFR (tEGFR) polypeptide, lacking signal peptide
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 18 - DNA, 2586 bp, Artificial Sequence
  Synthetic sequence A042 CAR (FMC63 scFv_STII_IgG4 hinge_CD28tm_41BB_CD3z_T2A_tEGR) expression cassette
atgctgctgctggtgaccagcctgctgctgtgcgagctgccccaccctgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcctagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatcaggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 19 - protein, 861 aa, Artificial Sequence
  Synthetic sequence A042 CAR cassette protein sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSNWSHPQFEKGGGGSGGGGSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 20 - DNA, 2583 bp, Artificial Sequence
  Synthetic sequence A043CAR (FMC63 scFv_STII_IgG4 hinge_CD28tm_CD28icd_CD3z_T2A_tEGFR) expression cassette
atgctgctgctggtgaccagcctgctgctgtgcgagctgccccaccctgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcctagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatcaggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtccgcagcaagcggagcagaggcggccacagcgactacatgaacatgacccctagacggcctggccccaccagaaagcactaccagccctacgcccctccccgggactttgccgcctacagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 21 - protein, 860 aa, Artificial Sequence
  Synthetic sequence A043 CAR cassette protein sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSNWSHPQFEKGGGGSGGGGSESKYGPPCPPCPMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 22 - DNA, 2595 bp, Artificial Sequence
  Synthetic sequence A044 CAR (R12 scFv_STII_IgG4 hinge_CD28tm_41BB_CD3z_T2A_tEGFR) expression cassette
atgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 23 - protein, 864 aa, Artificial Sequence
  Synthetic sequence A044 CAR cassette protein sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGNWSHPQFEKGGGGSGGGGSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 24 - DNA, 2592 bp, Artificial Sequence
  Synthetic sequence A045 CAR (R12 scFv_STII_IgG4 hinge_CD28tm_CD28icd_CD3z_T2A_tEGFR) expression cassette
atgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtccgcagcaagcggagcagaggcggccacagcgactacatgaacatgacccctagacggcctggccccaccagaaagcactaccagccctacgcccctccccgggactttgccgcctacagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 25 - protein, 863 aa, Artificial Sequence
  Synthetic sequence A045 CAR cassette protein sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGNWSHPQFEKGGGGSGGGGSESKYGPPCPPCPMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 26 - DNA, 2583 bp, Artificial Sequence
  Synthetic sequence A142 CAR (FMC63 scFv_STII_IgG4 hinge_CD28tm_CD28icd (Y218F)_CD3z_T2A_tEGFR) expression cassette
atgctgctgctggtgaccagcctgctgctgtgcgagctgccccaccctgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcctagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatcaggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtccgcagcaagcggagcagaggcggccacagcgactacatgaacatgacccctagacggcctggccccaccagaaagcactaccagccctacgcccctccccgggactttgccgccttcagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 27 - protein, 860 aa, Artificial Sequence
  Synthetic sequence A142 CAR cassette protein sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSNWSHPQFEKGGGGSGGGGSESKYGPPCPPCPMFWVLVVVGGVLACYSLLVTVAFIIFWVRSKRSRGGHSDYMNMTPRRPGPTRKHYQPYAPPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 28 - DNA, 2583 bp, Artificial Sequence
  Synthetic sequence A143 CAR (FMC63 scFv_STII_IgG4 hinge_CD28tm_CD28icd (Y206F, Y209F, Y218F)_CD3z_T2A_tEGFR) expression cassette
atgctgctgctggtgaccagcctgctgctgtgcgagctgccccaccctgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcctagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatcaggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtccgcagcaagcggagcagaggcggccacagcgactacatgaacatgacccctagacggcctggccccaccagaaagcacttccagcccttcgcccctccccgggactttgccgccttcagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 29 - protein, 860 aa, Artificial Sequence
  Synthetic sequence A143 CAR cassette protein sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSNWSHPQFEKGGGGSGGGGSESKYGPPCPPCPMFWVLVVVGGVLACYSLLVTVAFIIFWVRSKRSRGGHSDYMNMTPRRPGPTRKHFQPFAPPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 30 - DNA, 2592 bp, Artificial Sequence
  Synthetic sequence A144 CAR (R12 scFv_STII_IgG4 hinge_CD28tm_CD28icd (Y218F)_CD3z_T2A_tEGFR) expression cassette
atgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtccgcagcaagcggagcagaggcggccacagcgactacatgaacatgacccctagacggcctggccccaccagaaagcactaccagccctacgcccctccccgggactttgccgccttcagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 31 - protein, 863 aa, Artificial Sequence
  Synthetic sequence A144 CAR cassette protein sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGNWSHPQFEKGGGGSGGGGSESKYGPPCPPCPMFWVLVVVGGVLACYSLLVTVAFIIFWVRSKRSRGGHSDYMNMTPRRPGPTRKHYQPYAPPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 32 - DNA, 2592 bp, Artificial Sequence
  Synthetic sequence A145 (R12 scFv_STII_IgG4 hinge_CD28tm_CD28icd (Y206F, Y209F, Y218F)_CD3z_T2A_tEGFR) expression cassette
atgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtccgcagcaagcggagcagaggcggccacagcgactacatgaacatgacccctagacggcctggccccaccagaaagcacttccagcccttcgcccctccccgggactttgccgccttcagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 33 - protein, 863 aa, Artificial Sequence
  Synthetic sequence A145 CAR cassette protein sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGNWSHPQFEKGGGGSGGGGSESKYGPPCPPCPMFWVLVVVGGVLACYSLLVTVAFIIFWVRSKRSRGGHSDYMNMTPRRPGPTRKHFQPFAPPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 34 - DNA, 2583 bp, Artificial Sequence
  Synthetic sequence A175 CAR (FMC63 scFv_STII_IgG4 hinge_CD28tm_CD28icd (Y191F, Y206F, Y209F, Y218F)_CD3z_T2A_tEGFR) expression cassette
atgctgctgctggtgaccagcctgctgctgtgcgagctgccccaccctgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcctagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatcaggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtccgcagcaagcggagcagaggcggccacagcgacttcatgaacatgacccctagacggcctggccccaccagaaagcacttccagcccttcgcccctccccgggactttgccgccttcagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 35 - protein, 860 aa, Artificial Sequence
  Synthetic sequence A175 CAR cassette protein sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSNWSHPQFEKGGGGSGGGGSESKYGPPCPPCPMFWVLVVVGGVLACYSLLVTVAFIIFWVRSKRSRGGHSDFMNMTPRRPGPTRKHFQPFAPPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 36 - DNA, 2592 bp, Artificial Sequence
  Synthetic sequence A176 CAR (R12 scFv_STII_IgG4_CD28tm_CD28icd (Y191F, Y206F, Y209F, Y218F)_CD3z_T2A_tEGFR) expression cassette
atgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtccgcagcaagcggagcagaggcggccacagcgacttcatgaacatgacccctagacggcctggccccaccagaaagcacttccagcccttcgcccctccccgggactttgccgccttcagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 37 - protein, 863 aa, Artificial Sequence
  Synthetic sequence A176 CAR cassette protein sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGNWSHPQFEKGGGGSGGGGSESKYGPPCPPCPMFWVLVVVGGVLACYSLLVTVAFIIFWVRSKRSRGGHSDFMNMTPRRPGPTRKHFQPFAPPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 38 - protein, 41 aa, Artificial Sequence; identical to SEQ ID NO: 3
  Synthetic sequence human  CD28 intracellular costimulatory domain (L186G/L187G)

SEQ ID NO: 39 - protein, 8 aa, Artificial Sequence
  Synthetic sequence Strep-Tag peptide
WRHPQFGG

SEQ ID NO: 40 - protein, 8 aa, Artificial Sequence
  Synthetic sequence Strep-Tag II peptide, 8-mer
WSHPQFEK

SEQ ID NO: 41 - protein, 27 aa, Artificial Sequence
  Synthetic sequence wildtype human CD28 transmembrane domain
FWVLVVVGGVLACYSLLVTVAFIIFWV

SEQ ID NO: 42 - protein, 16 aa, Artificial Sequence
  Synthetic sequence ITAM motif
  VARIANT (2)...(3): Xaa = any amino acid
  VARIANT 4: Xaa = L or I
  VARIANT (5)...(15): Xaa = any amino acid
  VARIANT 16: Xaa = L or I
  VARIANT (10)...(12): any one or all of amino acids can 10-12 either be present or absent.
YXXXXXXXXXXXYXXX

SEQ ID NO: 43 - protein, 114 aa, Artificial Sequence
  Synthetic sequence Human CD8alpha IgV-like domain
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPA

SEQ ID NO: 44 - protein, 111 aa, Artificial Sequence
  Synthetic sequence Human CD8beta IgV-like domain
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQL

SEQ ID NO: 45 - protein, 41 aa, Artificial Sequence
  Synthetic sequence human CD28 intracellular costimulatory domain (P208A, P211A)
RSKRSRGGHSDYMNMTPRRPGPTRKHYQAYAAPRDFAAYRS

SEQ ID NO: 46 - protein, 41 aa, Artificial Sequence
  Synthetic sequence human CD28 intracellular costimulatory domain (P208A, P211A, Y218F)
RSKRSRGGHSDYMNMTPRRPGPTRKHYQAYAAPRDFAAFRS

SEQ ID NO: 47 - protein, 41 aa, Artificial Sequence
  Synthetic sequence CD28 intracellular costimulatory domain (Y206F, P208A, Y209F, P211A, Y218F)
RSKRSRGGHSDYMNMTPRRPGPTRKHFQAFAAPRDFAAFRS

SEQ ID NO: 48 - DNA, 2583 bp, Artificial Sequence
  Synthetic sequence A192 CAR (FMC63 scFv_STII_IgG4 hinge_CD28tm_CD28icd (P208A, P211A)_CD3z_T2A_tEGFR) expression cassette
atgctgctgctggtgaccagcctgctgctgtgcgagctgccccaccctgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcctagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatcaggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtccgcagcaagcggagcagaggcggccacagcgactacatgaacatgacccctagacggcctggccccaccagaaagcactaccaggcctacgccgctccccgggactttgccgcctacagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 49 - protein, 859 aa, Artificial Sequence
  Synthetic sequence A192 CAR cassette protein sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSNWSHPQFEKGGGGSGGGGSESKYGPPCPPCPMFWVLVVVGGVLACYSLLVTVAFIIFWVRSKRSRGGHSDYMNMTPRRPGPTRKHYQAYAAPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 50 - DNA, 2583 bp, Artificial Sequence
  Synthetic sequence A193 CAR (FMC63 scFv_STII_IgG4 hinge_CD28tm_CD28icd (P208A, P211A, Y218F)_CD3z_T2A_tEGFR) expression cassette
atgctgctgctggtgaccagcctgctgctgtgcgagctgccccaccctgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcctagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatcaggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtccgcagcaagcggagcagaggcggccacagcgactacatgaacatgacccctagacggcctggccccaccagaaagcactaccaggcctacgccgctccccgggactttgccgccttcagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 51 - protein, 860 aa, Artificial Sequence
  Synthetic sequence A193 CAR cassette protein sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSNWSHPQFEKGGGGSGGGGSESKYGPPCPPCPMFWVLVVVGGVLACYSLLVTVAFIIFWVRSKRSRGGHSDYMNMTPRRPGPTRKHYQAYAAPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 52 - DNA, 2583 bp, Artificial Sequence
  Synthetic sequence A194 CAR (FMC63 scFv_STII_IgG4 hinge_CD28tm_CD28icd (Y206F, P208A, Y209F, P211A, Y218F)_CD3z_T2A_tEGFR) expression cassette
atgctgctgctggtgaccagcctgctgctgtgcgagctgccccaccctgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcctagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatcaggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtccgcagcaagcggagcagaggcggccacagcgactacatgaacatgacccctagacggcctggccccaccagaaagcacttccaggccttcgccgctccccgggactttgccgccttcagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 53 - protein, 860 aa, Artificial Sequence
  Synthetic sequence A194 CAR cassette protein sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSNWSHPQFEKGGGGSGGGGSESKYGPPCPPCPMFWVLVVVGGVLACYSLLVTVAFIIFWVRSKRSRGGHSDYMNMTPRRPGPTRKHFQAFAAPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 54 - DNA, 2592 bp, Artificial Sequence
  Synthetic sequence A195 CAR (R12 scFv_STII_IgG4 hinge_CD28tm_CD28icd (P208A, P211A)_CD3z_T2A_tEGFR) expression cassette
atgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtccgcagcaagcggagcagaggcggccacagcgactacatgaacatgacccctagacggcctggccccaccagaaagcactaccaggcctacgccgctccccgggactttgccgcctacagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 55 - protein, 863 aa, Artificial Sequence
  Synthetic sequence A195 CAR cassette protein sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGNWSHPQFEKGGGGSGGGGSESKYGPPCPPCPMFWVLVVVGGVLACYSLLVTVAFIIFWVRSKRSRGGHSDYMNMTPRRPGPTRKHYQAYAAPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 56 - DNA, 2592 bp, Artificial Sequence
  Synthetic sequence A196 CAR (R12 scFv_STII_IgG4 hinge_CD28tm_CD28icd (P208A, P211A, Y218F)_CD3z_T2A_tEGFR) expression cassette
atgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtccgcagcaagcggagcagaggcggccacagcgactacatgaacatgacccctagacggcctggccccaccagaaagcactaccaggcctacgccgctccccgggactttgccgccttcagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 57 - protein, 863 aa, Artificial Sequence
  Synthetic sequence A196 CAR cassette protein sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGNWSHPQFEKGGGGSGGGGSESKYGPPCPPCPMFWVLVVVGGVLACYSLLVTVAFIIFWVRSKRSRGGHSDYMNMTPRRPGPTRKHYQAYAAPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 58 - DNA, 2592 bp, Artificial Sequence
  Synthetic sequence A1974 CAR (R12FMC63 scFv_STII_IgG4 hinge_CD28tm_CD28icd (Y206F, P208A, Y209F, P211A, Y218F)_CD3z_T2A_tEGFR) expression cassette
atgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtccgcagcaagcggagcagaggcggccacagcgactacatgaacatgacccctagacggcctggccccaccagaaagcacttccaggccttcgccgctccccgggactttgccgccttcagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 59 - protein, 863 aa, Artificial Sequence
  Synthetic sequence A197 CAR cassette protein sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGNWSHPQFEKGGGGSGGGGSESKYGPPCPPCPMFWVLVVVGGVLACYSLLVTVAFIIFWVRSKRSRGGHSDYMNMTPRRPGPTRKHFQAFAAPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 60 - protein, 480 aa, Artificial Sequence
  Synthetic sequence A042 CAR protein sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSNWSHPQFEKGGGGSGGGGSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 479 aa, Artificial Sequence
  Synthetic sequence A043 CAR protein sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSNWSHPQFEKGGGGSGGGGSESKYGPPCPPCPMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 483 aa, Artificial Sequence
  Synthetic sequence A044 CAR protein sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGNWSHPQFEKGGGGSGGGGSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 482 aa, Artificial Sequence
  Synthetic sequence A045 CAR protein sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGNWSHPQFEKGGGGSGGGGSESKYGPPCPPCPMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 479 aa, Artificial Sequence
  Synthetic sequence A142 CAR protein sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSNWSHPQFEKGGGGSGGGGSESKYGPPCPPCPMFWVLVVVGGVLACYSLLVTVAFIIFWVRSKRSRGGHSDYMNMTPRRPGPTRKHYQPYAPPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 479 aa, Artificial Sequence
  Synthetic sequence A143 CAR protein sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSNWSHPQFEKGGGGSGGGGSESKYGPPCPPCPMFWVLVVVGGVLACYSLLVTVAFIIFWVRSKRSRGGHSDYMNMTPRRPGPTRKHFQPFAPPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 482 aa, Artificial Sequence
  Synthetic sequence A144 CAR protein sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGNWSHPQFEKGGGGSGGGGSESKYGPPCPPCPMFWVLVVVGGVLACYSLLVTVAFIIFWVRSKRSRGGHSDYMNMTPRRPGPTRKHYQPYAPPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 482 aa, Artificial Sequence
  Synthetic sequence A145 CAR protein sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGNWSHPQFEKGGGGSGGGGSESKYGPPCPPCPMFWVLVVVGGVLACYSLLVTVAFIIFWVRSKRSRGGHSDYMNMTPRRPGPTRKHFQPFAPPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 479 aa, Artificial Sequence
  Synthetic sequence A175 CAR protein sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSNWSHPQFEKGGGGSGGGGSESKYGPPCPPCPMFWVLVVVGGVLACYSLLVTVAFIIFWVRSKRSRGGHSDFMNMTPRRPGPTRKHFQPFAPPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 482 aa, Artificial Sequence
  Synthetic sequence A176 CAR protein sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGNWSHPQFEKGGGGSGGGGSESKYGPPCPPCPMFWVLVVVGGVLACYSLLVTVAFIIFWVRSKRSRGGHSDFMNMTPRRPGPTRKHFQPFAPPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 478 aa, Artificial Sequence
  Synthetic sequence A192 CAR protein sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSNWSHPQFEKGGGGSGGGGSESKYGPPCPPCPMFWVLVVVGGVLACYSLLVTVAFIIFWVRSKRSRGGHSDYMNMTPRRPGPTRKHYQAYAAPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 479 aa, Artificial Sequence
  Synthetic sequence A193 CAR protein sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSNWSHPQFEKGGGGSGGGGSESKYGPPCPPCPMFWVLVVVGGVLACYSLLVTVAFIIFWVRSKRSRGGHSDYMNMTPRRPGPTRKHYQAYAAPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 479 aa, Artificial Sequence
  Synthetic sequence A194 CAR protein sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSNWSHPQFEKGGGGSGGGGSESKYGPPCPPCPMFWVLVVVGGVLACYSLLVTVAFIIFWVRSKRSRGGHSDYMNMTPRRPGPTRKHFQAFAAPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 482 aa, Artificial Sequence
  Synthetic sequence A195 CAR protein sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGNWSHPQFEKGGGGSGGGGSESKYGPPCPPCPMFWVLVVVGGVLACYSLLVTVAFIIFWVRSKRSRGGHSDYMNMTPRRPGPTRKHYQAYAAPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 482 aa, Artificial Sequence
  Synthetic sequence A196 CAR protein sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGNWSHPQFEKGGGGSGGGGSESKYGPPCPPCPMFWVLVVVGGVLACYSLLVTVAFIIFWVRSKRSRGGHSDYMNMTPRRPGPTRKHYQAYAAPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 482 aa, Artificial Sequence
  Synthetic sequence A197 CAR protein sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGNWSHPQFEKGGGGSGGGGSESKYGPPCPPCPMFWVLVVVGGVLACYSLLVTVAFIIFWVRSKRSRGGHSDYMNMTPRRPGPTRKHFQAFAAPRDFAAFRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 31 aa, Artificial Sequence
  Synthetic sequence
SPARLGSQHSPGRTASLNQRPRTHSGSSGGS

SEQ ID NO: 77 - protein, 31 aa, Artificial Sequence
  Synthetic sequence
DLSTQLSRTGTLSRKSIKAPATPASATLGRP

SEQ ID NO: 78 - protein, 31 aa, Artificial Sequence
  Synthetic sequence
DDETGKLQGSGVSLASKKSRLSSSSSNDSGN

SEQ ID NO: 79 - protein, 31 aa, Artificial Sequence
  Synthetic sequence
EARGGLGAPPLQSARSLPGPAPCLKHFPLDL

SEQ ID NO: 80 - protein, 31 aa, Artificial Sequence
  Synthetic sequence
KCCSIFQQEAPERASSVYTRSTGEQEISVGL

SEQ ID NO: 81 - protein, 20 aa, Artificial Sequence
  Synthetic sequence
KCPRPVVKSGDKPSLSARYV

SEQ ID NO: 82 - protein, 28 aa, Artificial Sequence
  Synthetic sequence
MSFRGKVFKREPSEFWKKRRTVRRVNQE

SEQ ID NO: 83 - protein, 31 aa, Artificial Sequence
  Synthetic sequence
EANTSPRLSQTFLQLSDGDKKTLTRKKVNQF

SEQ ID NO: 84 - protein, 31 aa, Artificial Sequence
  Synthetic sequence
IPGLDEANTSPRLSQTFLQLSDGDKKTLTRK

SEQ ID NO: 85 - protein, 31 aa, Artificial Sequence
  Synthetic sequence
NKERTSESRGLSRLFSSFLKRPKSQVSEEEG

SEQ ID NO: 86 - protein, 31 aa, Artificial Sequence
  Synthetic sequence
KERTSESRGLSRLFSSFLKRPKSQVSEEEGK

SEQ ID NO: 87 - protein, 31 aa, Artificial Sequence
  Synthetic sequence
TKKGTSSKKVIYSQPSARSEGEFKQTSSFLV

SEQ ID NO: 88 - protein, 31 aa, Artificial Sequence
  Synthetic sequence
GSSTETSDSHLTKALSTFIHAEDFARRRKRS

SEQ ID NO: 89 - protein, 27 aa, Artificial Sequence
  Synthetic sequence
SSRPDRETRASVIKKTSDITQARVKSC

SEQ ID NO: 90 - protein, 31 aa, Artificial Sequence
  Synthetic sequence
RQQQQSGAWGAPRKDSLLKPGLRAVVGGAAA

SEQ ID NO: 91 - protein, 31 aa, Artificial Sequence
  Synthetic sequence
HSQQPPPPQQERSKPSFHAPEPKAHYEPADR

SEQ ID NO: 92 - protein, 31 aa, Artificial Sequence
  Synthetic sequence
PRHKIISIFSGTEKGSKKKEKERPEISPPSD

SEQ ID NO: 93 - protein, 31 aa, Artificial Sequence
  Synthetic sequence
KMCHLPEPELNKERPSLQIKLKIEDFILHKM

SEQ ID NO: 94 - protein, 31 aa, Artificial Sequence
  Synthetic sequence
LESHRAVYIGVHVPFSKESRRRHRHRGHKHH

SEQ ID NO: 95 - protein, 22 aa, Artificial Sequence
  Synthetic sequence
GKREKDKEKDKEKRFSLFGKKK

SEQ ID NO: 96 - protein, 29 aa, Artificial Sequence
  Synthetic sequence
MAFANFRRILRLSTFEKRKSREYEHVRRD

SEQ ID NO: 97 - protein, 31 aa, Artificial Sequence
  Synthetic sequence
ITKKLEDVKNSPTFKSFEEKVENLKSKVGGT

SEQ ID NO: 98 - protein, 21 aa, Artificial Sequence
  Synthetic sequence
MAAGETQLYAKVSNKLKSRSS

SEQ ID NO: 99 - protein, 31 aa, Artificial Sequence
  Synthetic sequence
FKEPEKRTISRPAVGSTKYFGTAKARYDFCA

SEQ ID NO: 100 - protein, 31 aa, Artificial Sequence
  Synthetic sequence
KEPEKRTISRPAVGSTKYFGTAKARYDFCAR

SEQ ID NO: 101 - protein, 31 aa, Artificial Sequence
  Synthetic sequence
PAALPQSSEKLFQGYSFVAPSILFKRNAAVI

SEQ ID NO: 102 - protein, 28 aa, Artificial Sequence
  Synthetic sequence
MAFANFRRILRLSTFEKRKSREYEHVRR

SEQ ID NO: 103 - protein, 29 aa, Artificial Sequence
  Synthetic sequence
MAFANFRRILRLSTFEKRKSREYEHVRRD

SEQ ID NO: 104 - protein, 31 aa, Artificial Sequence
  Synthetic sequence
VTGIRLLSLGAGEFKSQEHAKHKGPKVERDQ

SEQ ID NO: 105 - protein, 31 aa, Artificial Sequence
  Synthetic sequence
QSSEKLFQGYSFVAPSILFKRNAAVIDPLQF

SEQ ID NO: 106 - protein, 25 aa, Artificial Sequence
  Synthetic sequence
MSRRRFDCRSISGLLTTTPQIPIKM

SEQ ID NO: 107 - protein, 31 aa, Artificial Sequence
  Synthetic sequence
RSYSPDHRQKKHRKLSELERPHKVSHGHERR

SEQ ID NO: 108 - protein, 31 aa, Artificial Sequence
  Synthetic sequence
YFIDFVARETTCSKESNEELTESCETKKLGQ

SEQ ID NO: 109 - protein, 31 aa, Artificial Sequence
  Synthetic sequence
RSRSRTSPVTRRRSRSRTPPAIRRRSRSRTP

SEQ ID NO: 110 - protein, 31 aa, Artificial Sequence
  Synthetic sequence
RSRTSPVTRRRSRSRTPPAIRRRSRSRTPLL

SEQ ID NO: 111 - protein, 31 aa, Artificial Sequence
  Synthetic sequence
VKKEARTLSDFNSLISSPHLGREKKKVKSQK

SEQ ID NO: 112 - protein, 25 aa, Artificial Sequence
  Synthetic sequence
QEQIAKRRRLSSLRASTSKSESSQK

SEQ ID NO: 113 - protein, 31 aa, Artificial Sequence
  Synthetic sequence
NPNRFITLLLPGGAQTAVRPGSPSTSTMRLD

SEQ ID NO: 114 - protein, 31 aa, Artificial Sequence
  Synthetic sequence
ATVTAFSFEDDTVPLSPLKYMAQRQQREKTR

SEQ ID NO: 115 - protein, 31 aa, Artificial Sequence
  Synthetic sequence
LKGAILTTMLATRNFSAAKSLLKKPDGVKES

SEQ ID NO: 116 - protein, 31 aa, Artificial Sequence
  Synthetic sequence
ILTTMLATRNFSAAKSLLKKPDGVKESTESS

SEQ ID NO: 117 - protein, 31 aa, Artificial Sequence
  Synthetic sequence
VLKHPYPRKVEEPSIYESVRVHTAMQTGRTE

SEQ ID NO: 118 - protein, 31 aa, Artificial Sequence
  Synthetic sequence
TAVLAPWPRPPPRRFSPPRRMLPPPPMWRRS

SEQ ID NO: 119 - protein, 31 aa, Artificial Sequence
  Synthetic sequence
LSPGETPERSLRLAESREQSPRRKEVESRLS

SEQ ID NO: 120 - protein, 31 aa, Artificial Sequence
  Synthetic sequence
ETPERSLRLAESREQSPRRKEVESRLSPGES

SEQ ID NO: 121 - protein, 31 aa, Artificial Sequence
  Synthetic sequence
AQPLELNQHSRFIIGSVSEDNSEDEISNLVK

SEQ ID NO: 122 - protein, 27 aa, Artificial Sequence
  Synthetic sequence
MTPRRPGPTRKHYQPYAPPRDFAAYRS

SEQ ID NO: 123 - protein, 31 aa, Artificial Sequence
  Synthetic sequence
VKRRKPRGDVVKVIVSVQRKRQEAEGEATVI

SEQ ID NO: 124 - protein, 31 aa, Artificial Sequence
  Synthetic sequence
HHDIVRLLDEYNVTPSPPGTVLTSALSPVIC

SEQ ID NO: 125 - protein, 23 aa, Artificial Sequence
  Synthetic sequence
MDNYADLSDTELTTLLRRYNIPH

SEQ ID NO: 126 - protein, 62 aa, Artificial Sequence
  Synthetic sequence
DLDVSNTTTAQKRKCSQTQCPRKVIKMESEEGEDVSNTTTAQKRKCSQTQCPRKVIKMESEE

SEQ ID NO: 127 - protein, 31 aa, Artificial Sequence
  Synthetic sequence
IRASSSSSSIRQRISSFETFGSSQLPDKGAQ

SEQ ID NO: 128 - protein, 31 aa, Artificial Sequence
  Synthetic sequence
LVRLSLTERLGKRKFSAGGDSDPPLKRSLAQ

SEQ ID NO: 129 - protein, 31 aa, Artificial Sequence
  Synthetic seqeunce
AQQAASSSGQGQQAQTPTGKQTDKTKSNMKG

SEQ ID NO: 130 - protein, 31 aa, Artificial Sequence
  Synthetic sequence
SSKVKYSRLSSTDDGYIDLQFKKTPPKIPYK

SEQ ID NO: 131 - protein, 31 aa, Artificial Sequence
  Synthetic seqeunce
RKERELRERLLSILLSKKPDDSHTHDELGVA

SEQ ID NO: 132 - protein, 26 aa, Artificial Sequence
  Synthetic sequence
LLQDLSEVSAPPLPPTSPGRDVAQDP

SEQ ID NO: 133 - protein, 29 aa, Artificial Sequence
  Synthetic sequence
LPGPPAPSPLPATPLSAKEDASKEDVIFF

SEQ ID NO: 134 - protein, 31 aa, Artificial Sequence
  Synthetic seqeunce
MDGAIASGVSKFATLSLHDRKERHHEKDHKR

SEQ ID NO: 135 - protein, 27 aa, Artificial Sequence
  Synthetic sequence
SSRPDRETRASVIKKTSDITQARVKSC

SEQ ID NO: 136 - protein, 31 aa, Artificial Sequence
  Synthetic sequence
YAYRHPLIREKPRHKSDVEIPATVTAFSFED

SEQ ID NO: 137 - protein, 31 aa, Artificial Sequence
  Synthetic sequence
LTERLGKRKFSAGGDSDPPLKRSLAQRLGKK

SEQ ID NO: 138 - protein, 31 aa, Artificial Sequence
  Synthetic sequence
LAMAAKRKAENPSPRSPSSQTPNSRRQSGKK

SEQ ID NO: 139 - protein, 31 aa, Artificial Sequence
  Synthetic sequence
NVTPSPPGTVLTSALSPVICGPNRSFLSLKH

SEQ ID NO: 140 - protein, 31 aa, Artificial Sequence
  Synthetic sequence
HDKKSQDFGNLFSFPSYSQKSEDDSAKFDSN

SEQ ID NO: 141 - protein, 31 aa, Artificial Sequence
  Synthetic sequence
KEARLARSSPEQPRPSTSKAVSPPHLDGPPS

SEQ ID NO: 142 - protein, 31 aa, Artificial Sequence
  Synthetic sequence
SLVAPALNKPKKPLTSSSAAPQRPISTQRTA

SEQ ID NO: 143 - protein, 31 aa, Artificial Sequence
  Synthetic sequence
AEIEKFDKSKLKKTETQEKNPLPSKETIEQE

SEQ ID NO: 144 - protein, 31 aa, Artificial Sequence
  Synthetic sequence
IEKYRMERPKIQQQFSDLKRKLAEVTEEEWL

SEQ ID NO: 145 - protein, 31 aa, Artificial Sequence
  Synthetic sequence
PPTHQASVGLLDTPRSRERSPSPLRGNVVPS

SEQ ID NO: 146 - protein, 31 aa, Artificial Sequence
  Synthetic sequence
ADGAPSAAPPDGLLASPDLGLLKLASPELER

SEQ ID NO: 147 - protein, 31 aa, Artificial Sequence
  Synthetic sequence
TRPKQEKAFSLKTISTSDPAEVLVKNSQPIK

SEQ ID NO: 148 - protein, 31 aa, Artificial Sequence
  Synthetic sequence
DDGVGNQLGALVHQRTVITEEFKVPDKMVGF

SEQ ID NO: 149 - protein, 16 aa, Artificial Sequence
  Synthetic sequence
HTDREATPDGGEDSDS

SEQ ID NO: 150 - protein, 31 aa, Artificial Sequence
  Synthetic sequence
PRLDFEGPDAKLSGPSLKMPSLEISAPKVTA

SEQ ID NO: 151 - protein, 31 aa, Artificial Sequence
  Synthetic sequence
KHEPDVSSKSSSVLDSSLDHRHKQKVLHDTM

SEQ ID NO: 152 - protein, 31 aa, Artificial Sequence
  Synthetic sequence
SEGSLQKGTEPSPGGTPQPSRPVSPAGPPEG

SEQ ID NO: 153 - protein, 31 aa, Artificial Sequence
  Synthetic sequence
LQKGTEPSPGGTPQPSRPVSPAGPPEGVPEE

SEQ ID NO: 154 - protein, 31 aa, Artificial Sequence
  Synthetic sequence
TEPSPGGTPQPSRPVSPAGPPEGVPEEAQPP

SEQ ID NO: 155 - protein, 30 aa, Artificial Sequence
  Synthetic sequence
SIHEALRRIKEASPESEDEEEALPCTDWEN

SEQ ID NO: 156 - protein, 31 aa, Artificial Sequence
  Synthetic sequence
KQAFATSPASKAARESLTEINRSFKEYTENM

SEQ ID NO: 157 - protein, 31 aa, Artificial Sequence
  Synthetic sequence
HIMQHHVLPIQARLGSIAEIDLGVPPPVMKT

SEQ ID NO: 158 - protein, 31 aa, Artificial Sequence
  Synthetic sequence
KHKKKGRQSRPANKQSPSPSEVSQSPGREVM

SEQ ID NO: 159 - protein, 31 aa, Artificial Sequence
  Synthetic sequence
YKVDYESQSTDTQNFSSESKRETEYGPCRRE

SEQ ID NO: 160 - protein, 31 aa, Artificial Sequence
  Synthetic sequence
KFRSLFFGSILAPVRSPQGPSPVLAEDSEGE

SEQ ID NO: 161 - protein, 31 aa, Artificial Sequence
  Synthetic sequence
KKKGRQSRPANKQSPSPSEVSQSPGREVMSY

SEQ ID NO: 162 - protein, 31 aa, Artificial Sequence
  Synthetic sequence
RRHERRHEAGLKRKPSQTLQPSEDLADGKAS

SEQ ID NO: 163 - protein, 31 aa, Artificial Sequence
  Synthetic sequence
FSQENKAPFEAVKRFSLDERSLACRQDSDSS

SEQ ID NO: 164 - protein, 19 aa, Artificial Sequence
  Synthetic sequence
MAETPPPPTAGAESCSEEP

SEQ ID NO: 165 - protein, 31 aa, Artificial Sequence
  Synthetic sequence
RSRSRTSPITRRRSRSRTSPVTRRRSRSRTS

SEQ ID NO: 166 - protein, 31 aa, Artificial Sequence
  Synthetic sequence
SRYRTTSSANNPNLMYQDECDRRLRGVKDGG

SEQ ID NO: 167 - protein, 31 aa, Artificial Sequence
  Synthetic sequence
GIPPSAGAHQLFRGFSFVATGLMEDDGKPRA

SEQ ID NO: 168 - protein, 31 aa, Artificial Sequence
  Synthetic sequence
DEKTTKIACKSPPPESVDTPTSTKQWPKRSL

SEQ ID NO: 169 - protein, 25 aa, Artificial Sequence
  Synthetic sequence
LQDLSEVSAPPLPPTSPGRDVAQDP

SEQ ID NO: 170 - protein, 31 aa, Artificial Sequence
  Synthetic sequence
LPWRPRGLRNLPRSRSQPCDLDARKTGVKRR

SEQ ID NO: 171 - protein, 27 aa, Artificial Sequence
  Synthetic sequence
MSRRRFDCRSISGLLTTTPQIPIKMEN

SEQ ID NO: 172 - protein, 31 aa, Artificial Sequence
  Synthetic sequence
PEEVLPSPTLQSLATSPRAILGSYRKKRKNS

SEQ ID NO: 173 - protein, 31 aa, Artificial Sequence; identical to SEQ ID NO: 79
  Synthetic sequence

SEQ ID NO: 174 - protein, 8 aa, Artificial Sequence
  Synthetic sequence Strep-Tag II peptide
WSHPQFEK

SEQ ID NO: 175 - protein, 50 aa, Artificial Sequence
  Synthetic sequence
  VARIANT (6)...(50): any one or all of amino acids 6-50 can either be present or absent.
GGGGSGGGGSGGGGSGGGGSGGGGSGGGGSGGGGSGGGGSGGGGSGGGGS

(19) United States
(12) Patent Application Publication
(10) Pub. No.: US 2021/0079061 A1
(43) Pub. Date: Mar. 18, 2021

(54) COMPOSITIONS AND METHODS FOR CELLULAR IMMUNOTHERAPY

(71) Applicant: FRED HUTCHINSON CANCER RESEARCH CENTER, Seattle, WA (US)

(72) Inventors: Alexander SALTER, Seattle, WA (US); Stanley RIDDELL, Sammamish, WA (US)

(21) Appl. No.: 16/976,040

(22) PCT Filed: Feb. 21, 2019
(86) PCT No.: PCT/US2019/019014

§ 371 Date: Aug. 26, 2020

Related U.S. Application Data
(60) Provisional application No. 62/635,450, filed on Feb. 26, 2018.
(60) Provisional application No. 62/676,787, filed on May 25, 2018.
(60) Provisional application No. 62/739,792, filed on Oct. 1, 2018.

(51) Int. Cl. C07K 14/705; C07K 16/28; C07K 14/725; A61K 35/17; A61K 45/06; A61P 35/00

(52) U.S. Cl. CPC C07K 14/70521; C07K 16/2803; C07K 14/7051; A61K 35/17; C07K 2317/622; A61P 35/00; C07K 2319/03; C07K 2319/33; A61K 45/06

(57) ABSTRACT

The present disclosure provides fusion proteins comprising a modified CD28 costimulatory signaling domain and uses thereof in immunotherapies for treating cancer. In son embodiments, host immune cells are provided that express the fusion proteins and have use in cellular immunotherapies for treating cancers and other diseases. In certain embodiments, a fusion protein comprises a chimeric antigen receptor (CAR) according to the present disclosure.

### STATEMENT OF GOVERNMENT INTEREST

This invention was made with government support under CA114536 awarded by the National Institutes of Health. The government has certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_458WO_SEQUENCE_LISTING.txt. The text file is 274 KB, was created on Feb. 20, 2019, and is being submitted electronically via EFS-Web.

### BACKGROUND

Adoptive immunotherapies using T cells modified with genetically engineered receptors targeted against cancer antigens has demonstrated clinical successes in hematological cancers and shown potential in the treatment of other cancers and diseases. Engineered receptors include chimeric antigen receptors (CARs) and enhanced affinity T cell receptors (TCRβ). See, e.g., Harris and Kranz, Trends Pharmacol. Sci. 37(3):220 (2016). Upon binding of the cancer antigen by the engineered receptor, the modified T cell mediates an anti-tumor response by inducing cytolysis of target tumor cells and releasing cytokines to stimulate the immune response.

In addition to antigen-driven stimulation, CARs and TCRs may also exhibit tonic (antigen-independent or constitutive) or excessive signaling in T cells. High surface expression, self-aggregating properties of the engineered receptors (e.g., scFv oligomerization), expression in gammaretroviral vectors, and the presence of certain costimulatory signaling domains may contribute to tonic and/or excessive signaling (Gomes-Silva et al., Cell Reports 21:17-26 (2017); Long et al., Nat. Med. 21:581-590 (2015); Frigault et al., Cancer Immunol. Res. 3:356-367 (2015)). Tonic and/or excessive signaling can lead to constitutive activation and proliferation of transduced T cells, accelerate T cell differentiation, limit T cell persistence, increase T cell exhaustion, increase T cell apoptosis, increase expression of immune checkpoint molecule receptors (e.g., PD-1, TIM-3, and LAG-3), and/or decrease anti-tumor activity of T cells (Gomes-Silva et al., supra; Frigault et al., supra; Long et al., supra; Eyquem et al., Nature 543:113 (2017)).

Moreover, CAR T cell therapy is also associated with significant toxicities, including cytokine release syndrome (CRS) and CAR T cell-related encephalopathy syndrome (CRES). Cytokine release syndrome refers to a systemic inflammatory response that is produced by elevated levels of cytokines associated with T cell activation and proliferation. CRS can be mild and self-limiting, with symptoms of fevers and myalgias, or more severe, with symptoms including vascular leakage, hypotension, respiratory and renal insufficiency, cytopenias, coagulopathy, multiorgan failure, and neurological toxicity. Neurological toxicity may present as a wide range of neurological and psychiatric symptoms, including seizure, delirium, aphasia, and hallucinations. CRES can occur concurrently with or after CRS, and may lead to fatal cerebral edema.

Accordingly, new strategies are needed in adoptive cell therapies for treating cancer. The presently disclosed embodiments address these needs and provide other related advantages.

### BRIEF DESCRIPTION OF THE DRAWINGS

FIGS. 1A-1G show that inclusion of a Strep-Tag® II (STII) tag in the spacer region of CD28/CD3ζ and 4-1BB/CD3 ζ CARs offers a precise method for activating chimeric antigen receptor (CAR) signaling. FIG. 1A: Schematic of STII CAR designs incorporating a single STII sequence in the extracellular hinge. CARs either contained the CD19-specific FMC63 scFv (SEQ ID NO:8) or ROR1-specific R12 scFv (SEQ ID NO:9). FIG. 1B: Schematic of a STII CAR T cell bound to a STII magnetic bead coated with STII mAb. FIG. 1C: Representative FACS plots of sort-purified CD19-specific CAR T cells after CD19⁺ LCL expansion. FIGS. 1D and 1E: Representative FACS plots show STII staining (D) of cell surface CAR and phenotyping (E) of sort-purified CD19-specific or ROR1-specific CAR T cells after expansion. Dashed line: CD28/CD3ζ CAR T cells, solid line: 4-1BB/CD3ζ CAR T cells, shaded histogram: isotype control. FIG. 1F: Representative FACS plots show DNA content staining of CAR T cells. Gate quantifies frequency of cells in G₀/G₁. FIG. 1G: Western blot analysis of lysates prepared from CAR T cells (4-1BB/CD3 ζ) after 45 minutes of co-culture with varying quantities of STII magnetic beads, K562 cells, or ROR1-transduced K562 (K562/ROR1) cells. Lysates were blotted for PLC-71 pY783, PLC-γ1, SLP-76 pS376, SLP-76, CD247 pY142, and CD247. Data in FIGS. 1C-1F are representative of four independent experiments. Blots in FIG. 1G are representative of two independent experiments.

FIGS. 2A-2G depict quantitative analysis of CAR phosphoprotein signaling, which shows that CARs signal through endogenous T cell signaling proteins. FIGS. 2A and 2B: CAR T cell stimulation conditions and experimental design. FIG. 2C: Bar graph shows the total number of unique phosphoserine (“pS”), phosphothreonine (“pT”) and phosphotyrosine (“pY”) sites identified in the three mass spectrometry (MS) experiments described in FIGS. 2A and 2B. FIG. 2D: Venn diagram showing the overlap among PO₄ sites detected in all MS experiments. FIGS. 2E and 2F: Scatter dot plots show average and range of log₂ fold change of canonical TCR signaling PO₄ events after 10 minutes (E) and 45 minutes (F) of CAR stimulation. FIG. 2G: Western blot analysis of lysates from the 10- and 45-minute stimulations of CD28/CD3ζ and 4-1BB/CD3ζ CAR T cells. Blots are representative of the three independent experiments described in FIGS. 2A and 2B.

FIGS. 3A-3F show that CD28/CD3ζ or 4-1BB/CD3ζ CAR stimulation modulates similar protein phosphorylation events with different kinetics and intensities. FIGS. 3A-3D: Volcano plots show log₂ fold change and false discovery rate (FDR) of PO₄ sites identified by MS in two or more experiments. Limma was used to define CAR stimulation-responsive PO₄ sites (gray dots at upper left (C, D) and upper-right (A, C, D) portions of volcano plots) meeting log₂ fold change and FDR cutoffs. FIG. 3E: Scatter plot comparing CD28/CD3ζ and 4-1BB/CD3ζ CAR stimulation-responsive PO₄ sites at the 45-minute time point. Gray dots in the middle upper and middle lower portions of the grid (as defined by the dashed lines; including the gray dot on the vertical “0” line) denote PO₄ sites more intensely modulated by 4-1BB/CD3ζ CAR stimulation. Light gray dots in the lower right and upper left portions of the grid denote CD28/CD3ζ CAR stimulation-responsive PO₄ sites that were modulated in an opposite direction by 4-1BB/CD3ζ CAR stimulation. FIG. 3F: Scatter dot plot shows average and range of the log₂ fold change of PO₄ sites on known CD28 and 4-1BB signaling pathway members after CAR stimulation.

FIG. 4 shows that CD28/CD3ζ or 4-1BB/CD3ζ CAR stimulation alters protein phosphorylation across similar signaling pathways and cellular compartments. Map of select proteins differentially phosphorylated after 45 minutes of CAR T cell stimulation from analysis of all MS experiments.

FIGS. 5A-5C show that CD28/CD3ζ CAR stimulation produces greater magnitude changes in protein PO₄ than 4-1BB/CD3ζ CAR stimulation. FIG. 5A: Dot plots show average log₂ fold change among the 20 most-phosphorylated sites after CAR stimulation. FIG. 5B: Dot plots show the absolute log₂ fold change for each PO₄ site detected on known KEGG TCR Signaling Pathway proteins after CAR stimulation. Absolute log₂ fold change was used to quantify the magnitude of PO₄ alterations because some sites were dephosphorylated after CAR activation. FIG. 5C: Western blot analysis of CAR T cells stimulated for the given times with STII magnetic beads. Blots are representative of 3 independent experiments. P-values in FIGS. 5A and 5B were calculated using an unpaired two-sample t-test.

FIGS. 6A-6K show that CD28/CD3ζ CAR signals promote an effector cell-like phenotype with reduced in vivo anti-tumor activity. FIGS. 6A-6B: Bar graphs show mean log₂ fold change values in gene expression comparing stimulated to control CD28/CD3ζ or 4-1BB/CD3ζ CAR T cells. FIG. 6C: Volcano plot shows log₂ fold change and false discovery rate (FDR) of gene expression comparing stimulated CD28/CD3ζ to 4-1BB/CD3ζ CAR T cells. Limma was used to define differentially expressed genes (gray dots in upper left and upper right portions of graph, as defined by the dashed lines) meeting log₂ fold change and FDR cutoffs. FIG. 6D: Bar graphs show log₂ fold change of IL7R, KLF2, and FOXO4 expression comparing CD28/CD3ζ and 4-1BB/CD3ζ CAR T cells as measured by qPCR. Graph depicts mean and standard deviation from n=3 biological replicates quantified together in a single qPCR run. FIG. 6E: Representative FACS plots show intracellular staining of cytokine production after 5 hours co-culture of ROR1-specific CAR T cells with K562 or K562/ROR1 cells. FIG. 6F: Bar graph shows mean and standard deviation of fold changes in cytokine production 24 hours after co-culture of ROR1-specific CAR T cells and K562/ROR1 cells. n=3-4 independent experiments. * denotes p<0.05 by Welch's t-test. FIG. 6G: FACS plot shows CAR T cell proliferation as measured by CFSE dye dilution 72 hours after STII bead stimulation. Histogram lines are as shown in the figure key. FIG. 6H: Survival analyses of NSG mice treated with a single infusion of CART cells (3.0×10⁶ cells, left, or 7.5×10⁵ T cells, right) 7 days after Raji/ffluc engraftment. Survival analyses of 6, 9, or 15 mice per group are pooled from two or three independent experiments. FIG. 6: Representative bioluminescence images show total radiance (p/sec/cm2/sr) of mice at the indicated time points. FIGS. 6J-6K: Bar graphs show CAR T cell frequency (J) in bone marrow or peripheral blood, or mean fluorescence intensity (MFI) (K) of PD-1, Lag-3, or Tim-3 expression on CAR T cells in the bone marrow on day 20 as measured by flow cytometry. n=5 mice per group. An unpaired two-tailed student's t test was used to compare group means. Data in FIG. 6G is representative of four independent experiments. Data in FIGS. 6H-6K are representative of two independent experiments

FIGS. 7A-7H show that CD28/CD3ζ CARs differentially associate with endogenous Lck and CD28 and that tyrosines in the CD28 domain contribute to unique attributes associated with CD28/CD3ζ CAR T cells. FIGS. 7A and 7B: Western blot analysis of whole cell lysates (L) and immunoprecipitated fractions (IP) from resting (A) or stimulated (B) CAR T cells. Blots in (A) are representative of 3 independent experiments; blots in (B) are representative of one experiment. FIG. 7C: Schematic of tyrosine mutations made to the CAR CD28 costimulatory signaling domain. FIG. 7D: FACS plots show CAR T cell proliferation as measured by CFSE dye dilution 72 hours after co-culture with K562/ROR1 cells. Histogram lines are as shown in the figure key. FIG. 7E: Bar graph shows mean and standard deviation of fold changes in cytokine production 24 hours after co-culture with K562/ROR1 cells. n=3 independent experiments. FIG. 7F: Western blot analysis of whole cell lysates (L) and immunoprecipitated fractions (IP) from resting CAR T cells. Blots are representative of 3 independent experiments. FIG. 7G: Western blot of CAR T cells stimulated for the given times with STII magnetic beads. FIG. 7H: Survival analysis of NSG mice treated with a single infusion of CAR T cells 7 days after Raji/filuc engraftment. Survival analyses of 6 or 15 mice per group are pooled from two to three independent experiments.

FIGS. 8A-8B show that mass spectrometric analysis of TCR signaling in Jurkat cells and primary T cells reveals marked differences in protein phosphorylation. FIG. 8A: Primary CD8⁺ T cells or Jurkat cells were stimulated with mouse anti-human CD3ε mAb and anti-mouse IgG or left unstimulated by treating with anti-mouse IgG alone. FIG. 8B: Heat map showing log 2 fold change of known PO₄ sites in the proximal TCR signaling pathway.

FIG. 9 shows that peptide labeling using tandem mass tag (TMT) reagents and multistep phosphopeptide enrichments enable detailed analysis of protein phosphorylation in primary CAR T cells. A schematic of an exemplary phosphopeptide enrichment strategy is provided. Briefly, lysates were reduced, alkylated, digested with trypsin, and then labeled with TMT reagents. Labeled peptides were pooled and subjected to an immunoprecipitation for phosphotyrosine (pTyr) peptides. The eluate was retained for LC-MS/MS. The flow through was separated into 13 fractions using basic reverse phase liquid chromatography followed by global phosphopeptide enrichment using immobilized metal affinity chromatography (IMAC). All 14 enriched fractions (1 p-Tyr+13 IMAC) were then analyzed by LC-MS/MS.

FIG. 10 shows PO₄ log 2 fold change summary statistics. Histogram shows the distribution of log 2 fold change (simulation vs control) values across three experiments. Standard deviations are noted in the legend.

FIGS. 11A-11D show that CAR-Lck association is partially responsible for the increased signal strength associated with CD28/CD3ζ CAR T cells. FIG. 11A: Schematic of mutations made to the CAR CD28 costimulatory signaling domain. FIG. 11B: Western blot analysis of whole cell lysates (L) and immunoprecipitated fractions (IP) from resting CAR T cells. Blots are representative of two independent experiments. FIG. 11C: Western blot analysis of CART cells stimulated for the given times with STII magnetic beads. Blots are representative of three independent experiments. FIG. 11D: Bar graph shows mean cytokine concentrations 24 hours after co-culture with K562/ROR1 cells. Data are representative of two independent experiments.

### DETAILED DESCRIPTION

The present disclosure generally relates to fusion proteins (e.g., chimeric antigen receptors, chimeric costimulatory receptors) comprising a modified CD28 costimulatory signaling domain. In certain aspects, the present disclosure provides fusion proteins comprising an extracellular component comprising a binding domain that specifically binds a target antigen; an intracellular component comprising a modified CD28 costimulatory signaling domain; and a hydrophobic portion disposed between the extracellular component and intracellular component, wherein the modified CD28 costimulatory signaling domain comprises at least one amino acid substitution that modulates a functional activity (i.e., one or more functional activity as provided herein) of the fusion protein as compared to a fusion protein comprising wild-type CD28 costimulatory signaling domain. Such fusion proteins, and host cells expressing the same, can be used in methods of, for example, adoptive immunotherapy to treat cancer.

By way of background, synthetic receptors that redirect T cell specificity and/or promote desired effector functions are being developed as immunotherapeutic reagents for cancer, autoimmunity, and infections (Sadelain et al., Nature 545:423-431 (2017). Exemplary receptors capable of redirecting T cell specificity and/or promoting desired effector functions include chimeric antigen receptors (CARs), chimeric chemokine receptors, chimeric costimulatory receptors, and engineered T cell receptors (TCRβ). For example, some CARs redirect T cell specificity to tumor cells by linking an extracellular antigen-specific single-chain variable (scFv) fragment to intracellular T cell signaling domains that mimic TCR activation (van der Stegen et al., Nat Rev Drug Discov 14:499-509 (2015). CAR-modified T cells have shown promise for treating a variety of malignancies and cancers (see, e.g., Kalos et al., Sci Transl Med 3:95ra73 (2011); Maude et al., N Engl J Med 371:1507-1517 (2014); Davila et. al., Sci Transl Med 6:224ra25 (2014); Lee et al., Lancet 385:517-528 (2015); Kochenderfer et al., J. Clin. Oncol. 33:540-549 (2015); Turtle et al., J. Clin. Invest. 126: 2123-2138 (2016); Turtle et al., Sci Transl Med 8, 355ra116 (2016); Turtle et al., J. Clin. Oncol. 35:3010-3020 (2017)).

T cell activation mediated by TCR engagement results in protein phosphorylation (PO₄) initiated by immunoreceptor tyrosine-based activating motif (ITAM)-containing CD3δ, ε, γ, and ζ chains (Brownlie et al., Nature Reviews Immunology 13:257-269 (2013)). Combined with PO₄ signals delivered in trans from costimulatory molecules, these events alter T cell transcriptional programs, promote cytokine release, and induce proliferation (Kaech and Cui, Nature Reviews Immunology 12:749-761 (2012)). CARs generally contain a CD3ζ endodomain in a single chain construct with a costimulatory domain from CD28 and/or 4-1BB. CD28/CD3ζ and 4-1BB/CD3ζ CAR T cells are both effective in treating tumors in patients, but exhibit functional differences in vitro and in preclinical mouse models (Kalos et al., Sci Transl Med 3, 95ra73 (2011); van der Stegen et al., Nat Rev Drug Discov 14:499-509 (2015); Cherkassky et al., J. Clin. Invest. 126:3130-3144 (2016); Kawalekar et al., Immunity 44:380-390 (2016)). For example, CD28/CD3ζ CAR T cells have been reported to exhibit a low level of tonic (antigen-independent) CD3ζ phosphorylation, and appear to be more likely to exhibit tonic signaling as compared to 4-1BB/CD3ζ CAR T cells. Tonic CAR signaling may have adverse effects on CAR T cells, including excessive cytokine release independent of binding to cognate antigens, limited persistence, exhaustion, apoptosis, increased expression of immune checkpoint molecule receptors, or decreased anti-tumor activity (Frigault et al., Cancer Immunol Res 3:356-367 (2015); Long et al., Nature Medicine 21:581-590 (2015).

The present disclosure provides fusion proteins containing a CD28 costimulatory signaling domain having alterations in one or more of certain amino acid residues that, when altered, modify tonic phosphorylation of a CAR's CD3ζ signaling domain and/or association with endogenous T cell signaling molecules. Moreover, CARs comprising a modified CD28 costimulatory signaling domain of this disclosure exhibit reduced levels of cytokine production as compared to those containing a wild-type CD28 costimulatory domain, which can result in reduced clinical toxicity of a CAR T cell therapy (e.g., cytokine release syndrome or CAR T cell-related encephalopathy syndrome).

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means 20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

The term “consisting essentially of” is not equivalent to “comprising” and refers to the specified materials or steps, or to those that do not materially affect the basic characteristics of a claimed invention. For example, a protein domain, region, module or cassette (e.g., a binding domain, hinge region, linker module, tag cassette) or a protein (which may have one or more domains, regions, modules or cassettes) “consists essentially of” a particular amino acid sequence when the amino acid sequence of a domain, region, module, cassette or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, cassette or protein and do not substantially affect (i.e., do not reduce the activity by more than 50%, such as no more than 40%, 30%, 25%, 20%, 15%, 10%, 5%, or 1%) the activity of the domain(s), region(s), module(s), cassette(s) or protein (e.g., the target binding affinity of a binding protein or tag cassette).

A “fusion protein” comprises a single-chain polypeptide having at least two distinct domains (e.g., antigen-binding domain and modified CD28 costimulatory signaling domain), wherein the domains are not naturally found together in a protein. A polynucleotide encoding a fusion protein may be constructed using PCR, recombinantly engineered, or the like, or such fusion proteins can be made synthetically. A fusion protein may further contain other components (e.g., covalently bound), such as a tag, linker, transduction marker, or bioactive molecule. In certain embodiments, a fusion binding protein is a chimeric antigen receptor (CAR), a T cell receptor based-CAR (TCR-CAR), or a chimeric costimulatory receptor (CCR).

As used herein, the term “chimeric antigen receptor” (CAR) refers to a fusion protein comprising two or more distinct domains that are linked together in an arrangement that does not occur naturally, can function as a receptor when expressed on the surface of a cell, and comprises: an extracellular component comprising an antigen binding domain specific for an antigen; an optional extracellular spacer domain; a hydrophobic portion or transmembrane domain; and an intracellular component comprising an intracellular activation domain (e.g., an immunoreceptor tyrosine-based activation motif (ITAM)-containing T cell activating motif), an intracellular costimulatory domain, or both. In certain embodiments, an intracellular signaling component of a CAR has an ITAM-containing T cell activating domain (e.g., CD3) and an intracellular costimulatory domain (e.g., CD28). In certain embodiments, a CAR is synthesized as a single polypeptide chain or is encoded by a nucleic acid molecule as a single chain polypeptide.

A “chimeric costimulatory receptor” (CCR) refers to a fusion protein comprising an extracellular component comprising an antigen-binding domain, an optional extracellular spacer domain, a hydrophobic portion or transmembrane domain, and at least one intracellular costimulatory domain, but does not comprise an intracellular activation domain (e.g., an immunoreceptor tyrosine-based activation motif (ITAM)-containing T cell activating motif). A CCR may be synthesized as a single polypeptide chain or can be encoded by a nucleic acid molecule as a single-chain polypeptide. In certain embodiments, a CCR further comprises a heterodimerization domain. A CCR construct comprising a heterodimerization domain may be co-expressed in a host cell with a second polypeptide comprising an intracellular activation domain (e.g., an immunoreceptor tyrosine-based activation motif (ITAM)-containing T cell activating motif) and a corresponding heterodimerization domain. Administration of a heterodimerizing agent (e.g., a small molecule) facilitates assembly of the CCR with the polypeptide comprising an intracellular activation domain via their corresponding heterodimerization domains. Such dual polypeptide heterodimerizing constructs may be referred to as “ON-switch CARs” or “split CARs” (see, e.g., Wu et al., Science 350:aab4077 (2015)), which constructs are incorporated herein by reference. A “T cell receptor-based chimeric antigen receptor” (TCR-CAR) refers to a heterodimeric fusion protein comprising an extracellular component comprising a soluble T cell receptor (TCR) specific for an antigen, a hydrophobic portion or transmembrane domain, and an intracellular component comprising an intracellular activation domain (e.g., an immunoreceptor tyrosine-based activation motif (ITAM)-containing T cell activating motif), an intracellular costimulatory domain, or both (see, e.g., Walseng et al., Scientific Reports 7:10713, (2017); the TCR-CAR constructs and methods of which are incorporated by reference in their entirety). In certain embodiments, a TCR-CAR comprises or consists of: a first polypeptide strand comprising an extracellular component comprising a TCR α chain variable domain (Vα) linked to a TCR α constant domain or a portion thereof; and a second polypeptide strand comprising an extracellular component comprising a TCR β chain variable domain (Vβ) linked to a TCR β chain constant domain or a portion thereof; a hydrophobic portion or transmembrane domain, and an intracellular component comprising an intracellular activation domain (e.g., an immunoreceptor tyrosine-based activation motif (ITAM)-containing T cell activating motif), an intracellular costimulatory domain, or both. In certain embodiments, an intracellular signaling component of a TCR-CAR has an ITAM-containing T cell activating domain (e.g., CD31) and an intracellular costimulatory domain (e.g., CD28). In certain embodiments, the extracellular portion of the TCR α chain constant domain (or a portion thereof) and the extracellular portion of the TCR β chain constant domain (or a portion thereof) are both modified to add a cysteine residue to increase dimerization.

A “single chain TCR” (scTCR or scTv) refers to a fusion protein comprising an extracellular component comprising a TCR Vα domain linked to a TCR Vβ domain with a flexible linker (e.g., with a (Gy₄Ser)₂₋₅, e.g., SEQ ID NO:175). It will be understood that a scTCR can be arranged so that the linker connects the C-terminal end of the TCR Vα domain to the N-terminal end of the TCR Vβ domain, or connects the N-terminal end of the TCR Vα domain to the C-terminal end of the TCR Vβ domain.

A “binding domain” (also referred to as an “antigen binding domain” or “binding region” or “binding moiety”), as used herein, refers to a molecule, such as a peptide, oligopeptide, polypeptide, or protein that possesses the ability to specifically and non-covalently associate, unite, or combine with a target molecule (e.g., viral antigen, bacterial antigen, cancer antigen, autoimmune disease antigen, self-antigen). A binding domain includes any naturally occurring, synthetic, semi-synthetic, or recombinantly produced binding partner for a biological molecule or other target of interest. In some embodiments, the binding domain is an antigen-binding domain, such as an antibody or T cell receptor (TCR) or functional binding domain or antigen-binding fragment thereof. Exemplary binding domains include single chain antibody variable regions (e.g., domain antibodies, sFv, scFv, Fab), antigen-binding regions of T cell receptors (TCRβ), such as single chain TCRs (scTCRβ) or soluble TCRβ, receptor ectodomains, ligands, or synthetic polypeptides selected for the specific ability to bind to a biological molecule. In certain embodiments, a binding domain is not a binding domain from a CD8 ectodomain or extracellular domain or any portion thereof that comprises a functional IgV-like domain (i.e., is not a binding domain specific for antigen peptide: MHC complex from a CD8α chain or a CD8β chain). There are two subunits of CD8, CD8α and CD8β, and a CD8 co-receptor can exist as an α homodimer or αβ heterodimer. In further embodiments, a binding domain is not a binding domain from a CD8α monomer, CD8β monomer, CD8aa homodimer, or CD8αβ heterodimer ectodomain or extracellular domain or any portion thereof that comprises a functional IgV-like domain. Reference to CD8α includes the “canonical” human CD8α protein (NP_001759.3) as well as splice isoform 2, which lacks an internal segment including the transmembrane domain resulting in a secreted protein (RefSeq NP_741969.1), and splice isoform 3, which uses an alternate promoter and 5′ UTR (RefSeq NP_001139345.1). Reference to CD8β includes the “canonical” human CD8β protein (RefSeq NP_004922) as well as isoforms 2-8, corresponding to RefSeq NP742099, RefSeq NP_742100, UniProt P10966-4, RefSeq NP_757362, Uniprot P10966-7, Uniprot P10966-8, and RefSeq NP_001171571. Exemplary IgV-like domains may be found at amino acids 22-135 of the human canonical CD8α protein (SEQ ID NO:43) and amino acids 22-132 of the human canonical CD8β protein (SEQ ID NO:44). In certain embodiments, a CD8 binding domain binds with an antigen peptide:MHC I complex in the context of a TCR, which may be naturally occurring, recombinant, or engineered, or any other recombinant binding molecule that comprises a binding domain from a TCR (e.g., a scTCR or a TCR-based CAR).

As used herein, “specifically binds” refers to an association or union of a binding domain, or a fusion binding protein thereof, to a target molecule with an affinity or Kₐ (i.e., an equilibrium association constant of a particular binding interaction with units of 1/M) equal to or greater than 10⁵ M⁻¹, while not significantly associating or uniting with any other molecules or components in a sample. Binding domains (or fusion binding proteins thereof) may be classified as “high affinity” binding domains (or fusion binding proteins thereof) or “low affinity” binding domains (or fusion binding proteins thereof). “High affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” binding domains refer to those binding domains with a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M). In certain embodiments, a binding domain may have “enhanced affinity,” which refers to a selected or engineered binding domain with stronger binding to a target antigen than a wild-type (or parent) binding domain. For example, enhanced affinity may be due to a Ka (equilibrium association constant) for the target antigen that is higher than the wild-type binding domain, or due to a K_{d} (dissociation constant) for the target antigen that is less than that of the wild-type binding domain, or due to an off-rate (K_{off}) for the target antigen that is less than that of the wild-type binding domain.

In certain embodiments, a T cell receptor, antibody, or binding domain or fragment thereof may have “enhanced affinity,” which refers to selected or engineered receptors or binding domains with stronger binding to a target antigen than a wild-type (or parent) binding domain. For example, enhanced affinity may be due to a Kₐ (equilibrium association constant) for the target antigen that is higher than the wild-type binding domain, due to a K_{d} (dissociation constant) for the target antigen that is less than that of the wild-type binding domain, due to an off-rate (k_{off}) for the target antigen that is less than that of the wild-type binding domain, or a combination thereof. In certain embodiments, fusion proteins of the present disclosure, such as, for example, CARs or TCRβ, may be codon optimized to enhance expression in a particular host cell, such as T cells (Scholten et al., Clin. Immunol. 119:135 (2006)).

A variety of assays are known for identifying binding domains of the present disclosure that specifically bind a particular target, as well as determining binding domain or fusion protein affinities, such as Western blot, ELISA, analytical ultracentrifugation, spectroscopy, surface plasmon resonance (Biacore®) analysis, and MHC tetramer assay (see, e.g., Scatchard et al., Ann. N.Y. Acad. Sci. 51:660 (1949); Wilson, Science 295:2103 (2002); Wolff et al., Cancer Res. 53:2560 (1993); Altman et al., Science 274:94-96 (1996); and U.S. Pat. Nos. 5,283,173 and 5,468,614, or the equivalent).

As used herein, “tag cassette” refers to a unique peptide sequence affixed to, fused to, or that is part of a protein of interest, to which a heterologous or non-endogenous cognate binding molecule (e.g., receptor, ligand, antibody, or other binding partner) is capable of specifically binding where the binding property can be used to detect, identify, isolate or purify, track, enrich for, or target a tagged protein or cells expressing a tagged protein, particularly when a tagged protein is part of a heterogeneous population of proteins or other material, or when cells expressing a tagged protein are part of a heterogeneous population of cells (e.g., a biological sample like peripheral blood). In certain embodiments, a cell expressing a tagged protein can be contacted with a heterologous or non-endogenous cognate binding molecule and induce a biological response, such as promote cell activation, cell proliferation or cell death. In the provided fusion binding proteins, the ability of the tag cassette(s) to be specifically bound by the cognate binding molecule(s) is distinct from or in addition to the ability of the binding domain(s) to specifically bind to the target molecule(s). The tag cassette generally is not an antigen-binding molecule, for example, is not an antibody or TCR or an antigen-binding portion thereof. Exemplary tag cassettes are provided herein. In some embodiments, a tag casette is comprised in an extracellular component of a fusion protein of the present disclosure, and may be located, for example, between the binding domain and the hydrophobic portion, or at an N-terminal or C-terminal end of a binding domain polypeptide (e.g., a VH, a VL, a TCRα, a TCRβ, or the like), or can be located within a binding domain of the fusion protein (e.g., between a VH and a VL, or between a TCRα and a TCRβ), provided that the tag does not interfere with, or does not substantially interfere with, binding to antigen.

As used herein, a “hinge region” or a “hinge” refers to (a) an immunoglobulin hinge sequence (made up of, for example, upper and core regions of an immunoglobulin hinge) or a functional fragment or variant thereof, (b) a type II C-lectin interdomain (stalk) region or a functional fragment or variant thereof, or (c) a cluster of differentiation (CD) molecule stalk region or a functional variant thereof. As used herein, a “wild-type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody. In certain embodiments, a hinge region is human, and in particular embodiments, comprises a human IgG hinge region. An IgG hinge region includes any one or more of an IgG1 hinge region, an IgG2 hinge region, an IgG3 hinge region, or IgG4 hinge region.

A “hydrophobic portion,” as used herein, means any amino acid sequence having a three-dimensional structure that is thermodynamically stable in a cell membrane, and generally ranges in length from about 15 amino acids to about 30 amino acids. The structure of a hydrophobic domain may comprise an alpha helix, a beta barrel, a beta sheet, a beta helix, or any combination thereof. In certain embodiments, a hydrophobic portion is a transmembrane domain, for example, a transmembrane domain derived from a CD8, CD28, or CD27 molecule.

As used herein, an “immunoreceptor tyrosine-based activation motif (ITAM) T cell activating domain” refers to an intracellular signaling domain or functional portion thereof which is naturally or endogenously present on an immune cell receptor or a cell surface marker and contains at least one immunoreceptor tyrosine-based activation motif (ITAM). ITAM refers to a conserved motif of YXXL/I-X₆₋₈-YXXL/I (SEQ ID NO:42), wherein X is any amino acid (i.e., a same or different amino acid over the length of the ITAM). In certain embodiments, an ITAM signaling domain contains one, two, three, four, or more ITAMs. An ITAM signaling domain may initiate T cell activation signaling following antigen binding or ligand engagement. ITAM-signaling domains include, for example, intracellular signaling domains of CD3γ, CD3δ, CD3ε, CD3ζ, CD79a, CD79b, gamma chain of FcεRI or FcγRI, FcRγ2a, FcRγ2b1, FcRγ2a1, FcRγ2b2, FcRγ3a, FcRγ3b, FcRβ1, FcεR), Natural Killer cell receptor proteins (e.g., DAP12), CD5, CD16a, CD16b, CD22, CD23, CD32, CD64, CD79a, CD79b, CD89, CD278, and CD66d. Exemplary amino acid sequences of these ITAM sequences and those from viruses (e.g., BLV gp30; EBV LMP2A) are described in Paul, Fundamental Immunology 307 (Wolters Kluwer; Lippincott; Wilkins & Wilkins; Seventh Ed., 2008). These ITAMs and functional fragments and variants thereof are also contemplated for use in the presently disclosed fusion proteins and host cells, and are hereby incorporated by reference.

As used herein, a “costimulatory signaling domain” refers to an intracellular signaling domain, or functional portion thereof, of a costimulatory molecule, which, when activated in conjunction with a primary or classic (e.g., ITAM-driven) activation signal (provided by, for example a CD3ζ intracellular signaling domain), promotes or enhances a T cell response, such as T cell activation, cytokine production, proliferation, differentiation, survival, effector function, or combinations thereof. Costimulatory signaling domains include, for example, CD28, CD40L, GITR, NKG2C, CARD1, CD2, CD7, CD27, CD30, CD40, CD54 (ICAM), CD83, CD134 (OX-40), CD137 (4-1BB), CD150 (SLAMF1), CD152 (CTLA4), CD223 (LAG3), CD226, CD270 (HVEM), CD273 (PD-L2), CD274 (PD-L1), CD278 (ICOS), DAP10, LAT, LFA-1, LIGHT, SLP76, TRIM, ZAP70, CD5, BAFF-R, SLAMF7, NKp80, CD160, B7-H3, a ligand that specifically binds with CD83, or any combination thereof.

As used herein, a “CD28 costimulatory signaling domain” refers to an intracellular signaling domain, or functional portion thereof, of CD28. CD28 is a costimulatory molecule that is constitutively expressed on all human CD4⁺ T cells and about 50% of human CD8⁺ T cells (Linsley et al., Annu. Rev. Immunol. 11:191-212 (1993); June et al. Immunol. Today 11:211-16 (1990)). CD28 is an “early” costimulatory molecule that has been shown to synergize with the TCR to lower the threshold of T cell activation, which, in some cases, is not attainable by TCR ligation alone, leading to enhanced survival and increased cytokine production (e.g., IL-2) needed for clonal expansion and differentiation (Bour-Jordan et al., Immunol. Rev. 241:180-205 (2011)). An exemplary “wild-type” or “endogenous” human CD28 costimulatory signaling domain comprises an amino acid sequence of SEQ ID NO:2. Modifications to the human CD28 costimulatory signaling domain (e.g., amino acid substitutions), as described herein, may refer to the position within the full-length wild-type human CD28 polypeptide sequence as set forth in SEQ ID NO:1.

Terms understood by those in the art of antibody technology are each given the meaning acquired in the art, unless expressly defined differently herein. The term “antibody” refers to an intact antibody comprising at least two heavy (H) chains and two light (L) chains inter-connected by disulfide bonds (though it will be understood that heavy chain antibodies, which lack light chains, are still encompassed by the term “antibody”), as well as an antigen-binding portion of an intact antibody that has or retains the capacity to bind a target molecule. Antibodies include polyclonal and monoclonal antibodies. An antibody may be naturally occurring, recombinantly produced, genetically engineered, or modified, and includes modified forms of immunoglobulins, such as, for example intrabodies, peptibodies, nanobodies, single domain antibodies, and multispecific antibodies (e.g., bispecific antibodies, diabodies, triabodies, tetrabodies, tandem di-scFV, tandem tri-scFv).

“Binding fragment,” “binding portion,” or “binding domain” from an antibody refers to an “antibody fragment” that comprises a portion of an intact antibody and contains the antigenic determining variable regions or complementary determining regions of an antibody. Examples of antibody fragments include Fab, Fab′, F(ab′)2, and Fv fragments, Fab′-SH, F(ab′)₂, diabodies, linear antibodies, single chain antibodies, scFv (i.e., a fusion protein of the variable heavy (V_{H}) and variable light (V_{L}) regions of an immunoglobulin (Ig) molecule, connected with a short linker peptide of generally about 10 to about 25 amino acids), VHH, single domain antibodies (e.g., sdAb, sdFv, nanobody), and multispecific antibodies comprising antibody fragments. A monoclonal antibody or antigen-binding portion thereof may be non-human, chimeric, humanized, or human, preferably humanized or human. Immunoglobulin structure and function are reviewed, for example, in Harlow et al., Eds., Antibodies: A Laboratory Manual, Chapter 14 (Cold Spring Harbor Laboratory, Cold Spring Harbor, 1988). An antibody may be of any class or subclass, including IgG and subclasses thereof (IgG1, IgG2, IgG3, IgG4), IgM, IgE, IgA, and IgD.

The terms “variable light chain” (V_{L}) and “variable heavy chain” (V_{H}) refer to the variable binding region from an antibody light and heavy chain, respectively. The variable binding regions are made up of discrete, well-defined sub-regions known as “complementarity determining regions” (CDRs, also referred to as HVRs (hypervariable regions)) and “framework regions” (FRs). CDRs refer to sequences of amino acids within antibody variable regions that confer antigen specificity and/or binding affinity and are separated by FRs. There are three CDRs in each antibody light chain variable region (LCDR1, LCDR2, LCDR3) and three CDRs in each antibody heavy chain variable region (HCDR1, HCR2, HCDR3).

The term “CL” refers to an “immunoglobulin light chain constant region” or a “light chain constant region,” i.e., a constant region from an antibody light chain.

The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM).

A “Fab” (fragment antigen binding) is the part of an antibody that binds to antigens and includes the variable region and CH1 of the heavy chain linked to the light chain via an inter-chain disulfide bond.

As used herein, “Fc region portion” refers to the heavy chain constant region segment of the Fc fragment (the “fragment crystallizable” region or Fc region) from an antibody, which can include one or more constant domains, such as CH2, CH3, CH4, or any combination thereof. In certain embodiments, an Fc region portion includes the CH2 and CH3 domains of an IgG, IgA, or IgD antibody or any combination thereof, or the CH3 and CH4 domains of an IgM or IgE antibody, or any combination thereof. In other embodiments, a CH2CH3 or a CH3CH4 structure has sub-region domains from the same antibody isotype and are human, such as human IgG1, IgG2, IgG3, IgG4, IgA1, IgA2, IgD, IgE, or IgM (e.g., CH2CH3 from human IgG1 or IgG4).

By way of background, an Fc region is responsible for the effector functions of an immunoglobulin, such as ADCC (antibody-dependent cell-mediated cytotoxicity), CDC (complement-dependent cytotoxicity) and complement fixation, binding to Fc receptors (e.g., CD16, CD32, FcRn), greater half-life in vivo relative to a polypeptide lacking an Fc region, protein A binding, and perhaps even placental transfer (see Capon et al., Nature 337:525 (1989)). In certain embodiments, an Fc region portion found in fusion binding proteins of the present disclosure will be capable of mediating one or more of these effector functions, or will lack one or more or all of these activities by way of, for example, one or more mutations known in the art. For example, amino acid modifications (e.g., substitutions) to modify (e.g., improve, reduce, or ablate) Fc functionalities include the T250Q/M428L; M252Y/S254T/T256E; H433K/N434F; M428L/N434S; E233P/L234V/L235A/G236+A327G/A330S/P331S; E333A; S239D/A330L/I332E; P2571/Q311; K326W/E333S; S239D/I332E/G236A; N297Q; K322A; S228P; L235E+E318A/K320A/K322A; L234A/L235A; and L234A/L235A/P329G mutations, which mutations are summarized and annotated in “Engineered Fc Regions”, published by InvivoGen (2011) and available online at www. invivogen.com/PDF/review/review-Engineered-Fc-Regions-invivogen.pdf?utm_source=review&utm_medium=pdf&utm_campaign=review&utm_content=Engineered-Fc-Regions, and are incorporated herein by reference.

As used herein, an “immune system cell” means any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells and natural killer (NK) cells). Exemplary immune system cells include a CD4⁺ T cell, a CD8⁺ T cell, a CD4⁻ CD8⁻ double negative T cell, a γδ T cell, a regulatory T cell, a stem cell memory T cell, a natural killer cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

A “T cell” (or “T lymphocyte”) is an immune system cell that matures in the thymus and produces T cell receptors (TCRβ), which can be obtained (enriched or isolated) from, for example, peripheral blood mononuclear cells (PBMCs) and are referred to herein as “bulk” T cells. After isolation of T cells, both cytotoxic (CD8⁺) and helper (CD4⁺) T cells can be sorted into naïve, memory, and effector T cell subpopulations, either before or after expansion. T cells can be naïve (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD54RA as compared to naïve T cells), stem cell memory T cells, and effector memory T cells (T_{EM}, decreased expression of CD62L, CCR7, CD28, CD45RA, and increased expression of CD127 as compared to naïve T cells or T_{CM}). Effector T cells (T_{E}) refers to antigen-experienced CD8⁺ cytotoxic T lymphocytes that have decreased expression of CD62L, CCR7, CD28, and are positive for granzyme and perforin as compared to Tc. Helper T cells (T_{H}) are CD4⁺ cells that influence the activity of other immune cells by releasing cytokines. CD4⁺ T cells can activate and suppress an adaptive immune response, and which action is induced will depend on presence of other cells and signals. T cells can be collected in accordance with known techniques, and the various subpopulations or combinations thereof can be enriched or depleted by known techniques, such as by affinity binding to antibodies, flow cytometry, or immunomagnetic selection.

“T cell receptor” (TCR) refers to a molecule found on the surface of T cells (or T lymphocytes) that, in association with CD3, is generally responsible for recognizing antigens bound to major histocompatibility complex (MHC) molecules. The TCR has a disulfide-linked heterodimer of the highly variable α and β chains (also known as TCRα and TCRβ, respectively) in most T cells. In a subset of T cells, the TCR is made up of a heterodimer of variable γ and δ chains (also known as TCRγ and TCRδ, respectively). Each chain of the TCR is a member of the immunoglobulin superfamily and possesses one N-terminal immunoglobulin variable domain, one immunoglobulin constant domain, a transmembrane region, and a short cytoplasmic tail at the C-terminal end (see Janeway et al., Immunobiology: The Immune System in Health and Disease, 3′P Ed., Current Biology Publications, p. 4:33, 1997). TCR, as used in the present disclosure, may be from various animal species, including human, mouse, rat, cat, dog, goat, horse, or other mammals. TCRs may be cell-bound (i.e., have a transmembrane region or domain) or in soluble form.

The term “variable region” or “variable domain” of a TCR α-chain (Vα) and β-chain (Vβ), or Vγ and Vδ for γδ TCRβ, refer to those portions of a TCR that are involved in binding of the TCR to antigen (e.g., in a peptide antigen:MHC complex). The V_{α} and V_{β} of a native TCR generally have similar structures, with each variable domain comprising four conserved FRs and three CDRs. The V_{α} domain is encoded by two separate DNA segments, the variable gene segment and the joining gene segment (V-J); the V_{β} domain is encoded by three separate DNA segments, the variable gene segment, the diversity gene segment, and the joining gene segment (V-D-J). A single V_{α} or V_{β} domain may be sufficient to confer antigen-binding specificity. Furthermore, TCRs that bind a particular antigen may be isolated using a V_{α} or V_{β} domain from a TCR that binds the antigen to screen a library of complementary V_{α} or V_{β} domains, respectively.

“Major histocompatibility complex molecules” (MHC molecules) refer to glycoproteins that deliver peptide antigens to a cell surface. MHC class I molecules are heterodimers consisting of a membrane-spanning a chain (with three a domains) and a non-covalently associated β2 microglobulin. MHC class II molecules are composed of two transmembrane glycoproteins, α and β, both of which span the membrane. Each chain has two domains. MHC class I molecules deliver peptides originating in the cytosol to the cell surface, where peptide:MHC complex is recognized by CD8⁺ T cells. MHC class II molecules deliver peptides originating in the vesicular system to the cell surface, where they are recognized by CD4⁺ T cells. An MHC molecule may be from various animal species, including human, mouse, rat, or other mammals.

“Antigen” or “Ag” as used herein refers to an immunogenic molecule that provokes an immune response. This immune response may involve antibody production, activation of specific immunologically-competent cells (e.g., T cells), or both. An antigen (immunogenic molecule) may be, for example, a peptide, glycopeptide, polypeptide, glycopolypeptide, polynucleotide, polysaccharide, lipid or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen, or that endogenously (e.g., without modification or genetic engineering by human intervention) express a mutation or polymorphism that is immunogenic. In certain embodiments wherein a binding domain of a fusion protein comprises antigen-binding regions from a T cell receptor (TCRβ) (e.g., TCRVα and Vβ), an antigen comprises a peptide:MHC complex and the binding domain contacts at least the peptide.

The term “epitope” or “antigenic epitope” includes any molecule, structure, amino acid sequence or protein determinant that is recognized and specifically bound by a cognate binding molecule, such as an immunoglobulin, T cell receptor (TCR), chimeric antigen receptor, or other binding molecule, domain or fusion protein. Epitopic determinants generally contain chemically active surface groupings of molecules, such as amino acids or sugar side chains, and can have specific three dimensional structural characteristics, as well as specific charge characteristics.

As used herein, “nucleic acid” or “nucleic acid molecule” refers to any of deoxyribonucleic acid (DNA), ribonucleic acid (RNA), oligonucleotides, polynucleotides, fragments thereof generated, for example, by the polymerase chain reaction (PCR) or by in vitro translation, and also to fragments generated by any of ligation, scission, endonuclease action, or exonuclease action. In certain embodiments, the nucleic acids of the present disclosure are produced by PCR. Nucleic acids can be composed of monomers that are naturally occurring nucleotides (such as deoxyribonucleotides and ribonucleotides), analogs of naturally occurring nucleotides (e.g., α-enantiomeric forms of naturally occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acid molecules can be either single stranded or double stranded.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such a nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region (“leader and trailer”) as well as intervening sequences (introns) between individual coding segments (exons).

The term “construct” refers to any polynucleotide that contains a recombinant nucleic acid molecule. A construct may be present in a vector (e.g., a bacterial vector, a viral vector) or may be integrated into a genome.

A “vector” is a nucleic acid molecule that is capable of transporting another nucleic acid. Vectors may be, for example, plasmids, cosmids, viruses, phage, a RNA vector, or a linear or circular DNA or RNA molecule that may include chromosomal, non-chromosomal, semi-synthetic or synthetic nucleic acid molecules. Exemplary vectors are those capable of autonomous replication (episomal vector) or expression of nucleic acid molecules to which they are linked (expression vectors).

“Retroviruses” are viruses having an RNA genome. “Gammaretrovirus” refers to a genus of the retroviridae family. Exemplary gammaretroviruses include mouse stem cell virus, murine leukemia virus, feline leukemia virus, feline sarcoma virus, and avian reticuloendotheliosis viruses.

“Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

“Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

The term “expression”, as used herein, refers to the process by which a polypeptide is produced based on the encoding sequence of a nucleic acid molecule, such as a gene. The process can include transcription, post-transcriptional control, post-transcriptional modification, translation, post-translational control, post-translational modification, or any combination thereof.

As used herein, “expression vector” refers to a DNA construct containing a nucleic acid molecule that is operably-linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences that control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

The term “introduced” in the context of inserting a nucleic acid molecule into a cell, means “transfection”, or “transformation”, or “transduction” and includes reference to the incorporation of a nucleic acid molecule into a eukaryotic or prokaryotic cell wherein the nucleic acid molecule can be incorporated into the genome of a cell (e.g., a chromosome, a plasmid, a plastid, or a mitochondrial DNA), converted into an autonomous replicon, or transiently expressed (e.g., transfected mRNA).

As used herein, “heterologous” nucleic acid molecule, construct or sequence refers to a nucleic acid molecule or portion of a nucleic acid molecule that is not native to a host cell, but can be homologous to a nucleic acid molecule or portion of a nucleic acid molecule from the host cell. The source of the heterologous nucleic acid molecule, construct or sequence can be from a different genus or species. In certain embodiments, a heterologous nucleic acid molecule is added (i.e., not endogenous or native) to a host cell or host genome by, for example, conjugation, transformation, transfection, transduction, electroporation, or the like, wherein the added molecule can integrate into the host genome or exist as extra-chromosomal genetic material (e.g., as a plasmid or other form of self-replicating vector), and can be present in multiple copies. In addition, “heterologous” refers to a non-native enzyme, protein or other activity encoded by a non-endogenous nucleic acid molecule introduced into the host cell, even if the host cell encodes a homologous protein or activity.

The term “homologous” or “homolog” refers to a molecule or activity found in or derived from a host cell, species or strain. For example, a heterologous molecule or gene encoding the molecule may be homologous to a native host or host cell molecule or gene that encodes the molecule, respectively, and may optionally have an altered structure, sequence, expression level or combinations thereof.

As used herein, the term “endogenous” or “native” refers to a gene, protein, compound, molecule or activity that is normally present in a host or host cell. Moreover, a gene, protein or activity that is mutated, overexpressed, shuffled, duplicated or otherwise altered as compared to a parent gene, protein or activity is still considered to be endogenous or native to that particular host cell. For example, an endogenous control sequence from a first gene (e.g., a promoter, translational attenuation sequences) can be used to alter or regulate expression of a second native gene or nucleic acid molecule, wherein the expression or regulation of the second native gene or nucleic acid molecule differs from normal expression or regulation in a parent cell.

As used herein, the term “engineered,” “recombinant,” “modified” or “non-natural” refers to an organism, microorganism, cell, nucleic acid molecule, or vector that has been modified by introduction of an heterologous nucleic acid molecule, or refers to a cell or microorganism that has been genetically engineered by human intervention—that is, modified by introduction of a heterologous nucleic acid molecule, or refers to a cell or microorganism that has been altered such that expression of an endogenous nucleic acid molecule or gene is controlled, deregulated or constitutive, where such alterations or modifications can be introduced by genetic engineering. Human-generated genetic alterations can include, for example, modifications introducing nucleic acid molecules (which may include an expression control element, such as a promoter) encoding one or more proteins, fusion binding proteins, or enzymes, or other nucleic acid molecule additions, deletions, substitutions, or other functional disruption of or addition to a cell's genetic material. Exemplary modifications include those in coding regions or functional fragments thereof heterologous or homologous polypeptides from a reference or parent molecule. Additional exemplary modifications include, for example, modification s in non-coding regulatory regions in which the modifications alter expression of a gene or operon.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

“Sequence identity,” as used herein, refers to the percentage of amino acid residues (or nucleotides) in one sequence that are identical with the amino acid residues (or nucleotides) in another reference polypeptide (or nucleotide) sequence after aligning the sequences and introducing gaps, if necessary, to achieve the maximum percent sequence identity, and not considering any conservative substitutions (for amino acid sequences) as part of the sequence identity. The percentage sequence identity values can be generated using the NCBI BLAST 2.0 software as defined by Altschul et al., Nucl. Acids Res. 25:3389-3402 (1997), with the parameters set to default values.

“Adoptive cellular immunotherapy” or “adoptive immunotherapy” refers to the administration of naturally occurring or genetically engineered, disease antigen-specific immune cells (e.g., T cells). Adoptive cellular immunotherapy may be autologous (immune cells are from the recipient), allogeneic (immune cells are from a donor of the same species) or syngeneic (immune cells are from a donor genetically identical to the recipient).

“Treat” or “treatment” or “ameliorate” refers to medical management of a disease, disorder, or condition of a subject (e.g., a human or non-human mammal, such as a primate, horse, dog, mouse, rat). In general, an appropriate dose or treatment regimen comprising a host cell expressing a fusion binding protein, the fusion binding protein comprising an extracellular component comprising a binding domain that specifically binds a target antigen and an intracellular component comprising a modified CD28 costimulatory signaling domain of this disclosure, and a hydrophobic portion disposed between the extracellular component and intracellular component, is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; prolonged survival; or any combination thereof.

A “therapeutically effective amount” or “effective amount” of a fusion binding protein or cell expressing a fusion binding protein of this disclosure refers to that amount of compound or cells sufficient to result in amelioration of one or more symptoms of the disease being treated in a statistically significant manner. When referring to an individual active ingredient or a cell expressing a single active ingredient, administered alone, a therapeutically effective dose refers to the effects of that ingredient or cell expressing that ingredient alone. When referring to a combination, a therapeutically effective dose refers to the combined amounts of active ingredients or combined adjunctive active ingredient with a cell expressing an active ingredient that results in a therapeutic effect, whether administered serially or simultaneously. Another combination may be a cell expressing more than one active ingredient, such as two different fusion proteins, or other relevant therapeutic.

As used herein, the term “tonic” refers to a “basal” level of, or “antigen-independent”, signaling, which includes protein phosphorylation, activation, cytokine expression, proliferation, or a combination thereof, that occurs in an immune cell (e.g., T cell) in the absence of target antigen-specific activation via its cognate TCR or fusion protein (e.g., CAR).

### Fusion Proteins

Fusion proteins for use as adoptive immunotherapy compositions disclosed herein comprise a modified functional CD28 costimulatory signaling domain. The modified functional CD28 costimulatory signaling domain comprises at least one amino acid substitution. A fusion protein comprising such a modified functional CD28 costimulatory signaling domain has a functional activity that differs from a fusion protein comprising a wildtype CD28 costimulatory domain. For example, modifications to the CD28 costimulatory signaling domain provided herein may allow tailoring of functional activities including the fusion protein's activity, signaling kinetics, or signaling strength, thereby improving clinical efficacy, reducing toxicity (e.g., of a fusion protein-expressing host cell when administered to a subject), or both.

In certain aspects, the present disclosure provides a fusion protein, comprising an extracellular component comprising a binding domain that specifically binds a target antigen; an intracellular component comprising a modified functional CD28 costimulatory signaling domain, wherein the modified functional CD28 costimulatory signaling domain comprises at least one amino acid substitution; and a hydrophobic portion disposed between the extracellular component and intracellular component, wherein the fusion protein has a functional activity that differs from a fusion protein comprising wildtype CD28 costimulatory signaling domain. In certain embodiments, the fusion protein is expressed by a host cell and the functional activity comprises signaling kinetics (e.g., the timing, order, sequence, or rate of signaling), signaling intensity, cytokine production, cell proliferation, cell persistence, anti-antigen (e.g., anti-tumor cell) activity, tonic signaling, expression of immunosuppression component genes, or any combination thereof.

A binding domain suitable for use in a fusion binding protein of the present disclosure can be any antigen-binding polypeptide. A binding domain may comprise a natural antibody, synthetic or recombinant antibody construct, or an antigen-binding fragment thereof. For example, a binding domain may comprise a full length heavy chain, Fab fragment, Fab′, F(ab′)₂, variable heavy chain domain (V_{H} domain), variable light chain domain (V_{L} domain), domain antibody (dAb), single domain camelid antibody (VHH), complementary determining region (CDR), or single chain antibody fragment (scFv), and can, in some embodiments, be multispecific. Other examples of binding domains include single chain T cell receptors (scTCRs), soluble TCRβ, variable alpha chain domain (V_{α}), variable beta chain domain (V_{β}), extracellular binding domains of receptors, ligands for cell surface receptors/molecules, tumor binding proteins/peptides, and cytokines. In certain embodiments, a binding domain of a fusion binding protein of the present disclosure does not comprise an extracellular binding domain or moiety of CD8 or any portion thereof that comprises a functional IgV-like domain (i.e., an IgV-like domain capable binding a cognate ligand, such as a peptide:MHC complex). In particular embodiments, a binding domain of a fusion binding protein of the present disclosure does not comprise a binding domain from a CD8α chain, a binding domain from a CD8β chain, a binding domain from a CD8α homodimer, or a binding domain from a CD8αβ heterodimer. In further embodiments, a binding domain of a fusion binding protein of the present disclosure does not comprise a CD8α IgV-like domain as set forth in SEQ ID NO:43 or a CD80 IgV-like domain as set forth in SEQ ID NO:44.

In certain embodiments, a binding domain is murine, lapine, camelid, from a cartilaginous fish, chimeric, human, or humanized.

In certain embodiments, the binding domain comprises an scFv derived from anti-CD19 antibody FMC63 or anti-ROR1 antibody R12. In some embodiments, the binding domain comprises an a FMC63 scFv amino acid sequence as set forth in SEQ ID NO:8 or a R12 scFv amino acid sequence as set forth in SEQ ID NO:9.

Additional exemplary binding domains specific for ROR1 include those from antibodies disclosed in, for example, Yang et al., PLoS One 6:e21018 doi:10.1371, 2011; Paredes-Moscosso et al., Blood 128:2052, 2016; PCT Publication Nos. WO 2014/031174, WO 2016/094873, and WO2017072361A1; and U.S. Patents/Pre-Grant Publication Nos. US 2013/0251642, U.S. Pat. Nos. 9,316,646, 9,217,040, 9,242,014, 8,212,009, 9,226,952, 9,228,023, and 9,150,647. These antibodies and the binding domains thereof, including the amino acid sequences thereof, are incorporated herein by reference.

In certain embodiments, a binding domain that binds to a ROR1 antigen is derived from R12 antibody, R11 antibody, 2A2 antibody, R12 antibody, UC-961 antibody, D10 antibody, Y31 antibody, or H10 antibody.

An extracellular component of a fusion protein optionally comprises an extracellular, non-signaling spacer or linker region, which, for example, can position the binding domain away from the host cell (e.g., T cell) surface to enable proper cell/cell contact, antigen binding and activation (Patel et al., Gene Therapy 6: 412-419 (1999)). An extracellular spacer region of a fusion binding protein is generally located between a hydrophobic portion or transmembrane domain and the extracellular binding domain. Spacer region length may be varied to maximize antigen recognition (e.g., tumor recognition) based on the selected target molecule, selected binding epitope, or antigen-binding domain size and affinity (see, e.g., Guest et al., J. Immunother. 28:203-11 (2005); PCT Publication No. WO 2014/031687). In certain embodiments, a spacer region comprises an immunoglobulin hinge region. An immunoglobulin hinge region may be a wild-type immunoglobulin hinge region or an altered wild-type immunoglobulin hinge region. In certain embodiments, an immunoglobulin hinge region is a human immunoglobulin hinge region. An immunoglobulin hinge region may be an IgG, IgA, IgD, IgE, or IgM hinge region. An IgG hinge region may be an IgG1, IgG2, IgG3, or IgG4 hinge region. An exemplary altered IgG4 hinge region is described in PCT Publication No. WO 2014/031687, which hinge region, including the amino acid sequence thereof, is incorporated herein by reference in its entirety. In certain embodiments, an altered IgG4 hinge region comprises an amino acid sequence as set forth in SEQ ID NO:12. Other examples of hinge regions used in the fusion binding proteins described herein include the hinge region present in the extracellular regions of type 1 membrane proteins, such as CD8a, CD4, CD28 and CD7, which may be wild-type or variants thereof.

In certain embodiments, an extracellular spacer region comprises all or a portion of an Fc domain selected from: a CH1 domain, a CH2 domain, a CH3 domain, a CH4 domain, or any combination thereof (see, e.g., PCT Publication WO 2014/031687, which spacers are incorporated herein by reference in their entirety). The Fc domain or portion thereof may be wildtype of altered (e.g., to reduce antibody effector function). In certain embodiments, the extracellular component comprises an immunoglobulin hinge region, a CH2 domain, a CH3 domain, or any combination thereof disposed between the binding domain and the hydrophobic portion. In certain embodiments, the extracellular component comprises an IgG1 hinge region, an IgG1 CH2 domain, and an IgG1 CH3 domain. In further embodiments, the IgG1 CH2 domain comprises (i) a N297Q mutation, (ii) substitution of the first six amino acids (APEFLG) with APPVA, or both of (i) and (ii). In certain embodiments, the immunoglobulin hinge region, Fc domain or portion thereof, or both are human.

In certain embodiments, an extracellular spacer region further comprises a tag. A tag may be useful for determining whether cells expressing tagged fusion proteins used in adoptive cell therapies were successfully transferred to a subject in need thereof, or whether the cells expressing tagged fusion proteins proliferated, persisted, or localized to sites of interest in a subject receiving the adoptive cell therapy. A cell expressing a tagged fusion protein may be detected using an antibody or binding fragment thereof that specifically binds to the tag peptide. A tag may also be useful for enriching for or isolating a cell or population of cells expressing a tagged fusion protein from a subject or sample thereof (e.g., from whole blood, from PBMCs, or from a tumor tissue or site). A tag may also be useful for activating or expanding a cell or population of cells expressing a tagged fusion protein. A tag may be an enzyme, a dye, a fluorescent label, or a peptide tag. Exemplary tag peptides include Strep-Tag (WRHPQFGG, SEQ ID NO:39), Strep-Tag II (WSHPQFEK, SEQ ID NO:40), and Strep-Tag II 9-mer (NWSHPQFEK, SEQ ID NO:10), which bind the bacterial protein Streptavidin, and its derivative Strep-Tactin, with high affinity. See, e.g., U.S. Pat. No. 7,981,632 (Strep tags from which are incorporated herein by reference). Tagged fusion proteins (e.g., chimeric antigen receptors) containing one or more tag peptides and methods of detection, isolation, enrichment, activation, or expansion are described in PCT Publication No. WO 2015/095895, the tags, tagged fusion proteins, and methods of which are incorporated herein by reference. Other examples of tags include enzymes comprising a chromogenic reporter enzyme, such as horseradish peroxidase or alkaline phosphatase, cyanine dyes, coumarins, rhodamines, xanthenes, fluoresceins or sulfonated derivatives thereof, PE, Pacific blue, Alexa fluor, APC, FITC, fluorescent proteins, Myc tag, His tag, Flag tag, Xpress tag, Avi tag, Calmodulin tag, Polyglutamate tag, HA tag, Nus tag, S tag, X tag, SBP tag, Softag, V5 tag, CBP, GST, MBP, GFP, Thioredoxin tag, or any combination thereof.

A hydrophobic portion or transmembrane domain is disposed between the extracellular component and the intracellular component of the fusion protein. A transmembrane domain is a hydrophobic alpha helix that transverses and anchors the fusion protein in a host cell membrane (e.g., T cell). In certain embodiments, a transmembrane domain is selected from the same molecule from which the intracellular component is derived, such as CD28, an ITAM-containing T cell activating domain (e.g., CD3ζ, FcRγ) if present, or from another type I transmembrane protein, such as CD4, CD8, CD27. In certain embodiments, a transmembrane domain is selected from a different molecule from which the intracellular component is derived. In certain embodiments, the transmembrane domain comprises a transmembrane domain of CD28, CD2, CD3ε, CD3δ, CD3ζ, CD25, CD27, CD40, CD79A, CD79B, CD80, CD86, CD95 (Fas), CD134 (OX40), CD137 (4-1BB), CD150 (SLAMF1), CD152 (CTLA4), CD200R, CD223 (LAG3), CD270 (HVEM), CD272 (BTLA), CD273 (PD-L2), CD274 (PD-L1), CD278 (ICOS), CD279 (PD-1), CD300, CD357 (GITR), A2aR, DAP10, FcRα, FcRβ, FcRγ, Fyn, GAL9, KIR, Lck, LAT, LRP, NKG2D, NOTCH1, NOTCH2, NOTCH3, NOTCH4, PTCH2, ROR2, Ryk, Slp76, SIRPα, pTα, TCRα, TCRβ, TIM3, TRIM, LPA5, or Zap70. An exemplary CD28 transmembrane domain comprises an amino acid sequence of SEQ ID NO:13 or SEQ ID NO:41.

By way of background, robust activation of a T cell generally involves two distinct signaling events: (1) an antigen-specific signal provided through recognition of antigen by a T cell receptor (TCR) complex, which promotes T cell activation, and (2) a non antigen-specific “costimulatory signal” provided by the interaction between or the ligation of costimulatory molecules expressed on an antigen-presenting cell and a T cell. T cell activation in the absence of costimulation may result in anergy, apoptosis, or immune tolerance. A costimulatory signal stimulates T cells in conjunction with the antigen and promotes T cell proliferation, differentiation, and persistence.

An intracellular component refers to the portion of a fusion binding protein that transduces a signal to the inside of the host cell (e.g., T cell) in response to binding of the fusion protein to the target antigen, eliciting an effector function, e.g., activation, cytokine production, proliferation, differentiation, persistence, cytotoxic activity, homing, entry into the microenvironment of a tumor, or any combination thereof.

The intracellular component of fusion proteins of the present disclosure comprises a modified CD28 costimulatory signaling domain. CD28 is the major costimulatory receptor for naïve T cells and is involved in initiating T cell responses. CD28 binds to CD80 and CD86 expressed mainly on antigen presenting cells (e.g., dendritic cells, macrophages, B cells). Binding of CD28 with its ligands, in conjunction with T cell receptor signaling, promotes expansion of antigen-stimulated T cells and differentiation into effector and memory cells. CD28 signaling enhances cytokine production (e.g., IL-2), up-regulates cell survival genes (e.g., Bcl-xL), promotes energy metabolism, and facilitates cell cycle progression. A CD28 costimulatory signaling domain may refer to the full-length intracellular domain of CD28 or a truncated portion of the intracellular signaling domain, provided that the truncated portion retains signal transduction activity (e.g., at least about 25%, about 30%, about 35%, about 40%, about 45%, about 50%, about 55%, about 60%, about 65%, about 70%, about 75%, about 80%, about 85%, about 90%, about 95%, or substantially similar activity to wildtype CD28). An exemplary wildtype, full length human CD28 costimulatory signaling domain comprises an amino acid sequence of SEQ ID NO:2. A modified functional CD28 costimulatory signaling domain comprises at least one amino acid substitution, wherein a fusion protein comprising the modified functional CD28 costimulatory signaling domain will have a functional activity that differs from a fusion protein comprising a wildtype CD28 costimulatory signaling domain. In certain embodiments, a modified CD28 costimulatory signaling domain comprises at least 1, at least 2, at least about 3, at least about 4, at least about 5, at least about 6, at least about 7, at least about 8, at least about 9, at least about 10, at least about 11, at least about 12, at least about 13, at least about 14, or at least about 15 amino acids substitutions, provided that the modified CD28 costimulatory domain retains sufficient signal transduction activity (i.e., is a functional variant) to promote T cell activation In certain embodiments, a modified CD28 costimulatory signaling domain comprises at least about 15, about 16, about 17, about 18, about 19, about 20, about 25, or about 30 amino acid substitutions, provided that the modified CD28 costimulatory domain retains sufficient signal transduction activity (i.e., is a functional variant) to promote T cell activation. Exemplary assays for measuring functionality of a fusion protein include assays for measuring cytokine production (e.g., cytokine ELISA), T cell proliferation (e.g., by FACS), tumor cell killing activity (e.g., using an in vitro labeled chromium release assay (CRA), or by imaging tumor size in vivo), T cell surface expression of costimulatory markers, and absence (or presence) of T cell exhaustion markers in response to antigen stimulation (e.g., by using detectably labeled antibodies).

In certain embodiments, an amino acid of the CD28 costimulatory signaling domain is substituted with any amino acid other than the amino acid that is naturally occurring at that position in the wild-type CD28 costimulatory signaling domain. In certain embodiments, an amino acid is substituted with a naturally occurring amino acid or a non-naturally occurring amino acid.

A “conservative substitution” is recognized in the art as a substitution of one amino acid for another amino acid that has similar properties (e.g., another naturally occurring or a synthetically produced amino acid or a mimetic thereof). In certain embodiments, an amino acid substitution is a conservative amino acid substitution. Exemplary conservative amino acid substitutions comprise ones in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e.g., alanine, valine, leucine, isoleucine, proline, phenylalanine, methionine, tryptophan); amino acids with beta-branched side chains (e.g., threonine, valine, isoleucine), and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In certain circumstances, substitution of glutamine for glutamic acid or asparagine for aspartic acid may be considered a similar substitution in that glutamine and asparagine are amide derivatives of glutamic acid and aspartic acid, respectively. Further exemplary conservative substitutions are well known in the art (see, e.g., PCT Publication No. WO 97/09433 at page 10; Lehninger, Biochemistry, 2^{nd} Edition; Worth Publishers, Inc. NY, N.Y., pp. 71-77, 1975; Lewin, Genes IV, Oxford University Press, NY and Cell Press, Cambridge, Mass., p. 8, 1990), which conservative substitutions are incorporated herein by reference in their entirety.

In certain embodiments, although the full-length CD28 molecule may not be present in the fusion protein, for the purpose of reference, an amino acid substitution may refer to the position of the amino acid residue within the full-length CD28 polypeptide. In specific embodiments, an amino acid substitution refers to the position of the amino acid residue within the full-length human CD28 polypeptide as set forth in UniProt: P10747 (SEQ ID NO:1).

In certain embodiments, the modified CD28 costimulatory signaling domain comprises at least one amino acid substitution, wherein: at least one (i.e., one or more) tyrosine residue is substituted with a different amino acid residue, at least one proline residue is substituted with a different amino acid residue, or both. In some embodiments, at least one, two, three, or four tyrosine residues are substituted. For modified CD28 costimulatory signaling domains comprising two or more tyrosine substitutions, each tyrosine substitution may be the same or different. In further embodiments, the at least one tyrosine residue is substituted with a conservative amino acid. In a specific embodiment, the at least one tyrosine residue is substituted with a phenylalanine residue. In another specific embodiment, the at least one tyrosine residue is substituted with a tryptophan residue. In some embodiments, at least one tyrosine residue is substituted with a tryptophan residue and at least one tyrosine residue is substituted with a phenylalanine residue.

In some embodiments, at least one, two, three, or four proline residues are substituted. For modified CD28 costimulatory signaling domains comprising two or more proline substitutions, each proline substitution may be the same or different. In further embodiments, the at least one proline residue is substituted with a conservative amino acid. In a specific embodiment, the at least one proline residue is substituted with an alanine residue. In some embodiments, at least one proline residue is substituted with an alanine residue and at least one proline residue is substituted with a different (non-alanine) amino acid.

In certain embodiments, at least one tyrosine residue for substitution is selected from any one of positions 191, 206, 209, and 218 (positions are referencing the full length, wild-type human CD28 as set forth in SEQ ID NO:1). In some embodiments, at least two, three, or four tyrosine residues selected from any one of positions 191, 206, 209, and 218 are substituted. In some embodiments, the at least one tyrosine residue is substituted with a conservative amino acid, e.g., phenylalanine.

In certain embodiments, the at least one proline residue for substitution is selected from any one of positions 196, 199, 208, and 211 (positions are referencing the full length, wild-type human CD28 as set forth in SEQ ID NO:1). In some embodiments, at least two, three, or four proline residues selected from any of positions 196, 199, 208, and 211 are substituted. In some embodiments, the at least one proline residue is substituted with a conservative amino acid, e.g., alanine.

In some embodiments, the modified CD28 costimulatory signaling domain comprises a Y191, Y206, Y209, Y218, Y191/Y206, Y191/Y209, Y191/Y218, Y206/Y209, Y206/Y218, Y209/Y218, Y191/Y206/Y209, Y191/Y206/Y218, Y191/Y209/Y218, Y206/Y209/Y218, or Y191/Y206/Y209/Y218 substitution(s).

In some embodiments, the modified CD28 costimulatory signaling domain comprises a P196, P199, P208, P211, P196/P199, P196/P208, P196/P211, P199/P208, P199/P211, P208/P211, P196/P199/P208, P196/P199/P211, P196/P208/P211, P199/P208/P211, or P196/P199/P208/P211 substitution(s).

In some embodiments, the modified CD28 costimulatory domain comprises at least one tyrosine substitution selected from any one of: Y191, Y206, Y209, Y218, Y191/Y206, Y191/Y209, Y191/Y218, Y206/Y209, Y206/Y218, Y209/Y218, Y191/Y206/Y209, Y191/Y206/Y218, Y191/Y209/Y218, Y206/Y209/Y218, or Y191/Y206/Y209/Y218; and at least one proline substitution selected from any one of: P196, P199, P208, P211, P196/P199, P196/P208, P196/P211, P199/P208, P199/P211, P208/P211, P196/P199/P208, P196/P199/P211, P196/P208/P211, P199/P208/P211, or P196/P199/P208/P211.

In further embodiments, the modified CD28 costimulatory signaling domain comprises a Y191F, Y206F, Y209F, Y218F, Y191F/Y206F, Y191F/Y209F, Y191F/Y218F, Y206F/Y209F, Y206F/Y218F, Y209F/Y218F, Y191F/Y206F/Y209F, Y191F/Y206F/Y218F, Y191F/Y209F/Y218F, Y206F/Y209F/Y218F, or Y191F/Y206F/Y209F/Y218F substitution(s).

In some embodiments, the modified CD28 costimulatory signaling domain comprises a P196A, P199A, P208A, P211A, P196A/P199A, P196A/P208A, P196A/P211A, P199A/P208A, P199A/P211A, P208A/P211A, P196A/P199A/P208A, P196A/P199A/P211A, P196A/P208A/P211A, P199A/P208A/P211A, or P196A/P199A/P208A/P211A substitution(s).

In some embodiments, the modified CD28 costimulatory domain comprises at least one tyrosine substitution selected from any one of: Y191F, Y206F, Y209F, Y218F, Y191F/Y206F, Y191F/Y209F, Y191F/Y218F, Y206F/Y209F, Y206F/Y218F, Y209F/Y218F, Y191F/Y206F/Y209F, Y191F/Y206F/Y218F, Y191F/Y209F/Y218F, Y206F/Y209F/Y218F, or Y191F/Y206F/Y209F/Y218F; and at least one proline substitution selected from any one of: P196A, P199A, P208A, P211A, P196A/P199A, P196A/P208A, P196A/P211A, P199A/P208A, P199A/P211A, P208A/P211A, P196A/P199A/P208A, P196A/P199A/P211A, P196A/P208A/P211A, P199A/P208A/P211A, or P196A/P199A/P208A/P211A.

In any of the modified CD28 costimulatory signaling domains described herein, the modified CD28 costimulatory signaling domain may further comprise a substitution at each of positions L186 and L187. In some embodiments, the modified CD28 costimulatory domain comprises a L186G/L187G substitution. The di-leucine to di-glycine substitutions at positions 186 and 187 have been shown to increase fusion protein expression in the host immune cell (see, Nguyen et al., Blood 102:4320-4325 (2003), which substitution mutations are incorporated herein by reference).

In certain embodiments, the modified CD28 costimulatory signaling domain does not comprise a substitution at Y191, P208, P211, or any combination thereof.

Exemplary amino acid sequences of modified CD28 costimulatory signaling domains are provided in SEQ ID NOs:4-6 and 45-47.

A fusion protein comprising a modified CD28 costimulatory signaling domain described in the present disclosure exhibits modulated functional activity in an immune cell expressing said fusion protein as compared to a fusion protein comprising a “wild-type” CD28 costimulatory signaling domain. Modulation of functional activity of an immune cell expressing the fusion protein may include modulation of signaling kinetics (e.g., the timing, order, sequence, or rate of signaling), signaling intensity, cytokine production, cell proliferation, cell persistence, anti-tumor activity, tonic signaling, expression of immunosuppression component genes, or any combination thereof. In certain embodiments, a modified CD28 costimulatory signaling domain reduces cytokine production in an immune cell expressing the fusion protein. Examples of cytokines whose expression may be reduced include IL-2 and TNF-α. Methods of measuring cytokine levels are known in the art and include quantification by ELISA, Western blot, antibody array, flow cytometry, and cytometric bead array.

In certain embodiments, a modified CD28 costimulatory signaling domain reduces tonic signaling in an immune cell expressing the fusion protein. Tonic signaling may comprise tonic protein phosphorylation, activation, cytokine expression, proliferation, or a combination thereof. In a specific embodiment, a modified CD28 costimulatory signaling domain reduces tonic phosphorylation of CD3ζ, for instance, at position Y142, in a T cell expressing said fusion protein.

An intracellular component optionally further comprises an intracellular activation domain from a receptor, such as an ITAM-containing T cell activating domain. An ITAM-containing T cell activating motif used in fusion proteins of the instant disclosure can be identical to, or can be a functional variant of, an intracellular signaling domain or portion thereof of an immune cell receptor, or of a cell surface marker containing at least one ITAM. In general, an ITAM-containing T cell activating domain provides a T cell activation signal upon engagement of a fusion protein's binding domain with its target antigen. Non-limiting examples of ITAM-containing intracellular activating domains that may be used in the fusion proteins described herein include those present on CD3γ, CD3δ, CD3ε, CD3ζ, FcRγ, CD38, CD5, CD22, CD79a, CD79b and CD66d, gamma chain of FcεRI or FcγRI, FcRγ2a, FcRγ2b1, FcRγ2a1, FcRγ2b2, FcRγ3a, FcRγ3b, FcRβ1, FcεR), a Natural Killer cell receptor protein (e.g., DAP12), CD5, CD16a, CD16b, CD22, CD23, CD32, CD64, CD89, and CD278. In particular embodiments, the intracellular signaling component of a fusion protein of the present disclosure comprises a CD3ζ ITAM-containing T cell activating domain. An exemplary CD3ζ ITAM-containing T cell activating domain comprises the amino acid sequence of SEQ ID NO:15. In certain embodiments, an intracellular component of a fusion protein of the present disclosure comprises a modified CD28 costimulatory signaling domain and a CD3ζ ITAM-containing T cell activating domain.

An intracellular component optionally further comprises an additional costimulatory signaling domain other than the CD28 costimulatory signaling domain. The additional costimulatory signaling domain may comprise a full-length intracellular domain of a costimulatory molecule other than CD28 or a truncated portion of the intracellular signaling domain, provided that the truncated portion retains sufficient signal transduction activity. In certain embodiments, the additional costimulatory signaling domain is selected from CD27, CD40L, GITR, NKG2C, CARD1, CD2, CD7, CD27, CD30, CD40, CD54 (ICAM), CD83, CD134 (OX-40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, ZAP70, CD5, BAFF-R, SLAMF7, NKp80, CD160, B7-H3, a ligand that specifically binds with CD83, or a combination thereof. In certain embodiments, an intracellular component of a fusion protein of the present disclosure comprises a modified CD28 costimulatory signaling domain, a CD3ζ ITAM-containing T cell activating domain, and an additional costimulatory signaling domain selected from CD27, CD40L, GITR, NKG2C, CARD1, CD2, CD7, CD27, CD30, CD40, CD54 (ICAM), CD83, CD134 (OX-40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP 10, LAT, NKD2C SLP76, TRIM, ZAP70, CD5, BAFF-R, SLAMF7, NKp80, CD160, B7-H3, a ligand that specifically binds with CD83, or a combination thereof. In a specific embodiment, comprises a modified CD28 costimulatory signaling domain, a CD3ζ ITAM-containing T cell activating domain, and a 4-1BB costimulatory signaling domain. An exemplary 4-1BB costimulatory signaling domain comprises an amino acid sequence of SEQ ID NO:14.

Fusion proteins of the present disclosure may be in the form of a chimeric antigen receptor (CAR), chimeric costimulatory receptor (CCR), a split-CAR or on-switch CAR, a single chain T cell receptor (scTCR or scTv) linked to an intracellular signaling domain, or TCR-CAR. In some embodiments, a CAR generally has a single intracellular signaling domain providing an activation signal (e.g., intracellular signaling domain of CD3ζ or FcγRI or other ITAM-containing T cell activating domain). In some embodiments, CARs further include an intracellular costimulatory signaling domain (e.g., a costimulatory signaling domain from an endogenous T cell costimulatory receptor, such as CD28, 4-1BB, or ICOS). In some embodiments, CARs further include a second costimulatory domain. A CCR is similar in design to a CAR and provides a costimulation through a costimulatory signaling domain but does not comprise an ITAM-containing T cell activating domain. A CCR may further comprise a heterodimerization domain for co-expression in a host cell with a polypeptide comprising an intracellular activation domain and a corresponding heterodimerization domain for assembly upon administration of an appropriate heterodimerization agent (e.g., split-CAR or on-switch CAR design). A TCR-CAR is a heterodimeric fusion protein generally comprising a soluble TCR (a V_{α}C_{α} polypeptide chain and a V_{β}C_{β} polypeptide chain) wherein the V_{β}C_{β} polypeptide chain is linked to a transmembrane domain and an intracellular signaling component (e.g., comprising an ITAM-containing T cell activating domain and optionally a costimulatory signaling domain). A scTCR fusion protein comprises a binding domain comprising a scTCR (TCR V_{α} linked to V_{β}), an optional extracellular spacer, a transmembrane domain, and an intracellular component comprising a single intracellular signaling domain providing an T cell activation signal (e.g., a CD3ζ ITAM-containing T cell activating domain) and optionally a costimulatory signaling domain (see, Aggen et al., Gene Ther. 19:365-374 (2012); Stone et al., Cancer Immunol. Immunother. 63:1163-76 (2014)).

In certain embodiments, fusion proteins described herein comprise binding domains that target an antigen from a pathogen, an autoimmune disease associated antigen, a cancer antigen, or a self-antigen. Examples of pathogen-associated or pathogen-specific antigens include viral antigens (e.g., HIV antigens, HCV antigens, HBV antigens, CMV antigens, HPV antigens, EBV antigens, influenza antigens, respiratory syncytial virus antigens), parasitic antigens, and bacterial antigens. A cancer antigen may be any antigen of clinical interest against which it would be desirable to trigger a cell-mediated immune response that results in cancer cell or tumor killing. Non-limiting examples of cancer antigens that may be targeted by a fusion protein include BCMA, CD3, CEACAM6, c-Met, EGFR, EGFRvIII, ErbB2, ErbB3, ErbB4, EphA2, IGF1R, GD2, O-acetyl GD2, O-acetyl GD3, GHRHR, GHR, FLT1, KDR, FLT4, CD44v6, CD151, CA125, CEA, CTLA-4, GITR, BTLA, TGFBR2, TGFBR1, IL6R, gp130, Lewis A, Lewis Y, TNFR1, TNFR2, PD1, PD-L1, PD-L2, HVEM, MAGE-A (e.g., including MAGE-A1, MAGE-A3, and MAGE-A4), mesothelin, NY-ESO-1, PSMA, RANK, ROR1, TNFRSF4, CD40, CD137, TWEAK-R, HLA, tumor- or pathogen-associated peptide bound to HLA, hTERT peptide bound to HLA, tyrosinase peptide bound to HLA, WT-1 peptide bound to HLA, LTβR, LIFRβ, LRP5, MUC1, OSMRβ, TCRα, TCRβ, CD19, CD20, CD22, CD25, CD28, CD30, CD33, CD52, CD56, CD79a, CD79b, CD80, CD81, CD86, CD123, CD171, CD276, B7H4, TLR7, TLR9, PTCH1, WT-1, HA¹-H, Robol, α-fetoprotein (AFP), Frizzled, OX40, PRAME, and SSX-2.

In any of the embodiments provided herein, a fusion protein may be a “universal chimeric antigen receptor.” A universal CAR comprises a binding domain that binds to a tag, rather than to a disease-associated antigen. Modified immune cells comprising a universal CAR may be redirected to the disease-associated antigen by administering a tagged protein that binds to the disease-associated antigen (e.g., a tagged antibody that binds to a disease-associated antigen). A tag may be a protein, a peptide, a small molecule, or a hapten. Exemplary tags include a peptide derived from a hormone, a peptide derived from a ligand, a peptide derived from a cytokine, a peptide derived from a chemokine, a peptide derived from a growth factor, a peptide derived from a cell adhesion molecule, a signaling peptide, a peptide derived from a receptor, a cell surface peptide, fluorescein isothiocyanate (FITC), dinitrophenol, peridinin chlorophyll protein complex, green fluorescent protein, biotin, phycoerythrin (PE), histidine, streptavidin, horse radish peroxidase, palmitoylation, nitrosylation, alkaline phosphatase, glucose oxidase, Glutathione S-transferase, maltose binding protein, DOTA, dinitrophenol, quinone, biotin, aniline, atrazine, an aniline-derivative, o-aminobenzoic acid, p-aminobenzoic acid, m-aminobenzoic acid, hydralazine, halothane, digoxigenin, benzene arsonate, lactose, trinitrophenol, a hapten, a steroid, a vitamin, a vitamer, a metabolite, an antibiotic, a monosaccharide, a disaccharide, a lipid, a fatty acid, a nucleic acid, an alkaloid, a glycoside, a phenzine, a polyketide, a terpene, a tetrapyrrole, and a peptide derived from a human nuclear protein (e.g., human nuclear La protein (E5B9)). Universal CARs and methods of making and using the same are known in the art and described, for example, in U.S. Pat. No. 9,233,125; PCT Publication No. WO2013/044225; PCT Publication No. WO2016/168766; PCT Publication No. WO2016/168773; and U.S. Patent Publication No. 2017/0240612, each of which CARs and related methods is incorporated herein by reference in its entirety.

In some embodiments, a CAR of the present disclosure comprises an extracellular component comprising a binding domain specific for CD19 or ROR1; optionally a tag (i.e., a tag that is different than the tag bound by a universal CAR when the fusion protein is a universal CAR); an immunoglobulin hinge region; a hydrophobic portion; and an intracellular component comprising an ITAM-containing T cell activating domain and a modified CD28 costimulatory signaling domain or functional portion thereof, wherein the modified CD28 costimulatory signaling domain or functional portion thereof comprises a substitution as disclosed herein at one or more of positions L186, L187, Y191, Y206, Y209, Y218, P196, P199, P208, and P211. In particular embodiments, the modified CD28 costimulatory signaling domain or functional portion thereof comprises substitutions at positions: (a) L186, L187, and Y218; (b) L186, L187, Y206, Y209, and Y218; (c) L186, L187, Y191, Y206, Y209, and Y218; (d) L186G, L187G, and Y218F; (e) L186G, L187G, Y206F, Y209F, and Y218F; or (f) L186G, L187G, Y191F, Y206F, Y209F, and Y218F. In any of the aforementioned embodiments, the tag comprises a Strep tag II.

In certain embodiments, a CAR of the present disclosure comprises an extracellular component comprising a FMC63 (anti-CD19) scFv, a Strep tag II peptide, an altered IgG4 hinge region; a hydrophobic portion comprising a CD28 transmembrane domain; a modified CD28 costimulatory signaling domain comprising L186G, L187G, and Y218F substitutions; and an ITAM-containing T-cell activating domain comprising a CD3ζ intracellular signaling domain. Such a CAR (e.g., a polypeptide encoded by a CAR expression construct) may comprise the amino acid sequence of SEQ ID NO:27 (including the signal peptide at amino acids 1-22) or SEQ ID NO:27 without amino acids 1-22. In further embodiments, a CAR can lack the T2A and tEGFR amino acid sequences of SEQ ID NO:27.

In further embodiments, a CAR of the present disclosure comprises an extracellular component comprising a FMC63 (anti-CD19) scFv, a Strep tag II peptide, an altered IgG4 hinge region; a hydrophobic portion comprising a CD28 transmembrane domain; a modified CD28 costimulatory signaling domain comprising L186G, L187G, Y206F, Y209F, and Y218F substitutions; and an ITAM-containing T-cell activating domain comprising a CD3ζ intracellular signaling domain. Such a CAR (e.g., a polypeptide encoded by a CAR expression construct) may comprise the amino acid sequence of SEQ ID NO:29 (including the signal peptide at amino acids 1-22) or SEQ ID NO:29 without amino acids 1-22. In further embodiments, a CAR can lack the T2A and tEGFR amino acid sequences of SEQ ID NO:29.

In still further embodiments, a CAR of the present disclosure comprises an extracellular component comprising a FMC63 (anti-CD19) scFv, a Strep tag II peptide, an altered IgG4 hinge region; a hydrophobic portion comprising a CD28 transmembrane domain; a modified CD28 costimulatory signaling domain comprising L186G, L187G, Y191F, Y206F, Y209F, and Y218F substitutions; and an ITAM-containing T-cell activating domain comprising a CD3ζ intracellular signaling domain. Such a CAR (e.g., a polypeptide encoded by a CAR expression construct) may comprise the amino acid sequence of SEQ ID NO:35 (including the signal peptide at amino acids 1-22) or SEQ ID NO:35 without amino acids 1-22. In further embodiments, a CAR can lack the T2A and tEGFR amino acid sequences of SEQ ID NO:35.

In yet further embodiments, a CAR of the present disclosure comprises an extracellular component comprising a R12 (anti-ROR1) scFv, a Strep tag II peptide, an altered IgG4 hinge region; a hydrophobic portion comprising a CD28 transmembrane domain; a modified CD28 costimulatory signaling domain comprising L186G, L187G, and Y218F substitutions; and an ITAM-containing T-cell activating domain comprising a CD3ζ intracellular signaling domain. Such a CAR (e.g., a polypeptide encoded by a CAR expression construct) may comprise the amino acid sequence of SEQ ID NO:31 (including the signal peptide at amino acids 1-22) or SEQ ID NO:31 without amino acids 1-22. In further embodiments, a CAR can lack the T2A and tEGFR amino acid sequences of SEQ ID NO:31.

In more embodiments, a CAR of the present disclosure comprises an extracellular component comprising a R12 (anti-ROR1) scFv, a Strep tag II peptide, an altered IgG4 hinge region; a hydrophobic portion comprising a CD28 transmembrane domain; a modified CD28 costimulatory signaling domain comprising L186G, L187G, Y206F, Y209F, and Y218F substitutions; and an ITAM-containing T-cell activating domain comprising a CD3ζ intracellular signaling domain. Such a CAR (e.g., a polypeptide encoded by a CAR expression construct) may comprise the amino acid sequence of SEQ ID NO:33 (including the signal peptide at amino acids 1-22) or SEQ ID NO:33 without amino acids 1-22. In further embodiments, a CAR can lack the T2A and tEGFR amino acid sequences of SEQ ID NO:33.

In still more embodiments, a CAR of the present disclosure comprises an extracellular component comprising a R12 (anti-ROR1) scFv, a Strep tag II peptide, an altered IgG4 hinge region; a hydrophobic portion comprising a CD28 transmembrane domain; a modified CD28 costimulatory signaling domain comprising L186G, L187G, Y191F, Y206F, Y209F, and Y218F substitutions; and an ITAM-containing T-cell activating domain comprising a CD3ζ intracellular signaling domain. Such a CAR (e.g., a polypeptide encoded by a CAR expression construct) may comprise the amino acid sequence of SEQ ID NO:37 (including the signal peptide at amino acids 1-22) or SEQ ID NO:37 without amino acids 1-22. In further embodiments, a CAR can lack the T2A and tEGFR amino acid sequences of SEQ ID NO:37.

In some embodiments, a CAR of the present disclosure may comprise an amino sequence having at least 90%, 91%, 92%, 93%, 94%, 95%, 96%, 97%, 98%, 99%, or 100% identity to the amino acid sequence of any one of SEQ ID NOs:64-69 (including or without the signal peptide at amino acids 1-22 of SEQ ID NOs: 64-69, respectively).

In some embodiments, a CAR (e.g., a polypeptide encoded by a CAR expression construct) of the present disclosure can comprise an amino sequence having at least 90%, 91%, 92%, 93%, 94%, 95%, 96%, 97%, 98%, 99%, or 100% identity to the amino acid sequence of any one of SEQ ID NOs:49, 51, 53, 55, 57, or 59 (including or without the signal peptide at amino acids 1-22 of SEQ ID NOs: 49, 51, 53, 55, 57, or 59, respectively). In further embodiments, a CAR can lack the T2A and tEGFR amino acid sequences of SEQ ID NOs: 49, 51, 53, 55, 57, or 59.

### Polynucleotides, Vectors, and Host Cells

In certain aspects, nucleic acid molecules are provided that encode any one or more fusion proteins described herein. A polynucleotide encoding a desired fusion protein can be accomplished by using any suitable molecular biology engineering technique(s), including the use of restriction endonuclease digestion, ligation, transformation, plasmid purification, and DNA sequencing as described in, for example, Sambrook et al. (1989 and 2001 editions; Molecular Cloning: A Laboratory Manual, Cold Spring Harbor Laboratory Press, NY) and Ausubel et al. (Current Protocols in Molecular Biology, 2003). Alternatively, a sequence of interest can be produced synthetically. To obtain efficient transcription and translation, a polynucleotide in each recombinant expression construct includes at least one appropriate expression control sequence (also called a regulatory sequence), such as a leader sequence and particularly a promoter operably (i.e., operatively) linked to the nucleotide sequence encoding the immunogen.

A nucleic acid of this disclosure may refer to a single- or a double-stranded DNA, cDNA or RNA in any form, and may include a positive and a negative strand of the nucleic acid which complement each other, including anti-sense DNA, cDNA and RNA. Also included are siRNA, microRNA, RNA-DNA hybrids, ribozymes, and other various naturally occurring or synthetic forms of DNA or RNA.

In any of the embodiments described herein, a polynucleotide of the present disclosure may be codon optimized for efficient expression in a host cell containing the polynucleotide (see, e.g, Scholten et al., Clin. Immunol. 119:135-145 (2006)). As used herein, a “codon optimized” polynucleotide comprises a heterologous polynucleotide having codons modified with silent mutations corresponding to the abundances of tRNA levels in a host cell of interest.

In certain embodiments, a polynucleotide encoding a fusion protein of the present disclosure comprises a polynucleotide having at least about 75%, 80%, 85%, 90%, 91%, 92%, 93%, 94%, 95%, 96%, 97%, 98%, 99%, or 100% identity to the fusion protein-encoding nucleotide sequence set forth in any one of SEQ ID NOs:18, 20, 22, 24, 26, 28, 30, 32, 34, 36, 48, 50, 52, 54, 56, or 58, and optionally comprises or consists of a polynucleotide set forth in any one of SEQ ID NOs:18, 20, 22, 24, 26, 28, 30, 32, 34, 36, 48, 50, 52, 54, 56, or 58.

A single polynucleotide molecule may encode one, two, or more fusion proteins according to any of the embodiments disclosed herein. A polynucleotide encoding more than one transcript may comprise a sequence (e.g., a viral 2A peptide-encoding sequence) disposed between each transcript for multicistronic expression. In certain embodiments, a fusion protein-encoding polynucleotide of the present disclosure may be operatively linked to one or more certain elements of a vector. For example, polynucleotide sequences that are needed to effect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

Certain embodiments include a polynucleotide of the present disclosure contained in a vector. An exemplary vector may comprise a nucleic acid molecule capable of transporting another nucleic acid molecule to which it has been linked, or which is capable of replication in a host organism. Some examples of vectors include plasmids, viral vectors, cosmids, and others. Some vectors may be capable of autonomous replication in a host cell into which they are introduced (e.g. bacterial vectors having a bacterial origin of replication and episomal mammalian vectors), whereas other vectors may be integrated into the genome of a host cell or promote integration of the polynucleotide insert upon introduction into the host cell and thereby replicate along with the host genome (e.g., lentiviral vector). Additionally, some vectors are capable of directing the expression of genes to which they are operatively linked (these vectors may be referred to as “expression vectors”). According to related embodiments, it is further understood that, if one or more agents (e.g., polynucleotides encoding fusion proteins as described herein) is co-administered to a subject, that each agent may reside in separate or the same vectors, and multiple vectors (each containing a different agent or the same agent) may be introduced to a cell or cell population or administered to a subject.

A vector may be, for example, a plasmid, cosmid, virus, a RNA vector, or a linear or circular DNA or RNA molecule that may include chromosomal, non-chromosomal, semi-synthetic or synthetic nucleic acid molecules. Exemplary vectors are those capable of autonomous replication (episomal vector) or expression of nucleic acid molecules to which they are linked (expression vectors).

Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

The viral vector can, in certain embodiments, be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing CAR transgenes are known in the art and have been previously described, for example, in: U.S. Pat. No. 8,119,772; Walchli et al., PLoS One 6:327930 (2011); Zhao et al., J. Immunol. 174:4415 (2005); Engels et al., Hum. Gene Ther. 14:1155 (2003); Frecha et al., Mol. Ther. 18:1748 (2010); and Verhoeyen et al., Methods Mol. Biol. 506:97 (2009). Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517 (1998)).

Other vectors developed for gene therapy uses can also be used with the compositions and methods of this disclosure. Such vectors include those derived from baculoviruses and α-viruses (Jolly, D J. 1999. Emerging Viral Vectors. pp. 209-40 in Friedmann T. ed. The Development of Human Gene Therapy. New York: Cold Spring Harbor Lab), or plasmid vectors (such as sleeping beauty or other transposon vectors).

To obtain efficient transcription and translation, a polynucleotide in each recombinant expression construct includes at least one appropriate expression control sequence (also called a regulatory sequence), such as a leader sequence and particularly a promoter operably (i.e., operatively) linked to the nucleotide sequence encoding the immunogen.

Markers are sometimes used to identify or monitor expression of a heterologous polynucleotide by a host cell transduced with the same, or to detect cells expressing a fusion protein of interest. In certain embodiments, a polynucleotide encoding a fusion protein further comprises a polynucleotide that encodes a marker. A marker may be a selection marker, which confers drug resistance, or a detectable marker, such as a fluorescent marker or cell surface protein that can be detected by methods such as flow cytometry. In certain embodiments, the polynucleotide encoding the marker is located 3′ of the polynucleotide encoding the immunoglobulin binding protein or the fusion protein. In other embodiments, the polynucleotide encoding the marker is located 5′ of the polynucleotide encoding the immunoglobulin binding protein or the fusion protein. Exemplary markers include green fluorescent protein (GFP), an extracellular domain of human CD2, a truncated human EGFR (huEGFRt; see Wang et al., Blood 118:1255 (2011)), a truncated human CD19 (huCD19t), a truncated human CD34 (huCD34t); or a truncated human NGFR (huNGFRt). In certain embodiments, the encoded marker comprises EGFRt, CD19t, CD34t, or NGFRt. An exemplary truncated human EGFR sequence comprises an amino acid sequence of SEQ ID NO:17.

In certain embodiments, the vector may further comprise a suicide gene, where expression of the suicide gene results in the death of the host cell comprising the vector. For example, in some instances, prolonged expression of the fusion protein of the invention is not desirable. Inclusion of a suicide gene in the vector allows for finer control of fusion protein expression in a subject. In certain embodiments, expression of the suicide gene is inducible, for example, with the use of an inducible promoter regulating suicide gene expression. In a specific embodiment, a suicide gene is an inducible caspase-9 gene (see US Pre-Grant Patent Publication No. US 2013/0071414, which suicide genes are incorporated by reference herein). Other suicide genes include a gene that encodes any one or more of: a conformationally intact binding epitope for pharmaceutical-grade anti-EGFR monoclonal antibody, cetuximab (Erbitux); EGFRt, a caspase polypeptide (e.g., iCasp9; Straathof et al., Blood 105:4247-4254, 2005; Di Stasi et al., N. Engl. J. Med. 365:1673-1683, 2011; Zhou and Brenner, Exp. Hematol. pii:S0301-472X(16)30513-6. doi:10.1016/j.exphem.2016.07.011), RQR8 (Philip et al., Blood 124:1277-1287, 2014), a 10-amino acid tag of the human c-myc protein (Myc) (Kieback et al., Proc. Natl. Acad. Sci. USA 105:623-628, 2008), as discussed herein, and a marker/safety switch polypeptide, such as RQR (CD20+CD34; Philip et al., 2014).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

In any of the embodiments described herein, a polynucleotide can further comprise a polynucleotide that encodes a self-cleaving polypeptide, wherein the polynucleotide encoding the self-cleaving polypeptide is located between the polynucleotide encoding the fusion protein and the polynucleotide encoding the marker. In certain embodiments, a self-cleaving polypeptide comprises a 2A peptide from porcine teschovirus-1 (P2A), Thosea asigna virus (T2A), equine rhinitis A virus (E2A), foot-and-mouth disease virus (F2A), or variant thereof. In certain embodiments, an exemplary T2A peptide sequence comprises an amino acid sequence of SEQ ID NO:16. Further exemplary nucleic acid and amino acid sequences of 2A peptides are set forth in, for example, Kim et al. (PLOS One 6:e18556 (2011), which 2A nucleic acid and amino acid sequences are incorporated herein by reference in their entirety).

Fusion proteins of the present disclosure can, in certain aspects, be expressed on the surface of a host cell or be secreted by or isolated from a host cell. A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In addition to vectors, certain embodiments relate to host cells modified (i.e., genetically engineered) to contain a heterologous polynucleotide encoding a fusion protein (e.g., CAR) or a vector comprising a heterologous polynucleotide encoding a fusion protein (e.g., CAR) according to the present disclosure. A modified or genetically engineered host cell comprising a heterologous polynucleotide encoding at least one fusion protein expresses on its cell surface at least one fusion protein of the instant disclosure. A modified host cell may express a single type of fusion protein or two or more different types of fusion proteins of the present disclosure. Host cells can be modified ex vivo or in vivo. A host cell may include any individual cell or cell culture that may receive a vector or the incorporation of a nucleic acid or protein, as well as any progeny cells. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989). In any of the aforementioned embodiments, host cells containing polynucleotides encoding a fusion protein of this disclosure are comprised of cells that are autologous, allogeneic or syngeneic to the subject receiving the modified host cells, such as in an adoptive immunotherapy procedure.

In certain embodiments, the host cell transduced to express a fusion protein of the present disclosure is a hematopoietic progenitor cell or a human immune system cell. As used herein, a “hematopoietic progenitor cell” is a cell that can be derived from hematopoietic stem cells or fetal tissue and is capable of further differentiation into mature cells types (e.g., immune system cells). Exemplary hematopoietic progenitor cells include those with a CD24^{Lo} Lin⁻ CD117⁺ phenotype or those found in the thymus (referred to as progenitor thymocytes).

In certain embodiments, the host cell is an immune system cell, including, for example, a B cell, a T cell (e.g., a CD4⁺ T cell, a CD8⁺ T cell, a CD4⁻ CD8⁻ double negative T cell, a γδ T cell, a regulatory T cell), a natural killer cell (e.g., a NK cell or a NK-T cell), or a dendritic cell.

In certain embodiments, the host cell is a T cell. A T cell may be a naïve T cell, a memory T cell (T_{M}), a stem cell memory T cell, a helper T cell (T_{H}), an effector T cell (T_{E}), a γδ T cell, a regulatory T cell (Treg), or any combination thereof. T_{M} can be further divided into subsets of central memory T cells (T_{CM}, increased expression of CD62L, CCR7, CD28, CD127, CD45RO, and CD95, and decreased expression of CD54RA as compared to naïve T cells) and effector memory T cells (T_{EM}, decreased expression of CD62L, CCR7, CD28, CD45RA, and increased expression of CD127 as compared to naïve T cells or T_{CM}).

T cells can be collected using known techniques, and the various subpopulations or combinations thereof can be enriched or depleted by known techniques, such as by affinity binding to antibodies, flow cytometry, or immunomagnetic selection.

Methods for transfecting/transducing T cells with desired nucleic acids have been described (e.g., U.S. Patent Application Pub. No. US 2004/0087025; U.S. Pat. No. 6,410,319; PCT Publication No. WO 2014/031687; Brentjens et al., Clin. Cancer Res. 13:5426 (2007)) as have adoptive transfer procedures using T cells of desired target-specificity (e.g., Schmitt et al., Hum. Gen. 20:1240 (2009); Dossett et al., Mol. Ther. 17:742 (2009); Till et al., Blood 112:2261 (2008); Wang et al., Hum. Gene Ther. 18:712 (2007); Kuball et al., Blood 109:2331 (2007); US 2011/0243972; US 2011/0189141; Leen et al., Ann. Rev. Immunol. 25:243 (2007); Kalos et al., Sci Transl. Med. 3:95ra73 (2011); Porter et al., N. Engl. J. Med. 365:725-33 (2011)), such that adaptation of these methodologies to the presently disclosed embodiments is contemplated, based on the teachings herein, including those directed to fusion proteins of the present disclosure.

Eukaryotic host cells contemplated as an aspect of this disclosure when harboring a polynucleotide, vector, or protein according to this disclosure include, in addition to a human immune cells (e.g., a human patient's own immune cells), VERO cells, HeLa cells, Chinese hamster ovary (CHO) cell lines (including modified CHO cells capable of modifying the glycosylation pattern of expressed multivalent binding molecules, see US Pre-Grant Patent Publication No. 2003/0115614), COS cells (such as COS-7), W138, BHK, HepG2, 3T3, RIN, MDCK, A549, PC12, K562, HEK293 cells, HepG2 cells, N cells, 3T3 cells, Spodopterafrugiperda cells (e.g., Sf9 cells), Saccharomyces cerevisiae cells, and any other eukaryotic cell known in the art to be useful in expressing, and optionally isolating, a protein or peptide according to this disclosure. Also contemplated are prokaryotic cells, including Escherichia coli, Bacillus subtilis, Salmonella typhimurium, a Streptomycete, or any prokaryotic cell known in the art to be suitable for expressing, and optionally isolating, a protein or peptide according to this disclosure. In isolating protein or peptide from prokaryotic cells, in particular, it is contemplated that techniques known in the art for extracting protein from inclusion bodies may be used. Host cells that glycosylate the fusion proteins of this disclosure are contemplated.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media can also contain such components as growth factors or serum, as required. The growth medium will generally select for cells containing the heterologous polynucleotide by, for example, drug selection or deficiency in an essential nutrient which is complemented by the selectable marker carried on the expression vector or co-transfected into the host cell.

In certain embodiments, a fusion protein of the present disclosure is expressed on the surface of a host cell such that binding to a target antigen elicits an activity or response from the host cell. Such expressed proteins may be functionally characterized according to any of a large number of art-accepted methodologies for assaying host cell (e.g., T cell) activity, including determination of T cell binding, activation or induction and also including determination of T cell responses that are antigen-specific. Examples include determination of T cell proliferation, T cell cytokine release, antigen-specific T cell stimulation, MHC-restricted T cell stimulation, CTL activity (e.g., by detecting ⁵¹Cr or Europium release from pre-loaded target cells), changes in T cell phenotypic marker expression, and other measures of T cell functions. Procedures for performing these and similar assays are may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); Green and Reed, Science 281:1309 (1998) and references cited therein.

Levels of cytokines may be determined according to methods described herein and practiced in the art, including for example, ELISA, ELISPOT, intracellular cytokine staining, and flow cytometry and combinations thereof (e.g., intracellular cytokine staining and flow cytometry). Immune cell proliferation and clonal expansion resulting from an antigen-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1immune response and a Th2 immune response may be examined, for example, by determining levels of Th1 cytokines, such as IFN-γ, L-12, IL-2, and TNF-β, and Type 2 cytokines, such as IL-4, IL-5, IL-9, IL-10, and IL-13.

In certain embodiments, expression of an endogenous gene, such as TCR gene, a HLA gene, a β2M gene, an immunosuppression component gene (e.g., an immune checkpoint molecule gene), or any combination thereof is inhibited in the modified immune cell (e.g., T cell). In certain embodiments, the TCR gene is a T cell receptor α constant (TRAC) gene, a T cell receptor β constant (TRBC) gene, or both. In certain embodiments, the HLA gene is a HLA class I gene, an HLA class II gene, or both. In any of the embodiments provided herein, a modified immune cell can be modified to reduce or eliminate expression of one or more endogenous genes involved in an immune response. For example, a T cell may be modified to reduce or eliminate expression of one or more polypeptides of an HLA complex component, or a TCR or TCR complex component. Without wishing to be bound by theory, certain endogenously expressed immune cell proteins may be recognized as foreign by an allogeneic host that receives the modified immune cells, which may result in elimination of the modified immune cells (e.g., an HLA allele), may mediate graft versus host disease via an endogenously expressed receptor (e.g., TCR), may compete with a heterologous fusion protein of the present disclosure for expression by the host cell, or may interfere with the binding activity of a heterologously expressed fusion protein of the present disclosure (e.g., an endogenous TCR that binds to a non-tumor-associated antigen and interferes with the antigen-specific fusion protein of the modified immune cell specifically binding to the tumor-associated antigen). Accordingly, decreasing, inhibiting, or eliminating expression or activity of such endogenous genes or proteins can improve the activity, tolerance, or persistence of the modified immune cells in an allogeneic host setting, and can, in some embodiments, allow universal administration of the cells (e.g., to any recipient regardless of HLA type).

In certain embodiments, expression of an immunosuppression component gene (e.g., an immune checkpoint molecule gene) is inhibited in the modified immune cell. As used herein, the term “immunosuppression component” or “immune suppression component” refers to one or more cells, proteins, molecules, compounds or complexes providing inhibitory signals to assist in controlling or suppressing an immune response. For example, immunosuppression components include those molecules that partially or totally block immune stimulation; decrease, prevent or delay immune activation; or increase, activate, or up regulate immune suppression. Exemplary immunosuppression component targets include immune checkpoint molecules, such as PD-1, PD-L1, PD-L2, CD80, CD86, B7-H3, B7-H4, HVEM, adenosine, GAL9, VISTA, CEACAM-1, PVRL2, CTLA-4, BTLA, KIR, LAG3, TIM3, A2aR, CD244/2B4, CD160, TIGIT, LAIR-1, PVRIG/CD112R; metabolic enzymes, such as arginase, indoleamine 2,3-dioxygenase (IDO); immunosuppressive cytokines, such as IL-10, 1L-4, IL-IRA, IL-35; T_{reg} cells, or any combination thereof.

Expression of a TCR gene (e.g., a gene that encodes a TCR variable region or a TCR constant region; see, e.g., Torikai et al., Nature Sci. Rep. 6:21757 (2016); Torikai et al., Blood 119(24):5697 (2012); and Torikai et al., Blood 122(8):1341 (2013), the gene editing techniques, compositions, and adoptive cell therapies of which are incorporated herein in their entirety), HLA gene (e.g., a gene that encodes an α1 macroglobulin, an α2 macroglobulin, an α3 macroglobulin, a β1 microglobulin, or a β2 microglobulin), immunosuppression component gene, or any combination thereof may be knocked down, knocked out, or inhibited at the gene level, transcriptional level, translational level, or both in a modified immune cell. Exemplary inhibitors of expression of a TCR, HLA, or immunosuppression component gene include inhibitory nucleic acid molecules and endonucleases. Alterations resulting in a chromosomal gene knockout can include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

An “inhibitory nucleic acid” refers to a short, single stranded or double stranded nucleic acid molecule that has sequence complementary to a target gene or mRNA transcript and is capable of reducing expression of the target gene or mRNA transcript, or refers to a polynucleotide encoding such molecules. An inhibitory nucleic acid molecule includes antisense oligonucleotides, double stranded RNA (dsRNA) molecules, small interfering RNA (siRNA molecules, shRNA molecules, and endoribonuclease-prepared siRNA (esiRNA) molecules). Reduced expression may be accomplished via a variety of processes, including blocking of transcription or translation (e.g., steric hindrance), degradation of the target mRNA transcript, blocking of pre-mRNA splicing sites, blocking mRNA processing (e.g., capping, polyadenylation). In certain embodiments, inhibitory nucleic acid molecules may be used for gene knockdown methods. The genomic and mRNA sequences of TCR, HLA, and immunosuppression component genes are publicly available at, for example, the National Center for Biotechnology Information's GenBank database. Methods for making inhibitory nucleic acid molecules targeting mRNAs are known in the art and described, for example, in Ozcan et al. Adv. Drug Deliv. Rev. 87:108-119 (2016). Methods of inhibiting expression of a gene in an immune cell using an inhibitory nucleic acid molecule are known in the art and described, for example, in U.S. Pre-Grant Patent Publication Nos. US 2012/0321667 and US 2007/0036773; Condomines et al., PLoS ONE 10:e0130518 (2015); Ohno et al., J. Immunother. Cancer 1:21 (2013)).

Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene, thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for gene “knock-in” to inactivate a target gene. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, and meganucleases.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260 (1993); Wolfe et al., J. Mol. Biol. 285:1917-1934 (1999)). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a TCR gene, HLA gene, or immunosuppression component gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12^{th} and 13^{th} amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD sequence at positions 12 and 13 leads to a binding to cytosine (C), NG binds to T, NI to A, NN binds to G or A, and NG binds to T and non-canonical (atypical) RVDs are also known (see, e.g., U.S. Pre-Grant Patent Publication No. US 20110301073, which atypical RVDs are incorporated by reference herein in its entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In certain embodiments, a TCR gene, HLA gene, or immunosuppression component gene knockout comprises an insertion, a deletion, a mutation or a combination thereof, made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type IL, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology directed repair. The crRNA and tracrRNA can be engineered into a single guide RNA (sgRNA or gRNA) (see, e.g., Jinek et al., Science 337:816-21, 2012). Further, the region of the guide RNA complementary to the target site can be altered or programed to target a desired sequence (Xie et al., PLOS One 9:e100448, 2014; U.S. Pre-Grant Patent Pub. No. US 2014/0068797, U.S. Pre-Grant Patent Pub. No. US 2014/0186843; U.S. Pat. No. 8,697,359, and PCT Publication No. WO2015/071474; each of which is incorporated by reference). In certain embodiments, a TCR gene, HLA gene, or immunosuppression component gene knockout comprises an insertion, a deletion, a mutation or a combination thereof, made using a CRISPR/Cas nuclease system.

As used herein, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG, GIY-YIG, HNH, His-Cys box and PD-(D/E)XK. Exemplary meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998). In certain embodiments, naturally occurring meganucleases may be used to promote site-specific genome modification of a TCR gene, HLA gene, or immunosuppression component gene. In other embodiments, an engineered meganuclease having a novel binding specificity for a TCR gene, HLA gene, or immunosuppression component gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Pre-Grant Patent Publication Nos. US 2007/0117128; US 2006/0206949; US 2006/0153826; US 2006/0078552; and US 2004/0002092).

In further embodiments, a polynucleotide encoding a fusion protein of the present disclosure is inserted into a TCR gene, HLA gene, or immunosuppression component gene locus of an immune cell (e.g., T cell) via an endonuclease, e.g., a zinc finger nuclease, TALE-nuclease, CRISPR-Cas nuclease, or meganuclease. Without wishing to be bound by theory, targeting the fusion protein to a TCR gene locus may enhance anti-tumor activity of the fusion protein-expressing T cell (see, Eyquem et al., Nature, 543:113-117 (2017)).

In other aspects, kits are provided comprising (a) a vector or an expression construct as described herein and optional reagents for transducing the vector or the expression construct into a host cell, (b) an isolated polynucleotide, or expression vector as disclosed herein, and optional reagents for transducing the polynucleotide or expression vector into a host cell, or (c) a host cell of this disclosure.

### Methods of Treatment

In certain aspects, the compositions provided in the present disclosure may be used in methods for treating a disease in a subject, wherein the methods comprise administering to the subject: a fusion protein; a vector comprising a polynucleotide encoding a fusion protein; a modified host cell expressing a fusion protein; or a pharmaceutical composition thereof, and wherein the disease is associated with the presence of the antigen bound by the fusion protein. In certain embodiments, the disease is a viral infection, a bacterial infection, a cancer, an inflammatory disease, or an autoimmune disease.

As used, herein the term “cancer” includes solid tumors and hematological malignancies (e.g., leukemias). Exemplary cancers that may be treated include melanoma; non-small cell lung cancer; renal cell carcinoma; renal cancer; a hematological cancer; prostate cancer; castration-resistant prostate cancer; colon cancer; rectal cancer; gastric cancer; esophageal cancer; bladder cancer; head and neck cancer; thyroid cancer; breast cancer; triple-negative breast cancer; ovarian cancer; cervical cancer; lung cancer; urothelial cancer; pancreatic cancer; glioblastoma; hepatocellular cancer; brain cancer; CNS cancer; malignant glioma, sarcomas and carcinomas, including, for example, chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; synovial sarcoma; PNET; malignant hemangioendothelioma; malignant schwannoma; osteosarcoma; alveolar soft part sarcoma; angiosarcoma; cystosarcoma phyllodes; desmoid tumor; desmoplastic small round cell tumor; epithelioid sarcoma; extraskeletal chondrosarcoma; extraskeletal osteosarcoma; hemangiopericytoma; hemangiosarcoma; lymphangiosarcoma; lymphosarcoma; undifferentiated pleomorphic sarcoma; malignant peripheral nerve sheath tumor (MPNST); neurofibrosarcoma; rhabdomyosarcoma; undifferentiated pleomorphic sarcoma; Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); a lung carcinoma (e.g., Adenocarcinoma, Squamous Cell Carcinoma (Epidermoid Carcinoma), Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma); basal cell carcinoma, adenocarcinoma; linitis plastic; vipoma; cholangiocarcinoma; hepatocellular carcinoma; adenoid cystic carcinoma; renal cell carcinoma; Grawitz tumor, ependymoma; astrocytoma; oligodendroglioma; brainstem glioma; optice nerve glioma; ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, an adenocarcinoma of the prostate, chondrosarcoma; fibrosarcoma (fibroblastic sarcoma); Dermatofibrosarcoma protuberans (DFSP); osteosarcoma; rhabdomyosarcoma; pleomorphic sarcoma; or synovial sarcoma; lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma); ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

Exemplary hematological malignancies include acute lymphoblastic leukemia (ALL); acute myeloid leukemia (AML); chronic myelogenous leukemia (CML); chronic eosinophilic leukemia (CEL); myelodysplastic syndrome (MDS); Hodgkin's lymphoma, non-Hodgkin's lymphoma (NHL) (e.g., follicular lymphoma, diffuse large B-cell lymphoma, or chronic lymphocytic leukemia); myeloma; multiple myeloma (MM); plasmacytoma; plasma cell leukemia; Waldenström's macroglobulinemia; B cell lymphoma; lymphoplasmactyic lymphoma; Burkitt's lymphoma; small lymphocytic lymphoma (SLL); follicular lymphoma; immunoblastic large cell lymphoma; precursor B-lymphoblastic lymphoma; and mantle cell lymphoma; CD37⁺ dendritic cell lymphoma; lymphoplasmacytic lymphoma; splenic marginal zone lymphoma; extranodal marginal zone B-cell lymphoma of mucosa-associated (MALT)lymphoid tissue; nodal marginal zone B-cell lymphoma; mediastinal (thymic) large B-cell lymphoma; intravascular large B-cell lymphoma; primary effusion lymphoma; adult T-cell lymphoma; extranodal NK/T-cell lymphoma; nasal type; enteropathy-associated T-cell lymphoma; hepatosplenic T-cell lymphoma; blastic NK cell lymphoma; Sezary syndrome, angioimmunoblastic T cell lymphoma; anaplastic large cell lymphoma.

Other diseases that may be treated using the compositions provided herein include infections by pathogenic microorganisms, including viruses (e.g., HIV, BK polyomavirus, adenovirus, hepatitis C virus (HCV), hepatitis B virus (HBV), cytomegalovirus (CMV), Epstein-Barr virus (EBV), bacteria, and parasites. In another aspect, methods are provided for treating autoimmune diseases, including systemic lupus erythematosus, diabetes, rheumatoid arthritis, reactive arthritis, multiple sclerosis, pemphigus vulgaris, celiac disease, Crohn's disease, inflammatory bowel disease, ulcerative colitis, and autoimmune thyroid disease.

In certain embodiments, the subject is a human or non-human animal, such as a non-human primate, cow, horse, sheep, pig, cat, dog, goat, mouse, rat, rabbit, guinea pig. In one embodiment, the subject is a human, such as a human adult, adolescent, child, or infant.

In certain embodiments, the modified host cells administered to the subject are autologous, allogeneic, or syngeneic.

In certain embodiments, a subject treated with the compositions provided in the present disclosure exhibits low or mild cytokine release syndrome (CRS), low or mild CAR T-cell-related encephalopathy syndrome (CRES), or both. In certain embodiments, a subject treated with the compositions provided in the present disclosure exhibits a reduced cytokine release syndrome cell-related encephalopathy syndrome, or both, as compared to a subject (i.e., a reference subject or the same subject) that has been administered a reference host cell or composition wherein the fusion protein comprises a wild-type CD28 costimulatory signaling domain.

Data from clinical applications of CD19-specific CAR T cells suggests that CARs comprising CD28/CD3ζ signaling domains may be more likely to induce severe CRS in patients than CARs comprising 4-1BB/CD3 signaling domains (Davila et al., Sci. Transl. Med. 6:224ra25 (2014); Turtle et al., J. Clin. Invest. 126:2123-2138 (2016)). Modification of the CD28 costimulatory signaling domain according to the present disclosure may reduce the incidence or severity of CRS, CRES, or both, which in certain embodiments may be attained by reducing the intensity of signaling and cytokine production while retaining anti-tumor cytotoxicity. Without being bound by theory, CRS and CRES develop as a result of profound T cell proliferation and cytokine release that leads to excessive inflammation. Symptoms of CRS include fever, malaise, myalgia, anorexia, nausea, tachycardia, capillary leak, cardiac dysfunction, renal impairment, hepatic failure, disseminated intravascular coagulation, hypotension, hypoxia, and can affect any organ system in the body. CRES is a toxic encephalopathic condition characterized by confusion, delirium, seizures, and cerebral edema. CRS can evolve into fulminant hemophagocytic lymphohistiocytosis (HLH), which is characterized by severe immune activation, lymphohistiocytic tissue infiltration, and immune-mediated multiorgan failure. High plasma cytokines including interferon-7; IL-6; IL-8; sIL-2Rα; soluble glycoprotein 130 (gp130); sIL-6R; IL-15; IL-8; IL-10; Monocyte chemoattractant protein (MCP1); Macrophage inflammatory protein (MIP1)-α; MIP1-β and Granulocyte-macrophage colony-stimulating factor (GM-CSF), particularly early in CAR T cell treatment, are highly associated with severe CRS. CRS grading ranges from grade 1 to 4 based on the presence of fever, hypotension, hypoxia, and organ toxicity, wherein severe CRS is assigned a grade≥3 and exhibits organ toxicity and/or is potentially life threatening, requiring aggressive clinical intervention. Grade 1 CRS can manifest as fever and grade 1 organ toxicity, while for grades 2-4, any one of the criteria other than fever is sufficient. CRES is graded 1-4 based on neurological assessment score and the presence of raised intracranial pressure, and seizures or motor weakness. Methods for assessing CRS and CRES are known in the art (see, e.g., Neelapu et al., Nat. Rev. Clin. Onc. 15:47-62 (2018); Lee et al., Blood (2014); CTCAE v4.03); Porter et al. J. Hematol. Oncol. 11:35 (2018); and Liu and Zhao, J. Hematol. Oncol. 11:121 (2018)), which methods and grading systems are incorporated by reference herein). In certain embodiments, a subject treated with the compositions provided in the present disclosure does not thereafter exhibit CRS or CRES with a grade≥3. In certain embodiments, a low level or mild CRS or CRS refers to a CRS or CRES with a grade of less than 3, a grade of 2, or a grade of 1. Grading can be according to any art-accepted method, scale, or rubric, such as, for example, those described herein.

Antigen-specific T cell responses can be determined by comparison of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

A biological sample may be obtained from a subject for determining the presence and level of an immune response, e.g., cytokine release by a T cell expressing a CAR or a TCR. A “biological sample” as used herein may be a blood sample (from which serum or plasma may be prepared), biopsy specimen; body fluids (e.g., lung lavage, ascites, mucosal washings, synovial fluid); bone marrow; lymph nodes; tissue explant; organ culture; or any other tissue or cell preparation from the subject or a biological source. Biological samples may also be obtained from the subject prior to receiving any immunogenic composition, which biological sample is useful as a control for establishing baseline (i.e., pre-immunization) data.

Subjects that can be treated by the present invention are, in some embodiments, human and other primate subjects, such as monkeys and apes for veterinary medicine purposes. In any of the aforementioned embodiments, the subject may be a human subject. The subjects can be male or female and can be any suitable age, including infant, juvenile, adolescent, adult, and geriatric subjects. Cells according to the present disclosure may be administered in a manner appropriate to the disease, condition, or disorder to be treated as determined by persons skilled in the medical art. In any of the above embodiments, a cell comprising a cell as described herein is administered intravenously, intraperitoneally, intratumorally, into the bone marrow, into a lymph node, or into the cerebrospinal fluid. An appropriate dose, suitable duration, and frequency of administration of the compositions will be determined by such factors as the age, size, gender, and condition of the patient; the type and severity of the disease, condition, or disorder; the particular form of the active ingredient; and the method of administration.

Fusion proteins, polynucleotides, vectors, or modified host cells as described herein may be administered to a subject in a pharmaceutically or physiologically acceptable or suitable excipient or carrier. Pharmaceutically acceptable excipients are biologically compatible vehicles, e.g., physiological saline, which are described in greater detail herein, that are suitable for administration to a human or other non-human mammalian subject. A therapeutically effective dose, in the context of adoptive cell therapy, is an amount of host cells (expressing a binding protein according to the present disclosure) used in adoptive transfer that is capable of producing a clinically desirable result (e.g., a cytotoxic T cell response) in a statistically significant manner) in a treated human or non-human mammal. As is well known in the medical arts, the dosage for any one patient depends upon many factors, including the patient's size, weight, body surface area, age, the particular therapy to be administered, sex, time and route of administration, general health, and other drugs being administered concurrently. Doses will vary, but a preferred dose for administration of a host cell comprising a recombinant expression vector as described herein is about 10⁵ cells/M², about 5×10⁵ cells/m², about 10⁶ cells/m², about 5×10⁶ cells/m², about 10⁷ cells/M², about 5×10⁷ cells/M², about 10⁸ cells/m², about 5×10⁸ cells/m², about 10⁹ cells/M², about 5×10⁹ cells/M², about 10¹⁰ cells/M², about 5×10¹⁰ cells/m², or about 10¹¹ cells/M².

The number of cells will depend upon the ultimate use for which the composition is intended as well the type of cells included therein. For example, in certain embodiments, cells modified to contain a fusion protein will comprise a cell population containing at least 30%, 35%, 40%, 45%, 50%, 55%, 60%, 65%, 70%, 75%, 80%, 85%, 90%, 95% or 100% of such cells. For uses provided herein, cells are generally in a volume of a liter or less, 500 mls or less, 250 mls or less, or 100 mls or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells can be apportioned into multiple infusions that cumulatively equal or exceed 10⁵, 10⁶, 10⁷, 10⁸, 10⁹, 10¹⁰, or 10¹¹ cells.

In certain embodiments, a unit dose comprises (i) a composition comprising at least about 30%, at least about 40%, at least about 50%, at least about 60%, at least about 70%, at least about 80%, at least about 85%, at least about 90%, or at least about 95% modified or unmodified CD4⁺ T cells, combined with (ii) a composition comprising at least about 30%, at least about 40%, at least about 50%, at least about 60%, at least about 70%, at least about 80%, at least about 85%, at least about 90%, or at least about 95% modified or unmodified CD8⁺ T cells, in about a 1:1 ratio, wherein the unit dose contains a reduced amount or substantially no naïve T cells (i.e., has less than about 50%, less than about 40%, less than about 30%, less than about 20%, less than about 10%, less than about 5%, or less then about 1% the population of naïve T cells present in a unit dose as compared to a patient sample having a comparable number of PBMCs).

In some embodiments, a unit dose comprises (i) a composition comprising at least about 50% modified or unmodified CD4⁺ T cells, combined with (ii) a composition comprising at least about 50% modified or unmodified CD8⁺ T cells, in about a 1:1 ratio, wherein the unit dose contains a reduced amount or substantially no naïve T cells. In further embodiments, a unit dose comprises (i) a composition comprising at least about 60% modified or unmodified CD4⁺ T cells, combined with (ii) a composition comprising at least about 60% modified or unmodified CD8⁺ T cells, in about a 1:1 ratio, wherein the unit dose contains a reduced amount or substantially no naïve T cells. In still further embodiments, a unit dose comprises (i) a composition comprising at least about 70% modified or unmodified CD4⁺ T cells, combined with (ii) a composition comprising at least about 70% modified or unmodified CD8⁺ T cells, in about a 1:1 ratio, wherein the unit dose contains a reduced amount or substantially no naïve T cells. In some embodiments, a unit dose comprises (i) a composition comprising at least about 80% modified or unmodified CD4⁺ T cells, combined with (ii) a composition comprising at least about 80% modified or unmodified CD8⁺ T cells, in about a 1:1 ratio, wherein the unit dose contains a reduced amount or substantially no naïve T cells. In some embodiments, a unit dose comprises (i) a composition comprising at least about 85% modified or unmodified CD4⁺ T cells, combined with (ii) a composition comprising at least about 85% modified or unmodified CD8⁺ T cells, in about a 1:1 ratio, wherein the unit dose contains a reduced amount or substantially no naïve T cells. In some embodiments, a unit dose comprises (i) a composition comprising at least about 90% modified or unmodified CD4⁺ T cells, combined with (ii) a composition comprising at least about 90% modified or unmodified CD8⁺ T cells, in about a 1:1 ratio, wherein the unit dose contains a reduced amount or substantially no naïve T cells.

In any of the embodiments described herein, a unit dose comprises equal, or approximately equal numbers of modified or unmodified CD45RA⁻CD3⁺CD8⁺ and modified or unmodified CD45RA⁻CD3⁺CD4⁺ T_{M} cells.

Also contemplated are pharmaceutical compositions that comprise fusion proteins or cells expressing the fusion proteins as disclosed herein and a pharmaceutically acceptable carrier, diluents, or excipient. Suitable excipients include water, saline, dextrose, glycerol, or the like and combinations thereof. In embodiments, compositions comprising fusion proteins or host cells as disclosed herein further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's disease, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

The pharmaceutical compositions described herein may be presented in unit-dose or multi-dose containers, such as sealed ampoules or vials. Such containers may be frozen to preserve the stability of the formulation until. In certain embodiments, a unit dose comprises a recombinant host cell as described herein at a dose of about 10⁵ cells/M2 to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polyethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of recombinant cells or active compound calculated to produce the desired therapeutic effect in association with an appropriate pharmaceutical carrier.

In general, an appropriate dosage and treatment regimen provides the active molecules or cells in an amount sufficient to provide therapeutic or prophylactic benefit. Such a response can be monitored by establishing an improved clinical outcome (e.g., more frequent remissions, complete or partial, or longer disease-free survival) in treated subjects as compared to non-treated subjects. Increases in preexisting immune responses to a tumor protein generally correlate with an improved clinical outcome. Such immune responses may generally be evaluated using standard proliferation, cytotoxicity or cytokine assays, which may be performed using samples obtained from a subject before and after treatment.

In certain embodiments, methods of treating a disease comprise administering modified immune cells in combination with one or more additional agents.

In certain embodiments, a modified immune cell of the present disclosure is administered to a subject with an inhibitor of an immune suppression component.

As used herein, the term “immune suppression component” or “immunosuppression component” refers to one or more cells, proteins, molecules, compounds or complexes providing inhibitory signals to assist in controlling or suppressing an immune response. For example, immune suppression components include those molecules that partially or totally block immune stimulation; decrease, prevent or delay immune activation; or increase, activate, or up regulate immune suppression. Exemplary immunosuppression component targets are described in further detail herein and include immune checkpoint molecules, such as PD-1, PD-L1, PD-L2, CD80, CD86, B7-H3, B7-H4, HVEM, adenosine, GAL9, VISTA, CEACAM-1, PVRL2, CTLA-4, BTLA, KIR, LAG3, TIM3, A2aR, CD244/2B4, CD160, TIGIT, LAIR-1, PVRIG/CD112R; metabolic enzymes, such as arginase, indoleamine 2,3-dioxygenase (IDO); immunosuppressive cytokines, such as IL-10, I-4, IL-IRA, IL-35; T_{reg} cells, or any combination thereof.

An inhibitor of an immune suppression component may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In any of the embodiments disclosed herein, a method may comprise administering a modified immune cell with one or more inhibitor of any one of the following immune suppression components, singly or in any combination.

In certain embodiments, a modified immune cell is used in combination with a PD-1 inhibitor, for example a PD-1-specific antibody or binding fragment thereof, such as pidilizumab, nivolumab (Keytruda, formerly MDX-1106), pembrolizumab (Opdivo, formerly MK-3475), MEDIO680 (formerly AMP-514), AMP-224, BMS-936558 or any combination thereof.

In certain embodiments, a modified immune cell is used in combination with a PD-L1 specific antibody or binding fragment thereof, such as BMS-936559, durvalumab (MEDI4736), atezolizumab (RG7446), avelumab (MSB0010718C), MPDL3280A, or any combination thereof.

In certain embodiments, a modified immune cell is used in combination with a LAG3 inhibitor, such as LAG525, IMP321, IMP701, 9H12, BMS-986016, or any combination thereof.

In certain embodiments, a modified immune cell is used in combination with an inhibitor of CTLA4. In particular embodiments, a modified immune cell is used in combination with a CTLA4 specific antibody or binding fragment thereof, such as ipilimumab, tremelimumab, CTLA4-Ig fusion proteins (e.g., abatacept, belatacept), or any combination thereof.

In certain embodiments, a modified immune cell is used in combination with a B7-H3 specific antibody or an antigen-binding fragment thereof, such as enoblituzumab (MGA271), 376.96, or both.

In certain embodiments, a modified immune cell is used in combination with a B7-H4 specific antibody or binding fragment thereof, such as a scFv or fusion protein thereof, as described in, for example, Dangaj et al., Cancer Res. 73:4820, 2013, as well as those described in U.S. Pat. No. 9,574,000 and PCT Patent Publication Nos. WO 2016/40724 and WO 2013/025779.

In some embodiments, a modified immune cell is used in combination with an inhibitor of CD244.

In certain embodiments, a modified immune cell is used in combination with an inhibitor of BLTA, HVEM, CD160, or any combination thereof. Anti-CD160 antibodies are described in, for example, PCT Publication No. WO 2010/084158.

In more embodiments, a modified immune cell is used in combination with an inhibitor of TIM3.

In still more embodiments, a modified immune cell is used in combination with an inhibitor of Gal9.

In certain embodiments, a modified immune cell is used in combination with an inhibitor of adenosine signaling, such as a decoy adenosine receptor.

In certain embodiments, a modified immune cell is used in combination with an inhibitor of A2aR.

In certain embodiments, a modified immune cell is used in combination with an inhibitor of KIR, such as lirilumab (BMS-986015).

In certain embodiments, a modified immune cell is used in combination with an inhibitor of an inhibitory cytokine (typically, a cytokine other than TGFβ) or Treg development or activity.

In certain embodiments, a modified immune cell is used in combination with an IDO inhibitor, such as levo-I-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013; Apr. 6-10, 2013), 1-methyl-tryptophan (1-MT)-tira-pazamine, or any combination thereof.

In certain embodiments, a modified immune cell is used in combination with an arginase inhibitor, such as N(omega)-Nitro-L-arginine methyl ester (L-NAME), N-omega-hydroxy-nor-1-arginine (nor-NOHA), L-NOHA, 2(S)-amino-6-boronohexanoic acid (ABH), S-(2-boronoethyl)-L-cysteine (BEC), or any combination thereof.

In certain embodiments, a modified immune cell is used in combination with an inhibitor of VISTA, such as CA-170 (Curis, Lexington, Mass.).

In certain embodiments, a modified immune cell is used in combination with a LAIR1 inhibitor.

In certain embodiments, a modified immune cell is used in combination with an inhibitor of CEACAM-1, CEACAM-3, CEACAM-5, or any combination thereof.

In certain embodiments, a modified immune cell is used in combination with an agent that increases the activity (i.e., is an agonist) of a stimulatory immune checkpoint molecule. For example, a modified immune cell can be used in combination with a CD137 (4-1BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD122 agonist (such as, for example, IL-2), an agonist of GITR (such as, for example, humanized monoclonal antibodies described in PCT Patent Publication No. WO 2016/054638), or an agonist of ICOS (CD278) (such as, for example, GSK3359609, mAb 88.2, JTX-2011, Icos 145-1, or Icos 314-8), or any combination thereof. In any of the embodiments disclosed herein, a method may comprise administering a modified immune cell with one or more agonists of a stimulatory immune checkpoint molecule, including any of the foregoing, singly or in any combination.

In other embodiments, a method of this disclosure further comprises administering a secondary therapy comprising one or more of: an antibody or antigen binding fragment specific for a cancer antigen expressed by the solid tumor being targeted; a chemotherapeutic agent; surgery; radiation therapy treatment; a cytokine; an RNA interference therapy, or any combination thereof.

Exemplary monoclonal antibodies useful in cancer therapies include, for example, monoclonal antibodies described in Galluzzi et al., Oncotarget 5(24):12472-12508 (2014), which antibodies are incorporated by reference in their entirety.

In certain embodiments, a combination therapy method comprises administering a modified immune cell and further administering a radiation treatment or a surgery to a subject. Radiation therapy includes X-ray therapies, such as gamma-irradiation, and radiopharmaceutical therapies. Surgeries and surgical techniques appropriate to treating a given cancer or non-inflamed solid tumor may be used in a subject in combination with a modified immune cell of this disclosure.

In certain embodiments, a combination therapy method comprises administering a modified immune cell and a chemotherapeutic agent to a subject. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

Cytokines can be used to manipulate host immune response towards anticancer activity. See, e.g., Floros and Tarhini, Semin. Oncol. 42:539, 2015. Cytokines useful for promoting anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination.

Another cancer therapy approach involves reducing expression of oncogenes and other genes needed for growth, maintenance, proliferation, and immune evasion by cancer cells. RNA interference, and in particular the use of microRNAs (miRNAs) small inhibitory RNAs (siRNAs) provides an approach for knocking down expression of cancer genes. See, e.g., Larsson et al., Cancer Treat. Rev. 16:128, 2017.

In any of the embodiments disclosed herein, any of the therapeutic agents (e.g., a modified immune cell, an inhibitor of an immune suppression component, an agonist of a stimulatory immune checkpoint molecule, a chemotherapeutic agent, a radiation therapy, a surgery, a cytokine, or an inhibitory RNA) may be administered once or more than once to the subject over the course of a treatment, and, in combinations, may be administered to the subject in any order (e.g., simultaneously, concurrently, or in any sequence) or any combination. An appropriate dose, suitable duration, and frequency of administration of the compositions will be determined by such factors as a condition of the patient; size, type, spread, growth, and severity of the tumor or cancer; particular form of the active ingredient; and the method of administration.

In certain embodiments, a plurality of doses of a modified immune cell as described herein is administered to the subject, which may be administered at intervals between administrations of about two to about four weeks. In further embodiments, a cytokine (e.g., IL-2, 1L-15, 1L-21) is administered sequentially, provided that the subject was administered the recombinant host cell at least three or four times before cytokine administration. In certain embodiments, a cytokine is administered concurrently with the host cell. In certain embodiments, a cytokine is administered subcutaneously.

In still further embodiments, a subject being treated is further receiving immunosuppressive therapy, such as calcineurin inhibitors, corticosteroids, microtubule inhibitors, low dose of a mycophenolic acid prodrug, or any combination thereof. In yet further embodiments, a subject being treated has received a non-myeloablative or a myeloablative hematopoietic cell transplant, wherein the treatment may be administered at least two to at least three months after the non-myeloablative hematopoietic cell transplant.

An effective amount of a therapeutic or pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

### EXAMPLES

### Example 1

### Materials and Methods

Acquisition of Peripheral Blood T Cells from Healthy Donors

Healthy adults (>18 years-old) were enrolled in an Institutional Review Board-approved study for peripheral blood collection. Informed consent was obtained from all enrollees. Investigators were blinded to all personally identifiable information about study participants, and were provided only donor age and a nondescript donor ID number. 400 cc of peripheral blood was collected by venipuncture and mononuclear cells (PBMC) were isolated by density gradient using Lymphocyte Separation Media (Corning Cat #25-072-CV). CD4⁺ and CD8⁺ T cells were isolated using the EasySep Human CD8⁺ T Cell Isolation Kit (Stem Cell Technologies Cat. #s 17952 and 17953); CD8⁺CD62L⁺ T cells were further enriched by staining with CD62L-PE (ThermoFisher Cat #12-0629-42) followed by the EasySep Human PE Selection Kit (Stem Cell Technologies Cat #18551. Isolations were performed in accordance with manufacturer's instructions.

### Cell Culture

293T LentiX cells (Clontech Cat #632180) were cultured in DMEM (Gibco) supplemented with 10% fetal bovine serum, 1 mM L-glutamine (Gibco), and 100U/mL penicillin/streptomycin (Gibco). K562 (CCL-243) and Jurkat (TIB-152) cells were obtained from ATCC and cultured in RPMI-1640 (Gibco) supplemented with 5% fetal bovine serum and 100U/mL penicillin/streptomycin. Primary human T cells were cultured in CTL medium consisting of RPMI-1640 supplemented with 10% human serum, 2 mM L-glutamine, 100U/mL penicillin/streptomycin, 50 μM β-Mercaptoethanol (Sigma), and 50 U/mL IL-2 (Prometheus Proleukin/Aldesleukin). All cells were cultured at 37° C. and 5% CO₂, and tested bi-monthly for the absence of mycoplasma using MycoAlert Mycoplasma Detection Kit (Lonza Cat # LT07-318).

### Generation of Chimeric Antigen Receptors (CARs) and Recombinant Lentiviral Vectors

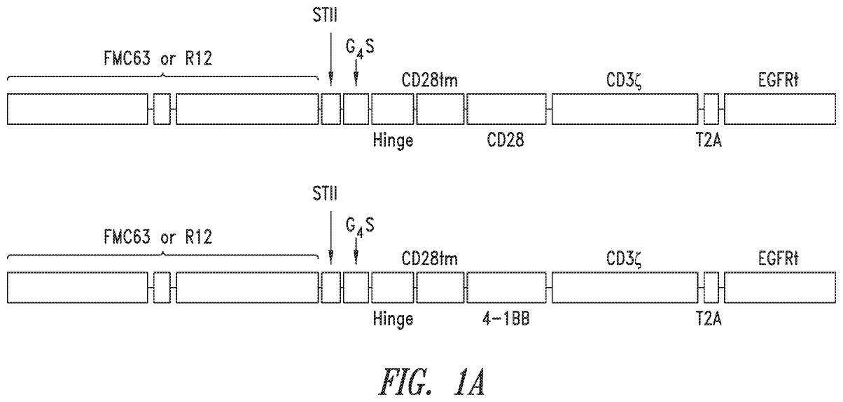

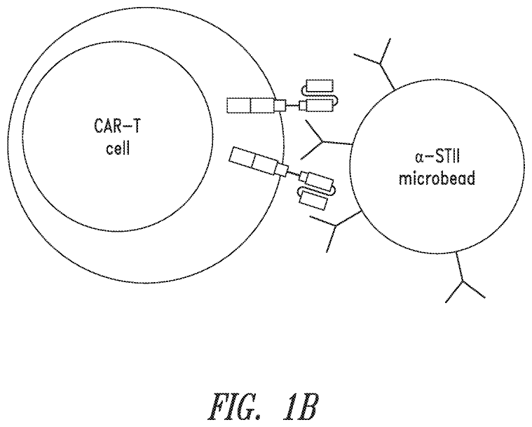

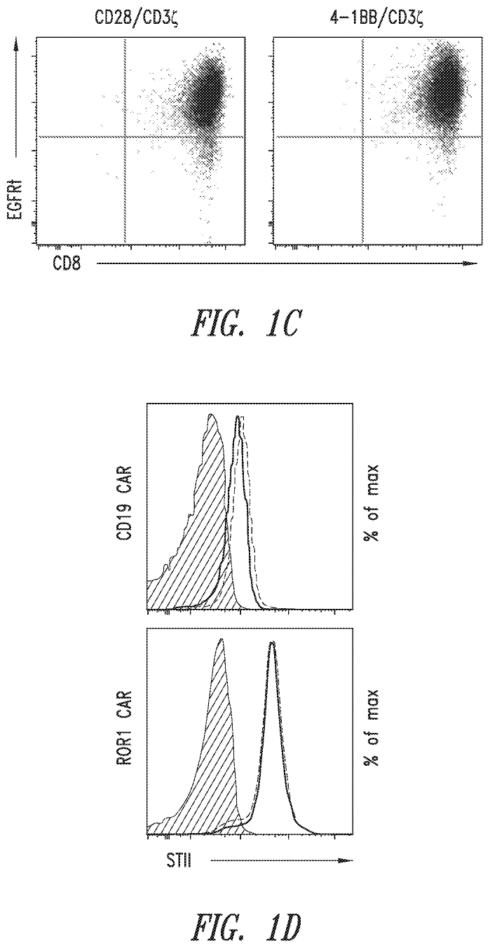

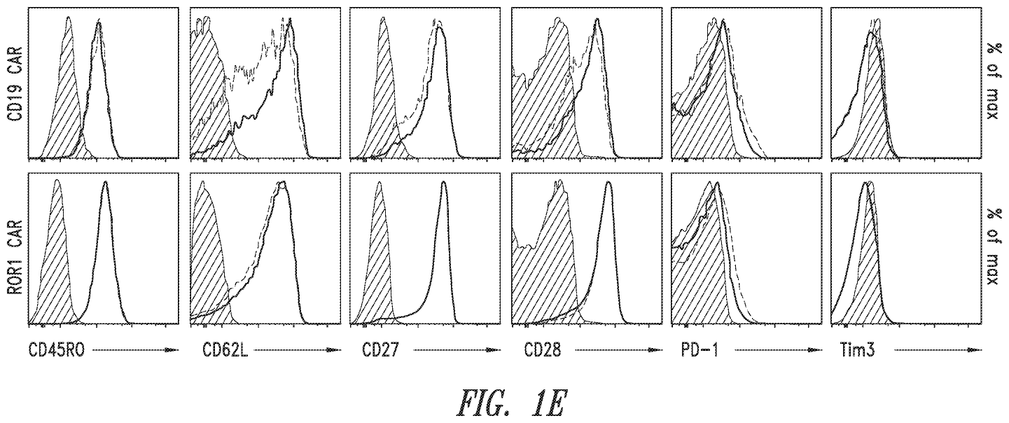

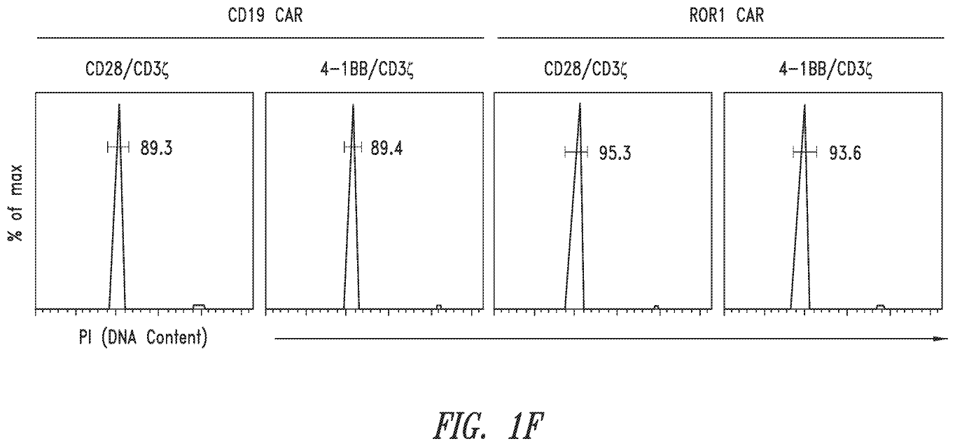

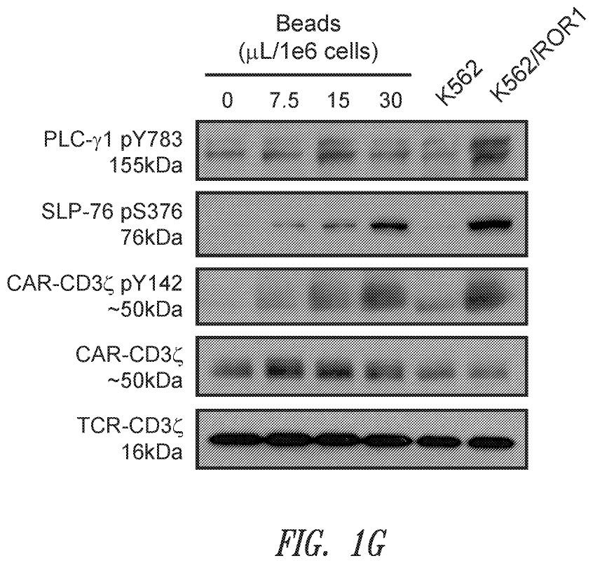

CD19-specific and ROR1-specific CAR constructs have been previously described (Hudecek et al., Clin. Cancer Res. 19:3153-3164 (2013); Sommermeyer et al., Leukemia 30:492-500 (2016) (FIG. 1A). A single Strep-tag II sequence (SEQ ID NO:10) and two G₄S linkers were inserted between the FMC63 (SEQ ID NO:8) or R12 single chain variable fragment (SEQ ID NO:9) and IgG4 hinge sequences (SEQ ID NO:12) (Liu et al., Nat. Biotechnol. 34:430-434 (2016)). These were linked to the 27 amino acid transmembrane domain of human CD28 (UniProt: P10747; SEQ ID NO:13) and to a signaling module comprising either (i) the 41 amino acid cytoplasmic domain of human CD28 with an LL→GG substitution located at positions 186-187 of the native CD28 protein (Nguyen et al., Blood 102:4320-4325 (2003) (SEQ ID NO:3), or (ii) the 42-amino acid cytoplasmic domain of human 4-1BB (UniProt: Q07011; SEQ ID NO:14), each of which was linked to the 112-amino acid cytoplasmic domain of isoform 3 of human CD3ζ (UniProt: P20963-3; SEQ ID NO:15). Mutant CD28/CD3ζ CARs with tyrosine to phenylalanine substitutions at CD28 UniProt positions 206, 209, and 218 were generated by site-directed mutagenesis. All CAR gene constructs were linked by a sequence encoding T2A (SEQ ID NO:16) to a sequence encoding truncated epidermal growth factor receptor (EGFRt; SEQ ID NO:17), codon-optimized, and cloned into the HIV7 lentiviral vector. To make antigen-positive K562 cells, amino acids 1-325 of human CD19 (UniProt: P15391) were cloned into the HIV7 lentiviral vector and amino acids 1-937 of human ROR1 (UniProt: Q01973) were cloned into the mp71 retroviral vector. All cloning was performed by PCR, enzyme digest, and/or Gibson assembly. Plasmids were verified by capillary sequencing and restriction digest.

### Lentivirus Preparation and Transduction

To prepare CAR T cells, LentiX cells were transiently transfected with the HIV7 CAR vector, as well as psPAX2 (Addgene Cat #12260) and pMD2.G (Addgene Cat #12259) packaging plasmids. One day later (day 1), primary T cells were activated using Dynabeads Human T-Activator CD3/CD28 (ThermoFisher Cat #11132D) and cultured in CTL supplemented with 50 U/mL IL-2. The next day (day 2), lentiviral supernatant was harvested from LentiX cells, filtered using 0.45 μm PES syringe filters (Millipore Cat # SLHP033RB), and added to activated T cells. Polybrene (Millipore Cat # TR-1003-G) was added to reach a final concentration of 4.4 μg/mL and cells were spinoculated at 800×g and 32° C. for 90 minutes. Viral supernatant was replaced 8 hours later with fresh CTL supplemented with 50 U/mL IL-2. Half-media changes were then performed every 48 hours using CTL supplemented with 50 U/mL IL-2. Dynabeads were removed on day 6; CD8⁺ EGFRt⁺ transduced T cells were FACS-sorted on a FACSAriaII (BD Biosciences) on day 9.

To prepare K562/CD19 cells, LentiX cells were transiently transfected with psPAX2, pMD2.G, and an HIV7 lentiviral vector encoding CD19. To prepare K562/ROR1 cells, LentiX cells were transiently transfected with MLV g/p, 10A1, and a mp71 retroviral vector encoding ROR1 (Uckert et al., Hum. Gene Ther. 11:1005-1014 (2000)). Two days later, viral supernatant was filtered using a 0.45 μm PES syringe filter, and added to K562 cells. Five days later, transduced K562 cells were stained monoclonal antibodies specific for CD19 (Biolegend Cat #302212) or ROR1 (Miltenyi Biotec Cat #130-098-317) and FACS-sorted on a FACSAria II.

### T Cell Expansion for Mass Spectrometric and Functional Analyses

FACS-sorted CD8⁺EGFRt⁺ cells were expanded over a single stimulation cycle prior to MS and/or functional analyses. CD19-specific CAR-T cells were expanded by co-culture with CD19⁺ lymphoblastoid cell lines (LCL) in a 1:7 (T cell:LCL) ratio and assayed 8 days after stimulation. ROR1-specific CAR-T cells were expanded using a rapid expansion protocol containing purified OKT3, LCL, irradiated PBMC and assayed 11 days after stimulation. During expansion, cultures were fed with fresh CTL media containing 50U/mL IL-2 every 2-3 days.

### Flow Cytometry and Cell Phenotyping

T cells were stained with a 1:100 dilution of fluorophore-conjugated monoclonal antibodies purchased from BD Biosciences, ThermoFisher, or Biolegend. Cetuximab (anti-EGFR, Bristol Myers Squibb) and 3E8 (anti-STII, FHCRC) mAb were biotinylated using the EZ-Link Sulfo-NHS-Biotin kit (ThermoFisher Cat #21217) followed by cleanup with a Zeba Spin Desalting Column (ThermoFisher Cat #89882) and used to stain T cells in conjunction with Streptavidin-APC (ThermoFisher Cat #17-4317-82). DNA content staining was performed by fixing T cells with 70% ice-cold ethanol, permeabilizing cells with 1% Triton-X (Sigma Cat # T8787), degrading RNA with 100 μg/mL RNAse A (ThermoFisher Cat # EN0531), and staining DNA with 20 μg/mL Propidium Iodide (ThermoFisher Cat # P3566). All data was collected on a FACSCanto II (BD Biosciences) and analyzed with FlowJo version 9 (Treestar).

### Anti-STII and Control Bead Preparation

1 mL Streptavidin Coated Magnetic Particles (Spherotech Cat # SVMS-30-10) was washed once in excess 1×PBS supplemented with 100 U/mL penicillin/streptomycin (PBS+P/S) using a benchtop magnet. Anti-STII beads were prepared by resuspending the bead pellet in 1 mL PBS+P/S. While vortexing, 16.67 μg anti-STII mAb (GenScript Cat # A01737) was added per 1 mL beads. Beads and anti-STII antibody were incubated overnight on a 3D orbital shaker, washed three times with excess PBS+P/S using a benchtop magnet, and resuspended in 4 mL PBS+P/S. To make control beads, 1 mL Streptavidin Coated Magnetic Particles were washed once using a benchtop magnet and the bead pellet was resuspended in 4 mL PBS+P/S. All beads were stored at 4° C.

### Cell Stimulations, Protein Lysates, and RNA Isolation

CAR T or Jurkat cells were washed and resuspended in warm CTL medium. For OKT3 stimulations, cells were incubated for 5 minutes on ice with 1.25 μg/mL OKT3 (Biolegend Cat #317303) and then with 5 μg/mL anti-mouse IgG (ThermoFisher Cat # A16068) for 10 minutes on ice. Cells were transferred to a 37° C. water bath to initiate signaling. For K562 cell-based stimulations, CAR T cells were co-cultured with K562 or K562/CD19 cells at a 4:1 effector to target ratio in a 37° C. water bath. For bead-based stimulations, T cells were incubated with STII or control microbeads at a ratio of 30 μL beads per 1×10⁶ cells in a 37° C. water bath (FIG. 1B). After the allotted time, cells were quickly washed twice using ice-cold PBS, then lysed in a 6M Urea, 25 mM Tris (pH 8.0), 1 mM EDTA, 1 mM EGTA solution supplemented with protease (Sigma Cat # P8340-1ML) and phosphatase inhibitors (Sigma Cat #s P5726-1ML and P0044-1ML) at a 1:100 dilution, hereon referred to as lysis buffer. Lysates were sonicated for 15 seconds prior to centrifuging at 10,000×g and 4° C. for 10 minutes. Beads were removed during lysate clearing. After 6 hours of stimulation, RNA isolations were performed using a NucleoSpin RNA kit (Macherey-Nagel Cat #740955) according to manufacturer's instructions. Beads were removed prior to cell lysis using a benchtop magnet.

Protein Digestion, TMT Labeling, and Phosphotyrosine (pTyr) Peptide Immunoprecipitation

Protein was quantified in lysates by Micro BCA Assay (ThermoFisher Cat #23235), and lysates were diluted to 2 mg/mL using lysis buffer. Lysates were reduced in 24 mM TCEP (ThermoFisher Cat #77720) for 30 minutes at 37° C. with shaking, followed by alkylation with 48 mM iodoacetamide (Sigma Cat # A3221) in the dark at room temperature for 30 minutes. Lysates were then diluted with 200 mM Tris (pH 8.0), to a urea concentration of 2M. Lys-C(Wako Cat #125-05061) was dissolved in 25 mM Tris (pH 8.0) at 200 ug/mL and added to lysates at 1:100 (enzyme:protein) ratio by mass and incubated for 2 hours at 37° C. with shaking. Samples were further diluted with 200 mM Tris (pH 8.0) to a urea concentration of 1M before adding trypsin at a 1:50 trypsin:protein ratio. After 2 hours, a second trypsin aliquot was added at a 1:100 trypsin:protein ratio. Digestion was carried out overnight at 37° C. with shaking. After 16 hours, the reaction was quenched with formic acid to a final concentration 1% by volume. Samples were desalted using Oasis HLB 96-well plates (Waters Cat #WAT058951) and a positive pressure manifold (Waters Cat #186005521). The plate wells were washed with 3×400 μL of 50% MeCN/0.1% FA, and then equilibrated with 4×400 μL of 0.1% FA. The digests were applied to the wells, then washed with 4×400 μL 0.1% FA before being eluted drop by drop with 3×400 μL of 50% MeCN/0.1% FA. The eluates were lyophilized, followed by storage at −80° C. until use. For TMT labeling (ThermoFisher, Cat #90406), desalted peptides were resuspended in 50 mM HEPES at 1 mg/mL based on starting protein mass. TMT reagents were resuspended in 257 μL MeCN and transferred to the peptide sample. Samples were incubated at room temperature for 1 hour with mixing. Labeling reactions were quenched by the addition of 50 μL of 5% hydroxyl Amine (Sigma Cat #438227) and incubated for 15 minutes at room temperature with mixing. The independent labeling reactions were then pooled together and lyophilized. The labeled peptides were desalted as above and then lyophilized and stored at −80° C. Immunoprecipitation of pTyr peptides (Cell Signaling Cat #8803) was then performed as per manufacturer's instructions. The enriched pTyr peptide fraction was purified using a C18 Spin Tip (ThermoFisher Cat #84850), lyophilized, and stored at −80° C. until analysis. The flow-through fraction was desalted, lyophilized, and stored at −80° C.

Basic (high pH) Reverse Phase Liquid Chromatography

The desalted and pTyr peptide-depleted flow-through was fractionated by high-pH reverse phase (RP) liquid chromatography. 4 mg of the protein digest was loaded onto a LC system consisting of an Agilent 1200 HPLC with mobile phases of 5 mM NH₄HCO₃ (pH 10) (A) and 5 mM NH₄HCO₃ in 90% MCN (pH 10) (B). The peptides were separated by a 4.6 mm×250 mm Zorbax Extend-C18, 3.5 μm, column (Agilent Cat #770953-902) over 96 minutes at a flow rate of 1.mL/min by the following timetable: hold 0% B for 9 minutes, gradient from 0 to 10% B for 4 minutes, 10 to 28.5% B for 50 minutes, 28.5 to 34% B for 5.5 minutes, 34 to 60% B for 13 minutes, hold at 60% B for 8.5 minutes, 60 to 0% B for 1 minute, re-equilibrate at 0% B for 5 minutes. 1-minute fractions were collected from 0-96 minutes by the shortest path by row in a 1 mL deep well plate (Thermo Cat #95040450). The high pH RP fractions were concatenated into 24 samples by every other plate column starting at minute 15 (e.g.: sample 1 contained fractions from wells B10, D10, F10, etc.). The remaining fractions were combined such that fractions from 12 to 14 minutes were added to sample 1, all fractions after 86 minutes were added to sample 24, and all fractions from 0 to 11 minutes were combined into sample ‘A’. 95% of every 12th fraction of the 24 samples was combined (1,13; 2,14; . . . ) to generate 12 more samples, which were dried down and stored at −80° C. prior to phosphopeptide enrichment by immobilized metal affinity chromatography.

### Immobilized Metal Affinity Chromatography (MAC)

IMAC enrichment was performed using Ni-NTA-agarose beads (Qiagen Cat #36113) stripped with EDTA and incubated in a 10 mM FeCl₃ solution to prepare Fe³⁺-NTA-agarose beads. Fractionated lysate was reconstituted in 200 μL of 0.1% TFA in 80% MCN and incubated for 30 minutes with 100 μL of the 5% bead suspension while mixing at room temperature. After incubation, beads were washed 3 times with 300 μL of 0.1% TFA in 80% MCCN. Phosphorylated peptides were eluted from the beads using 200 μL of 70% ACN, 1% Ammonium Hydroxide for 1 minute with agitation at room temperature. Samples were transferred into a fresh tube containing 60 uL of 10% FA, dried down and re-suspended in 0.1% FA, 3% MeCN. Samples were frozen at −80° C. until analysis.

### Nano-Liquid Chromatography-Tandem Mass Spectrometry

Phosphopeptide-enriched samples were analyzed by LC-MS/MS on an Easy-nLC 1000 (ThermoFisher) coupled to an LTQ-Orbitrap Fusion mass spectrometer (ThermoFisher) operated in positive ion mode. The LC system, configured in a vented format consisted of a fused-silica nanospray needle (PicoTip™ emitter, 50 μm ID×20 cm, New Objective) packed in-house with ReproSil-Pur C18-AQ, 3 μm and a trap (IntegraFrit™ Capillary, 100 μm ID×2 cm, New Objective) containing the same resin as in the analytical column with mobile phases of 0.1% FA in water (A) and 0.1% FA in MCCN (B). The peptide sample was diluted in 20 μL of 0.1% FA, 3% MCN, and 8.5 μL was loaded onto the column and separated over 210 minutes at a flow rate of 300 nL/min with a gradient from 5 to 7% B for 2 minutes, 7 to 35% B for 150 minutes, 35 to 50% B for 1 minute, hold 50% B for 9 minutes, 50 to 95% B for 2 minutes, hold 95% B for 7 minutes, 95 to 5% B for 1 minute, re-equilibrate at 5% B for 38 minutes. A spray voltage of 2000 V was applied to the nanospray tip. MS/MS analysis occurred over a 3 second cycle time consisting of 1 full scan MS from 350-1500 m/z at resolution 120,000 followed by data dependent MS/MS scans using HCD activation with 27% normalized collision energy of the most abundant ions. Selected ions were dynamically excluded for 45 seconds after a repeat count of 1.

### Western Blotting

Equal masses of protein lysate ranging from 5-13.3 μg were loaded into 4-12% Bis-Tris NuPAGE Gels (ThermoFisher) or 3-8% Tris-Acetate NuPAGE Gels (ThermoFisher). After protein transfer onto nitrocellulose membranes (ThermoFisher Cat # LC2001), membranes were blocked with Western Blocking Reagent (Sigma Cat#11921673001). Membranes were stained with primary and secondary mAbs diluted in SuperBlock supplemented with 0.1% Tween. The following antibodies were used in this study: anti-human CD247 (BD Biosciences Cat #551034), anti-human CD247 pY142 (BD Biosciences Cat #558402), anti-ZAP-70 pY319 (Cell Signaling Cat #2717S), anti-SLP-76 pS376 (Cell Signaling Cat #14745S), anti-PLC-g1 pY783 (Cell Signaling Cat #14008S), and anti-DAPP1 pY139 (Cell Signaling Cat #13703S), anti-mouse HRP (Cell Signaling Cat #7076S), and anti-rabbit HRP (Cell Signaling Cat #7074S). Typical antibody dilutions ranged from 1/10,000 to 1/2,500. Blots were developed using CL-XPosure Film (ThermoFisher Cat #34091) or ChemiDoc XRS+ system (Bio-Rad).

### In Vitro Functional Assays

CAR T cells were co-cultured with K562, K562/CD19, or K562/ROR1 cells at a T cell to tumor cell ratio of 2:1. In some experiments, CAR T cells were also incubated with control or anti-STII beads at a ratio of 30 μL beads per million cells. Cytokine concentrations in cellular supernatant were quantified by ELISA (ThermoFisher Cat #s 88-7316-88, 88-7025-88, 88-7346-88) 24 hours after stimulation. For intracellular cytokine staining, GolgiPlug (BD Bioscience Cat #555029) was added during assay setup and cells were harvested and stained after 5 hours of stimulation. T cell proliferation was quantified by staining CAR T cells with a 0.2 μM solution of carboxyfluorescein succinimidyl ester (CFSE) dye (ThermoFisher Cat # C34554) and incubating CAR T cells with K562/CD19, K562/ROR1 cells, control beads, or anti-STII beads for 72 hours.

### RNA Sequencing (RNA-Seq)

RNA was extracted from 24 samples from three donors. Total RNA integrity was checked using an Agilent 4200 TapeStation (Agilent Technologies) and quantified using a Trinean DropSense96 spectrophotometer (Caliper Life Sciences). RNA-seq libraries were prepared from total RNA using the TruSeq RNA Sample Prep Kit v2 (Illumina Cat # RS-122-2001) and a Sciclone NGSx Workstation (PerkinElmer). Library size distributions were validated using an Agilent 4200 TapeStation. Additional library quality control, blending of pooled indexed libraries, and cluster optimization were performed using a Qubit 2.0 Fluorometer (ThermoFisher). RNA-seq libraries were pooled (6-8-plex) and clustered onto a flow cell lane. Sequencing was performed using an Illumina HiSeq 2500 in rapid mode employing a paired-end, 50 base read length (PE50) sequencing strategy.

### Quantitative PCR

RNA was extracted and 500 ng was used to prepare cDNA with SuperScript III (ThermoFisher Cat #11752-250). 15 ng cDNA was added to a reaction with one of the following TaqMan assay probes: Hs00953178_ml (EPHA4), Hs00172973_ml (FOXO4), Hs00902234_ml (IL7R), Hs00360439_g1 (KLF2), or Hs99999907_ml (B2M). Reactions were run in duplicate or triplicate on one 384-well plate. ΔCt values were calculated by dividing mean Ct of technical triplicates from EPHA4, FOXO4, IL7R, and KLF2 probes by the mean Ct of the housekeeping gene β2 microglobulin (B2M). CD28/CD3, M1, or M3 versus 4-1BB/CD3ζ ratios for each donor were calculated and subjected to a log₂ transformation.

### NOD/SCID/γc^{-/-} (NSG) Mouse Model

Six- to eight-week-old male NSG mice were engrafted with 5×10⁵ Raji/ffluc cells via tail vein injection. One week later, PBS or a mixture (9×10⁵ cells) of CD8⁺ and CD4⁺ CAR T cells that had been expanded with CD19⁺ Epstein Barr virus lymphoblastoid cells for 9 days were injected intravenously. Bioluminescence imaging was performed as described in Sommermeyer et al. (Leukemia 30:492-500 (2016)). For experiments where differences between individual mice were expected, at least five mice per experimental group were used for data analysis to provide 81% power to detect an effect size of 1.75, based on a t-test with a one-sided 0.05 level of significance. Mice handlers were blinded to group allocation. The treatment groups were as follows: PBS/Sham (n=6 mice); 4-1BB/CD3ζ CAR T cells (n=15 mice); CD28/CD3ζ CAR T cells (n=15 mice); CD28/CD3ζ Y1 CAR T cells (n=15 mice); CD28/CD3ζ Y3 CAR T cells (n=15 mice).

### Shotgun Mass Spectrometry Data Analysis

Raw MS/MS spectra from each replicate experiment were searched together against the reviewed Human Universal Protein Resource (UniProt) sequence database (release 2016_01) with common laboratory contaminants using the MaxQuant/Andromeda search engine version 1.6.0.1 (Cox et al., Nat. Biotechnol. 26:1367-1372 (2008). The search was performed with a tryptic enzyme constraint for up to two missed cleavages. Variable modifications were oxidized methionine, phosphorylated serine, phosphorylated threonine, and phosphorylated tyrosine. Carbamidomethylated cysteine was set as a static modification. Peptide MH+ mass tolerances were set at 20 ppm. The overall FDR was set at ≤1% using a reverse database target decoy approach.

For the three TMT experiments, phosphopeptide site localization was determined by MaxQuant and converted to phosphorylation sites using Perseus version 1.6.0.7 (Tyanova et al., Nat. Methods 13:731-740 (2016)). At this step, reverse hits and potential contaminants were excluded from further analysis. Data normalization was performed by scaling each TMT channel to the channel median, followed by a log₂ transformation. Stimulation vs. control ratios were calculated by subtracting the appropriate control channels from stimulated channels. Due to incomplete MS sampling, some phosphorylation sites (features) were only found in one or two replicate experiments, and a much smaller minority (<1%) of sites were not found in every TMT channel.

Differential expression analyses over PO₄ sites were performed using the limma statistical framework and associated R package (G. K. Smyth, Stat Appl Genet MolBiol 3:Article3 (2004); Ritchie et al., Nucleic Acids Res. 43:e47 (2015)). For these analyses, only those features that had values in at least two experiments and all TMT channels were kept, leaving 14,490 quantified phosphorylation sites. A linear model was fitted to each PO₄ site, and empirical Bayes moderated t-statistics were used to assess differences in expression/abundance. Contrasts comparing stimulation vs control treatments were tested. Given that ROR1-specific and CD19-specific CARs displayed similar phenotypes across the two donors, and were stimulated identically in a ligand-independent manner (FIGS. 1D-1F), the corresponding measurements were considered replicates. Intraclass correlations were estimated using the duplicateCorrelation function of the limma package to account for measures originating from the same patients and the same antigens (Smyth et al., Bioinformatics 21:2067-2075 (2005)). An absolute log₂ fold change cutoff (stimulation versus control) of 0.7 and a false discovery rate (FDR) cutoff of 5% were used to determine differentially expressed PO₄ sites. Analyses of signaling networks and KEGG Pathways were performed using StringDB.

### RNA Sequencing Data Analysis

Image analysis and base calling were performed using Illumina's Real Time Analysis v1.18 software, followed by‘demultiplexing’ of indexed reads and generation of FASTQ files, using Illumina's bcl2fastq Conversion Software v1.8.4. The RNA-seq data were aligned to the human genome (UCSC Human Genome Assembly GRCh38 reference) using STAR, and gene quantification was performed using RSEM (Dobin et al., Bioinformatics 29:15-21 (2013); Li & Dewey, BMC Bioinformatics 12:323 (2011)). Genes with less than 10 non-zero read counts (taking into account technical replicates) were discarded, leaving 18,498 expressed genes. All libraries passed the quality control criteria (libraries with more than 200,000 reads, 12,000 detected genes and an exon range>60%). Raw count data were imported into R. edgeR was used to calculate the normalization factors to scale the raw library sizes, followed by a voom transformation from the limma Bioconductor package (Robinson & Oshlack, Genome Biol. 11:R25 (2010); Law et al., Genome Biol. 15:R29 (2014)). It transforms count data to log₂ counts per million and estimates the mean-variance relationship to compute appropriate observation-level weights. Linear models with subject random effects were again used for differential gene expression analysis as described above in Shotgun mass spectrometry data analysis. Contrasts comparing treatments (control versus stimulation) or CARs (CD28/CD3ζ versus 4-1BB/CD3ζ) were tested. An absolute log₂ fold change cutoff of 1 and a FDR cutoff of 1% were used to determine differentially expressed genes.

### Example 2

### Clinically Relevant Primary T Cell Model for Analysis of Car Signaling

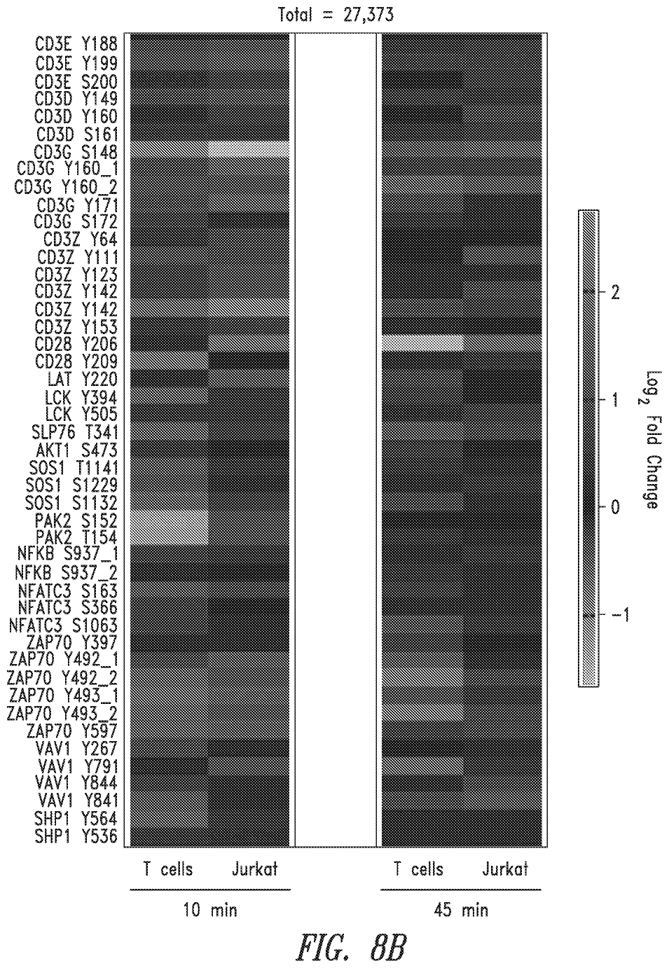

TCR signaling has been studied using LC-MS/MS analysis of transformed Jurkat T cells stimulated with anti-CD3 monoclonal antibody (mAb) (Mayya et al., Sci Signal 2:ra46-ra46 (2009); Nguyen et al., Mol. Cell Proteomics 8:2418-2431 (2009); Salek et al., PLoS ONE 8:e77423 (2013)). Jurkat cells were selected for signaling studies due to the ease with which they can be grown and manipulated using common molecular biology techniques. Introducing CARs into Jurkat cells to analyze signaling was considered, but a comparison of PO₄ of known proximal TCR signaling molecules by LC-MS/MS after anti-CD3 mAb stimulation revealed that immortalized Jurkat and cultured primary human T cells exhibited distinct patterns of protein PO₄ (FIGS. 8A-8B). In contrast to immortalized Jurkat cells, primary T cells displayed prolonged PO₄ of CD3 chains and ZAP-70, and differentially phosphorylated CD28, LAT, LCK, PAK2, SHP1, SOS1, and VAV1 after stimulation. Accordingly, CAR signaling was studied in primary human T cells to obtain clinically relevant data.

### Example 3

### Design of Tagged Cars for Analysis of Car Signaling

CAR antigen-expressing tumor cells can be used to stimulate primary CAR T cells for LC-MS/MS, but this approach requires prolonged culture of both tumor cells and T cells with isotope-labeled amino acids (SILAC) to be able to distinguish phosphopeptides from each cell type. Tumor cells can also engage accessory molecules on the T cell that contribute to phosphorylation of signaling proteins. To circumvent these drawbacks of cell based-stimulation, a cell-free method for activating CAR signaling was developed (FIGS. 1A-1B). Lentiviral vectors encoding CD19-specific and ROR1-specific CARs that contained either CD28/CD3ζ or 4-1BB/CD3ζ signaling domains and a nine-amino acid Strep-tag II (STII; SEQ ID NO:10) sequence in the spacer region were designed (SEQ ID NOS:19, 21, 23, and 25) (FIG. 1A). All constructs contained a truncated EGFR (EGFRt) transduction marker downstream of a T2A element for purification of CAR-expressing T cells (Wang et al., Blood 118:1255-1263 (2011). It was previously shown that inclusion of the STII sequence did not interfere with CAR T cell recognition or function and that STII CD28/CD3ζ or 4-1BB/CD3ζ CAR T cells could be efficiently activated and expanded in vitro by simulation with anti-STII mAb ((Liu et al., Nat. Biotechnol. 34, 430-434 (2016)).

Primary CD8⁺ T cells were transduced with each of the four lentiviral vectors, sorted for EGFRt⁺ expression, and expanded with a single cycle of stimulation to >1.6×10⁸ cells for subsequent analysis (FIG. 1C). CD28/CD3ζ and 4-1BB/CD3ζ CARs of each scFv specificity were present at similar levels on the cell surface (FIG. 1D). CAR T cells also expressed similar levels of CD45RO, CD62L, CD27, and CD28 (FIG. 1E), although a slightly greater frequency of CD19 4-1BB/CD3ζ CAR T cells expressed CD62L. PD-1 and Tim3 were not expressed by any CAR T cell population and >85% of CD28/CD3ζ and 4-1BB/CD3ζ CAR T cells were in the G₀/G₁ cell cycle phase, consistent with a lack of significant tonic signaling (FIGS. 1E-1F).

Prior to preparing lysates for MS analysis, canonical T cell signaling events induced by STII ligation were evaluated. ROR1-specific 4-1BB/CD3ζ CAR T cells were incubated for 45 minutes with various amounts of anti-STII mAb-coated microbeads and measured CD3ζ Y142, SLP-76 Y376, and PLC-γ1 Y783 by Western blot. For comparison, CAR T cells were stimulated with ROR1-transduced K562 (K562/ROR1) tumor cells. At the highest bead-to-cell ratio, anti-STII bead stimulation increased PO₄ of CD3ζ, SLP-76, and PLC-γ1 to a level similar to that observed in lysates from CAR T cells stimulated with K562/ROR1 cells, and this bead to cell ratio was used for all subsequent experiments (FIG. 1G). PO₄ of these substrates was also similar in CD19 CAR T cells stimulated with K562/CD19 or with anti-STII mAb-coated beads. Thus, anti-STII mAb-coated beads provide a precise method to selectively activate CAR signaling in primary T cells.

### Example 4

### Analysis of Protein Phosphorylation in T Cells Containing Cars with Distinct Co-Stimulatory Domains

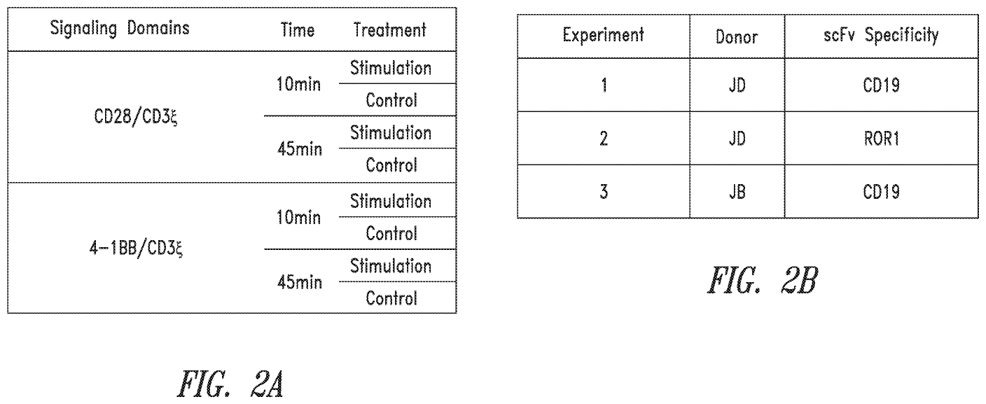

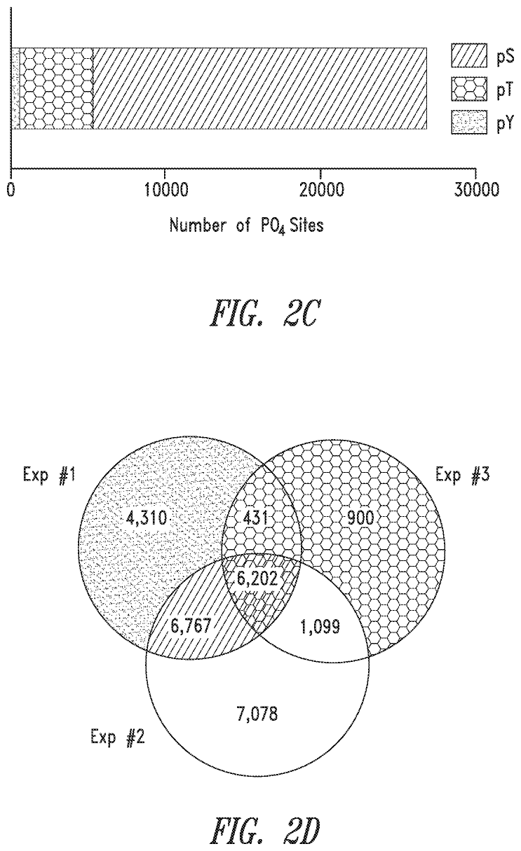

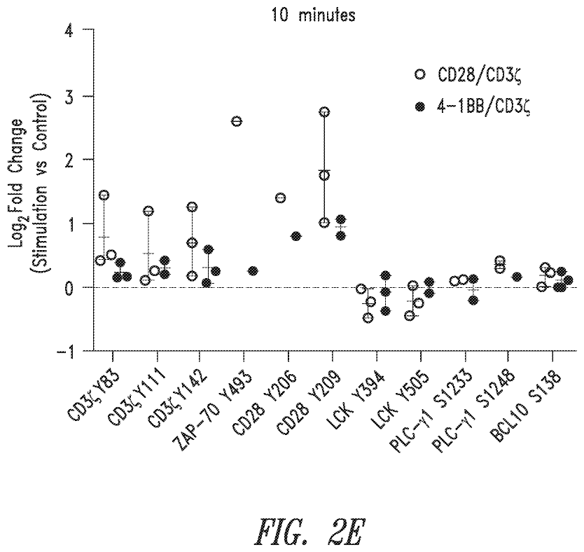

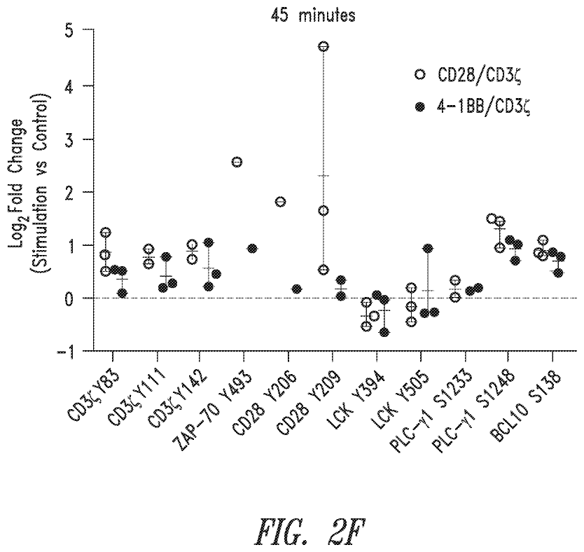

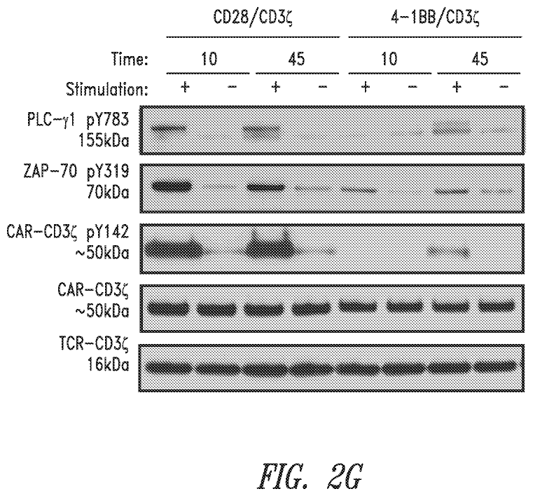

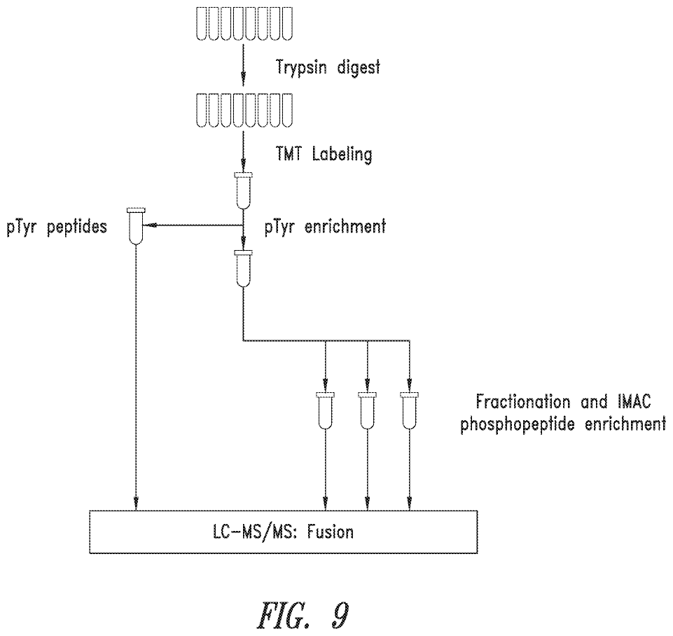

Three independent experiments were performed in which CD28/CD3ζ or 4-1BB/CD3ζ CAR T cells were incubated with anti-STII mAb-coated or uncoated (control) magnetic beads for 10 or 45 minutes to capture early and later PO₄ events initiated by CAR stimulation (FIG. 2A). CD19-specific CAR T cells generated from two different donors were used in two experiments; a third experiment utilized ROR1-specific CAR T cells derived from one of the two donors (FIG. 2B). To provide relative quantitation of phosphopeptides within each experiment, each trypsin-digested lysate was labeled with a unique isobaric Tandem Mass Tag (TMT) and enriched for phosphopeptides by anti-phosphotyrosine immunoprecipitation followed by immobilized metal affinity chromatography of the remaining eluate (Thompson et al., Anal. Chem. 75:1895-1904 (2003)) (FIG. 9).

Using these techniques, a total of 26,804 PO₄ sites were detected across the three experiments corresponding to 4,849 gene products. Among PO₄ sites, 571 (2.13%) were phosphotyrosines, 4,647 (17.33%) were phosphothreonines, and 21,586 (80.53%) were phosphoserines (FIG. 2C). There was considerable overlap in the captured phosphoproteome between replicate experiments (FIG. 2D). 99% of PO₄ sites detected in each experiment were present in both unstimulated and stimulated T cell lysates, enabling quantitation of changes induced by CAR activation (Navarro et al., Nat. Immunol. 12:352-361 (2011); van Oers et al, Mol Cell Biol 13:5771-5780 (1993))..

### Example 5

### Cars Alter Phosphorylation of Canonical T Cell Signaling Intermediates

Well-described TCR stimulation-induced PO₄ events were first analyzed within the MS dataset to determine whether these sites were also CAR stimulation-responsive (Brownlie and Zamoyska, Nature Reviews Immunology 13:257-269 (2013)). A log₂ fold change (log 2FC) value was calculated for each PO₄ site within each replicate experiment by comparing stimulated samples to appropriate controls (i.e. CD28/CD3ζ CAR 10-minute stimulation vs. CD28/CD3ζ CAR 10-minute control). At 10 minutes, increased PO₄ of CD3ζ at Y83, Y111, and Y142, as well as of ZAP-70 at Y493, was found in stimulated CAR T cells (FIG. 2E). Notably, both CD28/CD3ζ and 4-1BB/CD3ζ CAR T cells displayed increased PO₄ of CD28 at Y206 and Y209, demonstrating that ligation of the 4-1BB/CD3ζ CAR resulted in PO₄ of endogenous CD28. After 45 minutes of stimulation, PO₄ was observed to spread to downstream TCR signaling nodes, including increased PO₄ of PLC-γ1 at S1248 and BCL10 at S138 (FIG. 2F) (see also Rueda et al., J Immunol 178:4373-4384 (2007); Ishiguro et al., Mol. Immunol. 44:2095-2100 (2007)). For a majority of these known sites, stimulation of CD28/CD3ζ CARs initiated a greater magnitude log₂FC than 4-1BB/CD3ζ CARs at both 10 and 45 minutes. Reproducible changes in the PO₄ of CD3δ, ε, or γ ITAMs, which are known to be phosphorylated in T cells after anti-CD3 stimulation, were not observed at either time point.

To validate some of the differences in magnitude observed between CD28/CD3ζ and 4-1BB/CD3ζ CAR T cells in the MS dataset, cell lysates were evaluated for three CAR activation-induced phosphoprotein signaling events—CD3C pY142; ZAP-70 pY319; and PLC-γ1 pY783—by Western blot. As shown in FIG. 2G, CD28/CD3ζ or 4-1BB/CD3ζ CAR stimulation increased PO₄ of the CAR CD3C domain, ZAP-70 and PLC-γ1. In concordance with the MS data, more intense PO₄ of these proteins was observed in stimulated CD28/CD3ζ CAR T cells. Notably, a low level of basal CAR CD3ζ PO₄ was detected in unstimulated CD28/CD3ζ CD19 and ROR1 CAR T cells that was not present in 4-1BB/CD3ζ CAR T cells of either specificity. Constitutive CAR CD3ζ domain PO₄ has been described for several CARs and can be linked to adverse T cell phenotypes and upregulation of T cell exhaustion markers (see Frigault et al., Cancer Immunol Res 3:356-367 (2015); Long et al., Nature Medicine 21:581-590 (2015)). However, differences in cell cycle or in the expression of PD-1 or Tim3 in unstimulated CD28/CD3ζ or 4-1BB/CD3ζ CAR T cells were not observed (FIGS. 1E-1F). Thus, the low level of basal PO₄ detected in unstimulated CD28/CD3ζ CAR T cells was distinct from the more extreme tonic signaling observed in some CARs with different scFv specificities.

### Example 6

### Activation of Car T Cells with Distinct Co-Stimulatory Domains Induce Similar Protein Phosphorylation Events with Different Kinetics and Magnitude

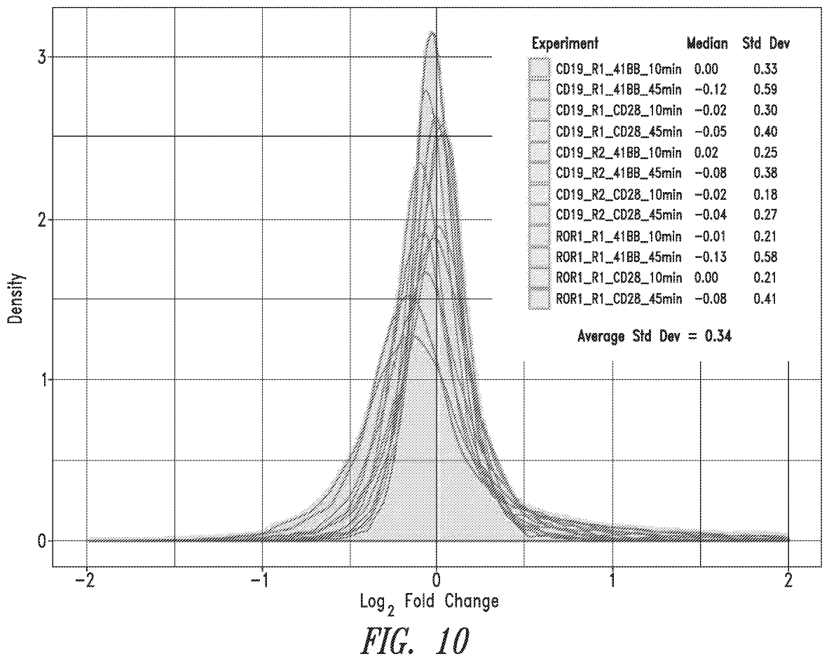

An advantage of shotgun MS is that it quantitatively measures thousands of PO₄ events to which there are no known experimentally validated antibodies. The limma statistical framework and associated R package were used to identify PO₄ sites that were modulated after CD28/CD3ζ and 4-1BB/CD3ζ CAR ligation (G. K. Smyth, Stat Appl Genet Mol Biol 3:Article3 (2004)). A PO₄ site was identified as CAR stimulation-responsive if it was detected in at least two of the three experiments, displayed an average |log₂FC|≥0.7 between stimulated and unstimulated conditions at 10 or 45 minutes, and met a 5% FDR cutoff. A log₂FC cutoff of 0.7 was chosen because this represents approximately two standard deviations of the distribution of log₂FC values (FIG. 10).

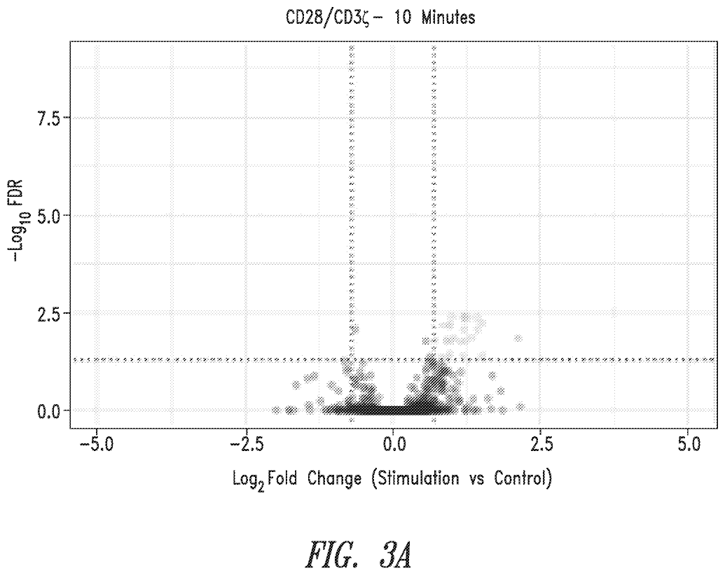

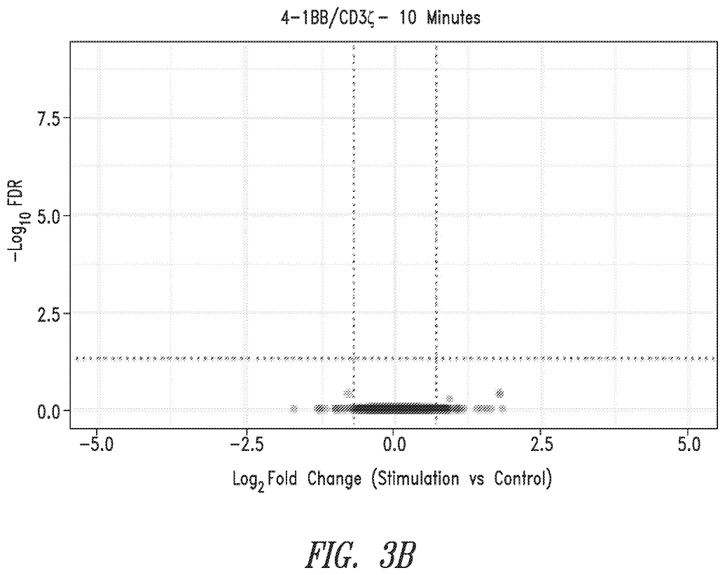

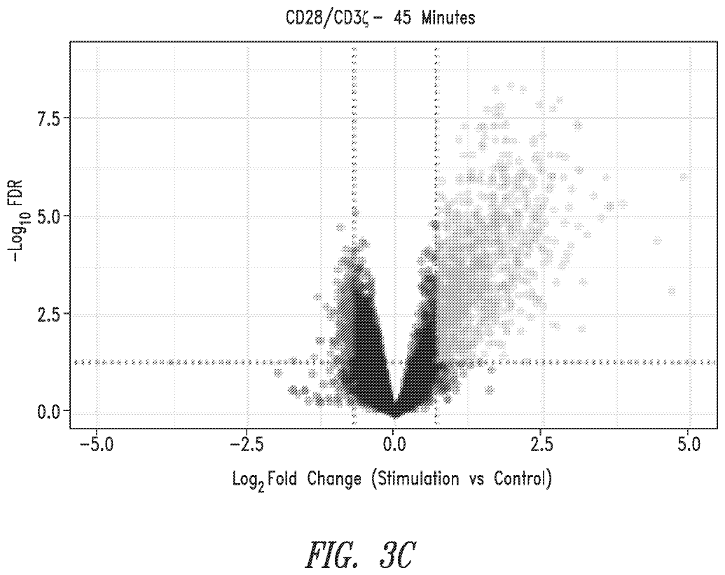

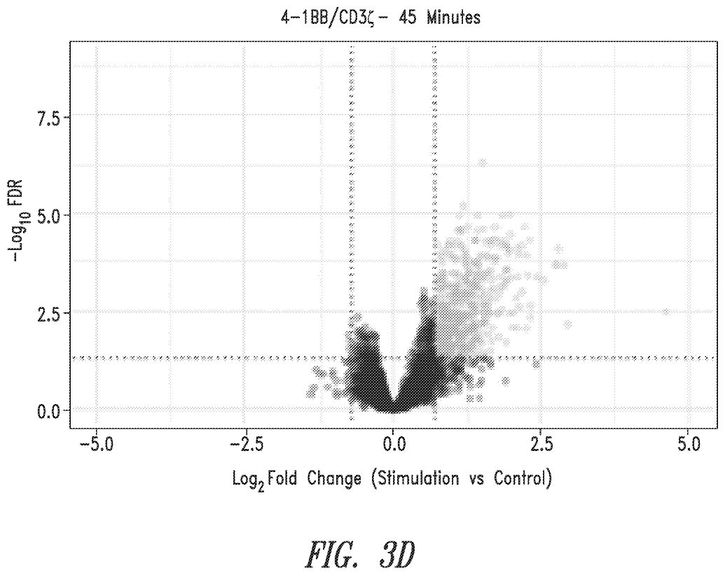

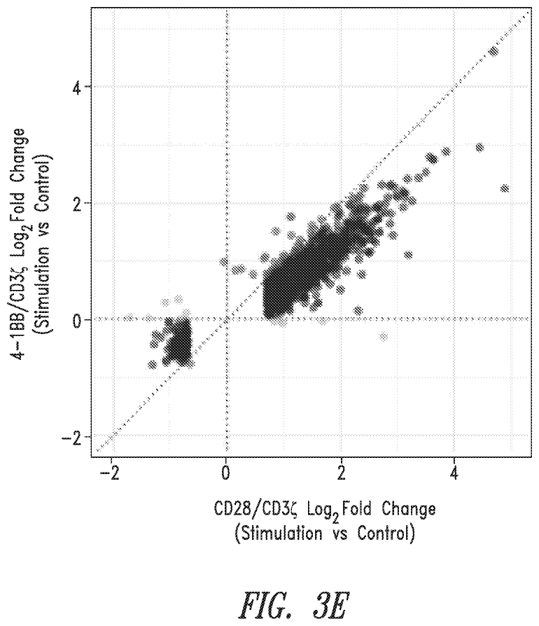

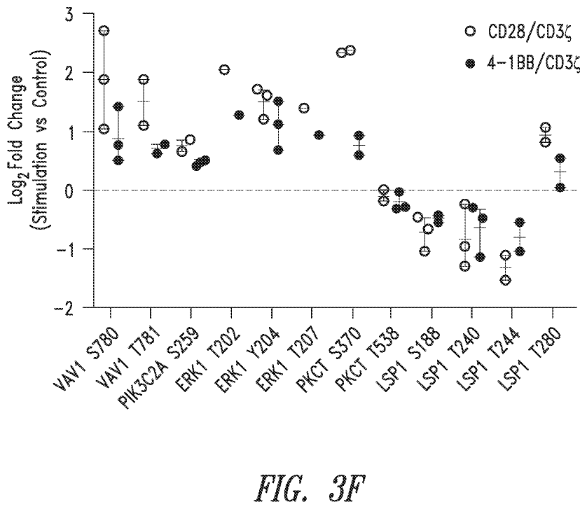

Using these stringent cut-offs, 26 PO₄ sites from 22 proteins in CD28/CD3ζ CAR T cells were identified as stimulation-responsive at the 10 minute time point. These differentially phosphorylated proteins were enriched for members of the KEGG TCR signaling pathway, and included increased PO₄ of p21 activated kinase 2 (PAK2) at S64, CD8 alpha (CD8A) at S231, protein kinase C θ (PKCT) at S370, and proto-oncogene vav (VAV1) at S748 and T749 (FIG. 3A and Tables 1A-1B). In contrast, no sites from 4-1BB/CD3ζ CAR T cells met the log₂FC and FDR criteria at the 10-minute time point (FIG. 3B). 74 PO₄ sites met the log₂FC cutoff, but not the FDR cutoff, and among these, increased PO₄ of neuroblast differentiation-associated protein (AHNAK) at S4903 and calcium/calmodulin-dependent protein kinase type II subunit delta (CAMK2D) at S315 and S319 were detected, indicative of modulated Ca²⁺ signaling (Tables 1A-1B). The lack of robust alterations in early protein PO₄ induced by the 4-1BB/CD3ζ CAR was unexpected but consistent with Western blot data showing minimal PO₄ of CAR CD3ζ and PLC-γ1 at 10 minutes (FIG. 2G).

After 45 minutes of stimulation, more intense protein PO₄ had occurred and 1,289 PO₄ sites met the log₂FC and FDR cutoffs from either CD28/CD3ζ or 4-1BB/CD3ζ CAR samples. These included 1,279 PO₄ sites from 743 gene products in CD28/CD3ζ CAR T cells and 522 sites from 346 gene products in 4-1BB/CD3ζ CAR T cells (FIGS. 3C-3D). Thus, stimulation of the CD28/CD3ζ CAR continued to result in alteration of a greater number of PO₄ sites. Strikingly, however, a strong correlation among the stimulation-responsive PO₄ sites was observed, whereby nearly all sites were modulated in similar fashions by CD28/CD3ζ and 4-1BB/CD3ζ CAR stimulation (FIG. 3E). Only 12 (0.93%) of the 1,289 sites that met the cut-offs in CD28/CD3ζ CAR samples were differentially modulated by 4-1BB/CD3ζ CAR stimulation, and only 43 (3.3%) sites exhibited a greater magnitude log₂FC after 4-1BB/CD3ζ CAR stimulation, consistent with prior data suggesting more intense signaling downstream of CD28/CD3ζ CARs (FIG. 3E). Furthermore, these subsets of 12 and 43 sites did not map to currently defined 4-1BB signaling networks.

Finding few differences in protein PO₄ events after stimulation of CD28/CD3C and 4-1BB/CD3ζ CARs was unexpected and educed the question of how proteins involved in canonical T cell costimulatory signaling pathways were affected. In agreement with the earlier finding that 4-1BB/CD3ζ CARs activated endogenous CD28, the CD28 signaling intermediates VAV1, PKCT, and PIK3C2A displayed increased PO₄ after stimulation of either CAR (FIG. 3F) (Acuto and Michel, Nature Reviews Immunology 3:939-951 (2003)). Within the 4-1BB signaling pathway, LSP1, a direct target of 4-1BB and TRAF2 signaling, was modulated by both CARs (Sabbagh et al., J. Leukoc. Biol. 93:713-721 (2013)). Despite these similarities, CD28/CD3ζ CARs modulated each PO₄ site, including those in the 4-1BB signaling pathway, by a greater magnitude than 4-1BB/CD3ζ CARs. Thus, rather than activating divergent signaling networks, as might be predicted from the distinct costimulatory domains in the receptors, ligation of CD28/CD3ζ and 4-1BB/CD3ζ CARs induced highly similar changes in intracellular protein PO₄ that encompassed both canonical CD28 and 4-1BB signaling intermediates.

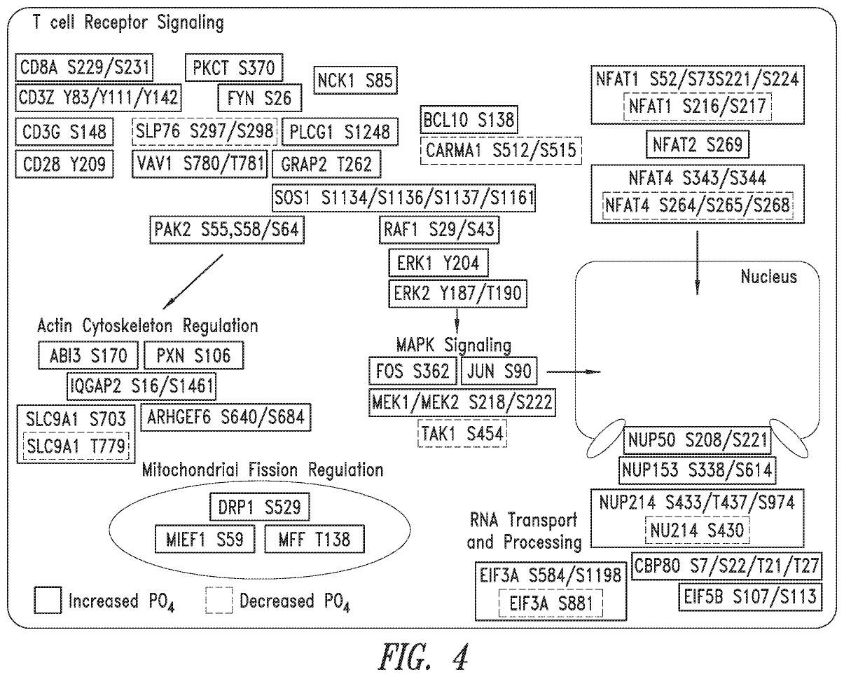

A map illustrating the major pathways and individual protein phosphorylation events affected by CD28/CD3ζ and 4-1BB/CD3ζ CAR stimulation includes proteins involved in canonical TCR signaling and mitogen-activated protein kinase (MAPK) signaling pathways (FIG. 4 and Table 1A).

### Example 7

### Differences in the Magnitude of Car Signaling Persist after Car Ligation

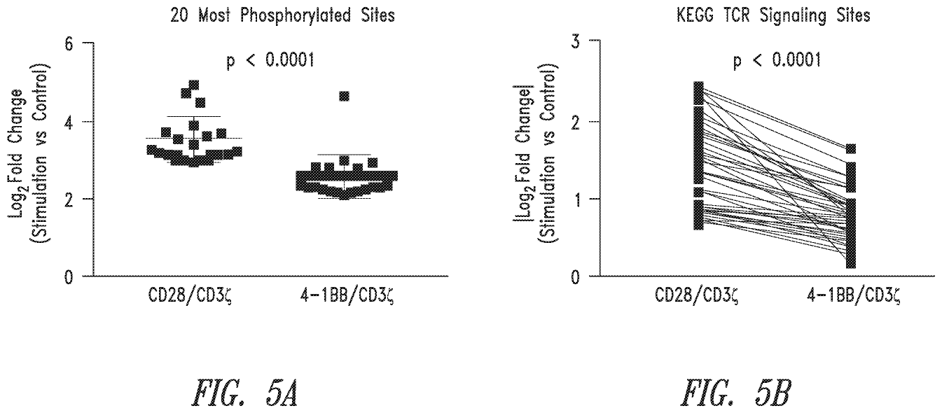

Protein PO₄ mediated by CD28/CD3ζ and 4-1BB/CD3ζ CAR stimulation was highly similar but differed in intensity at the vast majority of PO₄ sites. It was reasoned that quantifying changes in protein PO₄ after CAR stimulation could provide a holistic measure of CAR signal strength. Stimulation-responsive PO₄ sites at 45 minutes from CD28/CD3ζ and 4-1BB/CD3ζ CAR samples were arranged by decreasing log₂FC. In-line with prior results showing that both CARs modulated protein PO₄ in similar fashions, 15 of the 20 most-phosphorylated sites after stimulation were shared between CD28/CD3ζ and 4-1BB/CD3ζ CAR T cells (Tables 2A-2B). However, PO₄ of the top 20 sites increased by 11.15-fold on average in CD28/CD3ζ CAR samples, but only 5.8-fold on average in 4-1BB/CD3ζ CAR samples (FIG. 5A). Stratifying CAR stimulation-responsive PO₄ sites by signaling pathway further showed that the average PO₄ site within the KEGG TCR signaling pathway was modulated by 2.52-fold in CD28/CD3ζ CAR samples, but only 1.69-fold in 4-1BB/CD3ζ CAR samples (FIG. 5B).

To determine if 4-1BB/CD3ζ CAR signals reached a similar intensity to that of CD28/CD3ζ CARs at later times, CAR T cells were stimulated for 60, 120, or 180 minutes and PO₄ of canonical and newly identified signaling intermediates were measured by Western blot. As shown in FIG. 5C, CD28/CD3ζ CAR stimulation resulted in more intense PO₄ of SLP-76, PLC-γ1, and DAPP1 in all samples, indicating that 4-1BB/CD3ζ CAR stimulation never rivaled CD28/CD3ζ CAR signal intensity during this time frame.

### Example 8

### Car Signal Intensity is Positively Associated with an Effector Cell-Like Phenotype and Reduced In Vivo Anti-Tumor Activity

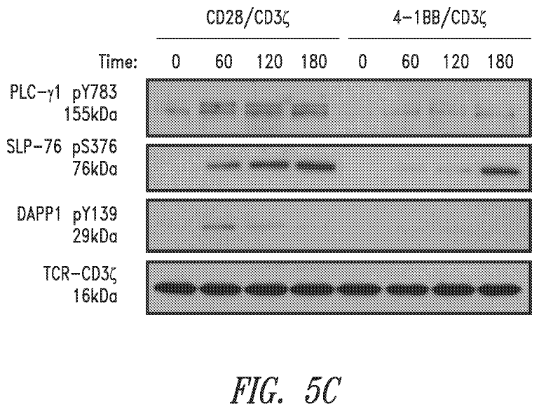

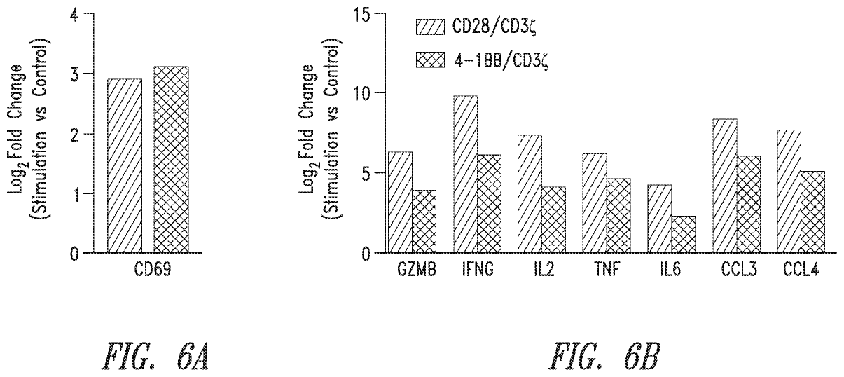

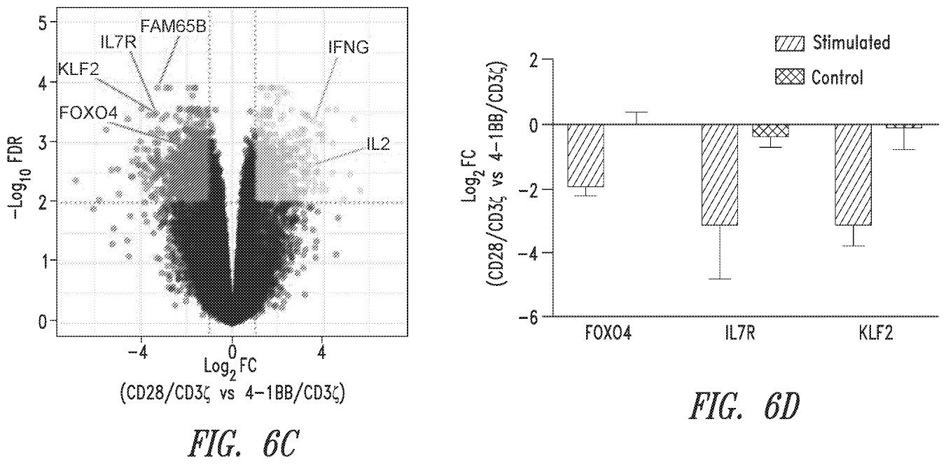

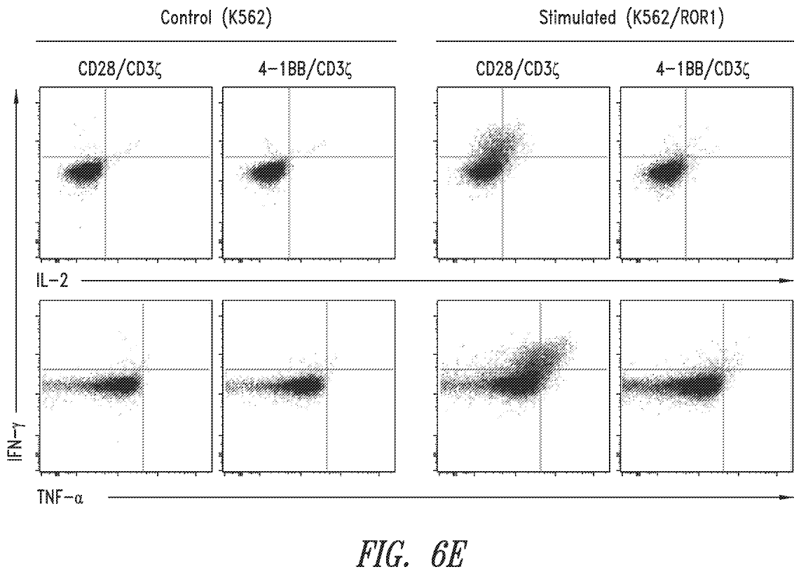

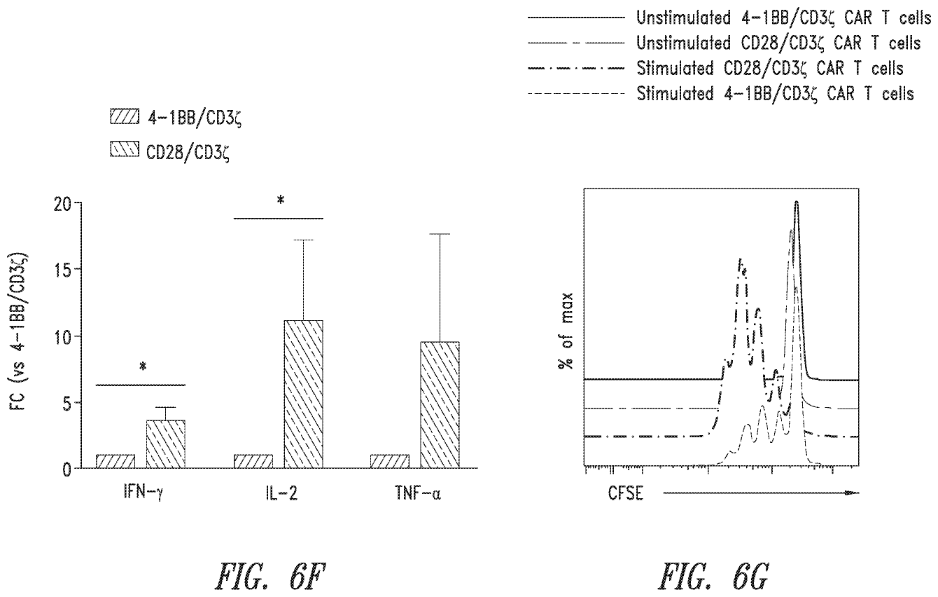

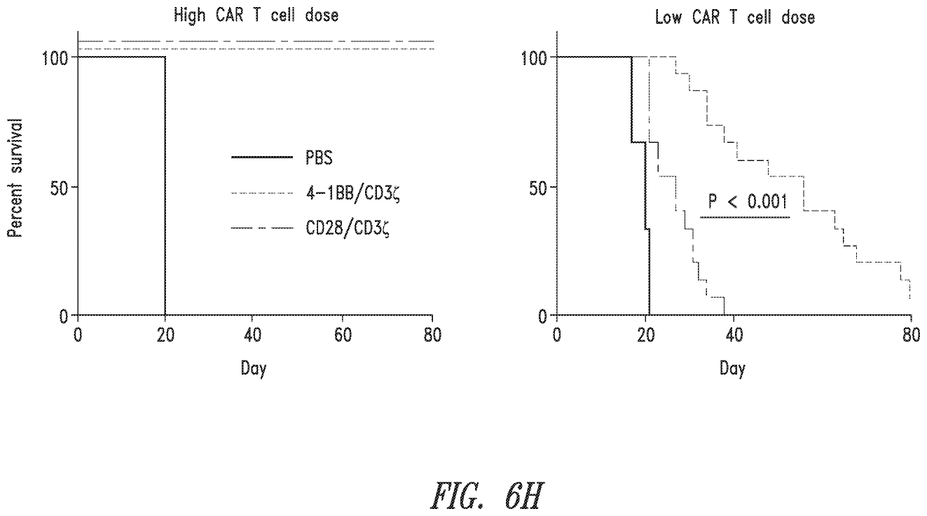

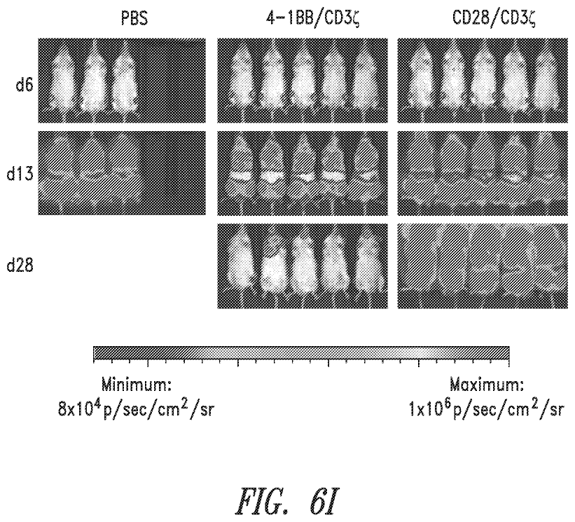

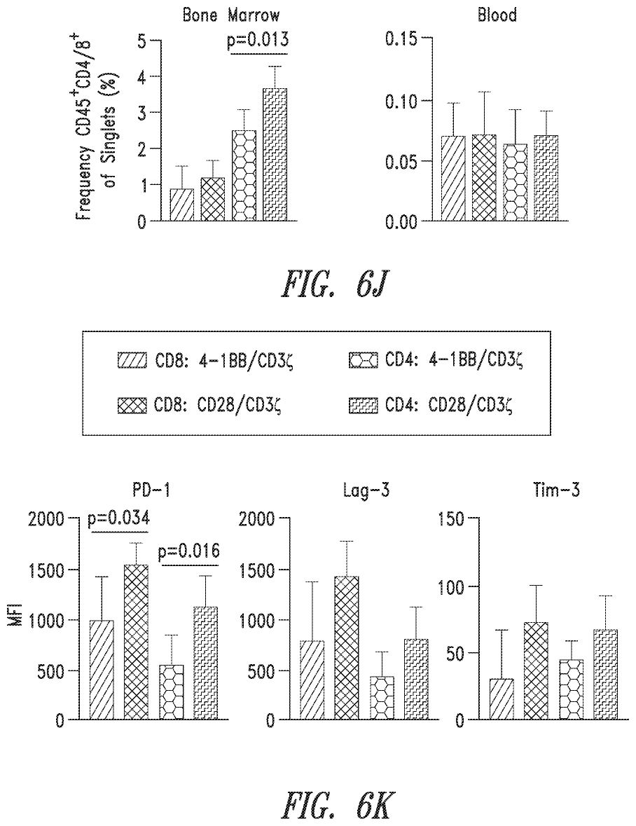

The strength of T cell activation and signal transduction leads to transcriptional differences that regulate effector cell differentiation and memory formation (Kaech and Cui, Nature Reviews Immunology 12:749-761 (2012)). RNA-Seq was used to analyze transcriptional programs in CD28/CD3ζ or 4-1BB/CD3ζ CAR T cells after 6 hours of STII bead stimulation and to identify differentially expressed genes meeting a log₂FC>1.0 and a FDR<1%. Consistent with the faster and more intense phosphoprotein signal, CD28/CD3ζ CAR stimulation initiated more marked early transcriptional changes. Using limma to compare stimulated to unstimulated CD28/CD3ζ CAR-T cells identified 4,894 differentially expressed genes, whereas 4-1BB/CD3ζ CAR stimulation resulted in 197 differentially expressed genes. The T cell activation marker CD69 was upregulated to similar degrees by CD28/CD3ζ or 4-1BB/CD3ζ CAR stimulation (FIG. 6A), but greater fold increases in expression of the effector molecules granzyme B (GZMB), interferon-γ (IFNG), interleukin-2 (IL2), tumor necrosis factor-α (TN), interleukin-6 (IL6), macrophage inflammatory protein 1a (CCL3), and macrophage inflammatory protein 10 (CCL4) were observed in activated CD28/CD3ζ CAR T cells compared with 4-1BB/CD3ζ CAR T cells (FIG. 6B).

Direct comparison of stimulated CD28/CD3ζ and 4-1BB/CD3ζ samples identified 1,673 differentially expressed genes (Table 3). Of these, Krüppel-like factor 2 (KLF2), interleukin-7 receptor (IL7R), and Rho family-interacting cell polarization regulator 2 (RIPOR2, previously known as FAM65B) were expressed at lower levels in CD28/CD3ζ CAR T cell samples (FIG. 6C). KLF2 and IL7R are associated with memory T cell formation and are targets of the FOXO family of transcription factors (Rougerie et al., J Immunol 190:748-755 (2013); Kaech et al., Nat Immunol 4:1191-1198 (2003); Kerdiles et al., Nat Immunol 10:176-184 (2009)). Along these lines, FOXO4 expression was reduced in stimulated CD28/CD3ζ CAR T cells. qPCR confirmed these expression differences and showed that these T cell memory-associated genes were not differentially expressed in unstimulated CD28/CD3ζ or 4-1BB/CD3C CAR T cells (FIG. 6D). Thus, CD28/CD3ζ CAR activation preferentially induced a transcriptional profile marked by increased expression of effector molecules and loss of FOXO family gene targets.

It was hypothesized that like TCR signaling, differences in signal quantity between CD28/CD3ζ and 4-1BB/CD3ζ CARs would affect T cell functions. To test this, CD28/CD3ζ and 4-1BB/CD3ζ CAR T cells were activated with CAR-antigen-expressing K562 cells or STII beads, and cytokine production and proliferation were measured at various time points. After 5 hours, a greater proportion of CD28/CD3ζ CAR T cells were producing IFN-γ, IL-2, and TNF-α (FIG. 6E), and after 24 hours, CD28/CD3ζ CAR T cells secreted markedly more IFN-γ, IL-2, and TNF-α than 4-1BB/CD3ζ CAR T cells (FIG. 6F). After 72 hours, a greater number of CD28/CD3C CAR T cells had divided and undergone more divisions than 4-1BB/CD3ζ CAR T cells (FIG. 6G).

Despite superior in vitro effector functions, CD28/CD3ζ CAR T cells proved less effective at in vivo tumor control. When 3×10⁶ CAR T cells were adoptively transferred into NOD/SCID/γc^{-/-} (NSG) mice bearing established CD19⁺ Raji lymphoma xenografts, both CD28/CD3ζ and 4-1BB/CD3ζ CAR T cells mediated complete tumor regression (FIG. 6H). However, infusion of a reduced (7.5-8×10⁵) cell dose showed that CD28/CD3ζ CAR T cells were much less potent than 4-1BB/CD3ζ CAR T cells, and all CD28/CD3ζ CAR T cell-treated mice died of tumor progression within 40 days. Tumor progression occurred in mice treated at the lower CD28/CD3ζ CAR T cell dose despite accumulation of CAR T cells to higher frequencies in tumor-involved bone marrow (FIG. 6I). CD28/CD3ζ CAR T cells in the bone marrow expressed higher levels of PD-1, Lag-3, and Tim-3 (FIG. 6J), consistent with the acquisition of an exhausted phenotype. Taken together, these data show that the rapid and intense signaling mediated by CD28/CD3ζ CAR activation led to an exhausted T cell phenotype with reduced anti-tumor activity, despite increased in vitro cytokine production and T cell proliferation.

### Example 9

### CD28/CD3z and 4-1BB/CD3z Cars Differentially Associate with Endogenous CD28 and LCK

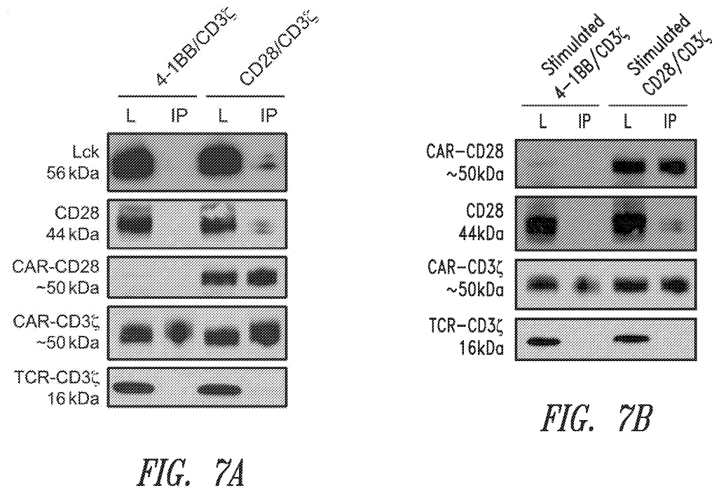

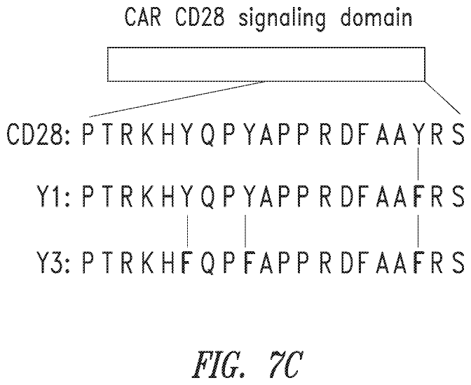

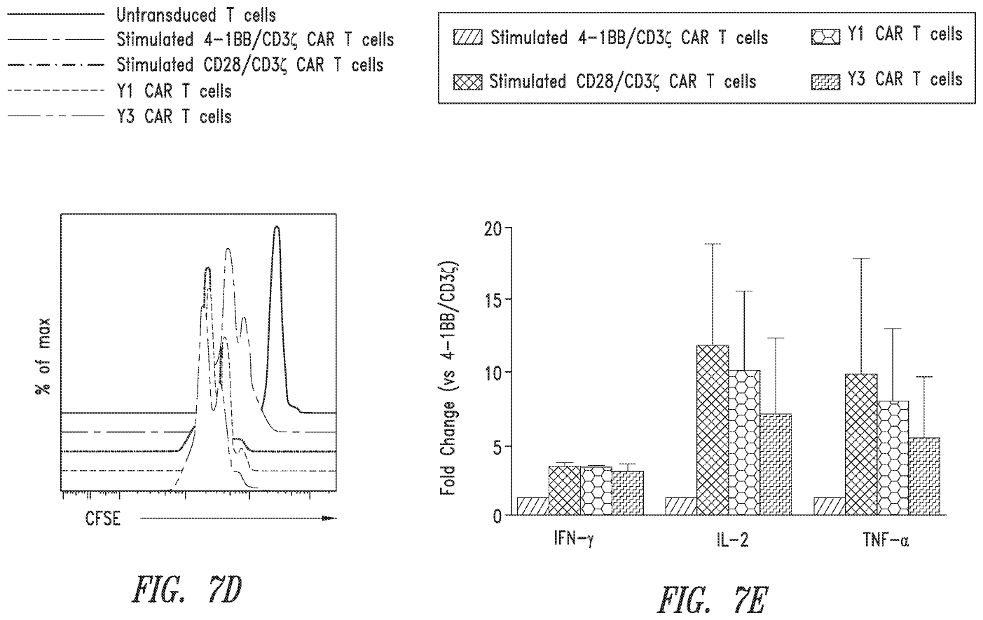

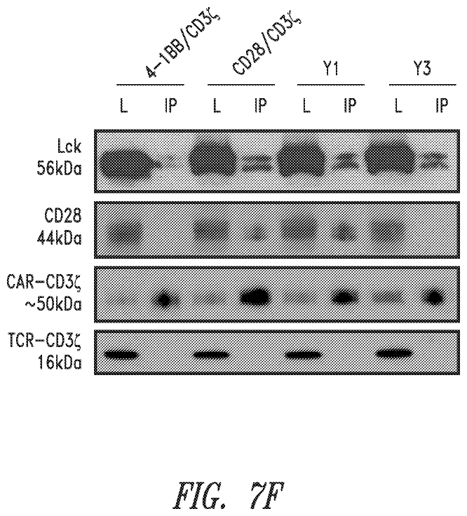

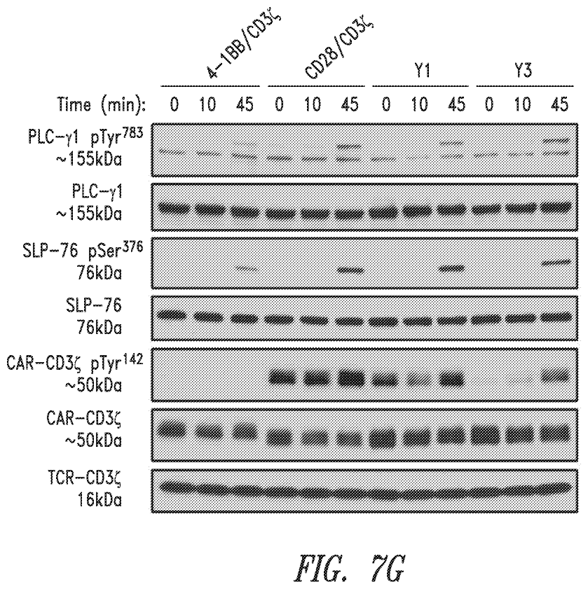

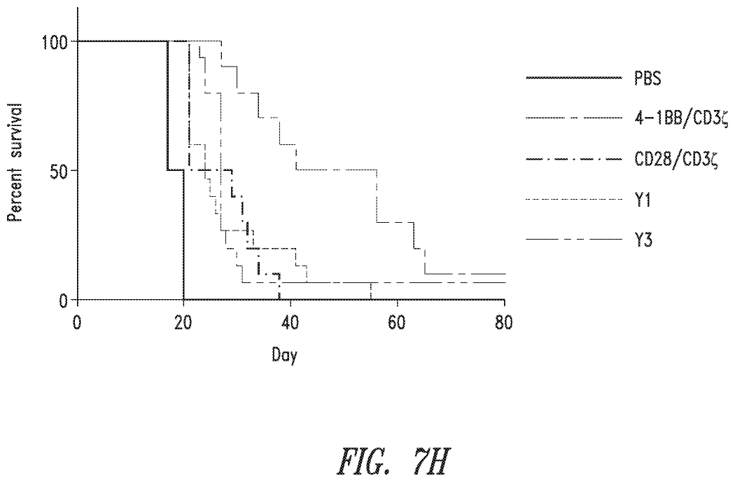

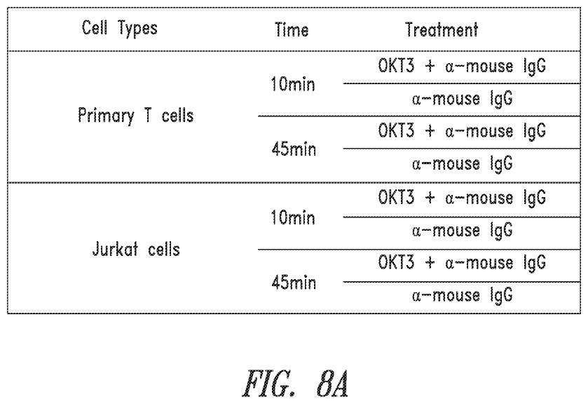

To interrogate possible causes of increased CD28/CD3ζ CAR signaling kinetics and strength, CAR complexes were immunoprecipitated from CD8⁺ T cells and probed for differences among associated T cell signaling proteins in the basal state. Western blot confirmed efficient CAR pull-down and showed association of endogenous CD28 and Lck with the CD28/CD3ζ CAR, but only minimal CD28 and Lck association with the 4-1BB/CD3ζ CAR (FIG. 7A). Immunoprecipitates from activated 4-1BB/CD3C CAR T cells after 45 minutes of CAR stimulation also failed to detect endogenous CD28 (FIG. 7B). Because basal CAR phosphorylation was conferred by the presence of the CD28 costimulatory domain and Y206, Y209, and Y218 were intensely phosphorylated after CAR stimulation (MS data), CD28/CD3ζ CARs with tyrosine to phenylalanine mutations at these residues were constructed (FIG. 7B). A CD19 specific CAR with Y218F substitution and CD19 CAR with Y206F, Y209F, and Y218F substitutions comprise the amino acid sequences set forth in SEQ ID NO:27 and 29, respectively. A ROR1 specific CAR with Y218F substitution and ROR1 CAR with Y206F, Y209F, and Y218F substitutions comprise the amino acid sequences set forth in SEQ ID NO:31 and 33, respectively.

CD19- and ROR1-specific CARs with mutations in one (Y1) or all three tyrosines (Y3) were efficiently expressed in T cells and functioned in vitro in response to co-culture with ROR1⁺ or CD19⁺ tumor cells by proliferating and producing interferon gamma (IFN-γ) (FIGS. 7C-7D). Notably, IL-2 and TNF-α secretion were reduced in a graded fashion by tyrosine mutations. Immunoprecipitations showed that Lck association was not abrogated by the Y1 and Y3 mutations, although endogenous CD28 did not associate with the Y3 CAR (FIG. 7F). Partial (Y1) or complete (Y3) abrogation of basal CAR CD3ζ PO₄ was observed in mutant CARs (FIG. 7G), but Y1 and Y3 CARs still phosphorylated SLP-76 and PLC-γ1 with similar kinetics and intensity as the wild-type CD28/CD3ζ CAR after STII bead stimulation (FIG. 7G). Consistent with Western blot data showing increased Y1 and Y3 signal strength, mice treated with Y1 or Y3 CAR T cells displayed median survival similar to mice treated with wild-type CD28/CD3ζ CAR T cells and less than that of 4-1BB/CD3ζ CAR T cells (FIG. 7H). Together, these results suggested that neither basal CAR CD3C phosphorylation nor endogenous CD28 association was responsible for the increased signal kinetics and strength of CD28/CD3ζ CARs.

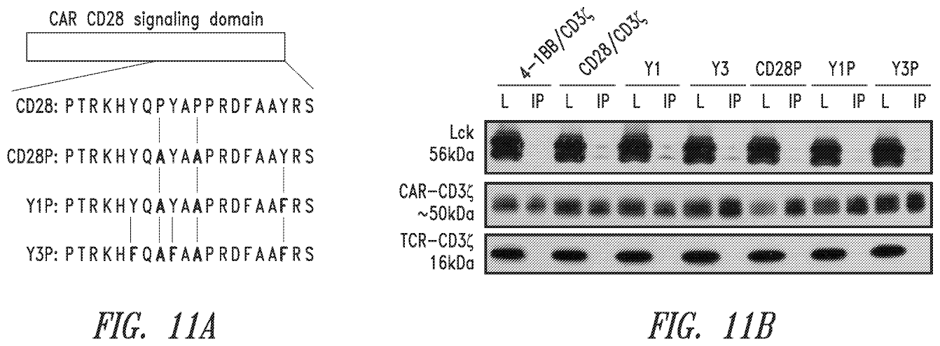

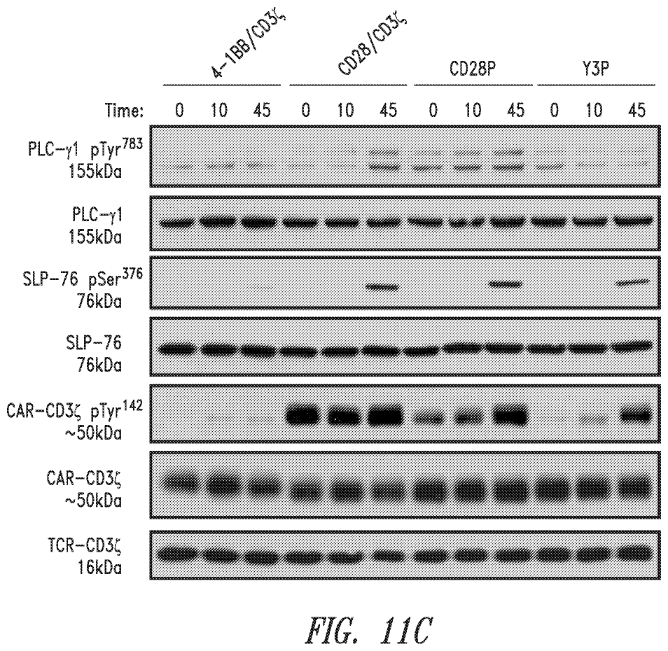

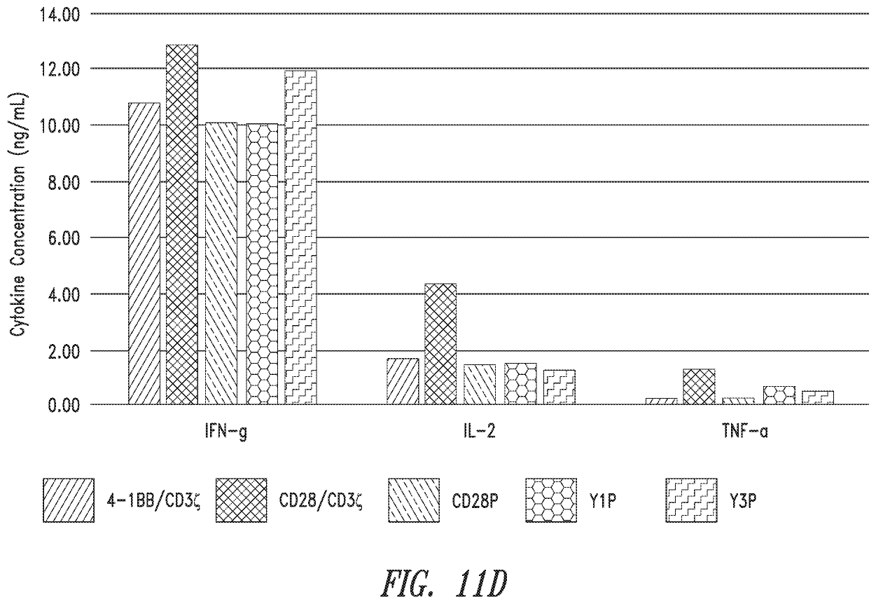

Constitutive Lck activation promotes T cell effector functions (Tavano et al., J Immunol 173:5392-5397 (2004)). To examine whether association of Lck with CD28/CD3ζ CARs mediates rapid and robust phosphorylation of signaling intermediates after CAR activation, proline-to-alanine mutations were generated at the Lck binding site of CD28 (FIG. 11A). Immunoprecipitation of wild-type and mutant CD28/CD3ζ CARs showed that Lck association was absent in CARs with proline-to-alanine mutations (FIG. 11B). Signaling analyses demonstrated that mutation of proline residues alone in CD28P CARs partially abrogated basal CAR CD3ζ phosphorylation, but did not reduce signal intensity (FIG. 11C). In contrast, simultaneous mutation of proline and tyrosine residues in Y3P CARs fully abrogated basal CAR phosphorylation and reduced the magnitude of SLP-76 and PLC-γ1 phosphorylation after stimulation. Notably, IFN-γ, IL-2 and TNF-α secretion were reduced by proline-to-alanine mutations such that the overall levels of cytokine production were similar to 4-1BB/CD3ζ CART cells (FIG. 11D). These data indicate differences in CAR signal intensity between CD28/CD3ζ CARs and 4-1BB/CD3ζ CARs are, in part, related to greater Lck association with CD28/CD3ζ CARs. Furthermore, CAR signal intensity and pro-inflammatory cytokine production can be altered by mutating tyrosine and proline residues in the CD28 signaling domain.

### Example 10

### Treatment of Cancer Using Modified CD28-Car T Cells

Autologous T cells are isolated from PBMCs of patients with solid or hematological cancers and sorted to isolate effector and helper T cells. T cell fractions are cultured with anti-CD3/anti-CD28 beads and IL-2 and transduced ex vivo with a lentiviral construct encoding a chimeric antigen receptor (CAR) that includes a scFv specific for a tumor antigen of interest, a spacer, a transmembrane domain, a modified CD28 costimulatory domain that includes Y206F, Y209F, and Y218F substitutions, and an intracellular signaling domain. The construct also encodes a cell surface marker for transduction. Transduced T cells are examined in vitro for functionality (proliferation, cytokine release) in response to stimulation with antigen-expressing tumor cells. Thereafter, CAR T cells are expanded in vitro.

Patients receive lymphodepleting chemotherapy and are thereafter administered a clinically relevant dose of CAR T cells via intravenous infusion. The CAR T cells expand in the patients after adoptive transfer and are measured by flow cytometry and q-PCR for vector sequences. Samples are taken from treated patients at the peak of in vivo expansion to measure phenotype, assess function, and determine antitumor persistence. Flow cytometry and gene expression profiling are performed to characterize gene expression in the cells. Reductions in tumor size, number, and distribution are monitored by MRI.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 62/635,450, filed Feb. 26, 2018, U.S. Provisional Patent Application No. 62/676,787, filed May 24, 2018, and U.S. Provisional Patent Application No. 62/739,792, filed Oct. 1, 2018, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### TABLES

TABLE 1A
26 CAR stimulation-responsive PQ4 sites
identified in CP28/CD3z CAR samples

| UniProt | Gene | log2 | | | UniProt | Amino | | Multi- | Sequence |
|---|---|---|---|---|---|---|---|---|---|
| Feature | Feature | FC | FDR | Gene | ID | Acid | Position | plicity | Window |
| Q8IZP0-2_S226_1 | ABI1_S231 | 0.718 | 0.016 | ABI1 | Q8IZP0-2 | S | 226 | _1 | SPARLGSQ |
| | | | | | | | | | HSPGRTAS |
| | | | | | | | | | LNQRPRTH |
| | | | | | | | | | SGSSGGS |
| | | | | | | | | | (SEQ ID |
| | | | | | | | | | NO: 76) |
| Q9P2A4-2_S164_1 | ABI3_S170 | 0.847 | 0.007 | ABI3 | Q9P2A4-2 | S | 164 | _1 | DLSTQLSR |
| | | | | | | | | | TGTLSRKS |
| | | | | | | | | | IKAPATPA |
| | | | | | | | | | SATLGRP |
| | | | | | | | | | (SEQ ID |
| | | | | | | | | | NO: 77) |
| Q09666_S5857_l | AHNAK_S5857 | 1.517 | 0.006 | AHNAK | Q09666 | S | 5857 | _1 | DDETGKLQ |
| | | | | | | | | | GSGVSLAS |
| | | | | | | | | | KKSRLSSS |
| | | | | | | | | | SSNDSGN |
| | | | | | | | | | (SEQ ID |
| | | | | | | | | | NO: 78) |
| Q01433__S100_1 | AMPD2_S100 | 1.035 | 0.014 | AMPD2 | Q01433 | S | 100 | _1 | EARGGLGA |
| | | | | | | | | | PPLQSARS |
| | | | | | | | | | LPGPAPCL |
| | | | | | | | | | KHFPLDL |
| | | | | | | | | | (SEQ ID |
| | | | | | | | | | NO: 79) |
| P51681_S337_1 | CCR5_S337 | 0.968 | 0.007 | CCR5 | P51681 | S | 337 | _1 | KCCSIFQQ |
| | | | | | | | | | EAPERASS |
| | | | | | | | | | VYTRSTGE |
| | | | | | | | | | QEISVGL |
| | | | | | | | | | (SEQ ID |
| | | | | | | | | | NO: 80) |
| P01732-2_S194_1 | CD8A_S231 | 1.341 | 0.013 | CD8A | P01732-2 | S | 194 | _1 | KCPRPVVK |
| | | | | | | | | | SGDKPSLS |
| | | | | | | | | | ARYV |
| | | | | | | | | | (SEQ ID |
| | | | | | | | | | NO: 81) |
| Q96BY6-3_S13_1 | DOCK10_NA | 0.997 | 0.004 | DOCK10 | Q96BY6-3 | S | 13 | _1 | MSFRGK |
| | | | | | | | | | VFKREPSE |
| | | | | | | | | | FWKKRRTV |
| | | | | | | | | | RRVNQE |
| | | | | | | | | | (SEQ ID |
| | | | | | | | | | NO: 82) |
| Q92608_S179l_2 | DOCK2_S1791 | 1.207 | 0.004 | DOCK2 | Q92608 | S | 1791 | _2 | EANTSPRL |
| | | | | | | | | | SQTFLQLS |
| | | | | | | | | | DGDKKTLT |
| | | | | | | | | | RKKVNQF |
| | | | | | | | | | (SEQ ID |
| | | | | | | | | | NO: 83) |
| Q92608_T1786_2 | DOCK2_T1786 | 1.207 | 0.004 | DOCK2 | Q92608 | T | 1786 | _2 | IPGLDEAN |
| | | | | | | | | | TSPRLSQT |
| | | | | | | | | | FLQLSDGD |
| | | | | | | | | | KKTLTRK |
| | | | | | | | | | (SEQ ID |
| | | | | | | | | | NO: 84) |
| P11171-2_S84_1 | EPB4I_S84 | 0.910 | 0.017 | EPB41 | P11171-2 | S | 84 | _1 | NKERTSES |
| | | | | | | | | | RGLSRLFS |
| | | | | | | | | | SFLKRPKS |
| | | | | | | | | | QVSEEEG |
| | | | | | | | | | (SEQ ID |
| | | | | | | | | | NO: 85) |
| P11171-2_S85_1 | EPB41_S85 | 0.990 | 0.017 | EPB41 | P11171-2 | S | 85 | _1 | KERTSESR |
| | | | | | | | | | GLSRLFSS |
| | | | | | | | | | FLKRPKSQ |
| | | | | | | | | | VSEEEGK |
| | | | | | | | | | (SEQ ID |
| | | | | | | | | | NO: 86) |
| Q9Y624_S284_1 | FIIR_S284 | 0.908 | 0.016 | F11R | Q9Y624 | S | 284 | _1 | TKKGTSSK |
| | | | | | | | | | KVIYSQPS |
| | | | | | | | | | ARSEGEFK |
| | | | | | | | | | QTSSFLV |
| | | | | | | | | | (SEQ ID |
| | | | | | | | | | NO: 87) |
| P49685__S342_1 | GPR15__S342 | 0.965 | 0.036 | GPR15 | P49685 | S | 342 | _1 | GSSTETSD |
| | | | | | | | | | SHLTKALS |
| | | | | | | | | | TFIHAEDF |
| | | | | | | | | | ARRRKRS |
| | | | | | | | | | (SEQ ID |
| | | | | | | | | | NO: 88) |
| P14316-2_T336_1 | IRF2_T338 | 1.204 | 0.040 | IRF2 | P14316-2 | T | 336 | _1 | SSRPDRET |
| | | | | | | | | | RASVIKKT |
| | | | | | | | | | SDITQARV |
| | | | | | | | | | KSC |
| | | | | | | | | | (SEQ ID |
| | | | | | | | | | NO: 89) |
| O75427_S432_1 | LRCH4_S432 | 1.432 | 0.004 | LRCH4 | O75427 | S | 432 | _1 | RQQ̅Q̅Q̅SGA |
| | | | | | | | | | WGAPRKDS |
| | | | | | | | | | LLKPGLRA |
| | | | | | | | | | VVGGAAA |
| | | | | | | | | | (SEQ ID |
| | | | | | | | | | NO: 90) |
| O95819-4_S519_1 | MAP4K4_S550 | 0.952 | 0.007 | MAP4K4 | O95819-4 | S | 519 | _1 | HSQQPPPP |
| | | | | | | | | | QQERSKPS |
| | | | | | | | | | FHAPEPKA |
| | | | | | | | | | HYEPADR |
| | | | | | | | | | (SEQ ID |
| | | | | | | | | | NO: 91) |
| Q13177_S64_I | PAK2_S64 | 1.233 | 0.017 | PAK2 | Q13177 | S | 64 | _1 | PRHKIISI |
| | | | | | | | | | FSGTEKGS |
| | | | | | | | | | KKKEKERP |
| | | | | | | | | | EISPPSD |
| | | | | | | | | | (SEQ ID |
| | | | | | | | | | NO: 92) |
| Q04759__S370_l | PRKCQ_S370 | 0.827 | 0.044 | PRKCQ | Q04759 | S | 370 | _1 | KMCHLPEP |
| | | | | | | | | | ELNKERPS |
| | | | | | | | | | LQIKLKIE |
| | | | | | | | | | DFILHKM |
| | | | | | | | | | (SEQ ID |
| | | | | | | | | | NO: 93) |
| Q9Y6M7-13_S61_1 | SLC4A7_S52 | 1.146 | 0.017 | SLC4A7 | Q9Y6M7-13 | S | 61 | _1 | LESHRAVY |
| | | | | | | | | | IGVHVPFS |
| | | | | | | | | | KESRRRHR |
| | | | | | | | | | HRGHKHH |
| | | | | | | | | | (SEQ ID |
| | | | | | | | | | NO: 94) |
| Q01082__S2358_1 | SPTBN1_S2358 | 1.212 | 0.008 | SPTBN1 | Q01082 | S | 2358 | _1 | GKREKDKE |
| | | | | | | | | | KDKEKRFS |
| | | | | | | | | | LFGKKK |
| | | | | | | | | | (SEQ ID |
| | | | | | | | | | NO: 95) |
| O94804_S13_2 | STK10_S13 | 2.125 | 0.014 | STK10 | O94804 | S | 13 | _2 | MAFANF |
| | | | | | | | | | RRILRLST |
| | | | | | | | | | FEKRKSRE |
| | | | | | | | | | YEHVRR |
| O94804_T14_2 | STK10_T14 | 2.125 | 0.014 | STK10 | O94804 | T | 14 | _2 | MAFANF |
| | | | | | | | | | RRILRLST |
| | | | | | | | | | FEKRKSRE |
| | | | | | | | | | YEHVRRD |
| | | | | | | | | | (SEQ ID |
| | | | | | | | | | NO: 96) |
| P55327-2_S136_1 | TPD52_S176 | 0.941 | 0.044 | TPD52 | P55327-2 | S | 136 | _1 | ITKKLEDV |
| | | | | | | | | | KNSPTFKS |
| | | | | | | | | | FEEKVENL |
| | | | | | | | | | KSKVGGT |
| | | | | | | | | | (SEQ ID |
| | | | | | | | | | NO: 97) |
| P57075-3_T6_1 | UBASH3A_T6 | 1.261 | 0.004 | UBASH3A | P57075-3 | T | 6 | _l | M̅A̅A̅G̅E̅T̅Q̅L |
| | | | | | | | | | YAKVSNKL |
| | | | | | | | | | KSRSS |
| | | | | | | | | | (SEQ ID |
| | | | | | | | | | NO: 98) |
| P15498-2_S748_1 | VAV1_S780 | 1.440 | 0.008 | VAV1 | P15498-2 | S | 748 | _1 | FKEPEKRT |
| | | | | | | | | | ISRPAVGS |
| | | | | | | | | | TKYFGTAK |
| | | | | | | | | | ARYDFCA |
| | | | | | | | | | (SEQ ID |
| | | | | | | | | | NO: 99) |
| P15498-2_T749_1 | VAV1_T781 | 1.518 | 0.040 | VAV1 | P15498-2 | T | 749 | _1 | KEPEKRTI |
| | | | | | | | | | SRPAVGST |
| | | | | | | | | | KYFGTAKA |
| | | | | | | | | | RYDFCAR |
| | | | | | | | | | (SEQ ID |
| | | | | | | | | | NO: 100) |

TABLE 1B
74 PO4 sites that met log2FC cutoffs after 4-1BB/CD3z CAR stimulation

| UniProt | Gene | log2 | | | UniProt | Amino | UniProt | Multi- | Sequence |
|---|---|---|---|---|---|---|---|---|---|
| Feature | Feature | FC | FDR | Gene | ID | Acid | Position | plicity | Window |
| O75582__S376J | RPS6KA5_S376 | 1.831 | 1.000 | RPS6KA5 | O75582 | S | 376 | _l | PAALPQSS |
| | | | | | | | | | EKLFQGYS |
| | | | | | | | | | FVAPSILF |
| | | | | | | | | | KRNAAVI |
| | | | | | | | | | (SEQ ID |
| | | | | | | | | | NO: 101) |
| O94804_S13_2 | STK10_S13 | 1.786 | 0.404 | STK10 | O94804 | S | 13 | _2 | MAFANF |
| | | | | | | | | | RRILRLST |
| | | | | | | | | | FEKRKSRE |
| | | | | | | | | | YEHVRR |
| | | | | | | | | | (SEQ ID |
| | | | | | | | | | NO: 102) |
| O94804_T14_2 | STK10_T14 | 1.786 | 0.404 | STK10 | O94804 | T | 14 | _2 | MAFANF |
| | | | | | | | | | RRILRLST |
| | | | | | | | | | FEKRKSRE |
| | | | | | | | | | YEHVRRD |
| | | | | | | | | | (SEQ ID |
| | | | | | | | | | NO: 103) |
| P12259_S859_1 | F5_S859 | −1.715 | 1.000 | F5 | P12259 | S | 859 | _l | VTGIRLLS |
| | | | | | | | | | LGAGEFKS |
| | | | | | | | | | QEHAKHKG |
| | | | | | | | | | PKVERDQ |
| | | | | | | | | | (SEQ ID |
| | | | | | | | | | NO: 104) |
| O75582_S381_l | RPS6KA5_S381 | 1.628 | 1.000 | RPS6KA5 | O75582 | S | 381 | _l | QSSEKLFQ |
| | | | | | | | | | GYSFVAPS |
| | | | | | | | | | ILFKRNAA |
| | | | | | | | | | VIDPLQF |
| | | | | | | | | | (SEQ ID |
| | | | | | | | | | NO: 105) |
| O94768_S10_1 | STK17B__S10 | 1.552 | 1.000 | STK17B | O94768 | S | 10 | _1 | MSRR |
| | | | | | | | | | RFDCRSIS |
| | | | | | | | | | GLLTTTPQ |
| | | | | | | | | | IPIKM |
| | | | | | | | | | (SEQ ID |
| | | | | | | | | | NO: 106) |
| Q14241__S163_1 | TCEB3_S163 | 1.470 | 1.000 | TCEB3 | Q14241 | S | 163 | _l | RSYSPDHR |
| | | | | | | | | | QKKHRKLS |
| | | | | | | | | | ELERPHKV |
| | | | | | | | | | SHGHERR |
| | | | | | | | | | (SEQ ID |
| | | | | | | | | | NO: 107) |
| Q8NFC6_S800_1 | BOD1L1_S800 | 1.402 | 1.000 | BOD1L1 | Q8NFC6 | S | 800 | _1 | DKTERKSK |
| | | | | | | | | | HRNERKLS |
| | | | | | | | | | VLGKDGKP |
| | | | | | | | | | VSEYIIK |
| | | | | | | | | | (SEQ ID |
| | | | | | | | | | NO: 107) |
| P01042_S332_1 | KNG1_S332 | −1.316 | 1.000 | KNG1 | P01042 | S | 332 | _1 | YFIDFVAR |
| | | | | | | | | | ETTCSKES |
| | | | | | | | | | NEELTESC |
| | | | | | | | | | ETKKLGQ |
| | | | | | | | | | (SEQ ID |
| | | | | | | | | | NO: 108) |
| Q9UQ35_S20202 | SRRM2_S2020 | −1.273 | 1.000 | SRRM2 | Q9UQ35 | S | 2020 | _2 | RSRSRTSP |
| | | | | | | | | | VTRRRSRS |
| | | | | | | | | | RTPPAIRR |
| | | | | | | | | | RSRSRTP |
| | | | | | | | | | (SEQ ID |
| | | | | | | | | | NO: 109) |
| Q9UQ35_T2022_2 | SRRM2_T2022 | −1.273 | 1.000 | SRRM2 | Q9UQ35 | T | 2022 | _1 | RSRTSPVT |
| | | | | | | | | | RRRSRSRT |
| | | | | | | | | | PPAIRRRS |
| | | | | | | | | | RSRTPLL |
| | | | | | | | | | (SEQ ID |
| | | | | | | | | | NO: 110) |
| Q9NRJ4_S1373_1 | TULP4_S1373 | −1.180 | 1.000 | TULP4 | Q9NRJ4 | S | 1373 | _l | VKKEARTL |
| | | | | | | | | | SDFNSLIS |
| | | | | | | | | | SPHLGREK |
| | | | | | | | | | KKVKSQK |
| | | | | | | | | | (SEQ ID |
| | | | | | | | | | NO: 111) |
| P62753_S240_2 | RPS6_S240 | 1.171 | 1.000 | RPS6 | P62753 | S | 240 | _1 | QEQIAKRR |
| | | | | | | | | | RLSSLRAS |
| | | | | | | | | | TSKSESSQ |
| | | | | | | | | | K |
| | | | | | | | | | (SEQ ID |
| | | | | | | | | | NO: 112) |
| Q9Y4A5-2_T1604_l | TRRAP_T1622 | 1.122 | 1.000 | TRRAP | Q9Y4A5-2 | T | 1604 | _1 | NPNRFITL |
| | | | | | | | | | LLPGGAQT |
| | | | | | | | | | AVRPGSPS |
| | | | | | | | | | TSTMRLD |
| | | | | | | | | | (SEQ ID |
| | | | | | | | | | NO: 113) |
| Q9UEY8_S423_2 | ADD3_S423 | 1.071 | 1.000 | ADD3 | Q9UEY8 | S | 423 | _1 | ATVTAFSF |
| | | | | | | | | | EDDTVPLS |
| | | | | | | | | | PLKYMAQR |
| | | | | | | | | | QQREKTR |
| | | | | | | | | | (SEQ ID |
| | | | | | | | | | NO: 114) |
| Q13557-8_S3152 | CAMK2D_S315 | 1.058 | 1.000 | CAMK2D | Q13557-8 | S | 315 | _1 | LKGAILTT |
| | | | | | | | | | MLATRNFS |
| | | | | | | | | | AAKSLLKK |
| | | | | | | | | | PDGVKES |
| | | | | | | | | | (SEQ ID |
| | | | | | | | | | NO: 115) |
| Q13557-8_S3192 | CAMK2D_S319 | 1.058 | 1.000 | CAMK2D | Q13557-8 | S | 319 | _1 | ILTTMLAT |
| | | | | | | | | | RNFSAAKS |
| | | | | | | | | | LLKKPDGV |
| | | | | | | | | | KESTESS |
| | | | | | | | | | (SEQ ID |
| | | | | | | | | | NO: 116) |
| Q9UN19-2_Y139_1 | DAPP1-Y139 | 1.016 | 1.000 | DAPP1 | Q9UNI9-2 | Y | 139 | _1 | VLKHPYPR |
| | | | | | | | | | KVEEPSIY |
| | | | | | | | | | ESVRVHTA |
| | | | | | | | | | MQTGRTE |
| | | | | | | | | | (SEQ ID |
| | | | | | | | | | NO: 117) |
| Q15287_S251_1 | RNPS1_S251 | −1.009 | 1.000 | RNPS1 | Q15287 | S | 251 | _1 | TAVLAPWP |
| | | | | | | | | | RPPPRRFS |
| | | | | | | | | | PPRRMLPP |
| | | | | | | | | | PPMWRRS |
| | | | | | | | | | (SEQ ID |
| | | | | | | | | | NO: 118) |
| Q6NYC8_S209_2 | PPP1R18_S209 | −1.003 | 1.000 | PPP1R18 | Q6NYC8 | s | 209 | _2 | LSPGETPE |
| | | | | | | | | | RSLRLAES |
| | | | | | | | | | REQSPRRK |
| | | | | | | | | | EVESRLS |
| | | | | | | | | | (SEQ ID |
| | | | | | | | | | NO: 119) |
| Q6NYC8_S213_2 | PPP1R18_S213 | −1.003 | 1.000 | PPP1R18 | Q6NYC8 | s | 213 | _1 | ETPERSLR |
| | | | | | | | | | LAESREQS |
| | | | | | | | | | PRRKEVES |
| | | | | | | | | | RLSPGES |
| | | | | | | | | | (SEQ ID |
| | | | | | | | | | NO: 120) |
| Q13085_S23_l | ACACA_S23 | 0.961 | 1.000 | ACACA | Q13085 | s | 23 | _1 | AQPLELNQ |
| | | | | | | | | | HSRFHGSV |
| | | | | | | | | | SEDNSEDE |
| | | | | | | | | | ISNLVK |
| | | | | | | | | | (SEQ ID |
| | | | | | | | | | NO: 121) |
| P10747-2_Y90_1 | CD28_Y209 | 0.931 | 1.000 | CD28 | P10747-2 | Y | 90 | _1 | MTPRRPGP |
| | | | | | | | | | TRKHYQPY |
| | | | | | | | | | APPRDFAA |
| | | | | | | | | | YRS |
| | | | | | | | | | (SEQ ID |
| | | | | | | | | | NO: 122) |
| Q92956-2_S156_1 | TNFRSF14_S240 | 0.930 | 0.560 | TNFRSF14 | Q92956-2 | S | 156 | _1 | VKRRKPRG |
| | | | | | | | | | DVVKVIVS |
| | | | | | | | | | VQRKRQEA |
| | | | | | | | | | EGEATVI |
| | | | | | | | | | (SEQ ID |
| | | | | | | | | | NO: 123) |
| Q04721__S2070_l | NOTCH2_S2070 | −0.925 | 1.000 | NOTCH2 | Q04721 | s | 2070 | _1 | HHDIVRLL |
| | | | | | | | | | DEYNVTPS |
| | | | | | | | | | PPGTVLTS |
| | | | | | | | | | ALSPVIC |
| | | | | | | | | | (SEQ ID |
| | | | | | | | | | NO: 124) |
| P50402_S8_1 | EMD_S8 | −0.921 | 1.000 | EMD | P50402 | s | 8 | _1 | MD |
| | | | | | | | | | NYADLSDT |
| | | | | | | | | | ELTTLLRR |
| | | | | | | | | | YNIPH |
| | | | | | | | | | (SEQ ID |
| | | | | | | | | | NO: 125) |
| P29590_S480_1 | PML_S480 | 0.917 | 1.000 | PML | P29590 | s | 480 | _l | DLDVSNTT |
| | | | | | | | | | TAQKRKCS |
| | | | | | | | | | QTQCPRKV |
| | | | | | | | | | IKMESEE; |
| | | | | | | | | | GEDVSNTT |
| | | | | | | | | | TAQKRKCS |
| | | | | | | | | | QTQCPRKV |
| | | | | | | | | | IKMESEE |
| | | | | | | | | | (SEQ ID |
| | | | | | | | | | NO: 126) |
| Q14005-3_S144_1 | 1L16_S845 | 0.904 | 1.000 | IL16 | Q14005-3 | s | 144 | _1 | IRASSSSS |
| | | | | | | | | | SIRQRISS |
| | | | | | | | | | FETFGSSQ |
| | | | | | | | | | LPDKGAQ |
| | | | | | | | | | (SEQ ID |
| | | | | | | | | | NO: 127) |
| O75152_S290_l | ZC3H11A_S290 | 0.888 | 1.000 | ZC3H11A | O75152 | s | 290 | _1 | LVRLSLTE |
| | | | | | | | | | RLGKRKFS |
| | | | | | | | | | AGGDSDPP |
| | | | | | | | | | LKRSLAQ |
| | | | | | | | | | (SEQ ID |
| | | | | | | | | | NO: 128) |
| P48729_T321_l | CSNK1A1_T321 | 0.880 | 1.000 | CSNK1A1 | P48729 | T | 321 | _1 | AQQAASSS |
| | | | | | | | | | GQGQQAQT |
| | | | | | | | | | PTGKQTDK |
| | | | | | | | | | TKSNMKG |
| | | | | | | | | | (SEQ ID |
| | | | | | | | | | NO: 129) |
| Q96A57_Y29_2 | TMEM230_Y29 | 0.874 | 1.000 | TMEM230 | Q96A57 | Y | 29 | _2 | SSKVKYSR |
| | | | | | | | | | LSSTDDGY |
| | | | | | | | | | IDLQFKKT |
| | | | | | | | | | PPKIPYK |
| | | | | | | | | | (SEQ ID |
| | | | | | | | | | NO: 130) |
| Q02040_S444_1 | AKAP17AS444 | 0.872 | 1.000 | AKAP17A | Q02040 | S | 444 | _1 | RKERELRE |
| | | | | | | | | | RLLSILLS |
| | | | | | | | | | KKPDDSHT |
| | | | | | | | | | HDELGVA |
| | | | | | | | | | (SEQ ID |
| | | | | | | | | | NO: 131) |
| Q8WVB3_T476_1 | HEXDCT476 | 0.869 | 1.000 | HEXDC | Q8WVB3 | T | 476 | _1 | LLQDLSEV |
| | | | | | | | | | SAPPLPPT |
| | | | | | | | | | SPGRDVAQ |
| | | | | | | | | | DP |
| | | | | | | | | | (SEQ ID |
| | | | | | | | | | NO: 132) |
| Q6ZSZ5_S1160_1 | ARHGEF18_S1160 | 0.864 | 1.000 | ARMGEF18 | Q6ZSZ5 | S | 1160 | _1 | LPGPPAPS |
| | | | | | | | | | PLPATPLS |
| | | | | | | | | | AKEDASKE |
| | | | | | | | | | DVIFF |
| | | | | | | | | | (SEQ ID |
| | | | | | | | | | NO: 133) |
| Q8TBZ34_S395_1 | WDR20_S465 | 0.862 | 1.000 | WDR20 | Q8TBZ3-I | S | 395 | _1 | MDGAIASG |
| | | | | | | | | | VSKFATLS |
| | | | | | | | | | LHDRKERH |
| | | | | | | | | | HEKDHKR |
| | | | | | | | | | (SEQ ID |
| | | | | | | | | | NO: 134) |
| P14316-2_T336_1 | 1RF2T338 | 0.853 | 1.000 | IRF2 | P14316-2 | T | 336 | _1 | SSRPDRET |
| | | | | | | | | | RASVIKKT |
| | | | | | | | | | SDITQARV |
| | | | | | | | | | KSC |
| | | | | | | | | | (SEQ ID |
| | | | | | | | | | NO: 135) |
| Q9UEY8_S402_2 | ADD3S402 | 0.853 | 1.000 | ADD3 | Q9UEY8 | S | 402 | _1 | YAYRHPLI |
| | | | | | | | | | REKPRHKS |
| | | | | | | | | | DVEIPATV |
| | | | | | | | | | TAFSFED |
| | | | | | | | | | (SEQ ID |
| | | | | | | | | | NO: 136) |
| O75152_S295_1 | ZC3H11A_S295 | 0.853 | 1.000 | ZC3H11A | O75152 | S | 295 | _1 | LTERLGKR |
| | | | | | | | | | KFSAGGDS |
| | | | | | | | | | DPPLKRSL |
| | | | | | | | | | AQRLGKK |
| | | | | | | | | | (SEQ ID |
| | | | | | | | | | NO: 137) |
| Q9NZJ0_S679_1 | DTL_S679 | −0.852 | 1.000 | DTL | Q9NZJ0 | s | 679 | _1 | LAMAAKRK |
| | | | | | | | | | AENPSPRS |
| | | | | | | | | | PSSQTPNS |
| | | | | | | | | | RRQSGKK |
| | | | | | | | | | (SEQ ID |
| | | | | | | | | | NO: 138) |
| Q04721_S2081_1 | NOTCH2_S2081 | 0.852 | 1.000 | NOTCH2 | Q04721 | s | 2081 | _1 | NVTPSPPG |
| | | | | | | | | | TVLTSALS |
| | | | | | | | | | PVICGPNR |
| | | | | | | | | | SFLSLKH |
| | | | | | | | | | (SEQ ID |
| | | | | | | | | | NO: 139) |
| Q02880-2_S1447_1 | TOP2B_S1452 | 0.848 | 1.000 | TOP2B | Q02880-2 | s | 1447 | _1 | HDKKSQDF |
| | | | | | | | | | GNLFSFPS |
| | | | | | | | | | YSQKSEDD |
| | | | | | | | | | SAKFDSN |
| | | | | | | | | | (SEQ ID |
| | | | | | | | | | NO: 140) |
| P29590_S512_2 | PML_S512 | 0.837 | 1.000 | PML | P29590 | s | 512 | _1 | KEARLARS |
| | | | | | | | | | SPEQPRPS |
| | | | | | | | | | TSKAVSPP |
| | | | | | | | | | HLDGPPS |
| | | | | | | | | | (SEQ ID |
| | | | | | | | | | NO: 141) |
| Q15691_S155_1 | MAPRE1_S155 | 0.828 | 1.000 | MAPRE1 | QI569I | S | 155 | _l | SLVAPALN |
| | | | | | | | | | KPKKPLTS |
| | | | | | | | | | SSAAPQRP |
| | | | | | | | | | ISTQRTA |
| | | | | | | | | | (SEQ ID |
| | | | | | | | | | NO: 142) |
| P62328_T23_1 | TMSB4X_T23 | 0.824 | 1.000 | TMSB4X | P62328 | T | 23 | _1 | AEIEKFDK |
| | | | | | | | | | SKLKKTET |
| | | | | | | | | | QEKNPLPS |
| | | | | | | | | | KETIEQE |
| | | | | | | | | | (SEQ ID |
| | | | | | | | | | NO: 143) |
| O94906_S143_1 | PRPF6_S143 | 0.818 | 0.975 | PRPF6 | O94906 | S | 143 | _1 | IEKYRMER |
| | | | | | | | | | PKIQQQFS |
| | | | | | | | | | DLKRKLAE |
| | | | | | | | | | VTEEEWL |
| | | | | | | | | | (SEQ ID |
| | | | | | | | | | NO: 144) |
| Q9Y2V2_S26_2 | CARHSP1_S26 | 0.816 | 1.000 | CARHSP1 | Q9Y2V2 | S | 26 | _1 | PPTHQASV |
| | | | | | | | | | GLLDTPRS |
| | | | | | | | | | RERSPSPL |
| | | | | | | | | | RGNVVPS |
| | | | | | | | | | (SEQ ID |
| | | | | | | | | | NO: 145) |
| P17535_S90_1 | JUND_S90 | 0.813 | 1.000 | JUND | P17535 | S | 90 | _1 | ADGAPSAA |
| | | | | | | | | | PPDGLLAS |
| | | | | | | | | | PDLGLLKL |
| | | | | | | | | | ASPELER |
| | | | | | | | | | (SEQ ID |
| | | | | | | | | | NO: 146) |
| Q86UE4_T495_1 | MTDH_T495 | 0.811 | 1.000 | MTDH | Q86UE4 | T | 495 | _1 | TRPKQEKA |
| | | | | | | | | | FSLKTIST |
| | | | | | | | | | SDPAEVLV |
| | | | | | | | | | KNSQPIK |
| | | | | | | | | | (SEQ ID |
| | | | | | | | | | NO: 147) |
| Q96124_T76_1 | FUBP3_T76 | 0.804 | 1.000 | FUBP3 | Q96124 | T | 76 | _1 | DDGVGNQL |
| | | | | | | | | | GALVHQRT |
| | | | | | | | | | VITEEFKV |
| | | | | | | | | | PDKMVGF |
| | | | | | | | | | (SEQ ID |
| | | | | | | | | | NO: 148) |
| Q96EV8_S351_1 | DTNBP1_S351 | 0.791 | 0.404 | DTNBP1 | Q96EV8 | S | 351 | _1 | HTDREATP |
| | | | | | | | | | DGGEDSDS |
| | | | | | | | | | (SEQ ID |
| | | | | | | | | | NO: 149) |
| Q09666_S4903_1 | AHNAK_S4903 | 0.787 | 1.000 | AHNAK | Q09666 | s | 4903 | _1 | PRLDFEGP |
| | | | | | | | | | DAKLSGPS |
| | | | | | | | | | LKMPSLEI |
| | | | | | | | | | SAPKVTA |
| | | | | | | | | | (SEQ ID |
| | | | | | | | | | NO: 150) |
| Q9P2B7_S329_1 | CFAP97_S329 | 0.784 | 1.000 | CFAP97 | Q9P2B7 | s | 329 | _1 | KHEPDVSS |
| | | | | | | | | | KSSSVLDS |
| | | | | | | | | | SLDHRHKQ |
| | | | | | | | | | KVLHDTM |
| | | | | | | | | | (SEQ ID |
| | | | | | | | | | NO: 151) |
| Q8WUZ0_T118_3 | BCL7C_T118 | 0.780 | 1.000 | BCL7C | Q8WUZ0 | T | 118 | _3 | SEGSLQKG |
| | | | | | | | | | TEPSPGGT |
| | | | | | | | | | PQPSRPVS |
| | | | | | | | | | PAGPPEG |
| | | | | | | | | | (SEQ ID |
| | | | | | | | | | NO: 152) |
| Q8WUZ0_S122_3 | BCL7C_S122 | 0.780 | 1.000 | BCL7C | Q8WUZ0 | S | 122 | _3 | LQKGTEPS |
| | | | | | | | | | PGGTPQPS |
| | | | | | | | | | RPVSPAGP |
| | | | | | | | | | PEGVPEE |
| | | | | | | | | | (SEQ ID |
| | | | | | | | | | NO: 153) |
| Q8WUZ0_S126_3 | BCL7C_S126 | 0.780 | 1.000 | BCL7C | Q8WUZ0 | S | 126 | _3 | TEPSPGGT |
| | | | | | | | | | PQPSRPVS |
| | | | | | | | | | PAGPPEGV |
| | | | | | | | | | PEEAQPP |
| | | | | | | | | | (SEQ ID |
| | | | | | | | | | NO: 154) |
| Q8N9B5-2_S962_1 | JMY_S974 | 0.775 | 1.000 | JMY | Q8N9B5-2 | S | 962 | _1 | SIHEALRR |
| | | | | | | | | | IKEASPES |
| | | | | | | | | | EDEEEALP |
| | | | | | | | | | CTDWEN |
| | | | | | | | | | (SEQ ID |
| | | | | | | | | | NO: 155) |
| Q9Y4F9_S175_1 | FAM65B_S175 | 0.768 | 1.000 | FAM65B | Q9Y4F9 | S | 175 | _1 | KQAFATSP |
| | | | | | | | | | ASKAARES |
| | | | | | | | | | LTEINRSF |
| | | | | | | | | | KEYTENM |
| | | | | | | | | | (SEQ ID |
| | | | | | | | | | NO: 156) |
| Q9BXP5-5_S1361 | SRRT_S136 | 0.766 | 1.000 | SRRT | Q9BXP5-5 | S | 136 | _1 | HIMQHHVL |
| | | | | | | | | | PIQARLGS |
| | | | | | | | | | IAEIDLGV |
| | | | | | | | | | PPPVMKT |
| | | | | | | | | | (SEQ ID |
| | | | | | | | | | NO: 157) |
| P55201-3_S75_2 | BRPF1_S75 | 0.764 | 1.000 | BRPF1 | P55201-3 | S | 75 | _2 | KJIKKKGR |
| | | | | | | | | | QSRPANKQ |
| | | | | | | | | | SPSPSEVS |
| | | | | | | | | | QSPGREVM |
| | | | | | | | | | (SEQ ID |
| | | | | | | | | | NO: 158) |
| P17936_S201_1 | IGFBP3_S201 | −0.762 | 0.975 | 1GFBP3 | P17936 | S | 201 | _1 | YKVDYESQ |
| | | | | | | | | | STDTQNFS |
| | | | | | | | | | SESKRETE |
| | | | | | | | | | YGPCRRE |
| | | | | | | | | | (SEQ ID |
| | | | | | | | | | NO: 159) |
| Q99638_S375_2 | RAD9A_S375 | 0.752 | 1.000 | RAD9A | Q99638 | S | 375 | _2 | KFRSLFFG |
| | | | | | | | | | SILAPVRS |
| | | | | | | | | | PQGPSPVL |
| | | | | | | | | | AEDSEGE |
| | | | | | | | | | (SEQ ID |
| | | | | | | | | | NO: 160) |
| P55201-3_S77_2 | BRPF1_S77 | 0.748 | 1.000 | BRPF1 | P55201-3 | S | 77 | _2 | KKKGRQSR |
| | | | | | | | | | PANKQSPS |
| | | | | | | | | | PSEVSQSP |
| | | | | | | | | | GREVMSY |
| | | | | | | | | | (SEQ ID |
| | | | | | | | | | NO: 161) |
| Q13029-5_S220_1 | PRDM2__S421 | 0.742 | 1.000 | PRDM2 | Q13029-5 | S | 220 | _l | RRHERRHE |
| | | | | | | | | | AGLKRKPS |
| | | | | | | | | | QTLQPSED |
| | | | | | | | | | LADGKAS |
| | | | | | | | | | (SEQ ID |
| | | | | | | | | | NO: 162) |
| Q7LBC6_S798_1 | KDM3B__S798 | 0.727 | 1.000 | KDM3B | Q7LBC6 | S | 798 | _l | FSQENKAP |
| | | | | | | | | | FEAVKRFS |
| | | | | | | | | | LDERSLAC |
| | | | | | | | | | RQDSDSS |
| | | | | | | | | | (SEQ ID |
| | | | | | | | | | NO: 163) |
| Q7L099-2_T4_1 | RUFY3_T4 | 0.727 | 1.000 | RUFY3 | Q7L099-2 | T | 4 | _1 | |
| | | | | | | | | | MAETPPP |
| | | | | | | | | | PTAGAESC |
| | | | | | | | | | SEEP |
| | | | | | | | | | (SEQ ID |
| | | | | | | | | | NO: 164) |
| Q9UQ35_S1984_1 | SRRM2_S1984 | −0.725 | 1.000 | SRRM2 | Q9UQ35 | S | 1984 | _l | RSRSRTSP |
| | | | | | | | | | ITRRRSRS |
| | | | | | | | | | RTSPVTRR |
| | | | | | | | | | RSRSRTS |
| | | | | | | | | | (SEQ ID |
| | | | | | | | | | NO: 165) |
| Q92841_Y580_1 | DDX17_Y580 | 0.718 | 1.000 | DDX17 | Q92841 | Y | 580 | _l | SRYRTTSS |
| | | | | | | | | | ANNPNLMY |
| | | | | | | | | | QDECDRRL |
| | | | | | | | | | RGVKDGG |
| | | | | | | | | | (SEQ ID |
| | | | | | | | | | NO: 166) |
| 015418-2_S389_1 | RPS6KA1_S380 | 0.716 | 1.000 | RPS6KA1 | Q15418-2 | S | 389 | _l | GIPPSAGA |
| | | | | | | | | | HQLFRGFS |
| | | | | | | | | | FVATGLME |
| | | | | | | | | | DDGKPRA |
| | | | | | | | | | (SEQ ID |
| | | | | | | | | | NO: 167) |
| P46013_S1136_1 | MK167_S1136 | 0.714 | 1.000 | MK167 | P46013 | S | 1136 | _l | DEKTTKIA |
| | | | | | | | | | CKSPPPES |
| | | | | | | | | | VDTPTSTK |
| | | | | | | | | | QWPKRSL |
| | | | | | | | | | (SEQ ID |
| | | | | | | | | | NO: 168) |
| Q8WVB3_S477_1 | HEXDC_S477 | 0.713 | 1.000 | HEXDC | Q8WVB3 | S | 477 | _1 | LQDLSEVS |
| | | | | | | | | | APPLPPTS |
| | | | | | | | | | PGRDVAQD |
| | | | | | | | | | P |
| | | | | | | | | | (SEQ ID |
| | | | | | | | | | NO: 169) |
| Q9NYF3_S273_1 | FAM53C_S273 | −0.706 | 1.000 | FAM53C | Q9NYF3 | s | 273 | _1 | LPWRPRGL |
| | | | | | | | | | RNLPRSRS |
| | | | | | | | | | QPCDLDAR |
| | | | | | | | | | KTGVKRR |
| | | | | | | | | | (SEQ ID |
| | | | | | | | | | NO: 170) |
| O94768_S12_1 | STK17B_S12 | 0.705 | 1.000 | STK17B | O94768 | S | 12 | _1 | MSRRR |
| | | | | | | | | | FDCRSISG |
| | | | | | | | | | LLTTTPQI |
| | | | | | | | | | PIKMEN |
| | | | | | | | | | (SEQ ID |
| | | | | | | | | | NO: 171) |
| Q96RK0_S1382_2 | CIC_S1382 | 0.702 | 1.000 | CIC | Q96RK0 | s | 1382 | _2 | PEEVLPSP |
| | | | | | | | | | TLQSLATS |
| | | | | | | | | | PRAILGSY |
| | | | | | | | | | RKKRKNS |
| | | | | | | | | | (SEQ ID |
| | | | | | | | | | NO: 172) |
| Q01433__S100_1 | AMPD2__S100 | 0.702 | 0.975 | AMPD2 | Q01433 | s | 100 | _1 | EARGGLGA |
| | | | | | | | | | PPLQSARS |
| | | | | | | | | | LPGPAPCL |
| | | | | | | | | | KHFPLDL |
| | | | | | | | | | (SEQ ID |
| | | | | | | | | | NO: 173) |

TABLE 2A
20 PO4 sites most upregulated by CD28/CD3z
CAR stimulation at 45 minutes

| Log2FC Rank | UniProt Feature | Gene Feature | Log2FC |
|---|---|---|---|
| 1 | O75582_S381_1 | RPS6KA5_S381 | 4.884 |
| 2 | O94768_S10_1 | STK17B_S10 | 4.690 |
| 3 | O75582_S376_1 | RPS6KA5_S376 | 4.436 |
| 4 | Q14241_S163_1 | TCEB3_S163 | 3.851 |
| 5 | O94804_S13_2 | STK10_S13 | 3.631 |
| 6 | O94804_T14_2 | STK10_T14 | 3.631 |
| 7 | Q8NFC6_S800_1 | BOD1L1_S800 | 3.568 |
| 8 | Q6ZSZ5_S1160_1 | ARHGEF18_S1160 | 3.496 |
| 9 | P29590_S480_1 | PML_S480 | 3.364 |
| 10 | Q02880-2_S1447_1 | TOP2B_S1452 | 3.248 |
| 11 | Q9UN19-2_Y139_1 | DAPP1_Y139 | 3.185 |
| 12 | P62753_S240_2 | RPS6_S240 | 3.168 |
| 13 | O75937_S35_1 | DNAJC8_S35 | 3.126 |
| 14 | P14316-2_T336_1 | IRF2_T338 | 3.121 |
| 15 | O75152_S290_1 | ZC3H11A_S290 | 3.107 |
| 16 | Q9Y4F9_S175_1 | FAM65B_S175 | 3.094 |
| 17 | Q5TDH0_S194_1 | DDI2_S194 | 3.085 |
| 18 | P19338_T583_1 | NCL_T583 | 2.993 |
| 19 | O75152_S295_1 | ZC3H11A_S295 | 2.981 |
| 20 | Q14005-3_S144_1 | IL16_S845 | 2.944 |

TABLE 2B
20 PO4 sites most upregulated by 4-1BB/CD3z
CAR stimulation at 45 minutes

| Log2FC Rank | UniProt Feature | Gene Feature | Log2FC |
|---|---|---|---|
| 1 | O94768_S10_1 | STK17B_S10 | 4.617 |
| 2 | O75582_S376_1 | RPS6KA5_S376 | 2.957 |
| 3 | Q14241_S163_1 | TCEB3_S163 | 2.886 |
| 4 | Q8NFC6_S800_1 | BOD1L1_S800 | 2.800 |
| 5 | O94804_S13_2 | STK10_S13 | 2.749 |
| 6 | O94804_T14_2 | STK10_T14 | 2.749 |
| 7 | Q6ZSZ5_S1160_1 | ARHGEF18_S1160 | 2.538 |
| 8 | P29590_S480_1 | PML_S480 | 2.442 |
| 9 | P62753_S240_2 | RPS6_S240 | 2.425 |
| 10 | Q5VWG9_S199_1 | TAF3_S199 | 2.323 |
| 11 | Q9Y4A5-2_S2059_1 | TRRAP_S2077 | 2.318 |
| 12 | Q15149_S4406_1 | PLEC_S4406 | 2.303 |
| 13 | Q14005-3_S144_1 | IL16_S845 | 2.275 |
| 14 | Q92841_S599_1 | DDX17_S599 | 2.262 |
| 15 | O75582_S381_1 | RPS6KA5_S381 | 2.252 |
| 16 | Q5TDH0_S194_1 | DDI2_S194 | 2.215 |
| 17 | O75152_S290_1 | ZC3H11A_S290 | 2.181 |
| 18 | O75937_S35_1 | DNAJC8_S35 | 2.172 |
| 19 | O75152_S295_1 | ZC3H11A_S295 | 2.135 |
| 20 | P62328_T23_1 | TMSB4X_T23 | 2.129 |

TABLE 3
1,673 Differentially expressed genes between stimulated
CD28/CD3z and 4-1BB/CD3z CAR T cells

| Gene | log2FC | FDR |
|---|---|---|
| GFPT2 | −6.84 | 4.27E−03 |
| LINC00891 | −5.83 | 9.03E−03 |
| EPHA4 | −5.51 | 6.11E−04 |
| NPIPB9 | −5.28 | 2.87E−03 |
| VIPR1 | −5.01 | 2.77E−03 |
| CACNA1I | −4.95 | 7.17E−03 |
| VSIG1 | −4.57 | 4.07E−04 |
| CTDSPL | −4.43 | 3.68E−03 |
| CTB-133G6.1 | −4.37 | 2.09E−03 |
| RP1-193H18.2 | −4.36 | 3.68E−03 |
| CTC-523E23.1 | −4.20 | 1.28E−03 |
| VIPR1-AS1 | −4.16 | 1.86E−03 |
| SBK1 | −4.11 | 2.32E−03 |
| CTC-523E23.4 | −4.08 | 2.36E−03 |
| ST8SIA1 | −4.02 | 9.68E−04 |
| METTL7A | −4.01 | 3.29E−03 |
| AC004067.5 | −3.96 | 2.73E−04 |
| GPR155 | −3.85 | 1.28E−03 |
| NUAK2 | −3.84 | 2.34E−03 |
| GPA33 | −3.84 | 2.67E−03 |
| RP11-1000B6.8 | −3.84 | 4.73E−03 |
| EDAR | −3.83 | 9.85E−03 |
| CTC-523E23.5 | −3.80 | 2.55E−03 |
| LMTK3 | −3.80 | 3.73E−03 |
| MGAT5B | −3.77 | 5.90E−03 |
| RP11-285F7.2 | −3.68 | 1.34E−03 |
| RP11-245D16.4 | −3.65 | 3.51E−04 |
| HPSE2 | −3.65 | 2.94E−03 |
| PLCD1 | −3.61 | 1.78E−03 |
| YPEL4 | −3.59 | 5.01E−03 |
| RP1-221C16.8 | −3.56 | 6.91E−04 |
| AMPH | −3.55 | 1.93E−03 |
| RP11-747H7.3 | −3.54 | 2.06E−03 |
| SOX13 | −3.53 | 7.59E−03 |
| ADAM23 | −3.52 | 8.06E−03 |
| RP11-730K11.1 | −3.51 | 3.73E−03 |
| LRRC2 | −3.50 | 4.19E−03 |
| KLF2 | −3.45 | 3.43E−04 |
| ADAM28 | −3.44 | 2.23E−03 |
| C6orf222 | −3.40 | 3.31E−03 |
| PTPRO | −3.38 | 1.00E−03 |
| ICAM4 | −3.37 | 7.14E−03 |
| DAPK2 | −3.36 | 3.93E−03 |
| TSC22D3 | −3.35 | 1.40E−03 |
| KRT81 | −3.35 | 1.96E−03 |
| MAP2K6 | −3.34 | 2.07E−03 |
| LINC00880 | −3.34 | 8.73E−03 |
| IL7R | −3.33 | 3.26E−04 |
| TMEM169 | −3.32 | 7.50E−04 |
| TCF7 | −3.30 | 1.69E−03 |
| RP11-147L13.8 | −3.29 | 1.35E−03 |
| FAM65B | −3.28 | 1.24E−04 |
| YPEL3 | −3.28 | 1.58E−03 |
| RP5-1184F4.7 | −3.28 | 3.34E−03 |
| RP13-890H12.2 | −3.28 | 3.56E−03 |
| RP11-182J1.3 | −3.28 | 6.08E−03 |
| LDLRAP1 | −3.27 | 1.68E−03 |
| CTC-523E23.11 | −3.27 | 1.97E−03 |
| TBXA2R | −3.26 | 2.31E−03 |
| RP11-203J24.8 | −3.26 | 2.75E−03 |
| RP11-44F14.6 | −3.25 | 6.11E−04 |
| PBXIP1 | −3.24 | 9.99E−04 |
| RP3-406P24.5 | −3.23 | 3.29E−03 |
| CTD-2284J15.1 | −3.22 | 2.32E−03 |
| MRVI1 | −3.18 | 1.93E−03 |
| OCM | −3.17 | 2.71E−03 |
| SULT1B1 | −3.11 | 3.15E−03 |
| RP11-666A8.8 | −3.10 | 3.14E−03 |
| RP11-435I10.4 | −3.09 | 7.10E−03 |
| SORL1 | −3.07 | 4.10E−04 |
| EEPD1 | −3.06 | 7.04E−04 |
| MAP6 | −3.06 | 6.31E−03 |
| KCNMB4 | −3.05 | 3.21E−03 |
| ZSWIM5 | −3.05 | 5.84E−03 |
| MPP7 | −3.03 | 2.41E−03 |
| RP1-47M23.3 | −3.02 | 2.75E−03 |
| RND1 | −3.00 | 2.29E−03 |
| NR5A2 | −2.99 | 3.08E−03 |
| GCNT4 | −2.99 | 7.06E−03 |
| C16orf74 | −2.97 | 3.70E−03 |
| BTBD8 | −2.94 | 4.41E−03 |
| KCNC1 | −2.93 | 9.93E−03 |
| YPEL1 | −2.92 | 4.41E−03 |
| NCF2 | −2.92 | 5.64E−03 |
| STK38 | −2.89 | 1.24E−04 |
| TMEM63A | −2.88 | 3.51E−04 |
| GPR146 | −2.88 | 8.13E−03 |
| SMIM14 | −2.87 | 1.96E−03 |
| PRAM1 | −2.85 | 2.41E−03 |
| RP11-374F3.5 | −2.84 | 2.46E−03 |
| GAS6 | −2.83 | 4.18E−03 |
| FCGBP | −2.81 | 1.75E−03 |
| AJ003147.11 | −2.80 | 7.11E−03 |
| AMZ1 | −2.79 | 2.58E−03 |
| KCNQ2 | −2.79 | 9.51E−03 |
| RP11-520H14.7 | −2.77 | 9.99E−03 |
| RASA3 | −2.76 | 1.24E−04 |
| C11orF21 | −2.76 | 7.50E−04 |
| GLIPR1L2 | −2.75 | 2.99E−03 |
| PPIAL4G | −2.73 | 7.04E−03 |
| TRGV2 | −2.73 | 7.74E−03 |
| ABTB1 | −2.71 | 1.30E−03 |
| GPR61 | −2.71 | 5.01E−03 |
| AMOT | −2.71 | 6.20E−03 |
| COL6A1 | −2.71 | 9.01E−03 |
| RP11-798M19.6 | −2.70 | 2.50E−03 |
| PASK | −2.67 | 7.27E−04 |
| HIP1 | −2.67 | 4.90E−03 |
| KRT73 | −2.66 | 3.60E−03 |
| RP11-775C24.3 | −2.66 | 5.70E−03 |
| RP11-405M12.4 | −2.66 | 6.65E−03 |
| RP11-401P9.4 | −2.66 | 7.37E−03 |
| RP11-483E17.1 | −2.65 | 1.47E−03 |
| PROCR | −2.65 | 1.78E−03 |
| LINC00886 | −2.65 | 2.41E−03 |
| C1orf21 | −2.65 | 6.86E−03 |
| LINC00426 | −2.64 | 8.64E−04 |
| FAM8A1 | −2.64 | 9.10E−04 |
| CTC-378H22.2 | −2.64 | 6.47E−03 |
| AC130469.1 | −2.63 | 2.15E−03 |
| C16orf86 | −2.63 | 2.89E−03 |
| CTD-2540F13.2 | −2.63 | 4.22E−03 |
| RP5-1009E24.9 | −2.62 | 2.59E−03 |
| INMT | −2.61 | 9.78E−03 |
| PLAG1 | −2.60 | 2.36E−03 |
| CTC-490E21.11 | −2.60 | 6.17E−03 |
| ATXN7L1 | −2.59 | 3.34E−03 |
| NHLRC4 | −2.59 | 4.49E−03 |
| JAKMIP3 | −2.59 | 7.10E−03 |
| PRODH | −2.58 | 5.42E−03 |
| ACACB | −2.58 | 5.90E−03 |
| C2orf15 | −2.57 | 4.48E−03 |
| AC008746.12 | −2.56 | 1.77E−03 |
| RP11-356J5.12 | −2.55 | 3.14E−03 |
| TCP11L2 | −2.54 | 1.50E−03 |
| ARHGAP5-AS1 | −2.54 | 4.41E−03 |
| ATP8B3 | −2.53 | 1.55E−03 |
| ZNF763 | −2.53 | 3.21E−03 |
| LINC00861 | −2.53 | 5.62E−03 |
| TP53INP1 | −2.53 | 6.31E−03 |
| FLNB-AS1 | −2.52 | 5.63E−03 |
| AGO4 | −2.51 | 1.78E−03 |
| RP11-55K13.1 | −2.51 | 4.05E−03 |
| RP4-761J14.10 | −2.51 | 6.40E−03 |
| FAM102A | −2.50 | 7.05E−04 |
| OLFM2 | −2.50 | 1.82E−03 |
| COLGALT2 | −2.50 | 4.28E−03 |
| NSG1 | −2.49 | 5.38E−03 |
| CTDSP2 | −2.48 | 3.66E−04 |
| RP5-1009E24.8 | −2.48 | 1.90E−03 |
| CTC-453G23.8 | −2.48 | 2.41E−03 |
| C5 | −2.48 | 2.98E−03 |
| EIF1B-AS1 | −2.46 | 9.52E−03 |
| APBA2 | −2.45 | 7.08E−04 |
| DBP | −2.45 | 3.09E−03 |
| MTSS1 | −2.45 | 4.42E−03 |
| CTC-523E23.3 | −2.44 | 3.04E−03 |
| ADAMTS6 | −2.44 | 7.07E−03 |
| RP11-540O11.1 | −2.43 | 4.30E−03 |
| RP3-510D11.2 | −2.43 | 4.72E−03 |
| RP11-539L10.2 | −2.42 | 2.02E−03 |
| TC2N | −2.41 | 9.03E−04 |
| SPOCK2 | −2.40 | 1.99E−03 |
| RP11-329N15.3 | −2.39 | 3.38E−03 |
| GIPC3 | −2.38 | 4.65E−03 |
| TSPAN2 | −2.38 | 7.26E−03 |
| NLRP1 | −2.37 | 8.64E−04 |
| NELL2 | −2.37 | 1.09E−03 |
| RAB37 | −2.36 | 1.69E−03 |
| PTGES2-AS1 | −2.35 | 6.59E−04 |
| AC109826.1 | −2.34 | 1.57E−03 |
| MT1F | −2.34 | 8.40E−03 |
| CALCOCO1 | −2.33 | 1.30E−03 |
| ITGA6 | −2.33 | 2.55E−03 |
| RP11-804H8.6 | −2.33 | 2.97E−03 |
| RP5-1092A3.4 | −2.33 | 4.72E−03 |
| RP5-837J1.6 | −2.33 | 8.13E−03 |
| RFX8 | −2.32 | 3.84E−03 |
| ATP8A2 | −2.32 | 4.15E−03 |
| PRRT1 | −2.32 | 5.49E−03 |
| CNPY4 | −2.31 | 2.85E−04 |
| FAM214A | −2.31 | 2.12E−03 |
| KLF3 | −2.31 | 2.58E−03 |
| RP11-401P9.5 | −2.31 | 7.84E−03 |
| HSD17B11 | −2.29 | 2.70E−04 |
| FLNB | −2.29 | 4.38E−04 |
| TRBV23-1 | −2.29 | 6.98E−03 |
| CACNA1C | −2.29 | 7.73E−03 |
| RP11-563J2.3 | −2.28 | 4.73E−03 |
| LINC00899 | −2.28 | 8.74E−03 |
| HVCN1 | −2.27 | 1.83E−03 |
| SCML4 | −2.27 | 3.52E−03 |
| OPRL1 | −2.27 | 5.98E−03 |
| NMT2 | −2.26 | 1.61E−03 |
| ABCD2 | −2.25 | 1.20E−03 |
| PRRT2 | −2.24 | 6.33E−03 |
| FOXE1 | −2.24 | 7.21E−03 |
| RP4-791M13.4 | −2.24 | 8.79E−03 |
| KLHL24 | −2.22 | 3.79E−03 |
| OR10G2 | −2.21 | 8.90E−03 |
| PIK3IP1-AS1 | −2.21 | 8.93E−03 |
| GIMAP4 | −2.20 | 1.97E−03 |
| ABLIM1 | −2.20 | 2.27E−03 |
| SPON2 | −2.20 | 2.97E−03 |
| RP11-455I9.1 | −2.20 | 9.98E−03 |
| DPEP2 | −2.19 | 1.57E−03 |
| P2RY4 | −2.19 | 6.25E−03 |
| TMC4 | −2.19 | 8.28E−03 |
| ABCA7 | −2.18 | 2.99E−03 |
| PIK3IP1 | −2.18 | 3.77E−03 |
| C1orf145 | −2.18 | 4.04E−03 |
| RP11-277P12.20 | −2.17 | 2.34E−03 |
| SUOX | −2.17 | 6.75E−03 |
| FOXO4 | −2.16 | 1.34E−03 |
| TRGV10 | −2.15 | 1.73E−03 |
| TMEM107 | −2.15 | 1.93E−03 |
| KB-1732A1.1 | −2.15 | 3.78E−03 |
| RP11-464F9.22 | −2.15 | 6.75E−03 |
| MFGE8 | −2.15 | 7.74E−03 |
| ABHD4 | −2.14 | 2.45E−03 |
| SNED1 | −2.14 | 6.81E−03 |
| AC009303.2 | −2.12 | 2.11E−03 |
| RARB | −2.12 | 5.49E−03 |
| RP11-731C17.2 | −2.11 | 9.94E−04 |
| GLCCI1 | −2.10 | 2.51E−03 |
| SEPSECS-AS1 | −2.10 | 4.04E−03 |
| KLRC4-KLRK1 | −2.09 | 4.59E−03 |
| LINC01024 | −2.08 | 1.43E−03 |
| AQP3 | −2.08 | 1.66E−03 |
| CDC42BPG | −2.08 | 3.60E−03 |
| SYTL2 | −2.08 | 3.84E−03 |
| EPB41 | −2.07 | 5.91E−04 |
| SERPINI1 | −2.07 | 6.46E−03 |
| MYO1F | −2.06 | 6.11E−04 |
| PAX6 | −2.06 | 6.14E−03 |
| ADAM22 | −2.05 | 1.14E−03 |
| SEMA6C | −2.05 | 2.34E−03 |
| C1QTNF3 | −2.05 | 2.71E−03 |
| TRGC1 | −2.05 | 4.92E−03 |
| CCDC109B | −2.04 | 5.91E−04 |
| BCL9L | −2.04 | 1.35E−03 |
| CCDC65 | −2.04 | 2.02E−03 |
| RP11-563J2.2 | −2.04 | 2.11E−03 |
| LINGO3 | −2.04 | 2.37E−03 |
| CTD-2540L5.6 | −2.04 | 2.58E−03 |
| RP11-177H2.1 | −2.04 | 4.72E−03 |
| KIAA1324L | −2.04 | 4.82E−03 |
| FMO4 | −2.04 | 7.97E−03 |
| TRGV4 | −2.04 | 9.14E−03 |
| EPHX2 | −2.03 | 1.03E−03 |
| ADHFE1 | −2.03 | 4.29E−03 |
| FBXO32 | −2.02 | 2.58E−03 |
| PPEF1 | −2.02 | 3.15E−03 |
| CCDC102B | −2.02 | 4.25E−03 |
| PKIG | −2.02 | 6.40E−03 |
| PAQR8 | −2.01 | 4.83E−04 |
| RCAN3 | −2.01 | 6.09E−04 |
| RP5-882C2.2 | −2.01 | 2.94E−03 |
| HSD17B6 | −2.01 | 8.08E−03 |
| CDC25B | −2.00 | 2.85E−04 |
| THEMIS2 | −2.00 | 9.76E−04 |
| FCMR | −2.00 | 3.65E−03 |
| FDXR | −2.00 | 3.68E−03 |
| TMEM191A | −1.99 | 3.27E−03 |
| SLC9A9 | −1.99 | 3.28E−03 |
| BBS2 | −1.98 | 1.97E−03 |
| STMN3 | −1.98 | 2.94E−03 |
| TMOD2 | −1.98 | 3.48E−03 |
| RP11-817I4.1 | −1.98 | 4.45E−03 |
| FYB | −1.97 | 6.11E−04 |
| TRG-AS1 | −1.96 | 6.78E−04 |
| APBB1 | −1.96 | 1.43E−03 |
| RP11-644F5.10 | −1.96 | 1.70E−03 |
| GDPD1 | −1.96 | 3.06E−03 |
| TET1 | −1.96 | 6.18E−03 |
| AC015971.2 | −1.96 | 7.17E−03 |
| BZRAP1-AS1 | −1.94 | 8.84E−04 |
| ACSF2 | −1.94 | 3.60E−03 |
| C4orf36 | −1.94 | 5.66E−03 |
| CEP128 | −1.93 | 1.03E−03 |
| HEATR9 | −1.93 | 6.42E−03 |
| ACKR3 | −1.92 | 6.20E−03 |
| AC002467.7 | −1.91 | 3.21E−03 |
| FAM13A | −1.91 | 6.50E−03 |
| IL24 | −1.91 | 7.30E−03 |
| NOL4L | −1.90 | 1.24E−04 |
| DOK4 | −1.90 | 6.51E−03 |
| HCG27 | −1.90 | 6.73E−03 |
| STX1B | −1.90 | 9.28E−03 |
| AC142472.6 | −1.89 | 3.65E−03 |
| TSPAN32 | −1.89 | 4.09E−03 |
| REM2 | −1.89 | 6.63E−03 |
| SLC16A5 | −1.89 | 9.27E−03 |
| ZC3H12D | −1.89 | 9.76E−03 |
| CCDC69 | −1.88 | 2.85E−04 |
| PECR | −1.88 | 7.24E−04 |
| MMP19 | −1.88 | 2.32E−03 |
| PDCD4 | −1.88 | 2.51E−03 |
| LINC00324 | −1.88 | 3.11E−03 |
| ZNF92 | −1.87 | 3.88E−04 |
| SUN2 | −1.87 | 6.11E−04 |
| PCMTD2 | −1.87 | 1.09E−03 |
| TRGC2 | −1.87 | 3.41E−03 |
| TRANK1 | −1.87 | 4.21E−03 |
| ALDH6A1 | −1.87 | 5.78E−03 |
| FAM214B | −1.86 | 7.20E−03 |
| LRRC46 | −1.86 | 7.93E−03 |
| LINC00205 | −1.86 | 9.33E−03 |
| LEF1 | −1.85 | 6.11E−04 |
| DFNB59 | −1.85 | 5.64E−03 |
| GS1-358P8.4 | −1.84 | 1.30E−03 |
| LINC01011 | −1.84 | 6.18E−03 |
| SESN1 | −1.82 | 4.29E−03 |
| KLHL3 | −1.82 | 7.07E−03 |
| SLFN13 | −1.81 | 3.14E−04 |
| CTD-2562J17.6 | −1.81 | 1.30E−03 |
| MANBA | −1.81 | 2.79E−03 |
| RALGPS1 | −1.81 | 6.03E−03 |
| MGAT4A | −1.80 | 6.11E−04 |
| SLC35D2 | −1.80 | 1.81E−03 |
| ATM | −1.80 | 1.97E−03 |
| TOB1 | −1.80 | 1.97E−03 |
| PPP2R5C | −1.79 | 2.70E−04 |
| ARL11 | −1.79 | 7.48E−03 |
| KRT72 | −1.79 | 9.12E−03 |
| ZNF563 | −1.78 | 4.14E−03 |
| TIFA | −1.77 | 2.85E−04 |
| RASGRP2 | −1.77 | 1.27E−03 |
| CROT | −1.77 | 2.41E−03 |
| AGPAT4 | −1.77 | 2.44E−03 |
| GM2A | −1.77 | 2.89E−03 |
| LA16C-390H2.4 | −1.77 | 4.41E−03 |
| PAIP2B | −1.77 | 6.06E−03 |
| FBXO48 | −1.76 | 1.20E−03 |
| SSBP2 | −1.76 | 1.69E−03 |
| LINC00921 | −1.76 | 3.07E−03 |
| RP11-18H21.1 | −1.76 | 5.62E−03 |
| TP53TG1 | −1.76 | 6.85E−03 |
| ASTN2 | −1.76 | 9.60E−03 |
| KATNAL1 | −1.75 | 7.05E−04 |
| ZBTB18 | −1.75 | 1.87E−03 |
| CREBRF | −1.75 | 2.28E−03 |
| AMIGO1 | −1.75 | 8.15E−03 |
| CDC14A | −1.74 | 1.67E−04 |
| AHNAK | −1.74 | 7.53E−04 |
| ADD3 | −1.74 | 9.43E−04 |
| AHCYL2 | −1.74 | 1.32E−03 |
| EFHC1 | −1.74 | 3.70E−03 |
| ELOVL4 | −1.74 | 6.38E−03 |
| STARD5 | −1.73 | 1.20E−03 |
| GIMAP8 | −1.73 | 1.69E−03 |
| ARID3A | −1.73 | 3.28E−03 |
| MAP1A | −1.73 | 9.20E−03 |
| LINC00649 | −1.72 | 2.13E−03 |
| MAGEE1 | −1.72 | 2.15E−03 |
| FAM227B | −1.72 | 2.88E−03 |
| SYCP2 | −1.72 | 3.02E−03 |
| CFAP44 | −1.72 | 9.55E−03 |
| ZMYND10 | −1.71 | 8.83E−03 |
| COL6A2 | −1.71 | 9.66E−03 |
| 9-Sep | −1.70 | 5.05E−04 |
| MAP3K12 | −1.70 | 9.43E−04 |
| GIMAP7 | −1.70 | 1.20E−03 |
| BTN3A1 | −1.70 | 1.31E−03 |
| HIST2H2BF | −1.70 | 4.28E−03 |
| LRRC37B | −1.70 | 5.35E−03 |
| DISC1 | −1.70 | 9.27E−03 |
| LRMP | −1.69 | 2.41E−03 |
| VASH1 | −1.69 | 4.31E−03 |
| LINC01232 | −1.69 | 4.87E−03 |
| KIAA1683 | −1.69 | 6.35E−03 |
| CREB3L3 | −1.69 | 7.83E−03 |
| ODF2L | −1.68 | 7.24E−04 |
| SEMA4C | −1.68 | 9.99E−04 |
| FAM229A | −1.68 | 2.86E−03 |
| AKTIP | −1.67 | 7.24E−04 |
| IL11RA | −1.67 | 1.43E−03 |
| TRIM3 | −1.67 | 2.19E−03 |
| SLC25A35 | −1.67 | 4.72E−03 |
| CTA-228A9.4 | −1.67 | 5.78E−03 |
| SIGIRR | −1.66 | 2.06E−03 |
| PIK3R5 | −1.65 | 1.24E−04 |
| EGLN3 | −1.65 | 1.97E−03 |
| KIAA1671 | −1.65 | 3.35E−03 |
| RP11-378A13.2 | −1.65 | 6.25E−03 |
| KRBA2 | −1.65 | 9.19E−03 |
| SLC44A2 | −1.64 | 6.11E−04 |
| CCDC28A | −1.64 | 8.84E−04 |
| GALNT12 | −1.64 | 1.85E−03 |
| PNRC1 | −1.64 | 3.77E−03 |
| FBXO36 | −1.64 | 4.65E−03 |
| RCSD1 | −1.63 | 1.24E−04 |
| SPTBN1 | −1.63 | 2.85E−04 |
| CALHM2 | −1.63 | 2.67E−03 |
| GNRHR | −1.63 | 9.49E−03 |
| ARRB1 | −1.62 | 3.14E−04 |
| FBXL20 | −1.62 | 2.29E−03 |
| PDE3B | −1.61 | 7.24E−04 |
| ACSS1 | −1.61 | 8.77E−04 |
| CBR3 | −1.61 | 1.20E−03 |
| FRAT1 | −1.61 | 2.41E−03 |
| RP11-667F14.1 | −1.61 | 8.06E−03 |
| RP13-977J11.2 | −1.61 | 9.50E−03 |
| CAMK4 | −1.60 | 7.05E−04 |
| PRMT2 | −1.60 | 7.27E−04 |
| KIAA1841 | −1.60 | 2.87E−03 |
| B3GAT2 | −1.60 | 3.04E−03 |
| SERPINB9P1 | −1.60 | 3.56E−03 |
| SLFN5 | −1.60 | 5.57E−03 |
| COLQ | −1.59 | 4.56E−03 |
| ITGB2-AS1 | −1.59 | 7.51E−03 |
| ARAP3 | −1.58 | 2.58E−03 |
| P2RY10 | −1.57 | 7.24E−04 |
| ARHGAP12 | −1.57 | 6.25E−03 |
| RP1-283E3.8 | −1.56 | 2.14E−03 |
| BTN3A3 | −1.55 | 6.37E−04 |
| ZNF831 | −1.55 | 3.62E−03 |
| LAIR1 | −1.55 | 4.12E−03 |
| IL9R | −1.55 | 5.51E−03 |
| F2R | −1.55 | 6.06E−03 |
| ZNF219 | −1.55 | 7.53E−03 |
| ABCB1 | −1.55 | 9.14E−03 |
| PRKCB | −1.54 | 3.51E−04 |
| LPAR5 | −1.54 | 1.57E−03 |
| AGER | −1.54 | 2.06E−03 |
| RP11-291B21.2 | −1.54 | 9.31E−03 |
| S100PBP | −1.53 | 7.19E−04 |
| ITGA5 | −1.53 | 1.02E−03 |
| OPTN | −1.53 | 1.20E−03 |
| FGD3 | −1.53 | 1.44E−03 |
| GIMAP1 | −1.53 | 2.36E−03 |
| PDK2 | −1.53 | 2.55E−03 |
| ZBTB22 | −1.53 | 2.58E−03 |
| CARF | −1.53 | 3.07E−03 |
| TMX4 | −1.52 | 4.90E−04 |
| TXNDC16 | −1.52 | 2.58E−03 |
| CLYBL | −1.52 | 2.75E−03 |
| ERICH6-AS1 | −1.52 | 3.51E−03 |
| CXorf21 | −1.52 | 4.25E−03 |
| KB-1572G7.2 | −1.52 | 6.17E−03 |
| CEP162 | −1.52 | 8.92E−03 |
| PATL2 | −1.52 | 9.06E−03 |
| BAD | −1.51 | 1.82E−03 |
| RP11-617F23.1 | −1.51 | 4.02E−03 |
| RP11-180M15.7 | −1.51 | 5.35E−03 |
| PLCL2 | −1.50 | 2.73E−04 |
| RBL2 | −1.50 | 7.05E−04 |
| SMAD3 | −1.50 | 8.84E−04 |
| CCM2 | −1.50 | 9.10E−04 |
| SH2D3C | −1.50 | 1.34E−03 |
| PYHIN1 | −1.50 | 3.15E−03 |
| SLC2A4RG | −1.50 | 4.60E−03 |
| NECAB3 | −1.50 | 6.06E−03 |
| TMEM116 | −1.49 | 2.56E−03 |
| SGSH | −1.49 | 2.94E−03 |
| TUBA1A | −1.49 | 5.96E−03 |
| LYRM9 | −1.49 | 7.44E−03 |
| OSER1-AS1 | −1.48 | 3.29E−03 |
| RP11-147L13.15 | −1.48 | 3.85E−03 |
| SLC35E2 | −1.48 | 4.20E−03 |
| RP11-134G8.7 | −1.48 | 8.05E−03 |
| LINC01089 | −1.47 | 2.94E−03 |
| SENP7 | −1.47 | 4.78E−03 |
| SH3BP5 | −1.47 | 5.30E−03 |
| TRGV3 | −1.47 | 8.66E−03 |
| PTPN4 | −1.46 | 7.48E−04 |
| CBX7 | −1.46 | 2.11E−03 |
| DIP2A | −1.46 | 3.06E−03 |
| SWAP70 | −1.46 | 5.00E−03 |
| ANKRA2 | −1.45 | 1.28E−03 |
| GIMAP6 | −1.45 | 2.02E−03 |
| ARHGAP10 | −1.45 | 3.14E−03 |
| KIZ | −1.45 | 3.21E−03 |
| C16orf54 | −1.45 | 3.35E−03 |
| RP11-347119.8 | −1.45 | 9.42E−03 |
| GNB5 | −1.44 | 2.85E−04 |
| NBPF19 | −1.44 | 1.72E−03 |
| TRGV5 | −1.44 | 2.14E−03 |
| SMPD1 | −1.44 | 4.25E−03 |
| EHD3 | −1.44 | 9.10E−03 |
| KIAA0430 | −1.43 | 1.04E−03 |
| ADA | −1.43 | 2.41E−03 |
| CRTAP | −1.43 | 3.18E−03 |
| DLG4 | −1.43 | 3.68E−03 |
| RAD51-AS1 | −1.43 | 3.87E−03 |
| CDK19 | −1.43 | 9.12E−03 |
| GPR19 | −1.43 | 9.62E−03 |
| CECR1 | −1.42 | 2.36E−03 |
| TTC28-AS1 | −1.42 | 2.51E−03 |
| AMPD3 | −1.42 | 8.84E−03 |
| RASSF3 | −1.41 | 5.04E−04 |
| P2RY8 | −1.41 | 1.81E−03 |
| HBP1 | −1.41 | 2.00E−03 |
| BACE1 | −1.41 | 2.79E−03 |
| KCTD7 | −1.41 | 3.15E−03 |
| FAM117A | −1.41 | 3.17E−03 |
| RP11-297D21.4 | −1.41 | 4.28E−03 |
| ZNF449 | −1.41 | 5.29E−03 |
| ZNF792 | −1.41 | 8.86E−03 |
| NPC1 | −1.40 | 1.43E−03 |
| KLC4 | −1.40 | 1.56E−03 |
| NLRC3 | −1.40 | 2.03E−03 |
| SYNE1 | −1.40 | 4.13E−03 |
| PLA2G6 | −1.40 | 5.63E−03 |
| HDAC5 | −1.40 | 7.54E−03 |
| SSH3 | −1.40 | 9.07E−03 |
| PLD2 | −1.39 | 1.56E−03 |
| CBLL1 | −1.39 | 2.36E−03 |
| TMEM91 | −1.39 | 5.46E−03 |
| TMEM143 | −1.39 | 5.62E−03 |
| SLC25A42 | −1.39 | 7.92E−03 |
| SELPLG | −1.38 | 3.51E−04 |
| UNKL | −1.38 | 1.08E−03 |
| ING4 | −1.38 | 1.27E−03 |
| APAF1 | −1.38 | 1.57E−03 |
| AMT | −1.38 | 7.48E−03 |
| ALPK1 | −1.38 | 9.10E−03 |
| TMEM110-MUSTN1 | −1.38 | 9.51E−03 |
| JAK1 | −1.37 | 3.26E−04 |
| BDH2 | −1.37 | 1.40E−03 |
| PHF21A | −1.37 | 1.43E−03 |
| RP11-541N10.3 | −1.37 | 1.67E−03 |
| CUL4B | −1.37 | 1.78E−03 |
| PCCA | −1.37 | 2.14E−03 |
| PAN2 | −1.37 | 2.28E−03 |
| HACD4 | −1.37 | 2.36E−03 |
| PRCP | −1.37 | 3.78E−03 |
| CBLN3 | −1.37 | 4.41E−03 |
| ZNF345 | −1.37 | 4.76E−03 |
| ATP8B2 | −1.36 | 9.43E−04 |
| SLC39A11 | −1.36 | 9.94E−04 |
| ALAD | −1.36 | 1.57E−03 |
| MAP4K2 | −1.36 | 1.64E−03 |
| ILK | −1.36 | 1.70E−03 |
| ZMYM3 | −1.36 | 2.19E−03 |
| CKAP2 | −1.36 | 3.73E−03 |
| GPSM3 | −1.36 | 3.79E−03 |
| TNFAIP8L1 | −1.35 | 7.14E−04 |
| LMBR1L | −1.35 | 1.43E−03 |
| ZER1 | −1.35 | 1.57E−03 |
| SERINC5 | −1.35 | 2.56E−03 |
| KIF22 | −1.35 | 4.53E−03 |
| ATG2A | −1.35 | 8.67E−03 |
| WHAMM | −1.34 | 7.75E−04 |
| KANSL1L | −1.34 | 3.09E−03 |
| ZNF862 | −1.34 | 3.62E−03 |
| IDH1 | −1.34 | 5.99E−03 |
| FAM63A | −1.34 | 6.44E−03 |
| 8-Mar | −1.33 | 1.69E−03 |
| ZC4H2 | −1.33 | 2.91E−03 |
| PCYOX1 | −1.33 | 2.98E−03 |
| AC009950.2 | −1.33 | 4.11E−03 |
| BBS9 | −1.33 | 4.71E−03 |
| RP11-111M22.2 | −1.33 | 6.12E−03 |
| BEND4 | −1.33 | 6.57E−03 |
| ANKRD26 | −1.32 | 9.43E−04 |
| PCMTD1 | −1.32 | 2.84E−03 |
| NMRK1 | −1.32 | 4.18E−03 |
| ZNF846 | −1.32 | 7.82E−03 |
| HERC1 | −1.31 | 1.26E−03 |
| PPP1R12B | −1.31 | 1.30E−03 |
| TBC1D10C | −1.31 | 1.85E−03 |
| CCDC28B | −1.31 | 2.41E−03 |
| MAP3K7CL | −1.31 | 5.81E−03 |
| DNAJC3-AS1 | −1.31 | 6.06E−03 |
| LINC00672 | −1.31 | 7.86E−03 |
| ANO8 | −1.31 | 7.91E−03 |
| KLF12 | −1.30 | 1.20E−03 |
| NDRG3 | −1.30 | 1.96E−03 |
| TTC9 | −1.30 | 3.56E−03 |
| GIMAP2 | −1.30 | 4.57E−03 |
| BTD | −1.30 | 7.44E−03 |
| NPHP3 | −1.30 | 7.99E−03 |
| RP11-196G18.22 | −1.30 | 9.55E−03 |
| PCNT | −1.29 | 4.10E−04 |
| CYTH1 | −1.29 | 7.24E−04 |
| NUMA1 | −1.29 | 2.28E−03 |
| CENPK | −1.29 | 3.43E−03 |
| TFDP2 | −1.29 | 3.90E−03 |
| MAP3K3 | −1.29 | 4.28E−03 |
| RP4-605O3.4 | −1.29 | 9.25E−03 |
| BTN2A2 | −1.28 | 6.11E−04 |
| ZNF33B | −1.28 | 9.51E−04 |
| BIN2 | −1.28 | 1.69E−03 |
| IL16 | −1.28 | 2.29E−03 |
| GTF2IRD2B | −1.28 | 3.28E−03 |
| DZIP3 | −1.28 | 5.90E−03 |
| SUSD3 | −1.27 | 1.28E−03 |
| KIAA0586 | −1.27 | 1.43E−03 |
| ZMYM1 | −1.27 | 4.12E−03 |
| CEP19 | −1.27 | 4.22E−03 |
| TTYH2 | −1.27 | 8.60E−03 |
| POLR3GL | −1.26 | 9.10E−04 |
| COG4 | −1.26 | 1.20E−03 |
| BTN3A2 | −1.26 | 1.66E−03 |
| LIMK2 | −1.26 | 1.69E−03 |
| IFT80 | −1.26 | 2.17E−03 |
| PLCB2 | −1.26 | 2.29E−03 |
| MEGF9 | −1.26 | 2.34E−03 |
| FAM111A | −1.26 | 4.16E−03 |
| C1QTNF6 | −1.26 | 8.68E−03 |
| MIS18BP1 | −1.25 | 1.12E−03 |
| BIN1 | −1.25 | 1.64E−03 |
| USP28 | −1.25 | 1.81E−03 |
| RAB29 | −1.25 | 1.85E−03 |
| RAB5B | −1.25 | 2.55E−03 |
| CNOT6L | −1.25 | 2.59E−03 |
| PPOX | −1.25 | 5.23E−03 |
| SLC2A11 | −1.25 | 7.06E−03 |
| ERP27 | −1.25 | 7.16E−03 |
| RARRES3 | −1.25 | 8.99E−03 |
| NT5DC1 | −1.24 | 9.14E−04 |
| MXD1 | −1.24 | 1.64E−03 |
| SMYD3 | −1.24 | 1.65E−03 |
| CCPG1 | −1.24 | 2.51E−03 |
| UBE2H | −1.24 | 2.94E−03 |
| PSIP1 | −1.23 | 4.73E−04 |
| GRK5 | −1.23 | 7.24E−04 |
| SH3BP1 | −1.23 | 1.26E−03 |
| ZFP36L2 | −1.23 | 1.59E−03 |
| DGKA | −1.23 | 2.32E−03 |
| CEP192 | −1.23 | 4.39E−03 |
| CTB-25B13.12 | −1.23 | 5.98E−03 |
| CCBL1 | −1.23 | 8.12E−03 |
| PHKB | −1.22 | 7.07E−04 |
| CCNG1 | −1.22 | 7.72E−04 |
| RBBP9 | −1.22 | 1.91E−03 |
| ARID4A | −1.22 | 2.03E−03 |
| KIAA1109 | −1.22 | 2.87E−03 |
| APH1B | −1.22 | 3.33E−03 |
| LINC00847 | −1.22 | 3.80E−03 |
| ADAM8 | −1.22 | 5.02E−03 |
| NTAN1 | −1.21 | 2.41E−03 |
| TMEM19 | −1.21 | 2.51E−03 |
| IQSEC1 | −1.21 | 3.34E−03 |
| TRAV9-2 | −1.21 | 4.56E−03 |
| CCDC14 | −1.21 | 5.12E−03 |
| DGCR6L | −1.21 | 6.15E−03 |
| HSDL2 | −1.21 | 6.46E−03 |
| HMGB2 | −1.21 | 7.75E−03 |
| NLRC5 | −1.20 | 7.27E−04 |
| CAMK2G | −1.20 | 7.76E−04 |
| OPN3 | −1.20 | 3.60E−03 |
| TRBV6-5 | −1.20 | 5.62E−03 |
| FAM63B | −1.20 | 7.16E−03 |
| TBC1D10A | −1.20 | 7.30E−03 |
| CEP57L1 | −1.20 | 8.13E−03 |
| ZYG11B | −1.20 | 8.76E−03 |
| SNRK | −1.19 | 7.32E−04 |
| HCP5 | −1.19 | 1.90E−03 |
| GTF2IRD2 | −1.19 | 2.02E−03 |
| SWT1 | −1.19 | 4.73E−03 |
| RP11-15H20.6 | −1.19 | 4.93E−03 |
| KIAA0319L | −1.18 | 2.30E−03 |
| GMDS-AS1 | −1.18 | 3.75E−03 |
| TPK1 | −1.18 | 4.47E−03 |
| BCKDHB | −1.17 | 1.40E−03 |
| RAD52 | −1.17 | 2.47E−03 |
| SIPA1 | −1.17 | 2.74E−03 |
| GBA | −1.17 | 2.88E−03 |
| MLLT11 | −1.17 | 5.26E−03 |
| C5orf42 | −1.17 | 5.48E−03 |
| RNASET2 | −1.16 | 1.97E−03 |
| LAPTM5 | −1.16 | 2.28E−03 |
| PIAS3 | −1.16 | 2.36E−03 |
| PHYH | −1.16 | 2.86E−03 |
| TCAF1 | −1.16 | 7.15E−03 |
| CTPS2 | −1.16 | 7.69E−03 |
| C14orf93 | −1.16 | 9.80E−03 |
| STK17A | −1.15 | 9.99E−04 |
| CBFA2T2 | −1.15 | 1.78E−03 |
| ARRDC2 | −1.15 | 2.45E−03 |
| FAM168A | −1.15 | 2.58E−03 |
| PPP3CB-AS1 | −1.15 | 4.20E−03 |
| LINC00869 | −1.15 | 4.47E−03 |
| SLC12A4 | −1.15 | 6.17E−03 |
| SIDT2 | −1.15 | 6.76E−03 |
| PBX2 | −1.14 | 2.85E−04 |
| ZBTB4 | −1.14 | 1.03E−03 |
| TXNIP | −1.14 | 2.41E−03 |
| CAT | −1.14 | 2.96E−03 |
| ING2 | −1.14 | 3.40E−03 |
| MROH1 | −1.14 | 3.44E−03 |
| MCF2L2 | −1.14 | 4.54E−03 |
| ZFYVE16 | −1.14 | 7.48E−03 |
| DBF4B | −1.14 | 9.72E−03 |
| ECI2 | −1.13 | 1.09E−03 |
| FLOT2 | −1.13 | 1.54E−03 |
| BIVM-ERCC5 | −1.13 | 4.41E−03 |
| ATP8A1 | −1.13 | 4.87E−03 |
| ZKSCAN3 | −1.13 | 5.82E−03 |
| SIPA1L3 | −1.13 | 6.09E−03 |
| TMEM204 | −1.13 | 7.06E−03 |
| CEP70 | −1.13 | 9.67E−03 |
| TMEM245 | −1.12 | 1.97E−03 |
| PGPEP1 | −1.12 | 2.68E−03 |
| ABCC5 | −1.12 | 3.10E−03 |
| S1PR4 | −1.12 | 4.51E−03 |
| RP11-819C21.1 | −1.12 | 9.15E−03 |
| LPXN | −1.11 | 3.51E−04 |
| NDE1 | −1.11 | 1.78E−03 |
| RP1-39G22.7 | −1.11 | 2.14E−03 |
| RP11-705C15.3 | −1.11 | 4.27E−03 |
| PCNXL2 | −1.11 | 4.47E−03 |
| MBD5 | −1.11 | 4.61E−03 |
| ZNF441 | −1.11 | 5.63E−03 |
| MBOAT1 | −1.11 | 6.15E−03 |
| RFX3 | −1.11 | 6.31E−03 |
| TOM1L2 | −1.11 | 6.34E−03 |
| UTRN | −1.11 | 7.15E−03 |
| BAZ2B | −1.11 | 8.90E−03 |
| CTC1 | −1.10 | 9.43E−04 |
| BCAS4 | −1.10 | 2.40E−03 |
| ACAP2 | −1.10 | 3.93E−03 |
| LRCH4 | −1.10 | 6.18E−03 |
| ARHGAP11B | −1.10 | 6.20E−03 |
| RP11-174G6.5 | −1.10 | 6.79E−03 |
| DEDD2 | −1.10 | 9.02E−03 |
| KAT6B | −1.09 | 1.25E−03 |
| NIPAL3 | −1.09 | 2.54E−03 |
| FAM200B | −1.09 | 2.96E−03 |
| MINK1 | −1.09 | 3.04E−03 |
| ZADH2 | −1.09 | 3.29E−03 |
| RP11-57H14.4 | −1.09 | 5.38E−03 |
| KBTBD7 | −1.09 | 6.78E−03 |
| RP11-399O19.9 | −1.09 | 6.99E−03 |
| ZCCHC11 | −1.08 | 2.85E−04 |
| AKT3 | −1.08 | 6.77E−04 |
| HDAC6 | −1.08 | 1.26E−03 |
| PRKACB | −1.08 | 1.92E−03 |
| ACADSB | −1.08 | 2.36E−03 |
| TNRC6C | −1.08 | 3.73E−03 |
| VAT1 | −1.08 | 4.45E−03 |
| NICN1 | −1.08 | 5.02E−03 |
| WBP1 | −1.07 | 3.63E−03 |
| ALOX12-AS1 | −1.07 | 4.39E−03 |
| GSTM2 | −1.07 | 4.72E−03 |
| PHC1 | −1.07 | 6.37E−03 |
| RP11-799D4.4 | −1.07 | 9.09E−03 |
| TTC7A | −1.06 | 6.43E−04 |
| SAP30L | −1.06 | 1.43E−03 |
| PIK3CB | −1.06 | 1.78E−03 |
| ZNF75D | −1.06 | 2.47E−03 |
| PITPNC1 | −1.06 | 2.58E−03 |
| UBA7 | −1.06 | 2.58E−03 |
| PARP8 | −1.06 | 2.81E−03 |
| FBXL17 | −1.06 | 2.94E−03 |
| CCHCR1 | −1.06 | 6.60E−03 |
| CEP295 | −1.06 | 7.52E−03 |
| CDC42SE2 | −1.05 | 3.54E−04 |
| SSH2 | −1.05 | 9.43E−04 |
| GNS | −1.05 | 1.09E−03 |
| TBCK | −1.05 | 1.20E−03 |
| MTERF4 | −1.05 | 2.00E−03 |
| SLC35E2B | −1.05 | 2.81E−03 |
| LSP1 | −1.05 | 2.91E−03 |
| SLFN12L | −1.05 | 3.19E−03 |
| PRIMPOL | −1.05 | 3.68E−03 |
| CYB5A | −1.05 | 3.75E−03 |
| ARHGAP19 | −1.05 | 4.30E−03 |
| TRAV21 | −1.05 | 7.16E−03 |
| RP11-244H3.1 | −1.05 | 7.51E−03 |
| SGTB | −1.05 | 9.52E−03 |
| UNC119 | −1.04 | 1.78E−03 |
| CDKN1B | −1.04 | 2.07E−03 |
| ZFAND2B | −1.04 | 2.59E−03 |
| SH3BP5-AS1 | −1.04 | 3.84E−03 |
| ZNF436 | −1.04 | 5.23E−03 |
| PLEKHA1 | −1.04 | 6.44E−03 |
| CD5 | −1.04 | 9.83E−03 |
| ITGB7 | −1.03 | 1.03E−03 |
| VPS26B | −1.03 | 1.03E−03 |
| KLHL22 | −1.03 | 1.19E−03 |
| CCDC82 | −1.03 | 1.66E−03 |
| CD3G | −1.03 | 1.93E−03 |
| ZNF226 | −1.03 | 2.32E−03 |
| HPS3 | −1.03 | 2.58E−03 |
| NHLRC3 | −1.03 | 3.20E−03 |
| KLRK1 | −1.03 | 3.91E−03 |
| SVBP | −1.03 | 4.61E−03 |
| 9-Mar | −1.03 | 4.69E−03 |
| IGIP | −1.03 | 6.06E−03 |
| STK10 | −1.02 | 7.24E−04 |
| DCK | −1.02 | 1.93E−03 |
| POMT1 | −1.02 | 2.46E−03 |
| SIRT3 | −1.02 | 3.87E−03 |
| CYTH2 | −1.02 | 4.47E−03 |
| CRYZ | −1.02 | 4.71E−03 |
| PIK3C2B | −1.02 | 7.16E−03 |
| ELMOD3 | −1.02 | 8.31E−03 |
| UPF3A | −1.01 | 1.51E−03 |
| ANKZF1 | −1.01 | 1.90E−03 |
| IKBKE | −1.01 | 1.98E−03 |
| PRKAB2 | −1.01 | 2.70E−03 |
| CERK | −1.01 | 3.35E−03 |
| MYO18A | −1.01 | 3.68E−03 |
| IFT122 | −1.01 | 3.73E−03 |
| UBL3 | −1.01 | 5.49E−03 |
| SPIDR | −1.00 | 1.20E−03 |
| SMC4 | −1.00 | 1.60E−03 |
| SYT11 | −1.00 | 2.66E−03 |
| CXorf57 | −1.00 | 5.73E−03 |
| RPS6KA5 | 1.00 | 1.30E−03 |
| HSP90AA1 | 1.00 | 3.14E−03 |
| SEC61B | 1.00 | 3.78E−03 |
| EFNB1 | 1.00 | 9.10E−03 |
| SYNJ2 | 1.01 | 1.01E−03 |
| SPATA5L1 | 1.01 | 1.78E−03 |
| PDIA6 | 1.01 | 2.34E−03 |
| EIF4E | 1.01 | 2.41E−03 |
| FTSJ1 | 1.01 | 2.58E−03 |
| AHSA1 | 1.01 | 2.91E−03 |
| SYNCRIP | 1.01 | 3.10E−03 |
| PLOD1 | 1.01 | 3.18E−03 |
| TIMM50 | 1.01 | 3.42E−03 |
| BMP1 | 1.01 | 4.52E−03 |
| SLC35C1 | 1.01 | 4.71E−03 |
| THOP1 | 1.01 | 5.23E−03 |
| C16orf91 | 1.01 | 5.32E−03 |
| IKBIP | 1.01 | 5.62E−03 |
| MPV17L2 | 1.01 | 5.90E−03 |
| EIF3C | 1.01 | 6.31E−03 |
| MSTO1 | 1.02 | 1.14E−03 |
| MRPS23 | 1.02 | 2.72E−03 |
| UTP6 | 1.02 | 2.75E−03 |
| QSOX2 | 1.02 | 3.30E−03 |
| PAM16 | 1.02 | 3.50E−03 |
| ATF5 | 1.02 | 3.60E−03 |
| NDUFV2 | 1.02 | 3.76E−03 |
| HSPA9 | 1.02 | 4.41E−03 |
| MRPL15 | 1.02 | 7.31E−03 |
| SPEN | 1.03 | 3.51E−04 |
| EIF4A3 | 1.03 | 1.03E−03 |
| CCT4 | 1.03 | 2.32E−03 |
| IPO5 | 1.03 | 3.29E−03 |
| CD44 | 1.03 | 3.32E−03 |
| TSFM | 1.03 | 3.34E−03 |
| IL4R | 1.03 | 3.40E−03 |
| ZNF771 | 1.03 | 4.80E−03 |
| OTUD6B | 1.03 | 5.01E−03 |
| DDX49 | 1.03 | 5.23E−03 |
| PHTF2 | 1.03 | 8.84E−03 |
| TMEM33 | 1.04 | 7.27E−04 |
| CBFB | 1.04 | 1.34E−03 |
| ZNF696 | 1.04 | 2.41E−03 |
| GALE | 1.04 | 4.41E−03 |
| MAPKAPK3 | 1.04 | 4.50E−03 |
| MRPL3 | 1.04 | 5.30E−03 |
| RP11-135F9.3 | 1.04 | 5.71E−03 |
| TXN | 1.04 | 5.78E−03 |
| RIOK1 | 1.04 | 6.33E−03 |
| MECR | 1.04 | 9.14E−03 |
| SLAMF1 | 1.05 | 6.11E−04 |
| GSR | 1.05 | 1.69E−03 |
| KTI12 | 1.05 | 2.06E−03 |
| CAD | 1.05 | 3.08E−03 |
| FXN | 1.05 | 3.22E−03 |
| RP3-461F17.3 | 1.05 | 3.70E−03 |
| EIF3B | 1.05 | 4.08E−03 |
| EIF1AX | 1.05 | 4.71E−03 |
| NANP | 1.05 | 4.91E−03 |
| PLCXD2 | 1.05 | 5.26E−03 |
| DUS3L | 1.05 | 5.32E−03 |
| MIF | 1.05 | 6.13E−03 |
| PDCD11 | 1.06 | 1.93E−03 |
| NAA15 | 1.06 | 2.36E−03 |
| TBL1X | 1.06 | 2.46E−03 |
| SMS | 1.06 | 2.51E−03 |
| TRMT10C | 1.06 | 3.15E−03 |
| SERTAD1 | 1.06 | 3.63E−03 |
| HEATR3 | 1.06 | 4.15E−03 |
| ATIC | 1.06 | 4.43E−03 |
| EIF3J | 1.06 | 4.49E−03 |
| DUSP14 | 1.06 | 6.75E−03 |
| ADAM19 | 1.06 | 7.31E−03 |
| FDXACB1 | 1.06 | 8.08E−03 |
| PEA15 | 1.07 | 1.14E−03 |
| YWHAG | 1.07 | 1.60E−03 |
| DNAJC25 | 1.07 | 2.36E−03 |
| MPZL3 | 1.07 | 2.40E−03 |
| ALDH18A1 | 1.07 | 2.94E−03 |
| SNTB1 | 1.07 | 3.35E−03 |
| CCT7 | 1.07 | 3.73E−03 |
| DNAJC2 | 1.07 | 3.79E−03 |
| RRAS2 | 1.07 | 6.26E−03 |
| LYRM1 | 1.08 | 1.02E−03 |
| KCNK6 | 1.08 | 1.04E−03 |
| HYOU1 | 1.08 | 2.07E−03 |
| FLOT1 | 1.08 | 2.36E−03 |
| EIF5B | 1.08 | 2.70E−03 |
| POFUT1 | 1.08 | 2.72E−03 |
| IPO11 | 1.08 | 2.79E−03 |
| BCCIP | 1.08 | 3.72E−03 |
| HSPA8 | 1.08 | 4.04E−03 |
| B3GALT6 | 1.08 | 4.18E−03 |
| ABCB6 | 1.08 | 5.41E−03 |
| PTMS | 1.08 | 8.69E−03 |
| ARPC5L | 1.09 | 1.73E−03 |
| IFT57 | 1.09 | 2.27E−03 |
| UTP11L | 1.09 | 2.70E−03 |
| CCT3 | 1.09 | 3.34E−03 |
| ERI1 | 1.09 | 3.36E−03 |
| CAPN15 | 1.09 | 4.19E−03 |
| SLC27A4 | 1.09 | 5.43E−03 |
| C3orf33 | 1.09 | 6.17E−03 |
| COQ3 | 1.09 | 7.99E−03 |
| FANCC | 1.10 | 1.26E−03 |
| SSSCA1 | 1.10 | 2.32E−03 |
| DCAF13 | 1.10 | 2.58E−03 |
| SERBP1 | 1.10 | 2.65E−03 |
| NAT10 | 1.10 | 2.88E−03 |
| SLC35F2 | 1.10 | 3.40E−03 |
| TIMM23 | 1.10 | 3.73E−03 |
| HSPBP1 | 1.10 | 6.33E−03 |
| TCEB1 | 1.11 | 7.05E−04 |
| UAP1 | 1.11 | 1.78E−03 |
| SCO2 | 1.11 | 2.13E−03 |
| PRPF19 | 1.11 | 2.17E−03 |
| SRFBP1 | 1.11 | 2.70E−03 |
| PEX5 | 1.11 | 2.71E−03 |
| TXNRD1 | 1.11 | 2.87E−03 |
| SRGAP2C | 1.11 | 3.15E−03 |
| ADO | 1.11 | 3.91E−03 |
| DNLZ | 1.11 | 5.98E−03 |
| FAM210A | 1.12 | 1.05E−03 |
| C8orf33 | 1.12 | 2.58E−03 |
| AARSD1 | 1.12 | 3.80E−03 |
| MYDGF | 1.12 | 4.70E−03 |
| TLE3 | 1.12 | 5.23E−03 |
| NIFK | 1.12 | 5.55E−03 |
| AEN | 1.12 | 7.61E−03 |
| LENG9 | 1.12 | 8.61E−03 |
| SLC5A6 | 1.13 | 1.69E−03 |
| AK2 | 1.13 | 2.55E−03 |
| MRPL36 | 1.13 | 2.89E−03 |
| AMIGO2 | 1.13 | 2.95E−03 |
| UBASH3B | 1.13 | 3.15E−03 |
| CENPV | 1.13 | 3.29E−03 |
| YIF1A | 1.13 | 3.47E−03 |
| ACACA | 1.13 | 3.66E−03 |
| FAAP100 | 1.13 | 3.69E−03 |
| MPHOSPH6 | 1.13 | 4.43E−03 |
| PEX10 | 1.13 | 6.06E−03 |
| CYSTM1 | 1.13 | 7.15E−03 |
| ITPR1 | 1.14 | 7.24E−04 |
| ATP2A2 | 1.14 | 1.28E−03 |
| POLR2K | 1.14 | 1.57E−03 |
| LRRC59 | 1.14 | 1.66E−03 |
| NOC2L | 1.14 | 2.27E−03 |
| NEDD9 | 1.14 | 2.59E−03 |
| IDH3A | 1.14 | 2.75E−03 |
| RANBP1 | 1.14 | 3.35E−03 |
| PRDX1 | 1.14 | 4.07E−03 |
| CCT5 | 1.14 | 4.18E−03 |
| ZNF587B | 1.14 | 4.43E−03 |
| GGCT | 1.14 | 4.44E−03 |
| GAR1 | 1.14 | 4.89E−03 |
| TESC | 1.14 | 5.78E−03 |
| YDJC | 1.14 | 5.89E−03 |
| COMTD1 | 1.14 | 5.90E−03 |
| MMACHC | 1.15 | 1.78E−03 |
| EIF4G1 | 1.15 | 1.96E−03 |
| MAFK | 1.15 | 2.34E−03 |
| ABCF2 | 1.15 | 2.44E−03 |
| POP7 | 1.15 | 2.91E−03 |
| SRGN | 1.15 | 3.09E−03 |
| SLC25A33 | 1.15 | 5.16E−03 |
| IMPDH2 | 1.15 | 6.41E−03 |
| ALKBH2 | 1.15 | 6.45E−03 |
| CD151 | 1.15 | 6.54E−03 |
| FBXW9 | 1.15 | 6.62E−03 |
| FRMD4B | 1.15 | 9.83E−03 |
| C16orf87 | 1.16 | 1.78E−03 |
| LARP1B | 1.16 | 1.97E−03 |
| RBM14 | 1.16 | 2.23E−03 |
| CBR1 | 1.16 | 2.41E−03 |
| GEMIN5 | 1.16 | 2.41E−03 |
| FDX1L | 1.16 | 4.26E−03 |
| RRP7A | 1.16 | 4.44E−03 |
| NACC1 | 1.16 | 5.55E−03 |
| ARMC6 | 1.16 | 5.55E−03 |
| AGMAT | 1.16 | 6.72E−03 |
| NPM1 | 1.16 | 7.26E−03 |
| TIMM13 | 1.16 | 9.25E−03 |
| ATP13A3 | 1.17 | 7.24E−04 |
| FAM98A | 1.17 | 1.03E−03 |
| MARS2 | 1.17 | 1.83E−03 |
| B3GLCT | 1.17 | 2.15E−03 |
| UGCG | 1.17 | 2.62E−03 |
| RCN1 | 1.17 | 2.80E−03 |
| ACOT7 | 1.17 | 2.91E−03 |
| ALG3 | 1.17 | 2.94E−03 |
| PHB | 1.17 | 3.46E−03 |
| MRPS26 | 1.17 | 3.71E−03 |
| TIMM10 | 1.17 | 4.13E−03 |
| WDR46 | 1.17 | 4.41E−03 |
| NOP14 | 1.17 | 4.50E−03 |
| PRKD3 | 1.18 | 6.14E−04 |
| TIMM23B | 1.18 | 1.03E−03 |
| AGO2 | 1.18 | 1.09E−03 |
| PA2G4 | 1.18 | 2.17E−03 |
| PRADC1 | 1.18 | 3.38E−03 |
| ABCE1 | 1.18 | 3.42E−03 |
| WDR36 | 1.18 | 3.78E−03 |
| BOLA2B | 1.18 | 6.06E−03 |
| NHP2 | 1.18 | 8.79E−03 |
| PTPN7 | 1.18 | 8.90E−03 |
| SLA | 1.19 | 3.35E−04 |
| FNDC3B | 1.19 | 1.57E−03 |
| MAN1A1 | 1.19 | 1.72E−03 |
| NIP7 | 1.19 | 1.78E−03 |
| POLR1B | 1.19 | 1.78E−03 |
| PPIL1 | 1.19 | 2.42E−03 |
| MAP2K3 | 1.19 | 2.58E−03 |
| HMBS | 1.19 | 3.04E−03 |
| TOMM5 | 1.19 | 3.10E−03 |
| MTFP1 | 1.19 | 3.78E−03 |
| MRPS12 | 1.19 | 4.18E−03 |
| PTRH1 | 1.19 | 4.52E−03 |
| BAIAP2 | 1.19 | 6.13E−03 |
| MCAT | 1.19 | 6.65E−03 |
| GPAT2 | 1.19 | 8.15E−03 |
| DSTYK | 1.20 | 1.24E−04 |
| GRAMD4 | 1.20 | 4.10E−04 |
| NDFIP1 | 1.20 | 1.72E−03 |
| EIF2S1 | 1.20 | 1.82E−03 |
| NXT1 | 1.20 | 1.91E−03 |
| FARP2 | 1.20 | 1.93E−03 |
| EIF4A1 | 1.20 | 2.19E−03 |
| TMA16 | 1.20 | 2.19E−03 |
| LTV1 | 1.20 | 2.53E−03 |
| MRPS17 | 1.20 | 2.60E−03 |
| NOP2 | 1.20 | 2.96E−03 |
| IL15RA | 1.20 | 3.38E−03 |
| MRPL4 | 1.20 | 6.28E−03 |
| POLR3C | 1.21 | 6.14E−04 |
| UHRF1BP1L | 1.21 | 1.64E−03 |
| COLGALT1 | 1.21 | 2.58E−03 |
| ETF1 | 1.21 | 2.71E−03 |
| SNRPD1 | 1.21 | 3.40E−03 |
| GNL3 | 1.21 | 3.75E−03 |
| CNKSR2 | 1.21 | 4.04E−03 |
| DOT1L | 1.21 | 4.27E−03 |
| AHCY | 1.21 | 5.91E−03 |
| EEF2KMT | 1.21 | 8.84E−03 |
| SFXN2 | 1.22 | 1.72E−03 |
| NUDCD1 | 1.22 | 1.82E−03 |
| PRMT5 | 1.22 | 2.51E−03 |
| TBL3 | 1.22 | 2.59E−03 |
| KDM6B | 1.22 | 2.79E−03 |
| NOTCH1 | 1.22 | 2.83E−03 |
| NOC4L | 1.22 | 3.11E−03 |
| CCDC58 | 1.22 | 3.90E−03 |
| SLC35G1 | 1.22 | 4.25E−03 |
| PSMG1 | 1.22 | 5.12E−03 |
| GNL2 | 1.23 | 1.98E−03 |
| IMP4 | 1.23 | 2.36E−03 |
| KLHL11 | 1.23 | 3.38E−03 |
| RPIA | 1.23 | 3.72E−03 |
| FAM216A | 1.23 | 5.63E−03 |
| ALDH1B1 | 1.23 | 5.98E−03 |
| STX1A | 1.23 | 9.13E−03 |
| ZSWIM6 | 1.24 | 1.24E−04 |
| FAM102B | 1.24 | 7.24E−04 |
| KLHL8 | 1.24 | 1.51E−03 |
| BTG3 | 1.24 | 2.34E−03 |
| PPA1 | 1.24 | 3.34E−03 |
| SLC46A1 | 1.24 | 3.48E−03 |
| ETS2 | 1.24 | 3.87E−03 |
| NUFIP1 | 1.25 | 1.93E−03 |
| CEP83 | 1.25 | 1.96E−03 |
| AMD1 | 1.25 | 2.84E−03 |
| WDR3 | 1.25 | 3.34E−03 |
| ST7 | 1.25 | 3.50E−03 |
| C1QBP | 1.25 | 4.65E−03 |
| VPS9D1-AS1 | 1.25 | 9.74E−03 |
| E2F6 | 1.26 | 1.20E−03 |
| C1orf216 | 1.26 | 2.02E−03 |
| RAB11FIP1 | 1.26 | 2.17E−03 |
| CEBPB | 1.26 | 2.25E−03 |
| KDSR | 1.26 | 2.62E−03 |
| ESF1 | 1.26 | 2.87E−03 |
| CHPF | 1.26 | 3.11E−03 |
| MEX3D | 1.26 | 3.73E−03 |
| DCAF4 | 1.26 | 5.38E−03 |
| CCDC85B | 1.27 | 1.44E−03 |
| PUS1 | 1.27 | 3.60E−03 |
| ZNF485 | 1.27 | 5.43E−03 |
| RP11-326C3.2 | 1.27 | 5.67E−03 |
| IARS | 1.27 | 6.20E−03 |
| RUNX1 | 1.27 | 7.75E−03 |
| IL12RB2 | 1.27 | 8.78E−03 |
| GEMIN4 | 1.28 | 1.85E−03 |
| SPATA24 | 1.28 | 2.23E−03 |
| MRM1 | 1.28 | 2.55E−03 |
| NDUFAF4 | 1.28 | 2.79E−03 |
| SLC16A1 | 1.28 | 3.19E−03 |
| RP11-77H9.2 | 1.28 | 3.63E−03 |
| ZNRF1 | 1.28 | 6.69E−03 |
| SLCO4A1 | 1.28 | 7.10E−03 |
| HSPA5 | 1.29 | 1.43E−03 |
| SRPRB | 1.29 | 1.78E−03 |
| JMJD4 | 1.29 | 1.99E−03 |
| GNPNAT1 | 1.29 | 2.36E−03 |
| FARSA | 1.29 | 2.94E−03 |
| C1orf198 | 1.29 | 4.23E−03 |
| NCLN | 1.29 | 4.27E−03 |
| TTLL12 | 1.29 | 4.50E−03 |
| CH507-9B2.5 | 1.29 | 6.01E−03 |
| RPL22L1 | 1.29 | 7.06E−03 |
| RCAN1 | 1.29 | 7.16E−03 |
| SETD7 | 1.29 | 8.73E−03 |
| EPT1 | 1.30 | 1.73E−03 |
| POLR1A | 1.30 | 1.93E−03 |
| AKAP1 | 1.30 | 2.06E−03 |
| SOGA3 | 1.30 | 2.19E−03 |
| CCT2 | 1.30 | 2.56E−03 |
| UCHL3 | 1.30 | 2.58E−03 |
| CD72 | 1.30 | 2.88E−03 |
| EEF1E1 | 1.30 | 2.94E−03 |
| TNFSF14 | 1.30 | 3.26E−03 |
| DESI1 | 1.30 | 3.45E−03 |
| CD320 | 1.30 | 5.63E−03 |
| NKG7 | 1.30 | 6.51E−03 |
| CDC42EP3 | 1.31 | 6.37E−04 |
| TMEM70 | 1.31 | 7.27E−04 |
| NAF1 | 1.31 | 1.29E−03 |
| URB1 | 1.31 | 1.66E−03 |
| FAM57A | 1.31 | 3.73E−03 |
| INAFM2 | 1.31 | 9.13E−03 |
| PDP2 | 1.32 | 5.04E−04 |
| ZC3H12C | 1.32 | 9.43E−04 |
| SFXN1 | 1.32 | 1.20E−03 |
| GRWD1 | 1.32 | 2.15E−03 |
| ZNF584 | 1.32 | 2.50E−03 |
| SNHG15 | 1.32 | 3.49E−03 |
| MLLT4 | 1.32 | 3.72E−03 |
| WDR74 | 1.32 | 3.72E−03 |
| FAM86C1 | 1.32 | 5.45E−03 |
| DUSP2 | 1.32 | 6.25E−03 |
| CRY1 | 1.33 | 3.51E−04 |
| STIP1 | 1.33 | 1.01E−03 |
| NOL10 | 1.33 | 1.30E−03 |
| POP1 | 1.33 | 1.57E−03 |
| MAPK6 | 1.33 | 2.06E−03 |
| WDR77 | 1.33 | 2.35E−03 |
| GTPBP4 | 1.33 | 2.41E−03 |
| NOL6 | 1.33 | 3.21E−03 |
| TRAP1 | 1.33 | 3.56E−03 |
| ENO1 | 1.33 | 3.60E−03 |
| DHRS11 | 1.33 | 4.24E−03 |
| SNX10 | 1.34 | 1.33E−03 |
| DDX31 | 1.34 | 1.57E−03 |
| SAMSN1 | 1.34 | 1.58E−03 |
| UTP15 | 1.34 | 2.12E−03 |
| RAN | 1.34 | 2.16E−03 |
| DHX37 | 1.34 | 2.29E−03 |
| BZW2 | 1.34 | 2.41E−03 |
| CYCS | 1.34 | 2.84E−03 |
| SIGMAR1 | 1.34 | 3.40E−03 |
| NOP58 | 1.34 | 4.09E−03 |
| KIF9 | 1.34 | 5.55E−03 |
| PTPRE | 1.35 | 2.69E−04 |
| STAT5A | 1.35 | 1.12E−03 |
| LONP1 | 1.35 | 2.02E−03 |
| PFDN2 | 1.35 | 2.28E−03 |
| MAT2A | 1.35 | 2.42E−03 |
| CAMKK2 | 1.35 | 2.70E−03 |
| NCL | 1.35 | 2.90E−03 |
| CHAC2 | 1.35 | 3.45E−03 |
| PNPT1 | 1.35 | 4.41E−03 |
| USP31 | 1.36 | 1.85E−03 |
| BRIX1 | 1.36 | 3.11E−03 |
| MAGI1 | 1.36 | 4.73E−03 |
| RP3-508I15.21 | 1.36 | 7.06E−03 |
| JARID2 | 1.37 | 1.24E−04 |
| LARP4 | 1.37 | 7.19E−04 |
| DCUN1D3 | 1.37 | 1.57E−03 |
| DHX33 | 1.37 | 1.85E−03 |
| PES1 | 1.37 | 2.79E−03 |
| UTP14A | 1.37 | 2.95E−03 |
| SLIRP | 1.37 | 2.96E−03 |
| WDR43 | 1.37 | 3.25E−03 |
| CLN6 | 1.37 | 3.68E−03 |
| PAK1IP1 | 1.37 | 3.72E−03 |
| SRXN1 | 1.37 | 4.04E−03 |
| POLD2 | 1.37 | 6.00E−03 |
| FGL2 | 1.37 | 8.15E−03 |
| TOP1 | 1.38 | 2.85E−04 |
| SPRY1 | 1.38 | 2.01E−03 |
| TIAM1 | 1.38 | 2.12E−03 |
| SMYD5 | 1.38 | 2.55E−03 |
| EIF2B3 | 1.38 | 2.72E−03 |
| SLC25A22 | 1.38 | 2.87E−03 |
| CDK4 | 1.38 | 3.22E−03 |
| SFXN4 | 1.38 | 6.31E−03 |
| GOLIM4 | 1.39 | 1.55E−03 |
| PAM | 1.39 | 1.70E−03 |
| POLR1C | 1.39 | 2.32E−03 |
| PMM2 | 1.39 | 2.58E−03 |
| PKM | 1.39 | 4.38E−03 |
| SLC9A7 | 1.40 | 1.77E−03 |
| PTRH2 | 1.40 | 1.85E−03 |
| PPRC1 | 1.40 | 3.11E−03 |
| RPF2 | 1.40 | 3.40E−03 |
| CTLA4 | 1.40 | 9.55E−03 |
| LDHA | 1.41 | 2.41E−03 |
| P4HA2 | 1.41 | 2.80E−03 |
| C12orf45 | 1.41 | 3.11E−03 |
| PFAS | 1.41 | 4.69E−03 |
| STARD4 | 1.41 | 4.81E−03 |
| SLC25A32 | 1.42 | 1.85E−03 |
| GCNT1 | 1.42 | 2.62E−03 |
| C10orf2 | 1.42 | 3.22E−03 |
| PDSS1 | 1.42 | 4.40E−03 |
| HSF5 | 1.42 | 5.30E−03 |
| TESK1 | 1.43 | 3.54E−04 |
| PFKP | 1.43 | 1.28E−03 |
| UTP20 | 1.43 | 1.53E−03 |
| DCUN1D5 | 1.43 | 1.73E−03 |
| EXOSC4 | 1.43 | 2.57E−03 |
| WDR4 | 1.43 | 2.58E−03 |
| IRS1 | 1.43 | 3.24E−03 |
| PDCD2L | 1.43 | 3.60E−03 |
| CCDC71L | 1.43 | 3.72E−03 |
| CRISPLD1 | 1.43 | 4.18E−03 |
| SLC39A8 | 1.44 | 9.68E−04 |
| NOP56 | 1.44 | 2.41E−03 |
| FARSB | 1.44 | 3.15E−03 |
| LRP5 | 1.44 | 3.56E−03 |
| PPARGC1B | 1.44 | 5.30E−03 |
| CARD9 | 1.44 | 6.44E−03 |
| ATP1B3 | 1.45 | 7.78E−04 |
| CDK6 | 1.45 | 9.03E−04 |
| NOCT | 1.45 | 1.81E−03 |
| MBOAT7 | 1.45 | 1.88E−03 |
| PIGW | 1.45 | 3.60E−03 |
| FHL1 | 1.45 | 5.56E−03 |
| RMDN2 | 1.45 | 7.50E−03 |
| TIMM8A | 1.46 | 1.27E−03 |
| PLAGL2 | 1.46 | 1.64E−03 |
| NUP35 | 1.46 | 1.97E−03 |
| ITGA2 | 1.46 | 3.15E−03 |
| RRP15 | 1.46 | 3.22E−03 |
| CISD3 | 1.46 | 4.27E−03 |
| SCD | 1.46 | 4.33E−03 |
| HSPE1 | 1.46 | 5.23E−03 |
| MIR17HG | 1.46 | 6.06E−03 |
| FAM3C | 1.47 | 1.26E−03 |
| PINX1 | 1.47 | 1.34E−03 |
| CSRNP1 | 1.47 | 1.43E−03 |
| DUSP5 | 1.47 | 1.57E−03 |
| ZNHIT2 | 1.47 | 1.96E−03 |
| DDX10 | 1.47 | 2.12E−03 |
| WDR12 | 1.47 | 2.16E−03 |
| TIAM2 | 1.47 | 2.56E−03 |
| TXNDC5 | 1.47 | 2.96E−03 |
| DPH2 | 1.47 | 3.38E−03 |
| RGCC | 1.48 | 9.43E−04 |
| TSR1 | 1.48 | 2.00E−03 |
| TFB2M | 1.48 | 2.20E−03 |
| HSPD1 | 1.48 | 2.58E−03 |
| BCL2L1 | 1.48 | 3.07E−03 |
| SLC25A19 | 1.48 | 3.70E−03 |
| GAREML | 1.48 | 6.17E−03 |
| RGS16 | 1.48 | 6.65E−03 |
| CMTM4 | 1.49 | 1.40E−03 |
| RCL1 | 1.49 | 1.93E−03 |
| MAK16 | 1.49 | 2.19E−03 |
| CTU2 | 1.49 | 3.17E−03 |
| FAM129B | 1.49 | 3.22E−03 |
| EDARADD | 1.49 | 3.29E−03 |
| POLR3H | 1.49 | 3.40E−03 |
| FBXO30 | 1.50 | 3.07E−04 |
| EED | 1.50 | 1.10E−03 |
| CIRH1A | 1.50 | 1.28E−03 |
| SDF2L1 | 1.50 | 1.72E−03 |
| DKC1 | 1.50 | 2.19E−03 |
| GPATCH4 | 1.50 | 2.41E−03 |
| EBNA1BP2 | 1.50 | 2.65E−03 |
| PIM3 | 1.50 | 3.29E−03 |
| ATAD3B | 1.50 | 4.33E−03 |
| FUOM | 1.50 | 7.48E−03 |
| PGAM1 | 1.51 | 8.93E−04 |
| KBTBD8 | 1.51 | 1.20E−03 |
| PPAT | 1.51 | 1.25E−03 |
| MRPL12 | 1.51 | 3.07E−03 |
| SORD | 1.51 | 6.17E−03 |
| RNF19A | 1.52 | 1.67E−04 |
| SNX8 | 1.52 | 4.90E−04 |
| MICAL2 | 1.52 | 1.28E−03 |
| PGAM4 | 1.52 | 2.36E−03 |
| CYP27B1 | 1.52 | 5.56E−03 |
| GRPEL1 | 1.53 | 1.47E−03 |
| MYBBP1A | 1.53 | 2.49E−03 |
| FASN | 1.53 | 2.56E−03 |
| AIMP2 | 1.53 | 2.58E−03 |
| FKBP4 | 1.53 | 2.87E−03 |
| NFIL3 | 1.54 | 2.01E−03 |
| ENDOD1 | 1.54 | 2.36E−03 |
| MZB1 | 1.54 | 5.01E−03 |
| MIR155HG | 1.54 | 9.14E−03 |
| DHODH | 1.55 | 1.28E−03 |
| NOLC1 | 1.55 | 2.91E−03 |
| HOMER1 | 1.55 | 3.46E−03 |
| HYAL2 | 1.55 | 3.57E−03 |
| SNHG3 | 1.55 | 3.78E−03 |
| NOP16 | 1.55 | 3.79E−03 |
| SLFNL1-AS1 | 1.55 | 8.12E−03 |
| COA7 | 1.56 | 3.34E−03 |
| PRDX4 | 1.56 | 5.60E−03 |
| TMEM97 | 1.57 | 1.23E−03 |
| NPM3 | 1.57 | 3.29E−03 |
| ERICD | 1.57 | 6.75E−03 |
| FAM89A | 1.58 | 2.94E−03 |
| SLC19A2 | 1.59 | 3.50E−04 |
| HNRNPAB | 1.59 | 1.55E−03 |
| CLECL1 | 1.59 | 1.86E−03 |
| SEH1L | 1.59 | 1.90E−03 |
| TRMT1 | 1.59 | 2.36E−03 |
| EAF2 | 1.59 | 3.90E−03 |
| IFI30 | 1.59 | 4.69E−03 |
| RP4-671O14.6 | 1.59 | 4.71E−03 |
| GEM | 1.59 | 5.66E−03 |
| MTHFD2 | 1.59 | 6.45E−03 |
| PTCH1 | 1.60 | 1.78E−03 |
| PLXNA1 | 1.60 | 1.97E−03 |
| PNO1 | 1.60 | 2.03E−03 |
| HSP90AB1 | 1.60 | 2.78E−03 |
| CD80 | 1.60 | 3.09E−03 |
| SLC19A1 | 1.60 | 7.30E−03 |
| HSPH1 | 1.61 | 2.85E−04 |
| LRRC8B | 1.61 | 3.66E−04 |
| SNX9 | 1.61 | 2.19E−03 |
| RRP1 | 1.61 | 2.58E−03 |
| PDIA5 | 1.61 | 3.73E−03 |
| CTNNA1 | 1.61 | 4.41E−03 |
| ITPRIPL2 | 1.62 | 3.51E−04 |
| PER2 | 1.62 | 7.48E−04 |
| DCTPP1 | 1.62 | 2.49E−03 |
| RRS1 | 1.62 | 3.29E−03 |
| SCARF1 | 1.62 | 5.49E−03 |
| PDF | 1.62 | 6.73E−03 |
| C5orf30 | 1.63 | 7.24E−04 |
| MPP6 | 1.64 | 7.24E−04 |
| SLC27A2 | 1.64 | 1.20E−03 |
| HECTD2 | 1.64 | 1.26E−03 |
| AK4 | 1.64 | 4.45E−03 |
| KLHL31 | 1.64 | 7.67E−03 |
| APBB2 | 1.64 | 8.87E−03 |
| NIPA1 | 1.65 | 1.24E−04 |
| OSBPL3 | 1.65 | 1.20E−03 |
| CLUH | 1.65 | 2.79E−03 |
| TOMM40 | 1.65 | 3.08E−03 |
| IPO4 | 1.65 | 3.40E−03 |
| AFAP1L2 | 1.65 | 9.60E−03 |
| ARSB | 1.66 | 2.85E−04 |
| TMEM2 | 1.66 | 2.85E−04 |
| PUM3 | 1.66 | 2.28E−03 |
| EHD4 | 1.66 | 3.73E−03 |
| SRM | 1.66 | 4.16E−03 |
| PPAN | 1.67 | 1.55E−03 |
| TBX21 | 1.67 | 2.28E−03 |
| ANKRD13B | 1.67 | 5.59E−03 |
| CEP170B | 1.67 | 6.18E−03 |
| URB2 | 1.68 | 1.28E−03 |
| KCNK5 | 1.68 | 2.67E−03 |
| PHLDA1 | 1.68 | 2.93E−03 |
| TRPM2 | 1.68 | 3.42E−03 |
| LINC00152 | 1.69 | 1.24E−04 |
| TNF | 1.69 | 2.49E−03 |
| SLC7A1 | 1.69 | 4.67E−03 |
| KCTD15 | 1.69 | 9.42E−03 |
| UCK2 | 1.70 | 1.82E−03 |
| PROB1 | 1.70 | 1.97E−03 |
| DYRK3 | 1.71 | 1.81E−03 |
| CITED4 | 1.71 | 1.81E−03 |
| DDX21 | 1.71 | 2.96E−03 |
| CRTAM | 1.71 | 6.63E−03 |
| NLN | 1.71 | 7.04E−03 |
| MRC2 | 1.71 | 8.62E−03 |
| IFRD2 | 1.72 | 1.96E−03 |
| MRTO4 | 1.72 | 2.36E−03 |
| ATAD3A | 1.72 | 2.55E−03 |
| NEFH | 1.72 | 2.68E−03 |
| TRMT61A | 1.72 | 3.35E−03 |
| CELSR3 | 1.72 | 4.42E−03 |
| ANKRD37 | 1.72 | 5.86E−03 |
| LAG3 | 1.73 | 2.11E−03 |
| YRDC | 1.73 | 2.40E−03 |
| BOP1 | 1.73 | 2.41E−03 |
| ZNF593 | 1.73 | 2.99E−03 |
| RBKS | 1.73 | 3.29E−03 |
| SOCS3 | 1.73 | 4.68E−03 |
| KLHL35 | 1.73 | 8.06E−03 |
| C4orf32 | 1.74 | 1.69E−03 |
| SDC4 | 1.74 | 2.28E−03 |
| DUSP4 | 1.74 | 3.22E−03 |
| QPCT | 1.74 | 6.42E−03 |
| SERPINE2 | 1.74 | 8.28E−03 |
| MPP1 | 1.75 | 5.04E−04 |
| BYSL | 1.75 | 1.54E−03 |
| PAICS | 1.75 | 2.79E−03 |
| GADD45G | 1.75 | 3.90E−03 |
| SNHG4 | 1.75 | 3.90E−03 |
| LRIG1 | 1.76 | 1.24E−04 |
| COPRS | 1.76 | 1.21E−03 |
| IL2RA | 1.76 | 1.24E−03 |
| CTNS | 1.76 | 1.43E−03 |
| RILP | 1.76 | 4.87E−03 |
| TUBB6 | 1.76 | 7.53E−03 |
| VDR | 1.77 | 9.86E−04 |
| ARMC9 | 1.77 | 1.28E−03 |
| CCDC86 | 1.77 | 2.12E−03 |
| TBC1D4 | 1.78 | 1.43E−03 |
| PRNP | 1.78 | 1.54E−03 |
| MICALCL | 1.78 | 5.64E−03 |
| CAMKK1 | 1.78 | 7.06E−03 |
| BEND3 | 1.79 | 2.40E−03 |
| CSF1 | 1.79 | 3.73E−03 |
| PVR | 1.80 | 1.02E−03 |
| GNPDA1 | 1.80 | 1.40E−03 |
| CTPS1 | 1.80 | 1.61E−03 |
| RRP9 | 1.80 | 2.18E−03 |
| C17orf51 | 1.80 | 5.64E−03 |
| RAB20 | 1.80 | 7.84E−03 |
| NEURL1 | 1.80 | 8.86E−03 |
| NT5DC3 | 1.81 | 1.28E−03 |
| MYC | 1.81 | 2.71E−03 |
| ADGRA3 | 1.81 | 2.87E−03 |
| MIR222HG | 1.81 | 2.99E−03 |
| PLD6 | 1.81 | 3.68E−03 |
| SLC1A5 | 1.83 | 6.10E−03 |
| SLC3A2 | 1.84 | 4.25E−03 |
| RP11-295D4.1 | 1.84 | 6.70E−03 |
| HK2 | 1.85 | 1.34E−03 |
| DERL3 | 1.85 | 1.85E−03 |
| SLC35E4 | 1.86 | 7.24E−04 |
| TMEM201 | 1.86 | 1.73E−03 |
| METTL1 | 1.86 | 2.81E−03 |
| LRP8 | 1.87 | 1.70E−03 |
| PODXL2 | 1.87 | 1.80E−03 |
| ECE2 | 1.87 | 2.97E−03 |
| BATF | 1.87 | 4.81E−03 |
| BCL2A1 | 1.88 | 7.27E−04 |
| SLC38A5 | 1.88 | 2.00E−03 |
| SMKR1 | 1.88 | 2.59E−03 |
| AC002454.1 | 1.88 | 9.57E−03 |
| CHSY1 | 1.89 | 1.24E−04 |
| WARS | 1.89 | 4.14E−03 |
| RHPN2 | 1.89 | 4.99E−03 |
| PTGFRN | 1.89 | 5.30E−03 |
| LAPTM4B | 1.90 | 2.65E−03 |
| AFAP1 | 1.92 | 3.11E−03 |
| GFOD1 | 1.92 | 3.40E−03 |
| FAM184A | 1.92 | 5.78E−03 |
| ZDHHC9 | 1.93 | 2.12E−03 |
| SGK223 | 1.94 | 9.43E−04 |
| NTRK1 | 1.94 | 1.78E−03 |
| SEMA7A | 1.95 | 2.12E−04 |
| NETO2 | 1.95 | 1.85E−03 |
| PUS7 | 1.95 | 3.02E−03 |
| P3H3 | 1.95 | 6.06E−03 |
| SLC17A9 | 1.96 | 4.43E−03 |
| XCL1 | 1.97 | 3.97E−03 |
| UBXN10 | 1.99 | 1.81E−03 |
| RP11-19P22.8 | 1.99 | 6.71E−03 |
| OTUD1 | 2.00 | 1.67E−04 |
| RRP12 | 2.00 | 2.44E−03 |
| SPINT1 | 2.02 | 6.25E−03 |
| TSHZ3 | 2.04 | 9.11E−03 |
| B4GALT2 | 2.05 | 5.24E−03 |
| ODC1 | 2.06 | 1.88E−03 |
| SLC39A14 | 2.06 | 2.55E−03 |
| RPP25 | 2.07 | 1.31E−03 |
| OXCT2 | 2.07 | 5.16E−03 |
| CCR1 | 2.08 | 2.99E−03 |
| HBEGF | 2.08 | 3.51E−03 |
| LINC01160 | 2.08 | 4.46E−03 |
| EGFL6 | 2.08 | 8.15E−03 |
| MYO1E | 2.09 | 1.03E−03 |
| FASLG | 2.09 | 1.20E−03 |
| KIFC3 | 2.09 | 3.15E−03 |
| MARVELD3 | 2.10 | 1.52E−03 |
| NME1 | 2.10 | 2.87E−03 |
| RHOB | 2.10 | 3.88E−03 |
| CCL3L3 | 2.10 | 5.45E−03 |
| RP5-862P8.2 | 2.10 | 9.37E−03 |
| RP11-803D5.1 | 2.11 | 1.72E−03 |
| NR2F6 | 2.11 | 1.92E−03 |
| CD160 | 2.11 | 2.09E−03 |
| ZBTB32 | 2.11 | 5.53E−03 |
| PALD1 | 2.11 | 7.38E−03 |
| NDST1 | 2.12 | 8.78E−03 |
| SPAG1 | 2.13 | 4.10E−04 |
| CD109 | 2.13 | 1.09E−03 |
| NEK6 | 2.13 | 2.16E−03 |
| TNFRSF9 | 2.13 | 2.79E−03 |
| OAF | 2.13 | 7.10E−03 |
| KCNH4 | 2.13 | 7.59E−03 |
| ZEB2 | 2.14 | 1.24E−04 |
| HES6 | 2.14 | 4.63E−03 |
| ERRFI1 | 2.14 | 8.23E−03 |
| C10orf128 | 2.15 | 9.43E−04 |
| KCNQ5-IT1 | 2.15 | 7.75E−03 |
| IRF4 | 2.16 | 1.72E−03 |
| LTA | 2.17 | 1.02E−03 |
| GOLGA8N | 2.17 | 9.15E−03 |
| NDFIP2 | 2.19 | 1.94E−03 |
| LINC00484 | 2.19 | 2.30E−03 |
| HIVEP3 | 2.20 | 3.50E−04 |
| FOSL2 | 2.20 | 1.96E−03 |
| LRRC32 | 2.20 | 3.19E−03 |
| IL4I1 | 2.21 | 1.26E−03 |
| NR1D1 | 2.22 | 1.30E−03 |
| SLC7A5 | 2.23 | 2.85E−03 |
| ANO7 | 2.24 | 1.69E−03 |
| FABP5 | 2.24 | 1.78E−03 |
| HBQ1 | 2.24 | 2.94E−03 |
| CABP1 | 2.24 | 3.73E−03 |
| RNF152 | 2.24 | 7.90E−03 |
| PIP5K1B | 2.25 | 1.48E−04 |
| RDH10 | 2.25 | 8.72E−04 |
| NAMPT | 2.25 | 1.51E−03 |
| RP11-290L1.3 | 2.25 | 2.41E−03 |
| GOLGA8R | 2.25 | 5.79E−03 |
| NCR3LG1 | 2.25 | 6.89E−03 |
| IL21 | 2.25 | 9.34E−03 |
| SPHK1 | 2.26 | 2.80E−03 |
| MIR3945HG | 2.26 | 5.38E−03 |
| AC058791.1 | 2.26 | 8.85E−03 |
| CTD-2060L22.1 | 2.26 | 9.34E−03 |
| ACVR1B | 2.27 | 7.06E−03 |
| SMN1 | 2.28 | 1.26E−03 |
| RP11-680G24.6 | 2.28 | 4.71E−03 |
| LUCAT1 | 2.28 | 5.32E−03 |
| AC133644.2 | 2.29 | 7.48E−03 |
| IER3 | 2.31 | 1.66E−03 |
| PDGFA | 2.31 | 2.41E−03 |
| RP11-626G11.6 | 2.31 | 8.82E−03 |
| IL17D | 2.32 | 5.17E−03 |
| AC005540.3 | 2.32 | 7.06E−03 |
| SLC43A3 | 2.33 | 1.61E−03 |
| RP11-932O9.9 | 2.33 | 7.07E−03 |
| SLC35F3 | 2.33 | 8.13E−03 |
| IRF8 | 2.34 | 1.13E−03 |
| ACTN1 | 2.35 | 2.68E−03 |
| SPR | 2.35 | 3.40E−03 |
| MIR4435-2HG | 2.37 | 1.46E−04 |
| RP11-132A1.4 | 2.37 | 9.97E−03 |
| GRID2IP | 2.38 | 1.66E−03 |
| MFSD2A | 2.38 | 1.85E−03 |
| FGF2 | 2.38 | 3.22E−03 |
| TNIP3 | 2.38 | 4.35E−03 |
| PDXP | 2.38 | 5.78E−03 |
| SMOX | 2.42 | 7.27E−04 |
| RP11-783K16.5 | 2.44 | 9.07E−03 |
| HPDL | 2.46 | 2.44E−03 |
| MAF | 2.49 | 1.28E−03 |
| C17orf96 | 2.49 | 1.65E−03 |
| SLC29A1 | 2.49 | 2.41E−03 |
| AATK | 2.49 | 9.23E−03 |
| SOCS1 | 2.50 | 2.58E−03 |
| RP11-254F7.4 | 2.50 | 6.79E−03 |
| POU2AF1 | 2.51 | 1.57E−03 |
| XCL2 | 2.51 | 1.76E−03 |
| IL1RAP | 2.52 | 7.98E−04 |
| RP11-295P9.13 | 2.52 | 7.82E−03 |
| SSC4D | 2.53 | 3.71E−03 |
| SHB | 2.53 | 9.59E−03 |
| CD3EAP | 2.54 | 1.57E−03 |
| SH2D4A | 2.54 | 4.25E−03 |
| STAB1 | 2.54 | 8.18E−03 |
| SAMD4A | 2.57 | 9.79E−03 |
| CEACAM1 | 2.58 | 1.24E−03 |
| SIGLEC6 | 2.58 | 7.48E−03 |
| CRIM1 | 2.59 | 2.85E−04 |
| LINC00843 | 2.62 | 4.63E−03 |
| SLC4A4 | 2.63 | 1.28E−03 |
| PACSIN3 | 2.63 | 5.62E−03 |
| ARFGEF3 | 2.66 | 2.41E−03 |
| RP11-542M13.3 | 2.66 | 3.56E−03 |
| CCL4L2 | 2.68 | 1.85E−03 |
| SMAD1 | 2.70 | 9.03E−03 |
| SETBP1 | 2.71 | 2.28E−03 |
| KIR3DX1 | 2.71 | 2.41E−03 |
| RP11-863P13.4 | 2.71 | 2.49E−03 |
| BATF3 | 2.71 | 5.26E−03 |
| PDCD1 | 2.72 | 6.77E−04 |
| CTC-251I16.1 | 2.72 | 2.40E−03 |
| OSBPL6 | 2.72 | 9.92E−03 |
| HSD11B1 | 2.74 | 1.35E−03 |
| RP11-47122.2 | 2.74 | 6.96E−03 |
| BICC1 | 2.75 | 4.35E−03 |
| RP11-843B15.4 | 2.77 | 5.95E−03 |
| BANK1 | 2.77 | 8.35E−03 |
| LDHAL6B | 2.77 | 8.79E−03 |
| GZMB | 2.78 | 1.03E−03 |
| PTGIS | 2.78 | 6.57E−03 |
| RP11-172H24.4 | 2.78 | 9.11E−03 |
| C22orf42 | 2.79 | 1.33E−03 |
| AP001189.4 | 2.79 | 3.99E−03 |
| CD200 | 2.81 | 7.73E−04 |
| C10orf67 | 2.82 | 6.12E−03 |
| RP11-822E23.8 | 2.83 | 6.77E−03 |
| CSF2 | 2.84 | 1.28E−03 |
| DUSP6 | 2.84 | 1.76E−03 |
| RP13-512J5.1 | 2.87 | 7.09E−03 |
| CTTN | 2.94 | 7.05E−04 |
| RP11-1000B6.3 | 2.98 | 5.99E−03 |
| TNFRSF21 | 2.99 | 9.99E−04 |
| KIAA1217 | 2.99 | 1.82E−03 |
| FOSL1 | 3.00 | 5.91E−04 |
| PLAU | 3.02 | 1.81E−03 |
| ARHGAP42 | 3.02 | 2.73E−03 |
| IGHV1-3 | 3.03 | 4.14E−03 |
| RP11-77K12.1 | 3.03 | 8.98E−03 |
| PRKAG2-AS1 | 3.04 | 6.18E−03 |
| AP001046.5 | 3.06 | 3.19E−03 |
| SPRY4 | 3.07 | 6.03E−04 |
| GNG12 | 3.08 | 6.72E−03 |
| SHC4 | 3.09 | 3.26E−04 |
| MAFG-AS1 | 3.09 | 3.15E−03 |
| LINC00892 | 3.10 | 7.24E−04 |
| TNFSF11 | 3.11 | 2.14E−03 |
| CYSLTR2 | 3.13 | 1.08E−03 |
| RP11-70D24.2 | 3.13 | 6.44E−03 |
| KITLG | 3.13 | 7.06E−03 |
| CCL3 | 3.16 | 3.40E−04 |
| IL4 | 3.16 | 3.92E−03 |
| TNS3 | 3.18 | 6.03E−04 |
| FZD9 | 3.18 | 1.70E−03 |
| IL22 | 3.18 | 3.29E−03 |
| HRH2 | 3.19 | 9.43E−04 |
| PRDM13 | 3.22 | 3.04E−03 |
| CCL4 | 3.23 | 3.55E−04 |
| TEX19 | 3.24 | 5.71E−03 |
| IL1RL1 | 3.26 | 2.77E−03 |
| CD40LG | 3.29 | 3.51E−04 |
| PLPP2 | 3.29 | 1.28E−03 |
| FGF9 | 3.29 | 4.55E−03 |
| IL2 | 3.30 | 2.36E−03 |
| RP11-505K9.5 | 3.34 | 1.52E−03 |
| NCS1 | 3.34 | 3.20E−03 |
| XIRP1 | 3.36 | 4.07E−04 |
| CIART | 3.37 | 9.43E−04 |
| GLDC | 3.44 | 2.36E−03 |
| G0S2 | 3.50 | 4.19E−03 |
| ASCL2 | 3.54 | 6.37E−03 |
| IFNG | 3.56 | 4.38E−04 |
| CD276 | 3.61 | 5.05E−03 |
| FLT1 | 3.62 | 1.72E−03 |
| GNAI1 | 3.62 | 3.15E−03 |
| TRIM6 | 3.72 | 2.96E−03 |
| RP11-1079K10.4 | 3.72 | 3.13E−03 |
| AC017002.1 | 3.73 | 2.18E−03 |
| SIPA1L2 | 3.75 | 7.27E−04 |
| KCNJ11 | 3.84 | 5.04E−04 |
| RP11-456K23.1 | 3.87 | 7.27E−04 |
| KCNG1 | 3.88 | 6.31E−03 |
| INSM1 | 3.93 | 4.97E−03 |
| F5 | 3.94 | 9.16E−04 |
| TFEC | 3.95 | 7.21E−04 |
| GOLGA8O | 4.09 | 6.12E−03 |
| PRSS23 | 4.18 | 2.85E−04 |
| HRH1 | 4.36 | 3.14E−03 |
| GPR87 | 4.38 | 1.02E−03 |
| EMP1 | 4.53 | 2.85E−04 |
| ZBED2 | 4.68 | 8.52E−07 |
| IL3 | 4.87 | 8.66E−03 |
| IL31 | 5.14 | 3.34E−03 |
| IGF2 | 5.29 | 4.10E−04 |
| IL10 | 5.58 | 6.25E−03 |

1. A fusion protein comprising:
(a) an extracellular component comprising a binding domain that specifically binds a target antigen;
(b) an intracellular component comprising a modified functional CD28 costimulatory signaling domain, wherein the modified functional CD28 costimulatory signaling domain comprises at least one amino acid substitution; and
(c) a hydrophobic portion disposed between the extracellular component and intracellular component,
wherein the fusion protein has one or more functional activity that differs from a fusion protein comprising wildtype CD28 costimulatory signaling domain.

2. The fusion protein of claim 1, wherein at least one tyrosine residue in the CD28 costimulatory signaling domain is substituted with a different amino acid residue.

3. The fusion protein of claim 2, wherein the at least one tyrosine residue is selected from any one of positions 191, 206, 209 and 218.

4. The fusion protein of claim 2, wherein at least two tyrosine residues selected from any of positions 191, 206, 209, and 218 are each substituted with a different amino acid residue, wherein the at least two tyrosine substitutions are with the same or a different amino acid.

5. The fusion protein of claim 2, wherein at least three tyrosine residues selected from any of positions 191, 206, 209, and 218 are each substituted with a different amino acid residue, wherein the at least three tyrosine substitutions are with the same or a different amino acid.

6. The fusion protein of claim 2, wherein four tyrosine residues at positions 191, 206, 209, and 218 are substituted with a different amino acid residue, wherein the four tyrosine substitutions are with the same or different amino acid.

7. The fusion protein of claim 2, wherein each of the at least one tyrosine residue is independently substituted with a tryptophan residue or a phenylalanine residue.

8. The fusion protein of claim 2, wherein each of the at least one tyrosine residue is substituted with a phenylalanine residue.

9. The fusion protein of claim 2, wherein each of the at least one tyrosine residue is substituted with a tryptophan residue.

10. The fusion protein of claim 2, wherein the modified CD28 costimulatory signaling domain comprises a Y218F substitution.

11. The fusion protein of claim 2, wherein the modified CD28 costimulatory signaling domain comprises Y206F, Y209F, and Y218F substitutions.

12. The fusion protein of claim 2, wherein the modified CD28 costimulatory signaling domain comprises Y191F, Y206F, Y209F, and Y218F substitutions.

13. The fusion protein of claim 1, wherein at least one proline residue in the CD28 costimulatory signaling domain is substituted with a different amino acid residue.

14. The fusion protein of claim 13, wherein the at least one proline residue is selected from any one of positions 196, 199, 208, and 211.

15. The fusion protein of claim 13, wherein at least two proline residues selected from any of positions 196, 199, 208, and 211 are each substituted with a different amino acid residue, wherein the at least two proline substitutions are with the same or different amino acid.

16. The fusion protein of claim 13, wherein at least three proline residues selected from any of positions 196, 199, 208, and 211 are each substituted with a different amino acid residue, wherein the at least three proline substitutions are with the same or different amino acid.

17. The fusion protein of claim 13, wherein four proline residues at positions 196, 199, 208, and 211 are substituted with a different amino acid residue, wherein the four proline substitutions are with the same or different amino acid.

18. The fusion protein of claim 17, wherein each proline residue is substituted with an alanine residue.

19. The fusion protein of claim 1, wherein the modified CD28 costimulatory signaling domain further comprises L186 and L187 substitutions.

20. The fusion protein of claim 19, wherein the L186 substitution is L186G and the L187 substitution is L187G.

21. The fusion protein of claim 1, wherein the modified CD28 costimulatory signaling domain does not comprise a substitution at Y191, P208, P211, or any combination thereof.

22. The fusion protein of claim 1, wherein the binding domain is a scFv, scTCR, receptor ectodomain, or ligand.

23. The fusion protein of claim 1, wherein the binding domain does not comprise an extracellular binding domain or moiety of CD8 or any portion thereof that comprises a functional IgV-like domain.

24. The fusion protein of claim 23, wherein the binding domain does not comprise a binding domain from a CD8α chain, a binding domain from a CD8β chain, a binding domain from a CD8α homodimer, or a binding domain from a CD8αβ heterodimer.

25. The fusion protein of claim 1, wherein the binding domain is chimeric, human, or humanized.

26. The fusion protein of claim 1, wherein the extracellular component further comprises a spacer disposed between the binding domain and the hydrophobic portion.

27. The fusion protein of claim 26, wherein the spacer comprises an immunoglobulin hinge region, a CH2 domain, a CH3 domain, or any combination thereof.

28. The fusion protein of claim 27, wherein the immunoglobulin hinge region is an IgG4 hinge region.

29. The fusion protein of claim 1, wherein the extracellular component further comprises a tag disposed between the binding domain and the hydrophobic portion.

30. The fusion protein of claim 29, wherein the tag has the amino acid sequence of Trp-Ser-His-Pro-Gln-Phe-Glu-Lys (SEQ ID NO:40).

31. The fusion protein of claim 1, wherein the intracellular component further comprises an ITAM-containing T-cell activating domain.

32. The fusion protein of claim 31, wherein the ITAM-containing T-cell activating domain comprises an intracellular signaling domain of CD3γ, CD3δ, CD3ε, CD3ζ, gamma chain of FcεRI, or gamma chain of FcγRI.

33. The fusion protein of claim 32, wherein the intracellular component further comprises a CD3ζ intracellular signaling domain.

34. The fusion protein of claim 1, wherein the intracellular component further comprises at least one additional costimulatory signaling domain.

35. The fusion protein of claim 34, wherein the at least one additional costimulatory signaling domain is selected from CD27, CD40L, GITR, NKG2C, CARD1, CD2, CD7, CD27, CD30, CD40, CD54 (ICAM), CD83, CD134 (OX-40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP 10, LAT, NKD2C SLP76, TRIM, ZAP70, CD5, BAFF-R, SLAMF7, NKp80, CD160, B7-H3, a ligand that specifically binds with CD83, or a combination thereof

36. The fusion protein of claim 1, wherein the hydrophobic portion is a transmembrane domain.

37. The fusion protein of claim 36, wherein the transmembrane domain comprises a transmembrane domain of CD28, CD2, CD3ε, CD3δ, CD3ζ, CD25, CD27, CD40, CD79A, CD79B, CD80, CD86, CD95 (Fas), CD134 (OX40), CD137 (4-1BB), CD150 (SLAMF1), CD152 (CTLA4), CD200R, CD223 (LAG3), CD270 (HVEM), CD272 (BTLA), CD273 (PD-L2), CD274 (PD-L1), CD278 (ICOS), CD279 (PD-1), CD300, CD357 (GITR), A2aR, DAP10, FcRα, FcRβ, FcRγ, Fyn, GAL9, KIR, Lck, LAT, LRP, NKG2D, NOTCH1, NOTCH2, NOTCH3, NOTCH4, PTCH2, ROR2, Ryk, Slp76, SIRPα, pTα, TCRα, TCRβ, TIM3, TRIM, LPA5, or Zap70.

38. The fusion protein of claim 1, wherein the target antigen is a cancer antigen, a viral antigen, a bacterial antigen, or a self-antigen.

39. The fusion protein of claim 36, wherein the target antigen is a cancer antigen selected from BCMA, CD3, CEACAM6, c-Met, EGFR, EGFRvIII, ErbB2, ErbB3, ErbB4, EphA2, IGFIR, GD2, O-acetyl GD2, O-acetyl GD3, GHRHR, GHR, FLT1, KDR, FLT4, CD44v6, CD151, CA125, CEA, CTLA-4, GITR, BTLA, TGFBR2, TGFBR1, IL6R, gp130, Lewis A, Lewis Y, TNFR1, TNFR2, PD1, PD-L1, PD-L2, HVEM, MAGE-A, mesothelin, NY-ESO-1, PSMA, RANK, ROR1, TNFRSF4, CD40, CD137, TWEAK-R, HLA, tumor or pathogen associated peptide bound to HLA, hTERT peptide bound to HLA, tyrosinase peptide bound to HLA, WT-1 peptide bound to HLA, LTβR, LIFRβ, LRP5, MUC1, OSMRβ, TCRα TCRβ, CD19, CD20, CD22, CD25, CD28, CD30, CD33, CD52, CD56, CD79a, CD79b, CD80, CD81, CD86, CD123, CD171, CD276, B7H4, TLR7, TLR9, PTCH1, WT-1, HA¹-H, Robol, α-fetoprotein (AFP), Frizzled, OX40, PRAME, and SSX-2.

40. A polynucleotide encoding a fusion protein claim 1.

41. The polynucleotide of claim 40, wherein the nucleic acid molecule is codon optimized.

42. The polynucleotide of claim 40, comprising a polynucleotide having at least about 75% identity to the fusion protein-encoding nucleotide sequence set forth in any one of SEQ ID NOs:18, 20, 22, 24, 26, 28, 30, 32, 34, 36, 48, 50, 52, 54, 56, or 58.

43. The polynucleotide of claim 40, further comprising a polynucleotide encoding a transduction marker, a suicide gene, or both.

44. The polynucleotide of claim 43, wherein the transduction marker is a truncated EGFR molecule.

45. The polynucleotide of claim 40, wherein the polynucleotide comprises or consists of a polynucleotide having at least about 75% identity to any one of SEQ ID NOs:18, 20, 22, 24, 26, 28, 30, 32, 34, 36, 48, 50, 52, 54, 56, or 58.

46. A vector, comprising the polynucleotide of claim 40.

47. The vector of claim 46, wherein the vector is a viral vector.

48. The vector of claim 47, wherein the viral vector is a lentiviral or retroviral vector.

49. A host cell, comprising the the polynucleotide of claim 40.

50. The host cell of claim 49, wherein the host cell is an immune system cell.

51. The host cell of claim 50, wherein the immune system cell is a T cell.

52. The host cell of claim 51, wherein the T cell is a CD4⁺ T cell or CD8⁺ T cell.

53. The host cell of claim 51, wherein the T cell is a memory T cell.

54. The host cell of claim 49, wherein the host cell is human cell.

55. The host cell of claim 49, wherein expression of an endogenous gene is inhibited in the host cell, wherein the inhibited endogenous gene is selected from a TCR gene, an HLA gene, an immunosuppression component gene, or any combination thereof.

56. The host cell of claim 55, wherein the TCR gene is a T cell receptor α constant (TRAC) gene, a T cell receptor β constant (TRBC) gene, or both.

57. The host cell of claim 55, wherein the polynucleotide is targeted to the TCR gene or HLA gene locus via an endonuclease system.

58. The host cell of claim 57, wherein the endonuclease system is CRISPR/Cas nuclease system, a zinc finger nuclease (ZFN) system, or a Transcription Activator Like Effector nuclease (TALEN) system.

59. The host cell of claim 49, wherein the host cell exhibits decreased tonic phosphorylation of the fusion protein, decreased signaling of the fusion protein, decreased cytokine expression, enhanced persistence, enhanced antigen-specific cytolytic activity, or any combination thereof.

60. The host cell of claim 49, wherein the host cell expresses a decreased level of target antigen-induced IL-2, TNF-α, or both, as compared to a host cell comprising a fusion protein comprising a wildtype CD28 costimulatory signaling domain.

61. A pharmaceutical composition, comprising the host cell of claim 49, and a pharmaceutically acceptable carrier, diluent, or excipient.

62. A method of treating a disease in a subject, comprising administering to the subject a host cell of claim 49, wherein the disease is associated with the presence of the antigen bound by the fusion protein.

63. The method of claim 62, wherein the disease is a viral infection, bacterial infection, cancer, inflammatory disease, or autoimmune disease.

64. The method of claim 62, wherein the subject is human.

65. The method of claim 62, wherein the host cell is allogeneic or autologous to the subject.

66. The method of claim 62, wherein the subject has a low level or mild cytokine release syndrome, a low level or mild cell-related encephalopathy syndrome, or both, following the treatment, and/or has a reduced cytokine release syndrome, a reduced cell-related encephalopathy syndrome, or both, as compared to a reference subject that has been administered a reference host cell or composition wherein the fusion protein comprises a wild-type CD28 costimulatory signaling domain.

67. The method of claim 62, wherein the disease is a cancer.

68. The method of claim 67, wherein the cancer is solid tumor, melanoma, non-small cell lung cancer, renal cell carcinoma, renal cancer, a hematological cancer, prostate cancer, castration-resistant prostate cancer, colon cancer, rectal cancer, gastric cancer, esophageal cancer, bladder cancer, head and neck cancer, thyroid cancer, breast cancer, triple-negative breast cancer, ovarian cancer, cervical cancer, lung cancer, urothelial cancer, pancreatic cancer, glioblastoma, hepatocellular cancer, myeloma, multiple myeloma, leukemia, Hodgkin's lymphoma, non-Hodgkin's lymphoma, myelodysplastic syndrome, brain cancer, CNS cancer, or malignant glioma.

69. The method of claim 62, further comprising administering a chemotherapeutic or an inhibitor of an immune suppression component.

70. A kit, comprising:
the isolated polynucleotide of claim 40, and reagents for transducing the polynucleotide into a host cell.
